(19) United States
(12) Patent Application Publication
(10) Pub. No.: US 2004/0142958 A1
(43) Pub. Date: Jul. 22, 2004

(54) Combination therapy for the treatment of pain

(75) Inventors: Uri Herzberg, Bridgewater, NJ (US); Daniel N. Cortright, Orange, CT (US); Mark M. Hurtt, Wallingford, CT (US); James E. Krause, Madison, CT (US)

Correspondence Address: EDWARDS & ANGELL, LLP, P.O. BOX 55874, BOSTON, MA, 02205, US

(73) Assignee: Neurogen Corporation

(21) Appl. No.: 10/718,034

(22) Filed: Nov. 19, 2003

Related U.S. Application Data
(60) Provisional application No. 60/433,363, filed on Dec. 13, 2002.

(51) Int. Cl. A61K 31/485

(52) U.S. Cl. 514/282; 424/10.2

(57) ABSTRACT

Compositions and methods are provided for the treatment of pain. Compositions and methods are further provided for inhibiting the development of tolerance to addictive therapeutic agents (especially narcotic analgesics) in patients treated with such agents; for minimizing adverse effects (e.g., dependence) resulting from treatment with such addictive agents; and for enhancing pain relief resulting from narcotic analgesic administration. The compositions generally comprise a nontoxic VR1 antagonist, optionally in combination with an addictive therapeutic agent. Patients may be treated with a VR1 antagonist before, during or after administration of the addictive therapeutic agent to prevent, decrease the severity of, delay or treat tolerance and/or other adverse effects of the addictive agent in the patient.

This application claims the benefit of U.S. Provisional Patent Application 60/433,363, filed Dec. 13, 2002, which is incorporated herein by reference in its entirety.

### FIELD OF THE INVENTION

This invention relates generally to compositions and methods for the treatment of pain. More specifically, the invention relates to compositions and methods for reducing and preventing the development of acquired drug tolerance and adverse effects such as dependence in patients treated with addictive therapeutic agents, such as narcotic analgesics or other neuroactive drugs. In addition, the invention provides compositions and methods for improving the efficacy of narcotic analgesic therapy.

### BACKGROUND OF THE INVENTION

Narcotic analgesic agents, such as morphine, are often the most effective drugs for the treatment of severe pain. Their usefulness is limited, however, by tolerance (the progressive loss in analgesic effectiveness) and physical dependence (behavioral and/or physical symptoms resulting from sudden withdrawal of the drug). Both tolerance and physical dependence have been found to develop rapidly (e.g., within two or three days) upon repeated or continuous administration. To avoid withdrawal symptoms, some patients suffering from severe pain are not treated with narcotic analgesics, or are treated with doses that are too low to provide optimal pain relief.

Therapies that permit extended use of these potent analgesics, while minimizing tolerance and dependence, are needed in order to improve the treatment of severe pain. The present invention fulfills this need, and provides further related advantages.

### SUMMARY OF THE INVENTION

The present invention provides compositions and methods useful in the treatment and management of pain, as well as for inhibiting tolerance to addictive agents and minimizing adverse effects (e.g., dependence) resulting from administration of such agents. Within certain aspects, compositions provided herein comprise an addictive therapeutic substance (preferably a narcotic analgesic) and at least one nontoxic type I vanilloid receptor (VR1) antagonist.

Packaged pharmaceutical compositions are also provided. Certain such packaged compositions comprise (i) a container holding a composition comprising a nontoxic VR1 antagonist; and (ii) instructions indicating that the VR1 antagonist is to be administered to a patient contemporaneously with administration of an addictive substance. In certain embodiments, the addictive substance is a narcotic analgesic. Within various embodiments, the instructions indicate that the VR1 antagonist is to be used for: (a) inhibiting the development of tolerance to an addictive substance in a patient; (b) inhibiting the development of dependence on an addictive substance in a patient; and/or (c) enhancing pain relief resulting from administration of an addictive substance to a patient. The VR1 antagonist may be present, for example, in a tolerance-reducing amount, a dependence-reducing amount and/or a pain relief-enhancing amount.

Further packaged pharmaceutical compositions comprise: (i) a nontoxic VR1 antagonist; (ii) a narcotic analgesic and (iii) instructions indicating that the VR1 antagonist and narcotic analgesic are to be administered to a patient for the treatment of pain. The VR1 antagonist may be present, for example, in a tolerance-reducing amount, a dependence-reducing amount and/or a pain relief-enhancing amount.

Within further aspects, methods are provided for treating pain in a patient, comprising administering to a patient, simultaneously or sequentially in either order, (i) a narcotic analgesic and (ii) a nontoxic VR1 antagonist.

Methods are further provided, within other aspects, for inhibiting the development of tolerance to an addictive substance, such as a narcotic analgesic, in a patient, comprising administering to a patient, simultaneously or sequentially in either order, (i) a narcotic analgesic and (ii) a tolerance-reducing amount of a nontoxic VR1 antagonist.

Within other aspects, methods are provided for inhibiting the development of dependence on an addictive substance, such as a narcotic analgesic, in a patient, comprising administering to a patient, simultaneously or sequentially in either order, (i) a narcotic analgesic and (ii) a dependence-reducing amount of a nontoxic VR1 antagonist.

Within still further aspects, methods are provided for enhancing narcotic analgesic-induced pain relief in a patient, comprising administering to a patient, simultaneously or sequentially in either order, (i) a narcotic analgesic and (ii) a pain relief-enhancing amount of a nontoxic VR1 antagonist.

Within further aspects, methods are provided for treating withdrawal symptoms resulting from prior administration of an addictive substance (preferably a narcotic analgesic) in a patient, comprising administering at least one nontoxic VR1 antagonist to a patient experiencing or susceptible to such withdrawal symptoms.

These and other aspects of the present invention will become apparent upon reference to the following detailed description.

### BRIEF DESCRIPTION OF THE DRAWINGS

FIG. 1 is a graph illustrating the effect of a representative VR1 antagonist on morphine-induced tolerance. The results are presented as percent of maximum potential efficacy (% MPE) in a von Frey fiber test as a function of days following treatment initiation, and are presented for vehicle alone (dark line with diamonds), VR1 antagonist (10 mg/kg body weight; light line with squares), morphine (3 mg/kg body weight; light line with circles) and VR1 antagonist in combination with morphine (dark line with triangles).

FIG. 2 is a graph illustrating the analgesic effect of a representative VR1 antagonist in combination with morphine (dotted line) as compared to morphine alone (3 mg/kg body weight; light line with squares) or VR1 antagonist alone (0.5 mg/kg body weight; dark line with diamonds). The results are presented as percent of maximum potential efficacy (% MPE) in a von Frey fiber test as a function of days following treatment initiation, and are normalized to treatment with vehicle alone.

FIG. 3 is a graph illustrating the effect of a representative VR1 antagonist on morphine-induced tolerance in rats. The results are presented as withdrawal threshold from a von Frey filament (in gram force) as a function of treatment over a 5 day period. Post CFA BL is the von Frey filament threshold 7 days after injection of CFA in the left ankle. Drugs were then administered, and results are shown for days 1-4 following treatment, for vehicle alone (squares), VR1 antagonist (0.3 mg/kg body weight; triangles), morphine (3 mg/kg body weight; circles) and VR1 antagonist in combination with morphine (X's).

FIG. 4 is a graph illustrating the analgesic effect of a representative VR1 antagonist in combination with morphine as compared to morphine alone, VR1 antagonist alone or vehicle, as indicated. The results are presented as the decrease in thermal paw withdrawal latency in seconds, as compared to the latency observed prior to treatment.

### DETAILED DESCRIPTION OF THE INVENTION

As noted above, the present invention provides combination therapy for the treatment of pain. In certain aspects, the present invention provides compositions and methods for inhibiting the development of tolerance to addictive substances, such as therapeutic agents, as well as for minimizing adverse effects (e.g., dependence) resulting from administration of such agents. In other words, the compositions and methods provided herein may be used to prevent, delay, decrease the magnitude of or treat tolerance and/or adverse effects such as dependence in a patient treated with an addictive substance. In other aspects, compositions and methods provided herein are used to enhance the efficacy of a narcotic analgesic (i.e., to improve the level of pain relief achieved by a specified amount of narcotic analgesic). Compositions provided herein generally comprise a nontoxic VR1 antagonist and (optionally) an addictive substance, in combination with a physiologically acceptable carrier or excipient. Methods provided herein generally involve the administration of a VR1 antagonist and an addictive substance to a patient, where the VR1 antagonist is administered before, during and/or after administration of the addictive substance.

### VR1 Antagonists

As used herein, a VR1 antagonist is any compound that detectably inhibits vanilloid ligand binding to VR1 and/or VR1-mediated signal transduction resulting from binding of a vanilloid ligand agonist (e.g., capsaicin or a capsaicin analogue such as olvanil or resiniferatoxin) to VR1. In general, a VR1 antagonist inhibits VR1 activation with an IC₅₀ value of less than 1 micromolar, preferably less than 100 nanomolar, and more preferably less than 10 nanomolar within the assay provided in Example 7. Preferably, a VR1 antagonist displays no detectable agonist activity within an assay as described in Example 7, herein. Within certain embodiments, a VR1 antagonist is multi-aryl (i.e., has a plurality of unfused and/or fused aryl groups), is non-peptide and is amino acid free. Prodrugs of VR1 antagonists may also be used within the compositions and methods provided herein.

VR1 antagonists include both capsaicin analogues, such as capsazepine and lodo-RTX, and compounds that are not vanilloid compounds. Preferably, a VR1 antagonist is not a vanilloid compound. A “vanilloid compound” is capsaicin or any capsaicin analogue or other compound that comprises a phenyl ring with two oxygen atoms bound to adjacent ring carbons (one of which oxygen atoms is located para to a point of attachment of the phenyl ring to another substituent), and that binds to VR1 with a Kᵢ value (determined as described herein) that is no greater than 10 mM.

Certain preferred VR1 antagonists for use as described herein are compounds that satisfy one or more formulas provided below, or are a pharmaceutically acceptable salt of such a compound. A pharmaceutically acceptable salt is an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)ₙ—COOH where n is 0-4, and the like. Similarly, pharmaceutically acceptable cations include, but are not limited to sodium, potassium, calcium, aluminum, lithium and ammonium. Those of ordinary skill in the art will recognize further pharmaceutically acceptable salts for the compounds provided herein, including those listed by Remington's Pharmaceutical Sciences, 17th ed., Mack Publishing Company, Easton, Pa., p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred.

A “prodrug” is a compound that may not fully satisfy the structural requirements of the formulas provided herein, but is modified in vivo, following administration to a patient, to produce a compound of one or more such formulas. For example, a prodrug may be an acylated derivative of a compound as provided herein. Prodrugs include compounds wherein hydroxy, amine or sulfhydryl groups are bonded to any group that, when administered to a mammalian subject, cleaves to form a free hydroxyl, amino, or sulfhydryl group, respectively. Examples of prodrugs include, but, are not limited to, acetate, formate and benzoate derivatives of alcohol and amine functional groups within the compounds provided herein. Prodrugs of the compounds provided herein may be prepared by modifying functional groups present in the compounds in such a way that the modifications are cleaved to the parent compounds. Prodrugs of the compounds specifically recited herein may be used in the compositions and methods described herein.

Certain VR1 antagonists satisfy the formula:

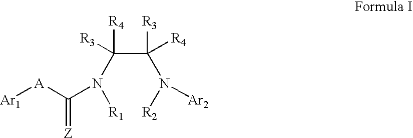

or a pharmaceutically acceptable salt thereof. Within Formula I, the variables are generally as described in PCT International Application Publication Number WO 02/08221, which published on Jan. 31, 2002. In general, A is chosen from O, S, NR_{A}, CR_{B}R_{B}′, NR_{A}CR_{B}R_{B}′, CR_{B}R_{B}′NR_{A}, —CR_{A}═CR_{B}—, and C₃H₄; where R_{A}, R_{B}, and R_{B}′ are independently selected at each occurrence from hydrogen or alkyl. Z is oxygen or sulfur. R₁ and R₂ independently represent hydrogen or lower alkyl; or R₁ and R₂ are taken together to form a 5 to 8 membered nitrogen containing ring of the formula:

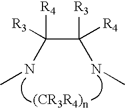

wherein n is 1, 2, or 3; and wherein R₃ and R4 are independently selected at each occurrence from hydrogen; halogen; hydroxy; amino; cyano; nitro; —COOH; —CHO, optionally substituted alkyl; optionally substituted alkenyl; optionally substituted alkynyl; optionally substituted alkoxy; optionally substituted mono or dialkylamino; optionally substituted alkylthio; optionally substituted alkyl ketone; optionally substituted alkylester; optionally substituted alkylsulfinyl; optionally substituted alkylsulfonyl; optionally substituted mono- or di-alkylcarboxamide; optionally substituted —S(O)ₙNHalkyl; optionally substituted —S(O)ₙN(alkyl)(alkyl); optionally substituted —NHC(═O)alkyl; optionally substituted —NC(═O)(alkyl)(alkyl); optionally substituted —NHS(O)ₙalkyl; optionally substituted —NS(O)ₙ(alkyl)(alkyl); optionally substituted saturated or partially unsaturated heterocycloalkyl of from 5 to 8 atoms, which saturated or partially unsaturated heterocycloalkyl contains 1, 2, or 3 heteroatoms selected from N, O, and S; optionally substituted aryl having from 1 to 3 rings; or optionally substituted heteroaryl, said heteroaryl having from 1 to 3 rings, 5 to 8 ring members in each ring and, in at least one of said rings, from 1 to about 3 heteroatoms per ring selected from the group consisting of N, O, and S; or any two R₃ and R₄ not attached to the same carbon may be joined to form an optionally substituted aryl ring; a saturated or partially unsaturated carbocyclic ring of from 5 to 8 members, which carbocyclic ring is optionally substituted; or a saturated, partially unsaturated, or aromatic heterocyclic ring of from 5 to 8 members, which heterocyclic ring is optionally substituted and contains 1, 2, or 3 heteroatoms selected from N, O, and S.

Ar₁ and Ar₂ of Formula I are the same or different and independently represent optionally substituted cycloalkyl; an optionally substituted heterocycloalkyl ring of from 5 to 8 atoms, which heterocycloalkyl ring contains 1, 2, or 3 heteroatoms selected from N, O, and S; optionally substituted aryl having from 1 to 3 rings; or optionally substituted heteroaryl, said heteroaryl having from 1 to 3 rings, 5 to 8 ring members in each ring and, in at least one of said rings, from 1 to about 3 heteroatoms per ring selected from the group consisting of N, O, and S.

Within specific embodiments, R₁ and R₂ of Formula I are joined to form a 5- to 7-membered heterocycloalkyl ring (e.g., R₁ and R₂ may be joined to form a piperazine ring). This 5- to 7-membered heterocycloalkyl ring is preferably unsubstituted or substituted at one or two positions with a C₁₋₆ alkyl group, such as methyl or ethyl. The variable “Z” is preferably oxygen and the variable “A” is generally NH, CH═CH or CH₂NH. Ar₁ and Ar₂ are preferably optionally substituted phenyl or optionally substituted pyridyl; optionally substituted 2-pyridyl is preferred for Ar₂ Substituents that may occur on Ar₁ and Ar₂ include, but are not limited to, butyl, isopropyl, trifluoromethyl, nitro, methyl, and halogen. Substitution at the 4 position of Ar₁ (when Ar₁ is phenyl or pyridyl) and substitution at the 3 position of Ar₂ (when Ar₂ is phenyl or pyridyl) are described in specific embodiments of the invention.

Other VR1 antagonists include substituted quinazolin-4-ylamine analogues. Certain such analogues are characterized by Formula II:

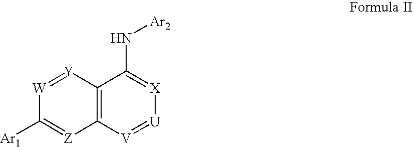

or a pharmaceutically acceptable salt thereof. Within Formula II, the variables are generally as described in PCT International Application Publication Number WO 03/062209, which published on Jul. 31, 2003.

In Formula II, V and X are each independently N or CR₁, with the proviso that at least one of V and X is N; U is N or CR₂, with the proviso that if V and X are N, then U is CR₂; and W, Y and Z are each independently N or CR₁.

R₁ of Formula II is independently selected at each occurrence from hydrogen, halogen, hydroxy, cyano, amino, C₁-C₈alkyl, haloC₁-C₈alkyl, C₁-C₈alkoxy, haloC₁-C₈alkoxy and mono- and di-(C₁-C₈alkyl)amino. Within certain embodiments, each R₁ is independently hydrogen, C₁-C₄alkyl or haloC₁-C₄alkyl; in other embodiments, each R₁ is H.

R₂ of Formula II is: (i) hydrogen, halogen, cyano or —COOH; (ii) C₂-C₈alkoxycarbonyl, C₁-C₈alkanoyl, C₂-C₈alkanone, C₁-C₈alkanoyloxy, C₁-C₈carbonate or C₁-C₈carbamate, each of which is unsubstituted or substituted with from 1 to 9 substituents independently selected from R_{b} or R_{d}; or (iii) a group of the formula —R_{c}-M-A-R_{y}, wherein: R_{c} is C₀-C₃alkyl; M is a bond, N(R_{z}), O, S, SO₂, —C(═O)ₚN(R_{z}), N(R_{z})C(═O)ₚ, SO₂N(R_{z}), or N(R_{z})SO₂, wherein p is 0 or 1; A is a bond or C₁-C₈alkyl optionally substituted with from 1 to 3 substituents independently chosen from R_{b} or R_{d}; and R_{y} and Rz are independently (a) hydrogen, C₁-C₈alkyl, C₂-C₈alkanone, C₂-C₈alkyl ether, C₂-C₈alkenyl, a 4- to 10-membered carbocycle or heterocycle, or (b) joined to R_{c} to form a 4- to 10-membered carbocycle or heterocycle, wherein each R_{y} and R_{z} is independently unsubstituted or substituted with from 1 to 9 substituents independently selected from R_{b} or R_{d}; or R_{y} and R_{z} are joined to form a 4- to 10-membered heterocycle that is unsubstituted or substituted with from 1 to 9 substituents independently selected from R_{b} or R_{d}. R_{b} is independently chosen at each occurrence from hydroxy, halogen, amino, aminocarbonyl, amido, cyano, nitro, oxo, C₁-C₈alkyl, C₁-C₈alkoxy, C₁-C₈alkylthio, C₁-C₈alkyl ether, hydroxyC₁-C₈alkyl, haloC₁-C₈alkyl, phenyl, phenyl(C₁-C₈alkyl), mono-and di-(C₁-C₆alkyl)amino, (SO₂)C₁-C₈alkyl, 5- to 7-membered heterocycle and (5- to 7-membered heterocycle)(C₁-C₈alkyl). R_{d} is independently selected at each occurrence from hydroxy, halogen, amino, aminocarbonyl, amido, cyano, nitro, C₁-C₈alkyl, C₁-C₈alkylthio, hydroxyC₁-C₈alkyl, haloC₁-C₈alkyl, phenyl, phenyl(C₁-C₈alkyl), mono-and di-(C₁-C₆alkyl)amino, (SO₂)C₁-C₈alkyl, 5- to 7-membered heterocycle and (5- to 7-membered heterocycle)(C₁-C₈alkyl).

Within certain compounds of Formula II, U is CR₂, and R₂ is: (i) hydrogen or halogen; or (ii) C₁-C₆alkyl, —(CH₂)ₙNH₂, —(CH₂)ₙNH(C₁-C₈alkyl), —(CH₂)ₙN(C₁-C₈alkyl)₂, —(CH₂)ₙ(5- to 8-membered heterocycloalkyl), or —(CH₂)ₙOH, each of which is unsubstituted or substituted with from 1 to 4 substituents independently chosen from halogen, cyano, hydroxy, amino, mono- and di-(C₁-C₆alkyl)amino, C₁-C₆alkyl, and haloC₁-C₆alkyl.

Ar₁ and Ar₂ are independently selected from 5- to 10-membered aromatic carbocycles and heterocycles, each of which is unsubstituted or substituted with from 1 to 3 substituents independently selected from groups of the formula LRₐ. L is independently selected at each occurrence from a bond, —O—, —C(═O)—, —OC(═O)—, —C(═O)O—, —O—C(═O)O—, —S(O)ₘ, —NRₓ—, —C(═O)NHRₓ—, —NHRₓC(═O)—, —NRₓS(O)ₘ—, —S(O)ₘNRₓ— and —N[S(O)ₘRₓ]S(O)ₘ—; wherein m is independently selected at each occurrence from 0, 1 and 2; and Rₓ is independently selected at each occurrence from hydrogen and C₁-C₈alkyl. Rₐ is independently selected at each occurrence from: (i) hydrogen, halogen, cyano and nitro; and (ii) C₁-C₈alkyl, C₂-C₈alkenyl, C₂-C₈alkynyl, C₂-C₈alkyl ether, 3- to 10-membered heterocycles, mono- and di-(C₁-C₈alkyl)amino and (3- to 10-membered heterocycle)C₁-C₆alkyl, each of which is optionally substituted with from 1 to 9 substituents independently selected from R_{b}. Within certain compounds of Formula I, Ar₂ is a 5- to 7-membered aromatic heterocycle, optionally substituted as described above.

Further VR1 antagonists that are substituted quinazolin-4-ylamine analogues are characterized by Formula III:

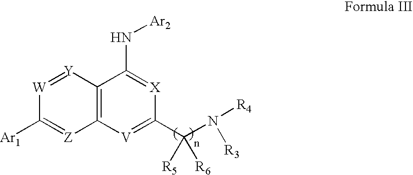

or a pharmaceutically acceptable salt thereof. Within Formula III, the variables are generally as described in PCT International Application Publication Number WO 03/062209, which published on Jul. 31, 2003. In general, V, X, W Y and Z are each independently N or CR₁, as described above.

Ar₁ and Ar₂ of Formula III are independently selected from phenyl and 5- to 7-membered aromatic heterocycles, each of which is unsubstituted or substituted with from 1 to 3 substituents independently selected from groups of the formula LRₐ, as described above.

R₃ and R₄ of Formula III are: (i) each independently selected from: (a) hydrogen; (b) C₁-C₈alkyl, C₂-C₈alkenyl, C₂-C₈alkynyl, C₁-C₈alkoxy, C₃-C₈alkanone, C₂-C₈alkanoyl, C₂-C₈alkyl ether, C₆-C₁₀arylC₀-C₈alkyl, 5- to 10-membered heterocycleC₀-C₈alkyl and —(SO₂)C₁-C₈alkyl, each of which is optionally substituted with from 1 to 9 substituents independently selected from R_{b}; and (c) groups that are joined to an R₅ or R₆ to form a 4- to 10-membered heterocyclic group that is unsubstituted or substituted with from 1 to 6 substituents independently selected from R_{b}; or (ii) joined to form, with the N to which they are bound, a 4- to 10-membered heterocyclic group that is unsubstituted or substituted with from 1 to 6 substituents independently selected from R_{b}, C₁-C₈alkanoyl, C₂-C₈alkanoyloxy, C₂-C₈alkoxycarbonyl, 4- to 7-membered heterocycloalkylC₀-C₄alkyl, and mono- and di-C₁-C₆alkylaminoC₁-C₆alkyl.

In certain compounds of Formula III, R₃ and R₄ are each independently: (i) hydrogen; or (ii) C₁-C₈alkyl, C₂-C₈alkenyl, phenylC₀-C₄alkyl, indanylC₀-C₄alkyl, 5- to 6-membered heteroarylC₀-C₄alkyl, or 4- to 7-membered heterocycloalkylC₀-C₄alkyl, each of which is unsubstituted or substituted with from 1 to 4 substituents independently selected from hydroxy, halogen, amino, C₁-C₆alkyl, haloC₁-C₆alkyl, C₁-C₆alkoxy and haloC₁-C₆alkoxy. In certain embodiments, R₃ and R₄ are each independently: (i) hydrogen; or (ii) C₁-C₆alkyl, C₂-C₆alkenyl, 5- to 7-membered heterocycloC₀-C₄alkyl, C₂-C₆alkyl ether, indanyl, benzyl, 1-phenyl-ethyl, 1-phenyl-propyl and 2-phenyl-ethyl, each of which is unsubstituted or substituted with from 1 to 3 substituents independently selected from hydroxy, halogen and C₁-C₄alkyl. For example, one of R₃ and R₄ may be pyridylC₀-C₄alkyl, pyrimidylC₀-C₄alkyl, imidazolylC₀-C₄alkyl or tetrazolylC₀-C₄alkyl, each of which is substituted with 0, 1 or 2 substituents.

In other compounds of Formula III, R₃ and R₄ are joined to form a 5 to 10-membered heterocyclic group that is substituted with from 0 to 4 substituents. In certain embodiments, the heterocyclic group is substituted with at least one substituent selected from hydroxy, halogen, C₁-C₄alkyl, haloC₁-C₄alkyl, C₁-C₄alkoxy, haloC₁-C₄alkoxy, C₁-C₄alkanoyl, and aminocarbonyl. In certain embodiments, the heterocyclic group comprises an aromatic ring. One heterocyclic group is 3,4-dihydro-1H-isoquinolin-2-yl, substituted with 0, 1 or 2 substituents. In other embodiments, the heterocyclic group is a 5- to 10-membered heterocycloalkyl, substituted with from 0 to 4 substituents. For example, the heterocycloalkyl may be piperadinyl, piperazinyl, pyrrolidinyl, azepanyl, azocinyl, decahydroquinolinyl or 1,4-dioxa-8-aza-spiro[4.5]dec-8-yl, each of which is unsubstituted or substituted with from 1 to 4 substituents independently selected from halogen, hydroxy, C₁-C₄alkyl, C₁-C₄alkoxy, haloC₁-C₄alkyl, haloC₁-C₄alkoxy, C₁-C₄alkanoyl and C₁-C₄alkoxycarbonyl. Still further heterocyclic groups include morpholino, thiomorpholino or 1,1-dioxo-thiomorpholin-4-yl, each of which is unsubstituted or substituted with from 1 to 4 substituents independently selected from halogen, hydroxy, C₁-C₄alkyl, C₁-C₄alkoxy, haloC₁-C₄alkyl, haloC₁-C₄alkoxy, C₁-C₄alkanoyl and C₁-C₄alkoxycarbonyl. Within certain compounds of Formula III in which R₃ and R₄ are joined to form a 5 to 10-membered heterocyclic group, the heterocyclic group is substituted with from 1 to 4 substituents independently selected from methyl and ethyl.

R₅ and R₆ of Formula III are, independently at each occurrence: (i) each independently selected from: (a) hydrogen and hydroxy; (b) C₁-C₈alkyl, unsubstituted or substituted with 1 or 2 substituents independently selected from R_{b}; and (c) groups that are joined to R₃ or R₄ to form a 4- to 10-membered heterocyclic group that is unsubstituted or substituted with from 1 to 6 substituents independently selected from R_{b}; (ii) taken together to form a keto group; or (iii) joined to form a 3- to 7-membered carbocyclic or heterocyclic ring, unsubstituted or substituted with from 1 to 4 substituents selected from R_{b}. R_{b} is as described above and n is 1, 2 or 3. Within certain compounds, each R₅ and R₆ is independently selected from hydrogen and C₁-C₆alkyl; in certain such compounds, R₅ and R₆ are hydrogen. Within further compounds, n is 1.

Still further substituted VR1 antagonists that are quinazolin-4-ylamine analogues are characterized by Formula IV:

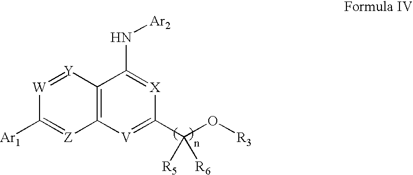

or a pharmaceutically acceptable salt thereof. In Formula IV, the variables are generally as described in PCT International Application Publication Number WO 03/062209, which published on Jul. 31, 2003.

V, X, W, Y, Z, R₅, R_{6,} Ar₁, Ar₂, and n of Formula IV are as defined for Formula III.

R₃ of Formula IV is selected from: (i) hydrogen; (ii) C₁-C₈alkyl, C₂-C₈alkenyl, C₂-C₈alkynyl, C₂-C₈alkanoyl, C₂-C₈alkyl ether, C₆-C₁₀arylC₀-C₈alkyl, and 5- to 10-membered heterocycleC₀-C₈alkyl, each of which is optionally substituted with from 1 to 9 substituents independently selected from R_{b}; and (iii) groups that are joined to an R₅ or R₆ to form a 5- to 10-membered heterocyclic group that is unsubstituted or substituted with from 1 to 6 substituents independently selected from R_{b}.

Within certain compounds of Formulas II-IV, V and/or X are N, or U and X are N. For example, both V and X may be N. In certain other embodiments, W, Y and Z are each CH or N; for example, all three may be CH or one of Y and Z may be N with the others CH.

Within certain compounds of Formulas I-IV, Ar₁ and Ar₂ are independently selected from phenyl and 6-membered aromatic heterocycles, each of which is substituted with 0, 1 or 2 substituents. In certain embodiments, (i) Ar₁ is phenyl or pyridyl, each of which is unsubstituted or substituted with 1 or 2 substituents selected from halogen, hydroxy, cyano, amino, nitro, mono- and di-(C₁-C₆alkyl)amino, C₁-C₆alkyl, haloC₁-C₆alkyl, C₁-C₆alkoxy and haloC₁-C₆alkoxy; and (ii) Ar₂ is phenyl or pyridyl, each of which is unsubstituted or substituted with 1 or 2 substituents independently selected from halogen, hydroxy, cyano, amino, mono- and di-(C₁-C₆alkyl)amino, C₁-C₆alkyl, haloC₁-C₆alkyl, C₁-C₆alkoxy, haloC₁-C₆alkoxy, C₂-C₆alkyl ether,C₁-C₆alkanoyl, —SO₂)R₇, —NRₓS(O)ₘ—, and —N(S(Oₘ)₂; wherein m is 1 or 2, Rₓ is hydrogen or C₁-C₆alkyl, and R₇ is C₁-C₆alkyl, haloC₁-C₆alkyl, amino, mono- or di-(C₁-C₆alkyl)amino or a 5- to 10-membered, N-linked heterocyclic group, each of which R₇ is optionally substituted with R_{b}. For example, in some embodiments, (i) Ar₁ is pyridyl, unsubstituted or substituted with halogen, C₁-C₄alkyl or haloC₁-C₄alkyl; and (ii) Ar₂ is phenyl or pyridyl, each of which is unsubstituted or substituted with halogen, cyano, C₁-C₄alkyl, haloC₁-C₄alkyl, C₂-C₄alkyl ether, C₁-C₄alkanoyl or —SO₂)Rₐ, wherein Rₐ is C₁-C₄alkyl or haloC₁-C₄alkyl. Certain such compounds are those in which (i) Ar₁ is pyridin-2-yl, 3-methyl-pyridin-2-yl, 3-trifluoromethyl-pyridin-2-yl or 3-halo-pyridin-2-yl; and (ii) Ar₂ is phenyl, 2-pyridyl or 3-pyridyl, each of which is substituted at the 4-position with trifluoromethanesulfonyl, propanesulfonyl, propane-2-sulfonyl, t-butyl, trifluoromethyl or 2,2,2-trifluoro-1-methyl-ethyl.

Within further compounds of Formulas II-IV, Ar₂ is selected from pyridyl, pyridazinyl, pyrimidinyl, pyrazinyl, pyrrolyl, imidazolyl, pyrazolyl, oxazolyl, isoxazolyl, thiazolyl, isothiazolyl and thiadiazolyl, each of which is unsubstituted or substituted with 1 or 2 substituents selected from halogen, cyano, C₁-C₆alkyl, haloC₁-C₆alkyl, hydroxyC₁-C₆alkyl, C₁-C₆alkyl ether, C₁-C₆alkanoyl, amino, mono- and di-(C₁-C₆alkyl)amino. In certain embodiments, Ar₂ is phenyl or a 6-membered aromatic heterocycle such as pyridyl, each of which is optionally substituted with 1 or 2 substituents selected from halogen, cyano, C₁-C₆alkyl and haloC₁-C₆alkyl. In other embodiments, Ar₂ is pyridyl, isoxazolyl, thiadiazolyl or pyrazolyl, each of which is unsubstituted or substituted with halogen, C₁-C₄alkyl or haloC₁-C₄alkyl. For example, Ar₁ and Ar₂ may each be pyridyl, substituted with 1 substituent independently chosen from halogen, C₁-C₄alkyl, C₁-C₄haloalkyl, and C₁-C₄alkoxy. In further embodiments, Ar₂ is phenyl, optionally substituted with halogen, C₁-C₄alkyl or haloC₁-C₄alkyl.

Certain representative compounds satisfying the above Formulas are described in more detail below. It will be apparent, however, that specific compounds recited herein are representative only, and that the scope of the present invention encompasses the use of any non-toxic VR1 antagonist, especially non-vanilloid VR1 antagonists. Other VR1 antagonists that may be used in the combination therapy described herein include, for example, those described in U.S. Pat. Nos. 6,476,076; 6,437,147; 6,248,788; 5,962,532; 5,840,730; 5,290,816; 5,232,684; 5,021,450; 4,812,446 and 4,424,205; published U.S. patent application Nos. 2003/0158198; 2003/0158188; 2003/0133951 and 2001/0036943; PCT International Application Publication Numbers WO 03/049702; WO 03/053945; WO 03/055848; WO 03/055484; WO 03/022809; WO 03/014064; WO 02/090326; WO 02/076946; WO 02/072536; WO 02/16319; WO 02/16318; WO 02/16317; WO 02/08221;WO 01/85158 and WO 99/00115; and Japanese Patent Application No. JP 2003-192673.

Within certain embodiments, VR1 antagonists for use as described herein do not substantially modulate ligand binding to other cell surface receptors, such as EGF receptor tyrosine kinase or the nicotinic acetylcholine receptor. In other words, such antagonists do not substantially inhibit activity of a cell surface receptor such as the human epidermal growth factor (EGF) receptor tyrosine kinase or the nicotinic acetylcholine receptor (e.g., the IC₅₀ or IC₄₀ at such a receptor is preferably greater than 1 micromolar, and most preferably greater than 10 micromolar). Preferably, a VR1 antagonist does not detectably inhibit EGF receptor activity or nicotinic acetylcholine receptor activity at a concentration of 0.5 micromolar, 1 micromolar or more preferably 10 micromolar. Assays for determining cell surface receptor activity are commercially available, and include the tyrosine kinase assay kits available from Panvera (Madison, Wis.).

In certain embodiments, preferred VR1 antagonists are non-sedating. In other words, a dose of VR1 antagonist that is twice the minimum dose sufficient to provide analgesia in an animal model for determining pain relief (such as a model provided in Example 11, herein) causes only transient (i.e., lasting for no more than ½ the time that pain relief lasts) or preferably no statistically significant sedation in an animal model assay of sedation (using the method described by Fitzgerald et al. (1988) Toxicology 49(2-3):433-9). Preferably, a dose that is five times the minimum dose sufficient to provide analgesia does not produce statistically significant sedation. More preferably, a VR1 antagonist provided herein does not produce sedation at intravenous doses of less than 25 mg/kg (preferably less than 10 mg/kg) or at oral doses of less than 140 mg/kg (preferably less than 50 mg/kg, more preferably less than 30 mg/kg).

If desired, VR1 antagonists may be selected for certain pharmacological properties including, but not limited to, oral bioavailability (preferred compounds are orally bioavailable to an extent allowing for therapeutically effective concentrations of the compound to be achieved at oral doses of less than 140 mg/kg, preferably less than 50 mg/kg, more preferably less than 30 mg/kg, even more preferably less than 10 mg/kg, still more preferably less than 1 mg/kg and most preferably less than 0.1 mg/kg), toxicity (a preferred VR1 antagonist is nontoxic when a capsaicin receptor modulatory amount, and preferably a tolerance-reducing amount, is administered to a subject), side effects (a preferred VR1 antagonist produces side effects comparable to placebo when a tolerance-reducing amount of the compound is administered to a subject), serum protein binding and in vitro and in vivo half-life (a preferred VR1 antagonist exhibits an in vitro half-life that is equal to an in vivo half-life allowing for Q.I.D. dosing, preferably T.I.D. dosing, more preferably B.I.D. dosing, and most preferably once-a-day dosing). In addition, differential penetration of the blood brain barrier may be desirable for VR1 antagonists used to reduce tolerization to systemic narcotic analgesics and other centrally acting therapeutic agents, such that total daily oral doses as described above provide a tolerance-reducing effect. Routine assays that are well known in the art may be used to assess these properties, and identify superior compounds for a particular use. For example, assays used to predict bioavailability include transport across human intestinal cell monolayers, including Caco-2 cell monolayers. Penetration of the blood brain barrier of a compound in humans may be predicted from the brain levels of the compound in laboratory animals given the compound (e.g., intravenously). Serum protein binding may be predicted from albumin binding assays. Compound half-life is inversely proportional to the frequency of dosage of a compound. In vitro half-lives of compounds may be predicted from assays of microsomal half-life as described within Example 8, herein.

Preferred VR1 antagonists are nontoxic. In general, the term “nontoxic” as used herein shall be understood in a relative sense and is intended to refer to any substance that has been approved by the United States Food and Drug Administration (“FDA”) or the European Medicines Evaluation Agency (“EMEA”) for administration to mammals (preferably humans) or, in keeping with established criteria, is susceptible to approval by the FDA or EMEA for administration to mammals (preferably humans). In addition, a highly preferred nontoxic compound generally satisfies one or more of the following criteria: (1) does not substantially inhibit cellular ATP production; (2) does not significantly prolong heart QT intervals; (3) does not cause substantial liver enlargement, and (4) does not cause substantial release of liver enzymes.

As used herein, a VR1 antagonist that “does not substantially inhibit cellular ATP production” is a compound that satisfies the criteria set forth in Example 9, herein. In other words, cells treated as described in Example 9 with 100 μM of such a compound exhibit ATP levels that are at least 50% of the ATP levels detected in untreated cells. In more highly preferred embodiments, such cells exhibit ATP levels that are at least 80% of the ATP levels detected in untreated cells.

A VR1 antagonist that “does not significantly prolong heart QT intervals” is a compound that does not result in a statistically significant prolongation of heart QT intervals (as determined by electrocardiography) in guinea pigs, minipigs or dogs upon administration of twice the minimum dose yielding a therapeutically effective in vivo concentration. In certain preferred embodiments, a dose of 0.01, 0.05. 0.1, 0.5, 1, 5, 10, 40 or 50 mg/kg administered parenterally or orally does not result in a statistically significant prolongation of heart QT intervals. By “statistically significant” is meant results varying from control at the p<0.1 level or more preferably at the p<0.05 level of significance as measured using a standard parametric assay of statistical significance such as a student's T test.

A VR1 antagonist “does not cause substantial liver enlargement” if daily treatment of laboratory rodents (e.g., mice or rats) for 5-10 days with twice the minimum dose that yields a therapeutically effective in vivo concentration results in an increase in liver to body weight ratio that is no more than 100% over matched controls. In more highly preferred embodiments, such doses do not cause liver enlargement of more than 75% or 50% over matched controls. If non-rodent mammals (e.g., dogs) are used, such doses should not result in an increase of liver to body weight ratio of more than 50%, preferably not more than 25%, and more preferably not more than 10% over matched untreated controls. Preferred doses within such assays include 0.01, 0.05. 0.1, 0.5, 1, 5, 10, 40 or 50 mg/kg administered parenterally or orally.

Similarly, a VR1 antagonist “does not promote substantial release of liver enzymes” if administration of twice the minimum dose yielding a therapeutically effective in vivo concentration does not elevate serum levels of ALT, LDH or AST in laboratory rodents by more than 100% over matched mock-treated controls. In more highly preferred embodiments, such doses do not elevate such serum levels by more than 75% or 50% over matched controls. Alternatively, a VR1 antagonist “does not promote substantial release of liver enzymes” if, in an in vitro hepatocyte assay, concentrations (in culture media or other such solutions that are contacted and incubated with hepatocytes in vitro) equivalent to two-fold the minimum in vivo therapeutic concentration of the compound do not cause detectable release of any of such liver enzymes into culture medium above baseline levels seen in media from matched mock-treated control cells. In more highly preferred embodiments, there is no detectable release of any of such liver enzymes into culture medium above baseline levels when such compound concentrations are five-fold, and preferably ten-fold the minimum in vivo therapeutic concentration of the compound.

In other embodiments, certain preferred VR1 antagonists do not inhibit or induce microsomal cytochrome P450 enzyme activities, such as CYP1A2 activity, CYP2A6 activity, CYP2C9 activity, CYP2C19 activity, CYP2D6 activity, CYP2E1 activity or CYP3A4 activity at a concentration equal to the minimum therapeutically effective in vivo concentration.

Certain preferred VR1 antagonists are not clastogenic (e.g., as determined using a mouse erythrocyte precursor cell micronucleus assay, an Ames micronucleus assay, a spiral micronucleus assay or the like) at a concentration equal to the minimum therapeutically effective in vivo concentration. In other embodiments, certain preferred VR1 antagonists do not induce sister chromatid exchange (e.g., in Chinese hamster ovary cells) at such concentrations.

### Addictive Substances and Addictive Therapeutic Agents

An addictive substance is any compound that, when taken (e.g., ingested, inhaled or injected) by an individual, induces detectable symptoms of tolerance and/or dependence in the individual. Addictive therapeutic agents are any compounds that, when administered to a patient for therapeutic purposes (e.g., pain relief, sleep induction, or treatment of anxiety, depression or other mental illness), induce detectable symptoms of tolerance and/or dependence. Tolerance refers to a lowered response to a drug over time (i.e., a need to increase the drug dosage to maintain the original pharmacological effect). Dependence, as used herein, refers to physical dependence, in which a patient who has been treated with an addictive substance is likely to experience a withdrawal reaction if the drug is abruptly withdrawn. Withdrawal symptoms may include transpiring, feeling cold, goose flesh/pimples, running nose, stomach cramps, aching muscles and/or diarrhea. Any agent that has been found to induce tolerance and/or dependence in a patient is considered an addictive therapeutic agent, regardless of whether psychological dependence occurs.

Certain addictive therapeutic agents are narcotic analgesic agents, which are natural or synthetic drugs that have morphine-like activity and typically act at one or more opioid receptor subtypes (e.g., μ, κ, and/or δ), preferably as agonists or partial agonists. Such agents include opiates, opiate derivatives and opioids, as well as pharmaceutically acceptable salts and hydrates thereof. Specific examples of narcotic analgesics include acetorphine, acetyldihydrocodeine, alfentanyl, acetylmethadol, allylprodine, alphracetylmethadol, alphameprodine, alphamethadol, alphaprodine, anileridine, benzethidine, benzylmorphine, betacetylmethadol, betameprodine, betamethadol, betaprodine, bezitramide, buprenorphine, butorphanol, clonitazene, codeine, codeine methylbromide, codeine-N-oxide, cyprenorphine, desomorphine, dextromoramide, dextropropoxyphene, diacetyldihydromorphine, diacetylmorphine, diampromide, diethylthiambutene, difenoxin, dihydrocodeine, dihydroetorpine, dihydromorphine, dimenoxadol, dimepheptanol, dimethylthiamubutene, diphenoxylate, dioxaphetyl butyrate, dipipanone, drotebanol, ethylmethylthiambutene, ethylmorphine, etonitazene, etorphine, etoxeridine, fentanyl, furethidine, heroin, hydrocodone, hydromorphinol, hydromorphone, hydroxypethidine, isomethadone, ketobemidone, levomethorphan, levomoramide, levophenacylmorphan, levorphane, levorphanol, meperidine, metazocine, methadone, methorphan, methyldihydromorphine, methyldesorphine, metopon, morpheridine, morphine, morphine methylbromide, morphine methylsulfonate, morphine-N-oxide, myrophin, nalbuphine, naloxone, naltrexone, nicocodeine, nicomorphine, noracymethadol, norlevorphanol, normethadone, normorphine, norpipanone, opium (e.g., opium extracts, opium fluid extracts, powdered opium, granulated opium, raw opium or tincture of opium), oxycodone, oxymorphone, paregoric, pentazocine, pethidine, phenadoxone, phenampromide, phenazocine, phenomorphan, phenoperidine, pholcodine, piminodine, piritramide, proheptazine, properidine, propiram, propoxyphene, racemethorphan, racemoramide, racemorphan, thebacon, thebaine, trimeperidine and pharmaceutically acceptable salts and hydrates of the foregoing agents. Certain narcotic analgesics are provided in combination with another narcotic analgesic and/or a non-narcotic agent such as acetaminophine or aspirin, and such combinations may also be used in the compositions and methods provided herein.

In certain embodiments, preferred narcotic analgesics include alfentanyl, alphaprodine, anileridine, bezitramide, codeine, dextropropoxyphene, dihydrocodeine, diphenoxylate, ethylmorphine, fentanyl, heroin, hydrocodone, hydromorphone, isomethadone, levomethorphan, levorphanol, metazocine, methadone, metopon, morphine, opium, oxycodone, oxymorphone, pethidine, phenazocine, piminodine, racemethorphan, racemorphan, thebaine, their mixtures and their pharmaceutically acceptable salts and hydrates. Particularly preferred narcotic analgesics for use in the compositions and methods provided herein are codeine, fentanyl, heroin, hydrocodone, morphine, oxycodone, their mixtures and their pharmaceutically acceptable salts and hydrates.

Addictive therapeutic agents may further include analgesic peptide morphine-like substances such as, for example, enkephalins (e.g., methionine enkephalin and leucine enkephalin); endorphins (e.g., α-endorphin, β-endorphin, and γ-endorphin); and dynorphins (e.g., dynorphin A and dynorphin B, and precursors thereof such as proenkephalins, propiomelanocortins and prodynorphins).

Further specific representative addictive therapeutic agents include, for example: TALWIN® Nx and DEMEROL® (both available from Sanofi Winthrop Pharmaceuticals; New York, N.Y.); LEVO-DROMORAN®; BUPRENEX® (Reckitt & Coleman Pharmaceuticals, Inc.; Richmond, Va.); MSIR® (Purdue Pharma L.P.; Norwalk, Conn.); DILAUDID® (Knoll Pharmaceutical Co.; Mount Olive, N.J.); SUBLIMAZE®; SUFENTA® (Janssen Pharmaceutica Inc.; Titusville, N.J.); PERCOCET®, NUBAIN® and NUMORPHAN® (all available from Endo Pharmaceuticals Inc.; Chadds Ford, Pa.) HYDROSTAT® IR, MS/S and MS/L (all available from Richwood Pharmaceutical Co. Inc; Florence, Ky.), ORAMORPH® SR and ROXICODONE® (both available from Roxanne Laboratories; Columbus Ohio) and STADOL® (Bristol-Myers Squibb; New York, N.Y.).

Other addictive substances include ethanol and the cannabinoids, including tetrahydrocannabinol (THC, including delta⁹THC, delta⁸THC, delta¹THC, delta¹⁽⁶⁾THC), cannabidiol (CBD), cannabinol (CBN), and metabolites thereof such as 7-hydroxy-delta¹⁽⁶⁾-THC). The use of VR1 antagonists to inhibit the development of tolerance to and/or dependence on such agents is also contemplated by the present invention.

### VR1 Antagonist Compositions

Compositions for use in the present invention generally comprise a VR1 antagonist in combination with at least one physiologically acceptable carrier or excipient. Suitable carriers and excipients include, for example, water, buffers (e.g., neutral buffered saline or phosphate buffered saline), ethanol, mineral oil, vegetable oil, dimethylsulfoxide, carbohydrates (e.g., glucose, mannose, sucrose or dextrans), mannitol, proteins, adjuvants, polypeptides or amino acids such as glycine, antioxidants, chelating agents such as EDTA or glutathione and/or preservatives. Certain compositions comprise a VR1 antagonist in combination with an addictive therapeutic agent (preferably a narcotic analgesic).

Pharmaceutical compositions may be formulated for any appropriate manner of administration, including, for example, topical, oral, nasal, rectal or parenteral administration. The term parenteral as used herein includes subcutaneous, intradermal, intravascular (e.g., intravenous), intramuscular, spinal, intracranial, intrathecal and intraperitoneal injection, as well as any similar injection or infusion technique. In certain embodiments, pharmaceutical compositions are formulated for oral delivery to humans or other animals (e.g., companion animals such as dogs). Such forms include, for example, tablets, troches, lozenges, aqueous or oily suspensions, dispersible powders or granules, emulsion, hard or soft capsules, or syrups or elixirs. Within yet other embodiments, compositions of the present invention may be formulated as a lyophilizate.

Compositions intended for oral use may further comprise one or more components such as sweetening agents, flavoring agents, coloring agents and preserving agents in order to provide appealing and palatable preparations. Tablets contain the active ingredient in admixture with physiologically acceptable excipients that are suitable for the manufacture of tablets. Such excipients include, for example, inert diluents (e.g., calcium carbonate, sodium carbonate, lactose, calcium phosphate or sodium phosphate), granulating and disintegrating agents (e.g., corn starch or alginic acid), binding agents (e.g., starch, gelatin or acacia) and lubricating agents (e.g., magnesium stearate, stearic acid or talc). The tablets may be uncoated or they may be coated by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules wherein the active ingredient is mixed with an inert solid diluent (e.g., calcium carbonate, calcium phosphate or kaolin), or as soft gelatin capsules wherein the active ingredient is mixed with water or an oilmedium (e.g., peanut oil, liquid paraffin or olive oil).

Aqueous suspensions comprise the active materials in admixture with excipients suitable for the manufacture of aqueous suspensions. Such excipients are suspending agents (e.g., sodium carboxymethylcellulose, methylcellulose, hydropropylmethylcellulose, sodium alginate, polyvinylpyrrolidone, gum tragacanth and gum acacia); and dispersing or wetting agents (e.g., naturally-occurring phosphatides such as lecithin, condensation products of an alkylene oxide with fatty acids such as polyoxyethylene stearate, condensation products of ethylene oxide with long chain aliphatic alcohols such as heptadecaethyleneoxycetanol, condensation products of ethylene oxide with partial esters derived from fatty acids and a hexitol such as polyoxyethylene sorbitol monooleate, or condensation products of ethylene oxide with partial esters derived from fatty acids and hexitol anhydrides such as polyethylene sorbitan monooleate). Aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil (e.g., arachis oil, olive oil, sesame oil or coconut oil) or in a mineral oil such as liquid paraffin. The oily suspensions may contain a thickening agent such as beeswax, hard paraffin or cetyl alcohol. Sweetening agents such as those set forth above, and/or flavoring agents may be added to provide palatable oral preparations. Such suspension may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions may also be in the form of oil-in-water emulsions. The oily phase may be a vegetable oil (e.g., olive oil or arachis oil) or a mineral oil (e.g., liquid paraffin) or mixtures thereof. Suitable emulsifying agents may be naturally-occurring gums (e.g., gum acacia or gum tragacanth), naturally-occurring phosphatides (e.g., soy bean, lecithin, and esters or partial esters derived from fatty acids and hexitol), anhydrides (e.g., sorbitan monoleate) and condensation products of partial esters derived from fatty acids and hexitol with ethylene oxide (e.g., polyoxyethylene sorbitan monoleate). The emulsions may also contain sweetening and/or flavoring agents.

Syrups and elixirs may be formulated with sweetening agents, such as glycerol, propylene glycol, sorbitol or sucrose. Such formulations may also comprise one or more demulcents, preservatives, flavoring agents and/or coloring agents.

Formulations for topical administration typically comprise a topical vehicle combined with active agent(s), with or without additional optional components. Suitable topical vehicles and additional components are well known in the art, and it will be apparent that the choice of a vehicle will depend on the particular physical form and mode of delivery. Topical vehicles include water; organic solvents such as alcohols (e.g., ethanol or isopropyl alcohol) or glycerin; glycols (e.g., butylene, isoprene or propylene glycol); aliphatic alcohols (e.g., lanolin); mixtures of water and organic solvents and mixtures of organic solvents such as alcohol and glycerin; lipid-based materials such as fatty acids, acylglycerols (including oils, such as mineral oil, and fats of natural or synthetic origin), phosphoglycerides, sphingolipids and waxes; protein-based materials such as collagen and gelatin; silicone-based materials (both non-volatile and volatile); and hydrocarbon-based materials such as microsponges and polymer matrices. A composition may further include one or more components adapted to improve the stability or effectiveness of the applied formulation, such as stabilizing agents, suspending agents, emulsifying agents, viscosity adjusters, gelling agents, preservatives, antioxidants, skin penetration enhancers, moisturizers and sustained release materials. Examples of such components are described in Martindale—The Extra Pharmacopoeia (Pharmaceutical Press, London 1993) and Martin (ed.), Remington's Pharmaceutical Sciences. Formulations may comprise microcapsules, such as hydroxymethylcellulose or gelatin-microcapsules, liposomes, albumin microspheres, microemulsions, nanoparticles or nanocapsules. Typical modes of delivery for topical compositions include application using the fingers; application using a physical applicator such as a cloth, tissue, swab, stick or brush; spraying (including mist, aerosol or foam spraying); dropper application; sprinkling; soaking; and rinsing. Controlled release vehicles can also be used.

A pharmaceutical composition may be prepared as a sterile injectible aqueous or oleaginous suspension. The active agent(s), depending on the vehicle and concentration used, can either be suspended or dissolved in the vehicle. Such a composition may be formulated according to the known art using suitable dispersing, wetting agents and/or suspending agents such as those mentioned above. Among the acceptable vehicles and solvents that may be employed are water, 1,3-butanediol, Ringer's solution and isotonic sodium chloride solution. In addition, sterile, fixed oils may be employed as a solvent or suspending medium. For this purpose any bland fixed oil may be employed, including synthetic mono- or diglycerides. In addition, fatty acids such as oleic acid find use in the preparation of injectible compositions, and adjuvants such as local anesthetics, preservatives and/or buffering agents can be dissolved in the vehicle.

Compositions may also be prepared in the form of suppositories (e.g., for rectal administration). Such compositions can be prepared by mixing the drug with a suitable non-irritating excipient that is solid at ordinary temperatures but liquid at the rectal temperature and will therefore melt in the rectum to release the drug. Suitable excipients include, for example, cocoa butter and polyethylene glycols.

Pharmaceutical compositions may be formulated as sustained release formulations (i.e., a formulation such as a capsule that effects a slow release of active agent(s) following administration). Such formulations may generally be prepared using well known technology and administered by, for example, oral, rectal or subcutaneous implantation, or by implantation at the desired target site. Carriers for use within such formulations are biocompatible, and may also be biodegradable; preferably the formulation provides a relatively constant level of active agent(s) release. The amount of active agent(s) contained within a sustained release formulation depends upon the site of implantation, the rate and expected duration of release and the nature of the condition to be treated or prevented.

In addition to or together with the above modes of administration, a VR1 antagonist may be conveniently added to food or drinking water (e.g., for administration to non-human animals including companion animals (such as dogs and cats) and livestock). Animal feed and drinking water compositions may be formulated so that the animal takes in an appropriate quantity of the composition along with its diet. It may also be convenient to present the composition as a premix for addition to feed or drinking water.

VR1 antagonists are generally present within a pharmaceutical composition in a capsaicin receptor modulatory amount, and preferably a tolerance-reducing amount, a dependence-reducing amount or a pain relief-enhancing amount. As used herein, a “capsaicin receptor modulatory amount” is an amount that, upon administration, achieves a concentration of VR1 antagonist at a capsaicin receptor that is sufficient to alter the binding of vanilloid ligand to VR1 in vitro (using the assay provided in Example 6) and/or VR1-mediated signal transduction (using an assay provided in Example 7). The capsaicin receptor may be present, or example, in a body fluid such as blood, plasma, serum, CSF, synovial fluid, lymph, cellular interstitial fluid, tears or urine.

A tolerance-reducing amount is an amount which, when administered once, continuously or repeatedly (contemporaneously with the repeated or continuous administration of an addictive substance) to a patient at a prescribed level or frequency, results in a decrease in tolerance to the addictive substance induced by the repeated or continuous administration of the addictive substance. “Contemporaneously,” as used herein, refers to a time frame such that the VR1 antagonist is present in a body fluid of a patient (at concentration that is sufficient to alter the binding of vanilloid ligand to VR1 and/or VR1-mediated signal transduction in vitro) at the same time as the addictive substance is present in a body fluid of a patient (at a concentration that results in a detectable effect, such as pain relief, tolerance and/or symptoms of dependence). In general, as repeated or continuous administration of an addictive substance induces tolerance, it becomes necessary to increase the dose of the addictive substance in order to maintain a level of benefit (e.g., pain relief). A decrease in tolerance may be evidenced by a delay in such a dosage increase and/or a decrease in the amount of additional addictive substance needed to maintain a level of benefit.

A dependence-reducing amount is an amount which, when administered once, continuously or repeatedly (contemporaneously with the continuous or repeated administration of an addictive substance) to a patient at a prescribed level or frequency, results in a decrease in dependence on the addictive substance induced by the repeated or continuous administration of the addictive substance. A decrease in dependence may be detected based on decrease in the number and/or severity of behavioral or physical symptoms as the patient withdraws from the addictive substance.

A pain relief-enhancing amount is an amount which, when administered to a patient contemporaneously with an addictive analgesic results in synergistic pain relief (i.e., pain relief that is greater than the sum of the pain relief that would be achieved by the separate administration of the same amounts of VR1 antagonist and addictive analgesic). Such synergism may be detected using any standard pain relief assay, including those described herein.

Preferred systemic doses are no higher than 200 mg per kilogram of body weight per day. In certain embodiments, compositions providing dosage levels ranging from about 0.1 mg to about 140 mg per kilogram of body weight per day are preferred (about 0.5 mg to about 7 g per human patient per day). Compositions providing intravenous dosages ranging from about 0.001 mg to about 50 mg per kilogram of body weight per day may also be preferred, with oral doses generally being about 5-20 fold higher than intravenous doses (e.g., ranging from 0.01 to 40 mg per kilogram of body weight per day).

The amount of active ingredient that may be combined with the carrier materials to produce a single dosage form will vary depending upon the host treated and the particular mode of administration. Dosage unit forms will generally contain between from about 1 mg to about 500 mg of an active ingredient. Optimal dosages may be established using routine testing, and procedures that are well known in the art. Dosages of addictive therapeutic agents can be found, for example, in the manufacturer's instructions set forth in the package insert for the agent, or in the Physician's Desk Reference.

Pharmaceutical compositions may be packaged for inhibiting the development of tolerance and/or dependence. Packaged pharmaceutical compositions generally include a container holding a tolerance-reducing and/or dependence reducing amount of at least one VR1 antagonist and instructions (e.g., labeling) indicating that the contained composition is to be used for inhibiting the development of tolerance to or dependence on an addictive substance in the patient. Alternatively, the instructions may indicate that the composition is to be administered in combination (ie., simultaneously or sequentially in either order) with an addictive therapeutic agent. Such packaged compositions may further comprise one or more addictive therapeutic agents (preferably a narcotic analgesic) in the same container or in a separate container within the package. Preferred mixtures are formulated for oral administration (e.g., as pills, capsules, tablets or the like). In certain embodiments, the package comprises a label bearing indicia indicating that the one or more VR1 antagonists and one or more addictive therapeutic agents are to be taken together for the treatment of a pain condition.

### Therapeutic Methods

The present invention provides methods for using a VR1 antagonist in combination with an addictive substance for the treatment of pain and/or to inhibit the development of tolerance and/or adverse effect(s) such as dependence in patients treated with an addictive substance. The VR1 antagonist may be administered to the patient at the same time as the addictive substance (e.g., as a single dosage unit), or may be administered separately (before or after the addictive substance). Within preferred embodiments, the VR1 antagonist and addictive substance are ultimately simultaneously present in effective amounts in a body fluid (e.g., blood) of the patient. Administration of the VR1 antagonist and addictive substance to the patient can be by way of any means discussed above, including oral, topical, nasal or transdermal administration, or intravenous, intramuscular, subcutaneous, intrathecal, epidural, intracerebroventrilcular or like injection. In certain embodiments, a mixture of one or more VR1 antagonists and one or more addictive therapeutic agents, as described above, is administered. Preferred mixtures are formulated for oral administration (e.g., as pills, capsules, tablets or the like) or intravenous administration.

A “patient,” as used herein, is any individual treated with a VR1 antagonist and an addictive substance. Patients include humans, as well as other animals such as companion animals (e.g., dogs and cats) and livestock. In certain embodiments, patients may be experiencing tolerance or other adverse effect(s) of addictive substance treatment, or may be considered to be at risk for such symptom(s).

The VR1 antagonist is generally administered in a capsaicin receptor modulatory amount, and preferably in a tolerance-reducing, dependence-reducing or pain relief-enhancing amount. Frequency of dosage may vary depending on the compound used and amount and nature of the particular addictive substance. In general, a dosage regimen of 4 times daily or less is preferred, as is the use of the minimum dosage that is sufficient to provide effective therapy. The preferred dose of nontoxic VR1 antagonist generally ranges from about 0.001 mg to about 50 mg, 0.01 mg to about 10 mg or 0.01 mg to about 1.0 mg per kilogram of body weight per day. For example, a dose ranging from 0.25 to about 250 mg/day may be suitable; actual doses will vary according to the particular active substances being used, the particular formulation containing the active substances and the state and circumstances of the patient. It will be apparent that administration may be by any conventional means, such as those described herein, including intravenous administration (continuously or in discrete doses) and oral administration.

Doses of addictive therapeutic agent may be found, for example, on the package insert for the agent. In certain embodiments, the combination administration of a VR1 antagonist with an addictive therapeutic agent results in a reduction of the dosage of the addictive therapeutic agent required to produce a therapeutic effect. Thus, the dose of addictive therapeutic agent in a combination or combination treatment method provided herein may be less than the maximum dose advised by the manufacturer for administration of the addictive therapeutic agent without combination administration of a VR1 antagonist. In certain embodiments, this dose is less than ¾, ½, ¼ or 10% of the maximum dose advised by the manufacturer for administration of the addictive therapeutic agent(s) when administered without combination administration of a VR1 antagonist. In further embodiments, the dose of addictive therapeutic agent is lower than the minimum dose suggested by the manufacturer.

Reduced dosages of certain preferred addictive therapeutic agents or narcotic analgesics which are appropriate for use in combination with a contemporaneously administered dose of a VR1 antagonist for the treatment of pain include:

alfenantyl administered intravenously at less than about 3 μg/kg (or more preferably administered intravenously at a dose of less than about 2.5 μg/kg, less than about 2 μg/kg, less than about 1.5 μg/kg, less than about 1 μg/kg, less than about 0.5 μg/kg, or intravenously at a dose of less than about 0.1 μg/kg),

anileridine administered in a single dose form (e.g., a pill, tablet, or other single use formulation) of at less than about 25 mg (or more preferably administered at a dose of less than about 20 mg, less than about 15 mg, less than about 10 mg, less than about 5 mg, or at a dose of less than about 2.5 mg),

codeine administered in a single dose form (e.g., a pill, tablet, or other single use formulation) of at less than about 30 mg (or more preferably administered at a dose of less than about 25 mg, less than about 20 mg, less than about 15 mg, less than about 10 mg, less than about 5 mg, or at a dose of less than about 3 mg),

Dextroproposyphene administered in a single dose form (e.g., a pill, tablet, or other single use formulation) of at less than about 50 mg (or more preferably administered at a dose of less than about 40 mg, less than about 30 mg, less than about 20mg, less than about 15 mg, less than about 10 mg, or at a dose of less than about 5 mg),

Dihydrocodeine administered in a single dose form (e.g., a pill, tablet, or other single use formulation) of at less than about 30 mg (or more preferably administered at a dose of less than about 25 mg, less than about 20 mg, less than about 15 mg, less than about 10 mg, less than about 5 mg, or at a dose of less than about 3 mg),

Diphenoxylate administered in a single dose form (e.g., a pill, tablet, or other single use formulation) of at less than about 5 mg (or more preferably administered at a dose of less than about 4 mg, less than about 3 mg, less than about 2 mg, less than about 1.5 mg, less than about 1 mg, or at a dose of less than about 0.5 mg),

Fenantyl administered in a single dose form (e.g., a pill, tablet, or other single use formulation) of at less than about 25 μg (or more preferably administered at a dose of less than about 25 μg, less than about 20 μg, less than about 15 μg, less than about 10 μg less than about 5 μg, or at a dose of less than about 2.5 μg),

Hydrocodone administered in a single dose form (e.g., a pill, tablet, or other single use formulation) of at less than about 2.5 mg (or more preferably administered at a dose of less than about 2 mg, less than about 1.5 mg, less than about 1 mg, less than about 0.5 mg, less than about 0.5 mg, or at a dose of less than about 0.25 mg),

Hydromorphone administered in a single dose form (e.g., a pill, tablet, or other single use formulation) of at less than about 2 mg (or more preferably administered at a dose of less than about 1.5 mg, less than about 1.25 mg, less than about 1 mg, less than about 0.8 mg, less than about 0.5 mg, or at a dose of less than about 0.2 mg),

Levorphanol administered in a single dose form (e.g., a pill, tablet, or other single use formulation) of at less than about 1 mg (or more preferably administered at a dose of less than about 0.8 mg, less than about 0.6 mg, less than about 0.4, less than about 0.25 mg, less than about 0.2 mg, or at a dose of less than about 0.1 mg),

Meperidine administered in a single dose form (e.g., a pill, tablet, or other single use formulation) of at less than about 25 mg (or more preferably administered at a dose of less than about 20 mg, less than about 15 mg, less than about 10 mg, less than about 5 mg, less than about 2.5 mg, or at a dose of less than about 1 mg),

Methadone administered in a single dose form (e.g., a pill, tablet, or other single use formulation) of at less than about 5 mg (or more preferably administered at a dose of less than about 4 mg, less than about 3 mg, less than about 2.5 mg, less than about 2 mg, less than about 1 mg, or at a dose of less than about 0.5 mg),

Morphine administered in a single dose form (e.g., a pill, tablet, or other single use formulation) of at less than about 10 mg (or more preferably administered at a dose of less than about 7.5 mg, less than about 5 mg, less than about 4 mg, less than about 2.5 mg, less than about 1 mg, or at a dose of less than about 0.5 mg),

Oxycodon administered in a single dose form (e.g., a pill, tablet, or other single use formulation) of at less than about 2.5 mg (or more preferably administered at a dose of less than about 2 mg, less than about 1.5 mg, less than about 1 mg, less than about 0.5 mg, less than about 0.25 mg, or at a dose of less than about 0.1 mg),

Oxymorphone administered in a single dose form (e.g., a pill, tablet, or other single use formulation) of at less than about 1 mg (or more preferably administered at a dose of less than about 0.8 mg, less than about 0.6 mg, less than about 0.5 mg, less than about 0.4 mg, less than about 0.25 mg, or at a dose of less than about 0.1 mg),

Pethidine administered in a single dose form (e.g., a pill, tablet, or other single use formulation) of at less than about 50 mg (or more preferably administered at a dose of less than about 40 mg, less than about 30 mg, less than about 25 mg, less than about 15 mg, less than about 10 mg, or at a dose of less than about 5 mg), or

Proposyphene administered in a single dose form (e.g., a pill, tablet, or other single use formulation) of at less than about 50 mg (or more preferably administered at a dose of less than about 40 mg, less than about 30 mg, less than about 25 mg, less than about 15 mg, less than about 10 mg, or at a dose of less than about 5 mg).

Other preferred addictive therapeutic agents or narcotic analgesics which may be administered in combination with a VR1 antagonist to a patient to prevent or treat pain at a reduced dosage amount for the addictive therapeutic agents or narcotic analgesics alone include alphaprodine, bezitramide, ethylmorphine, heroin, isomethadone, isomethadone, levomethorphan, metazocine, metopon, opium, phenazocine, piminodine, racemethorphan, racemorphan, thebaine and the like. Typically preferred dosages of the addictive therapeutic agents or narcotic analgesics when administered for the treatment of pain in combination with a VR1 antagonist is less than about 80% of the dosage necessary for pain reduction in the absence of VR1 antagonist administration. More preferably, the dosage is less than about 75%, 70%, 60%, 50%, 40%, 30%, 25%, 20%, 15%, or less than about 10% of the dosage necessary for pain reduction in the absence of VR1 antagonist administration.

Adverse effects of addictive therapeutic agents that may be reduced (e.g., delayed, prevented, or decreased in severity or duration) using the methods provided herein include, in addition to dependence, effects such as sedation, constipation, respiratory depression, dizziness, nausea, decreased appetite, immune system effects and other known adverse effects of the particular addictive therapeutic agent being administered.

Within certain embodiments, methods are provided for inhibiting the development of tolerance to a narcotic analgesic in a patient, comprising administering to a patient, simultaneously or sequentially in either order; (i) a narcotic analgesic; and (ii) a tolerance-reducing amount of a nontoxic VR1 antagonist. Within other embodiments, methods are provided for inhibiting the development of dependence on a narcotic analgesic in a patient, comprising administering to a patient, simultaneously or sequentially in either order; (i) a narcotic analgesic; and (ii) a dependence-reducing amount of a nontoxic VR1 antagonist.

Within further methods, the VR1 antagonists provided herein may be used to enhance the pain relief obtained from an addictive analgesic, preferably a narcotic analgesic. Within such methods, the VR1 antagonist and narcotic analgesic function synergistically, resulting in a decrease in the therapeutically effective dosage of narcotic analgesic (i.e., an increase in pain relief resulting from administration of a given dose of narcotic analgesic). Such methods generally comprise administering to a patient, simultaneously or sequentially in either order; (i) a narcotic analgesic; and (ii) a pain-relief enhancing amount of a nontoxic VR1 antagonist.

Suitable narcotic analgesics for use within the above methods are as described above and include, but are not limited to, alfentanyl, alphaprodine, anileridine, bezitramide, codeine, dihydrocodeine, diphenoxylate, ethylmorphine, fentanyl, heroin, hydrocodone, hydromorphone, isomethadone, levomethorphan, levorphanol, metazocine, methadone, metopon, meperidine, morphine, opium extracts, opium fluid extracts, powdered opium, granulated opium, raw opium, tincture of opium, oxycodone, oxymorphone, pethidine, phenazocine, piminodine, racemethorphan, racemorphan, thebaine, their mixtures and their pharmaceutically acceptable salts and hydrates.

VR1 antagonists may also be used to treat withdrawal symptoms resulting from prior administration of an addictive substance. Within such methods, a nontoxic VR1 antagonist is administered to a patient experiencing or susceptible to withdrawal symptoms. A patient is considered susceptible to withdrawal symptoms if the patient has previously taken (via any mode of administration described herein) an addictive substance in an amount generally considered sufficient to be likely to induce symptoms upon withdrawal of the substance). It will be apparent that the prior administration of the addictive substance may have been for therapeutic purposes, or the substance may have been self-administered by the patient for non-therapeutic purposes. In either case, the VR1 antagonist is administered in an amount sufficient to decrease the severity of withdrawal symptoms in the patient.

### Representative VR1 Antagonists

The present invention contemplates the use of any non-toxic VR1 antagonist in the methods and compositions provided herein. For illustrative purposes, certain representative VR1 antagonists are described more fully below. Compounds specifically recited herein are not intended to limit the scope of the present invention. In addition, it will be apparent that, within the general synthetic schemes provided herein, the starting materials may be varied and additional steps employed to produce a variety of VR1 antagonists.

Compounds are generally described herein using standard nomenclature. For compounds having asymmetric centers, it should be understood that (unless otherwise specified) all of the optical isomers and mixtures thereof are encompassed. In addition, compounds with carbon-carbon double bonds may occur in Z- and E-forms, with all isomeric forms of the compounds being included in the present invention unless otherwise specified. Where a compound exists in various tautomeric forms, a recited compound is not limited to any one specific tautomer, but rather is intended to encompass all tautomeric forms. Certain compounds are described herein using a general formula that includes variables (e.g., R₁, n, Ar₁). Unless otherwise specified, each variable within such a formula is defined independently of other variable, and any variable that occurs more than one time in a formula is defined independently at each occurrence.

As used herein, the term “alkyl” refers to a straight chain, branched chain or cyclic saturated aliphatic hydrocarbon. An alkyl group may be bonded to an atom within a molecule of interest via any chemically suitable portion. Alkyl groups include groups having from 1 to 8 carbon atoms (C₁-C₈alkyl), from 1 to 6 carbon atoms (C₁-C₆alkyl) and from 1 to 4 carbon atoms (C₁-C₄alkyl), such as methyl, ethyl, propyl, isopropyl, n-butyl, sec-butyl, tert-butyl, pentyl, 2-pentyl, isopentyl, neopentyl, hexyl, 2-hexyl, 3-hexyl, 3-methylpentyl, cyclopropyl, cyclopropylmethyl, cyclopentyl, cyclopentylmethyl, cyclohexyl, cycloheptyl and norbornyl. “C₀-C₄alkyl” refers to a bond or a C₁-C₄alkyl group; “C₀-C₈alkyl” refers to a bond or a C₁-C₈alkyl group.

Similarly, “alkenyl” refers to straight or branched chain alkene groups or cycloalkene groups. Within an alkenyl group, one or more unsaturated carbon-carbon double bonds are present. Alkenyl groups include C₂-C₈alkenyl, C₂-C₆alkenyl and C₂-C₄alkenyl groups, which have from 2 to 8, 2 to 6 or 2 to 4 carbon atoms, respectively, such as ethenyl, allyl or isopropenyl. “Alkynyl” refers to straight or branched chain alkyne groups, which have, one or more unsaturated carbon-carbon bonds, at least one of which is a triple bond. Alkynyl groups include C₂-C₈alkynyl, C₂-C₆alkynyl and C₂-C₄alkynyl groups, which have from 2 to 8, 2 to 6 or 2 to 4 carbon atoms, respectively.

By “alkoxy,” as used herein, is meant an alkyl, alkenyl or alkynyl group as described above attached via an oxygen bridge. Alkoxy groups include C₁-C₈alkoxy, C₁-C₆alkoxy and C₁-C₄alkoxy groups, which have from 1 to 8, 1 to 6 or 1 to 4 carbon atoms, respectively. Alkoxy groups include, for example, methoxy, ethoxy, propoxy, isopropoxy, n-butoxy, sec-butoxy, tert-butoxy, n-pentoxy, 2-pentoxy, 3-pentoxy, isopentoxy, neopentoxy, hexoxy, 2-hexoxy, 3-hexoxy, and 3-methylpentoxy.

The term “alkanoyl” refers to an acyl group in a linear, branched or cyclic arrangement (e.g., —(C═O)-alkyl). Alkanoyl groups include C₂-C₈alkanoyl, C₂-C₆alkanoyl and C₂-C₄alkanoyl groups, which have from 2 to 8, 2 to 6 or 2 to 4 carbon atoms, respectively.

An “alkanone” is a ketone group in which carbon atoms are in a linear, branched or cyclic alkyl arrangement. “C₃-C₈alkanone,” “C₃-C₆alkanone” and “C₃-C₄alkanone” refer to an alkanone having from 3 to 8, 6 or 4 carbon atoms, respectively.

Similarly, “alkyl ether” refers to a linear or branched ether substituent linked via a carbon-carbon bond. Alkyl ether groups include C₂-C₈alkyl ether, C₂-C₆alkyl ether and C₂-C₆alkyl ether groups, which have 2 to 8, 6 or 4 carbon atoms, respectively.

The term “alkoxycarbonyl” refers to an alkoxy group linked via a carbonyl (e.g., a group having the general structure —C(═O)—O-alkyl). Alkoxycarbonyl groups include C₂-C₈, C₂-C₆ and C₂-C₄alkoxycarbonyl groups, which have from 2 to 8, 6 or 4 carbon atoms, respectively.

“Alkanoyloxy,” as used herein, refers to an alkanoyl group linked via an oxygen bridge (e.g., a group having the general structure —O—C(═O)-alkyl). Alkanoyloxy groups include C₂-C₈, C₂-C₆ and C₂-C₄alkanoyloxy groups, which have from 2 to 8, 6 or 4 carbon atoms, respectively.

The term “aminocarbonyl” refers to an amide group (ie., —(C═O)NH₂).

The term “halogen” includes fluorine, chlorine, bromine and iodine. A “haloalkyl” is a branched, straight-chain or cyclic alkyl group, substituted with 1 or more halogen atoms (e.g., “haloC₁-C₈alkyl” groups have from 1 to 8 carbon atoms; “haloC₁-C₆alkyl” groups have from 1 to 6 carbon atoms). Examples of haloalkyl groups include, but are not limited to, mono-, di- or tri-fluoromethyl; mono-, di- or tri-chloromethyl; mono-, di-, tri-, tetra- or penta-fluoroethyl; and mono-, di-, tri-, tetra- or penta-chloroethyl. Typical haloalkyl groups are trifluoromethyl and difluoromethyl. Within certain compounds provided herein, not more than 5 or 3 haloalkyl groups are present. The term “haloalkoxy” refers to a haloalkyl group as defined above attached via an oxygen bridge. “HaloC₁-C₈alkoxy” groups have 1 to 8 carbon atoms.

A dash (“—”) that is not between two letters or symbols is used to indicate a point of attachment for a substituent. For example, —CONH₂ is attached through the carbon atom.

A “heteroatom,” as used herein, is oxygen, sulfur or nitrogen.

A “carbocycle” or “carbocyclic group” comprises at least one ring formed entirely by carbon-carbon bonds (referred to herein as a carbocyclic ring), and does not contain a heterocyclic ring. Unless otherwise specified, each carbocyclic ring within a carbocycle may be saturated, partially saturated or aromatic. A carbocycle generally has from 1 to 3 fused, pendant or spiro rings, carbocycles within certain embodiments have one ring or two fused rings. Typically, each ring contains from 3 to 8 ring members (i.e., C₃-C₈); C₅-C₇ rings are recited in certain embodiments. Carbocycles comprising fused, pendant or spiro rings typically contain from 9 to 14 ring members. Certain representative carbocycles are optionally substituted cycloalkyl (i.e., groups that comprise saturated and/or partially saturated rings, such as cyclopropyl, cyclobutyl, cyclopentyl, cyclohexyl, cycloheptyl, cyclooctyl, adamantyl, decahydro-naphthalenyl, octahydro-indenyl, and partially saturated variants of any of the foregoing, such as cyclohexenyl), as well as aromatic groups (ie., groups that contain at least one aromatic carbocyclic ring, such as phenyl, benzyl, naphthyl, phenoxyl, benzoxyl, phenylethanonyl, fluorenyl, indanyl and 1,2,3,4-tetrahydro-naphthyl. Carbon atoms present within a carbocyclic ring may, of course, be further bonded to zero, one or two hydrogen atoms and/or any of a variety of ring substituents, such as hydroxy, halogen, cyano, nitro, C₁-C₈alkyl, C₂-C₈alkenyl, C₂-Calkynyl, C₁-C₈alkoxy, C₂-C₈alkyl ether, C₃-C₈alkanone, C₁-C₈alkylthio, amino, mono- or di-(C₁-C₈alkyl)amino, C₃-C₇cycloalkylC₀-C₄alkyl, haloC₁-C₈alkyl, haloC₁-C₈alkoxy, aminoC₁-C₈alkyl, hydroxyC₁-C₈alkyl, C₂-C₈alkanoyl, C₂-C₈alkoxycarbonyl, —COOH, —C(═O)NH₂, mono- or di-(C₁-C₈alkyl)carboxamido, —S(O₂)NH₂, and/or mono- or di-(C₁-C₈alkyl)sulfonamido.

Certain carbocycles recited herein include C₆-C₁₀arylC₀-C₈alkyl groups (i.e., groups in which a carbocyclic group comprising at least one aromatic ring is linked via a direct bond or a C₁-C₈alkyl group). Such groups include, for example, phenyl and indanyl, as well as groups in which either of the foregoing is linked via C₁-C₈alkyl, preferably via C₁-C₄alkyl. Phenyl groups linked via a direct bond or alkyl group may be designated phenylC₀-C₈alkyl (e.g., benzyl, 1-phenyl-ethyl, 1-phenyl-propyl and 2-phenyl-ethyl).

A “heterocycle” or “heterocyclic group” has from 1 to 3 fused, pendant or spiro rings, at least one of which is a heterocyclic ring (i.e., one or more ring atoms is a heteroatom, with the remaining ring atoms being carbon). Typically, a heterocyclic ring comprises 1-4 heteroatoms; within certain embodiments each heterocyclic ring has 1 or 2 heteroatoms per ring. Each heterocyclic ring generally contains from 3 to 8 ring members (rings having from 5 to 7 ring members are recited in certain embodiments), and heterocycles comprising fused, pendant or spiro rings typically contain from 9 to 14 ring members. Heterocycles may be optionally substituted at nitrogen and/or carbon atoms with a variety of substituents, such as those described above for carbocycles. Unless otherwise specified, a heterocycle may be a heterocycloalkyl group (i.e., each ring is saturated or partially saturated) or a heteroaryl group (i.e., at least one ring within the group is aromatic). A heterocyclic group may generally be linked via any ring or substituent atom, provided that a stable compound results. N-linked heterocyclic groups are linked via a component nitrogen atom. A “heterocycleC₀-C₈alkyl” is a heterocyclic group linked via a direct bond or C₁-C₈alkyl group. Similarly, a “heterocycleC₂-C₈alkoxycarbonyl” is a heterocyclic group linked via a C₂-C₈alkoxycarbonyl group.

Certain heterocyclic groups are 3- to 10-membered or 5- to 10-membered groups that contain 1 heterocyclic ring or 2 fused or spiro rings, optionally substituted as described above. (C₃-C₁₀)heterocycloalkyls include, for example, piperidinyl, piperazinyl, pyrrolidinyl, azepanyl, 1,4-dioxa-8-aza-spiro[4.5]dec-8-yl, morpholino, thiomorpholino, and 1,1-dioxo-thiomorpholin-4-yl, as well as groups in which each of the foregoing is substituted with from 1 to 6 (preferably from 1 to 4) substituents independently selected from halogen, hydroxy, C₁-C₄alkyl, C₁-C₄alkoxy, haloC₁-C₄alkyl, haloC₁-C₄alkoxy, C₂-C₄alkanoyl and C₂-C₄alkoxycarbonyl. In certain embodiments, a heterocycloalkyl may be a 4- to 7-membered heterocycloalkylC₀-C₄alkyl group. Such groups comprise a 4- to 7-membered heterocycloalkyl group as described above, linked via a direct bond or a C₁-C₄ alkyl group.

Certain aromatic heterocycles include 5- to 10-membered heteroarylC₀-C₈alkyl groups (i.e., groups in which the heterocyclic group comprising at least one aromatic ring is linked via a direct bond or a C₁-C₈alkyl group). Such groups include, for example, the heteroaryl groups recited above, as well as groups in which any of the foregoing is linked via C₁-C₈alkyl, C₁-C₆alkyl or C₁-C₄alkyl. Representative aromatic heterocycles are azocinyl, pyridyl, pyrimidyl, imidazolyl, tetrazolyl and 3,4-dihydro-1H-isoquinolin-2-yl, as well as groups in which each of the foregoing is linked via C₁-C₄alkyl.

A “substituent,” as used herein, refers to a molecular moiety that is covalently bonded to an atom within a molecule of interest. For example, a “ring substituent” may be a moiety such as a halogen, alkyl group, haloalkyl group or other group discussed herein that is covalently bonded to an atom (preferably a carbon or nitrogen atom) that is a ring member. The term “substitution” refers to replacing a hydrogen atom in a molecular structure with a substituent as described above, such that the valence on the designated atom is not exceeded, and such that a chemically stable compound (i.e., a compound that can be isolated, characterized, and tested for biological activity) results from the substitution.

Groups that are “optionally substituted” are unsubstituted or are substituted by other than hydrogen at one or more available positions, typically 1, 2, 3, 4 or 5 positions, by one or more suitable groups (which may be the same or different). Such optional substituents include, for example, hydroxy, halogen, cyano, nitro, C₁-C₈alkyl, C₂-C₈alkenyl, C₂-C₈alkynyl, C₁-C₈alkoxy, C₂-C₈alkyl ether, C₃-C₈alkanone, C₁-C₈alkylthio, amino, mono- or di-(C₁-C₈alkyl)amino, haloC₁-C₈alkyl, haloC₁-C₈alkoxy, C₂-C₈alkanoyl, C₂-C₈alkanoyloxy, C₂-C₈alkoxycarbonyl, —COOH, —CONH₂, mono- or di-(C₁-C₈alkyl)carboxamido, —SO₂NH₂, and/or mono or di(C₁-C₈alkyl)sulfonamido, as well as carbocyclic and heterocyclic groups. Certain optionally substituted groups are substituted with from 0 to 3 independently selected substituents.

The terms “VR1,” “type 1 vanilloid receptor” and “capsaicin receptor” are used interchangeably herein. Unless otherwise specified, these terms encompass both rat and human VR1 receptors (e.g., GenBank Accession Numbers AF327067, AJ277028 and NM_{—}018727; sequences of certain human VR1 cDNAs are provided in SEQ ID NOs: 1-3, and the encoded amino acid sequences shown in SEQ ID NOs:4 and 5, of U.S. Pat. No. 6,482,611), as well as homologs thereof found in other species.

Compounds of Formula I. Certain compounds of Formula I are disclosed in pending U.S. patent application Ser. No. 09/910,442, entitled “Capsaicin Receptor Ligands,” filed Jul. 20, 2001 in the name of Rajagopal Bakthavatchalam et al. The corresponding PCT application published as WO 02/08221 on Jan. 31, 2002, is incorporated herein by reference for its teaching of specific compounds of Formula I and methods for preparing the same (pages 8-31, 40-47 and 54-106).

Compounds of Formulas II-IV. Certain compounds of Formulas II-IV are disclosed in PCT International Application No. WO 03/062209, which published on Jul. 31, 2003, and which is incorporated herein by reference for its teaching of substituted quinazolin-4-ylamine analogue VR1 antagonists and methods for preparing the same (pages 3-29, 30-40 and 50-79); and in pending U.S. patent application Ser. No. 10/347,210, entitled “Substituted Quinazolin-4-ylamine Analogues,” filed Jan. 17, 2003 in the name of Rajagopal Bakthavatchalam et al., which is incorporated herein by reference for its teaching of substituted quinazolin-4-ylamine analogue VR1 antagonists and methods for preparing the same (pages 4-8, 28-34, 39-53 and 69-239).

Substituted quinazolin-4-ylamine analogues of Formulas II-IV may generally be prepared using standard synthetic methods. In general, starting materials are commercially available from suppliers such as Sigma-Aldrich Corp. (St. Louis, Mo.), or may be synthesized from commercially available precursors using established protocols. By way of example, a synthetic route similar to that shown in any of Schemes II:1-13, III:1-7 and IV:1-10 may be used, together with synthetic methods known in the art of synthetic organic chemistry, or variations thereon as appreciated by those skilled in the art. “R,” in the following schemes, refers to any group consistent with the description of the compounds provided herein.

In the Schemes that follow, the term “catalyst” refers to a suitable transition metal catalyst such as, but not limited to, tetrakis(triphenylphosphine)palladium(0) or palladium(II) acetate. In addition, the catalytic systems may include ligands such as, but not limited to, 2-(Dicyclohexylphosphino)biphenyl and tri-tert-butylphosphine, and may also include a base such as K₃PO₄, Na₂CO₃ or sodium or potassium tert-butoxide. Transition metal-catalyzed reactions can be carried out at ambient or elevated temperatures using various inert solvents including, but not limited to, toluene, dioxane, DMF, N-methylpyrrolidinone, ethyleneglycol dimethyl ether, diglyme and acetonitrile. When used in conjunction with suitable metallo-aryl reagents, transition metal-catalyzed (hetero)aryl-aryl coupling reactions can be used to prepare the certain compounds shown in the following Schemes. Commonly employed reagent/catalyst pairs include aryl boronic acid/palladium(0) (Suzuki reaction; Miyaura and Suzuki (1995) Chemical Reviews 95:2457) and aryl trialkylstannane/palladium(0) (Stille reaction; T. N. Mitchell, Synthesis (1992) 803), arylzinc/palladium(0) and aryl Grignard/nickel(II).

The term “reduce” refers to the process of reducing a nitro functionality to an amino functionality. This transformation can be carried out in a number of ways well known to those skilled in the art of organic synthesis including, but not limited to, catalytic hydrogenation, reduction with SnCl₂ and reduction with titanium trichloride. For an overview of reduction methods see: Hudlicky, M. Reductions in Organic Chemistry, ACS Monograph 188, 1996.

The term “activate” refers to a synthetic transformation in which a carbonyl of an amide moiety is converted to a suitable leaving group. Reagents suitable for carrying out this transformation are well known to those skilled in the art of organic synthesis and include, but are not limited to, SOCl₂, POCl₃ and triflic anhydride.

The term “deprotection” generally refers to the process of liberating a functional group which had previously been protected with a blocking or masking agent. For an overview of protection and deprotection methods as used by those skilled in the art of organic synthesis, see: Greene, T. and Wuts, P. Protective Groups in Organic Synthesis, 3rd ed., John Wiley and Sons, 1999. In the Schemes that follow, “deprotection” refers to, for example, the process of cleaving the C—O bond of a benzylic ether to give a “deprotected” alcohol using various methods familiar to those who are skilled in the art of organic synthesis. Methods to effect this transformation include, but are not limited to, hydrogenolysis using hydrogen gas and an appropriate catalyst system such as palladium on carbon or Raney nickel. Deprotection conditions for other protected functional groups such as amines, carboxylates, and the like are well known to those skilled in the art.

The term “hydrolyze” refers to the conversion of a nitrile functionality to an amide functionality by reaction with water. The reaction with water can be catalyzed by a variety of acids or bases well known to those skilled in the art of organic synthesis.

The term “diazotize” refers to the synthetic transformation of an amino (—NH₂) to a diazonium salt (—N₂⁺) functionality. This transformation can be carried out in a variety of ways familiar to those skilled in the art of organic synthesis including, but not limited to, treatment with a mixture of nitrous acid (HNO₂) and sulfuric acid or a mixture of a nitrite salt (such as NaNO₂) in sulfuric acid.

The term “demethylation” refers to the cleavage of the Me-O bond in a methyl ether functionality. This transformation can be carried out in a variety of ways familiar to those skilled in the art of organic synthesis including, but not limited to, treatment with HBr, treatment with Lewis acid/nucleophile combinations, Trimethylsilyl iodide, etc.

The term “oxidize” refers to a synthetic transformation wherein a methyl group is converted to a carboxylic acid functionality. Various reagents familiar to those skilled in the art of organic synthesis may be used to carry out this transformation including, but not limited to, KMnO₄ in basic media (e.g., NaOH solution or aqueous pyridine) and K₂Cr₂O₇ in acidic media (e.g., H₂SO₄).

The term “cyclize” refers to a synthetic transformation in which ortho-amino-benzoic acids, ortho-amino-benzoic esters, and ortho-amino-benzonitriles are converted to the corresponding 3H-Quinazolin-4-ones: Methods for effecting the cyclization of ortho-amino-benzonitriles include, but are not limited to, reaction with refluxing formic acid containing sodium acetate. Methods for effecting the cyclization of ortho-amino-benzoic acids include, but are not limited to, reaction with formamide at elevated temperatures or reaction with formamidine acetate in an inert solvent, also at elevated temperatures. Methods for effecting the cyclization of ortho-amino-benzoic esters include, but are not limited to, reaction with formamidine acetate at elevated temperature in an inert solvent.

In Scheme 8, “H₂N-Prot” refers to a protected amino functionality, such as 4-methoxybenzylamine, and “deprotect” refers to a chemical method by which such a protecting group can be removed. For an overview of protection and deprotection methods as used by those skilled in the art of organic synthesis, see: Greene, T. and Wuts, P. Protective Groups in Organic Synthesis, 3rd ed., John Wiley and Sons, 1999.

In Scheme 9, the term “nucleophile” refers to a primary or secondary amine, or an alkoxide.

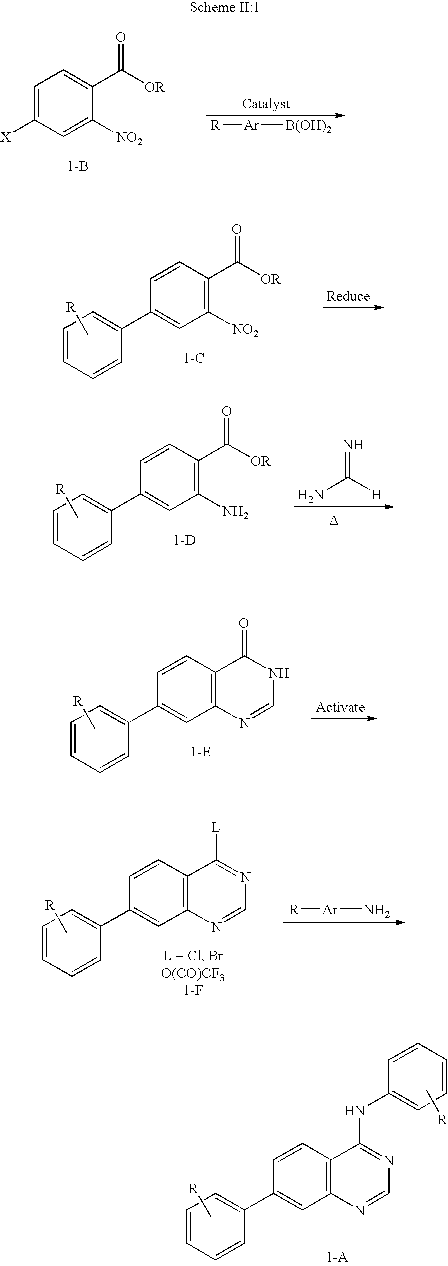

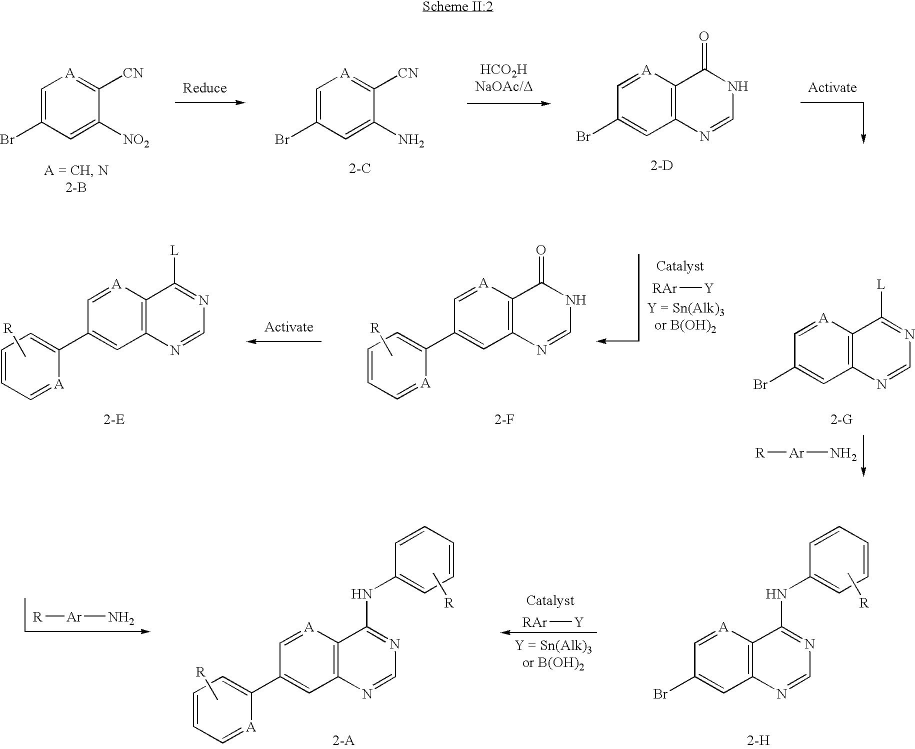

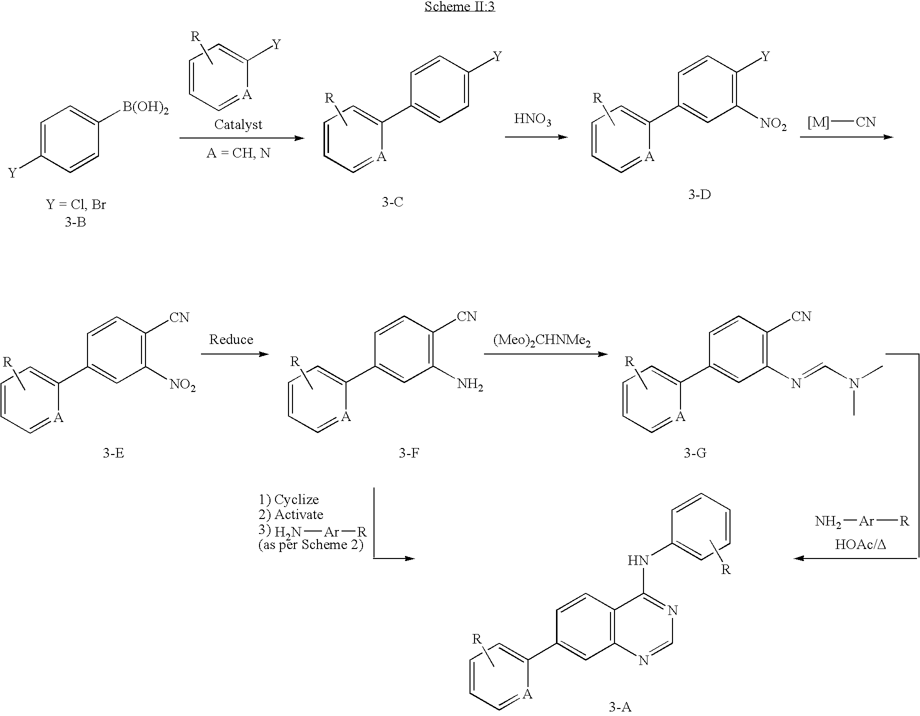

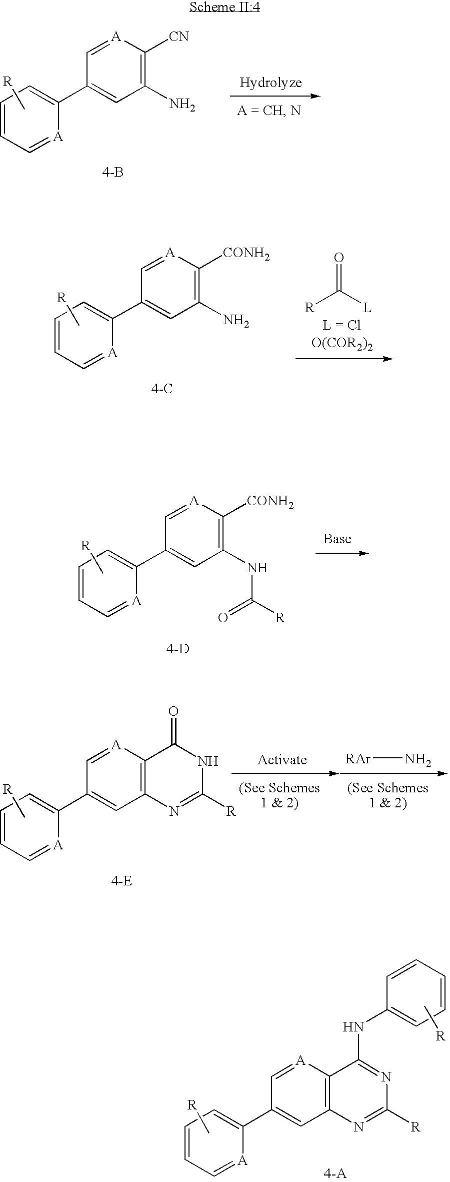

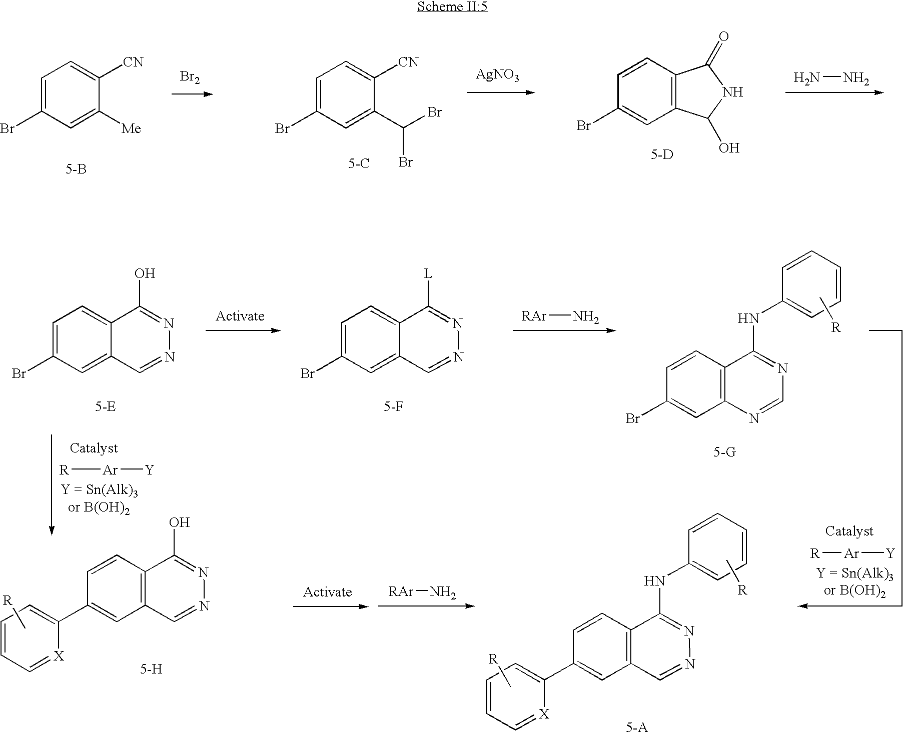

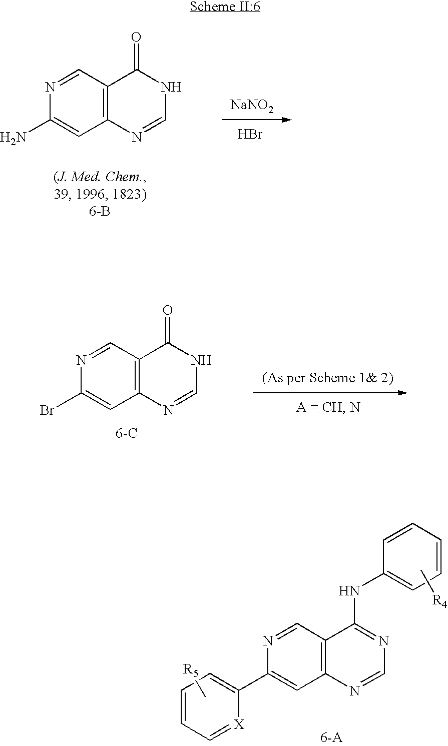

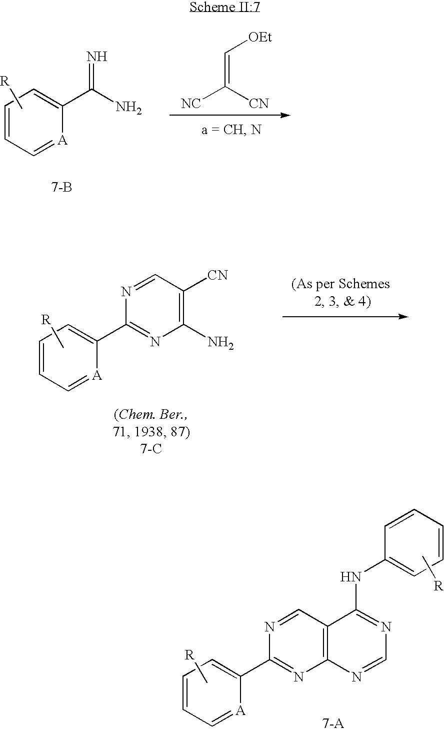

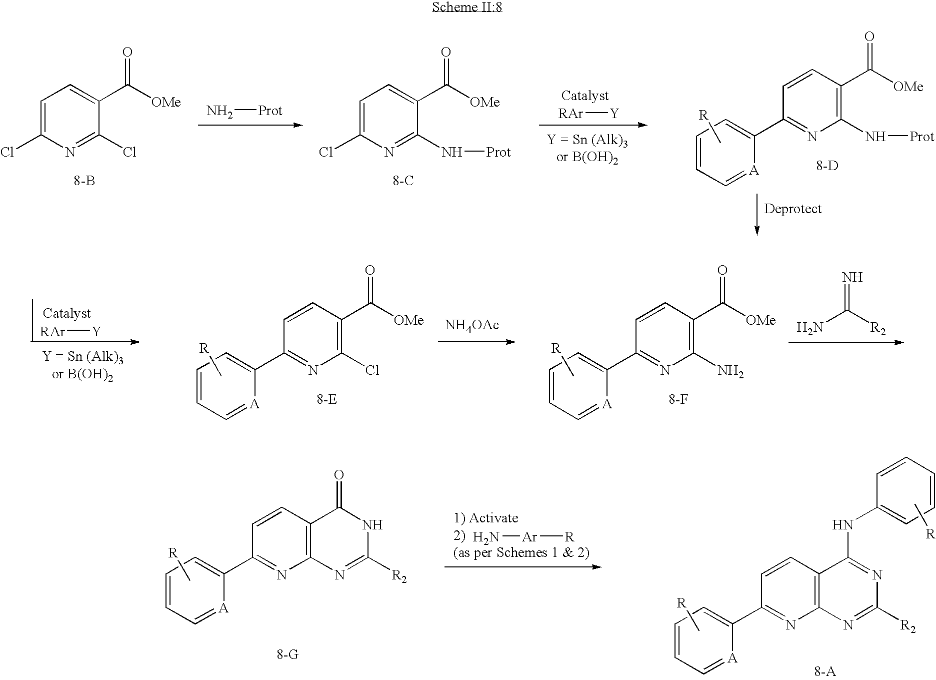

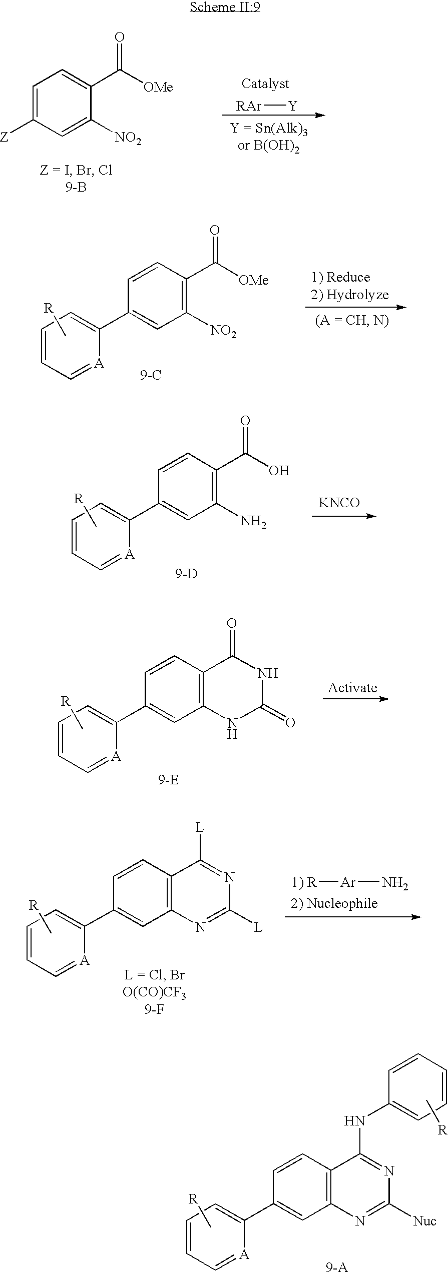

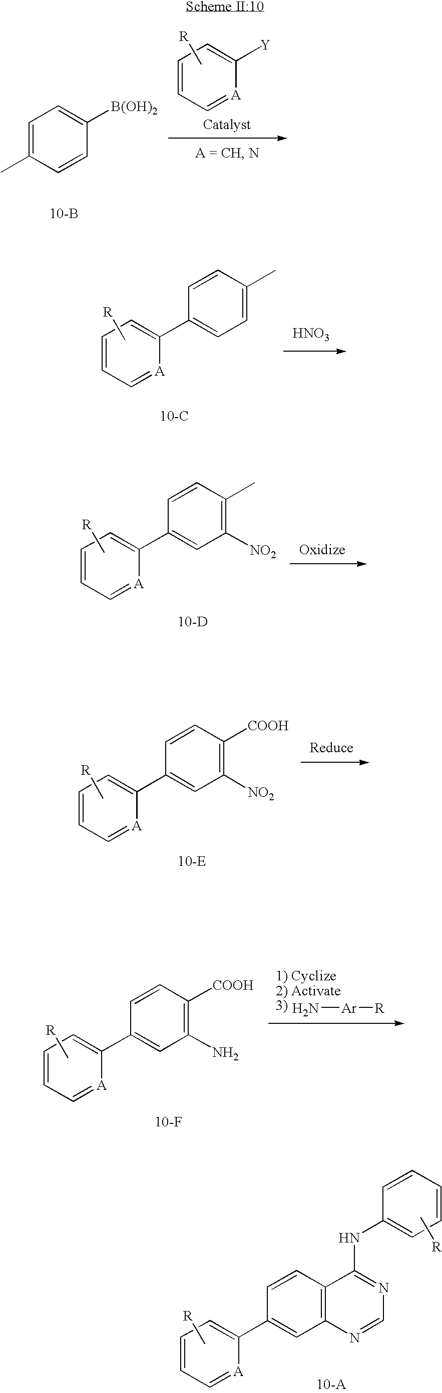

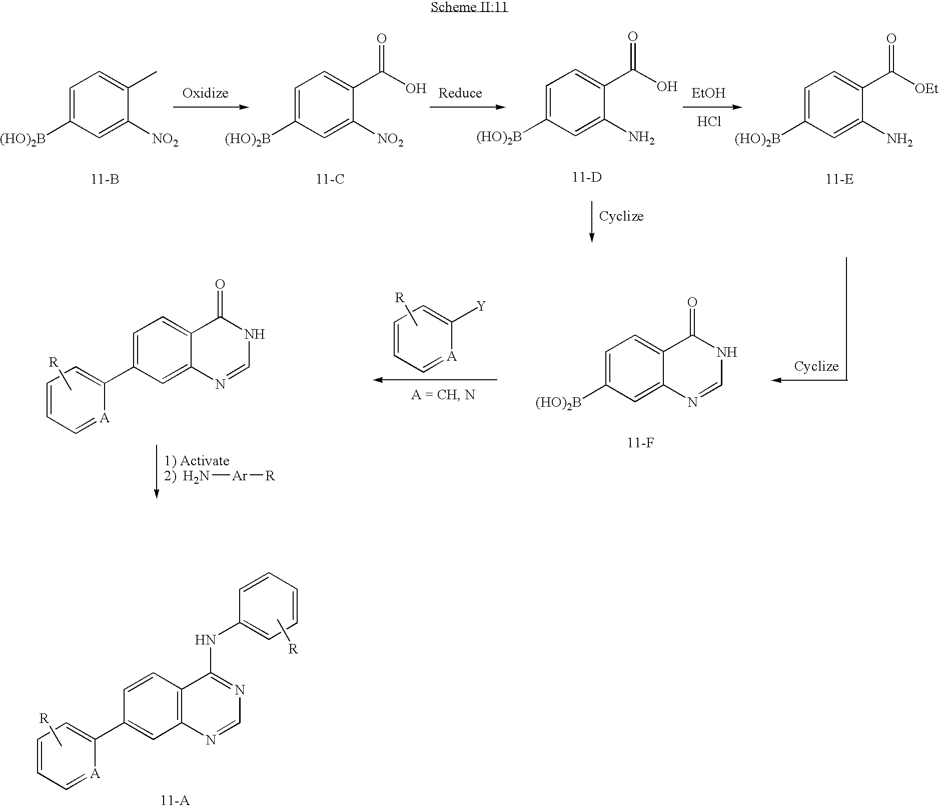

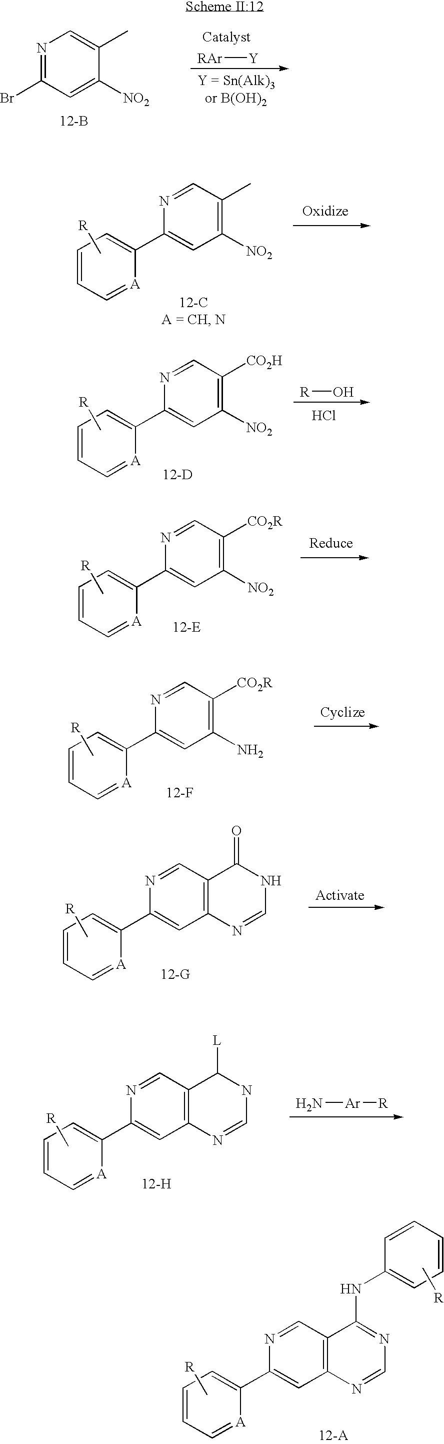

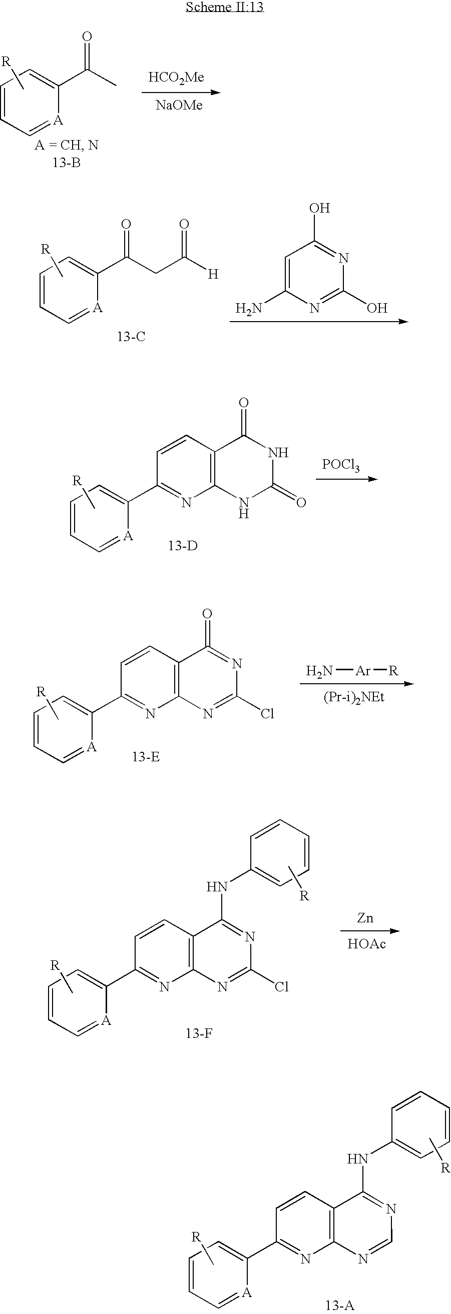

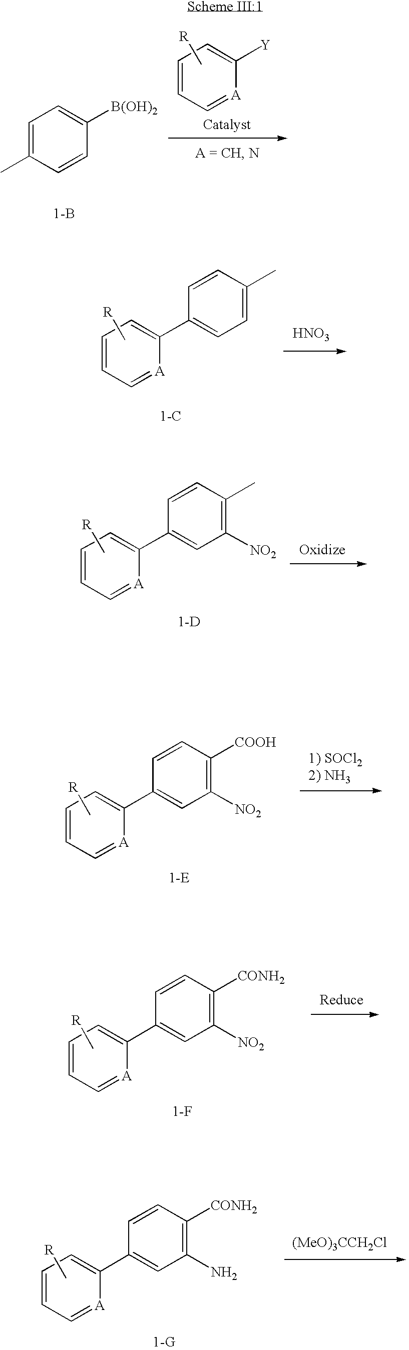

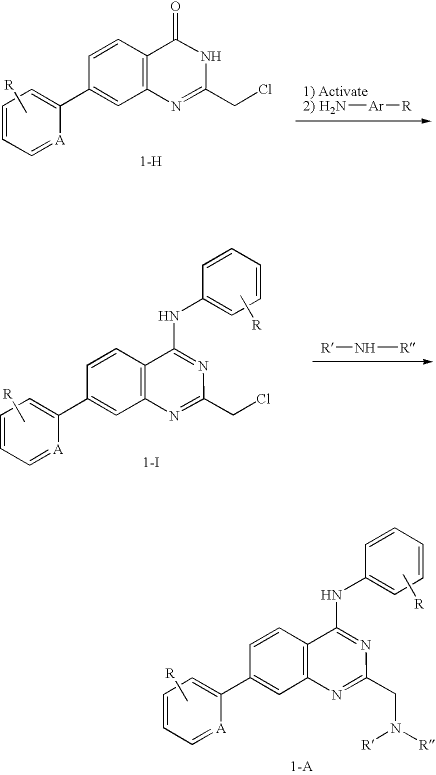

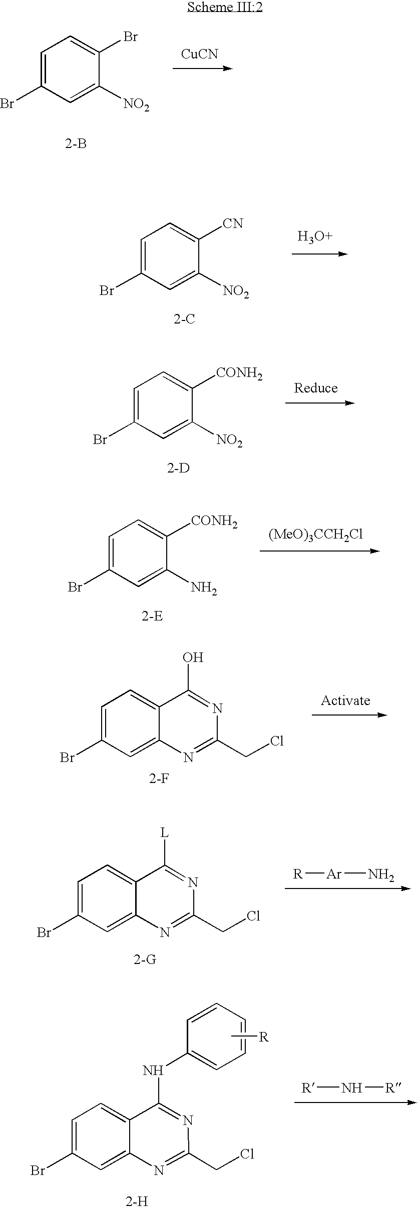

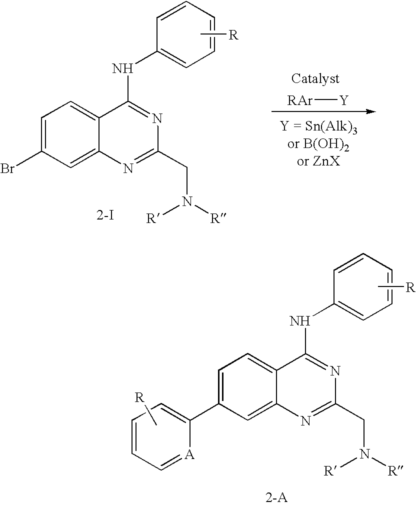

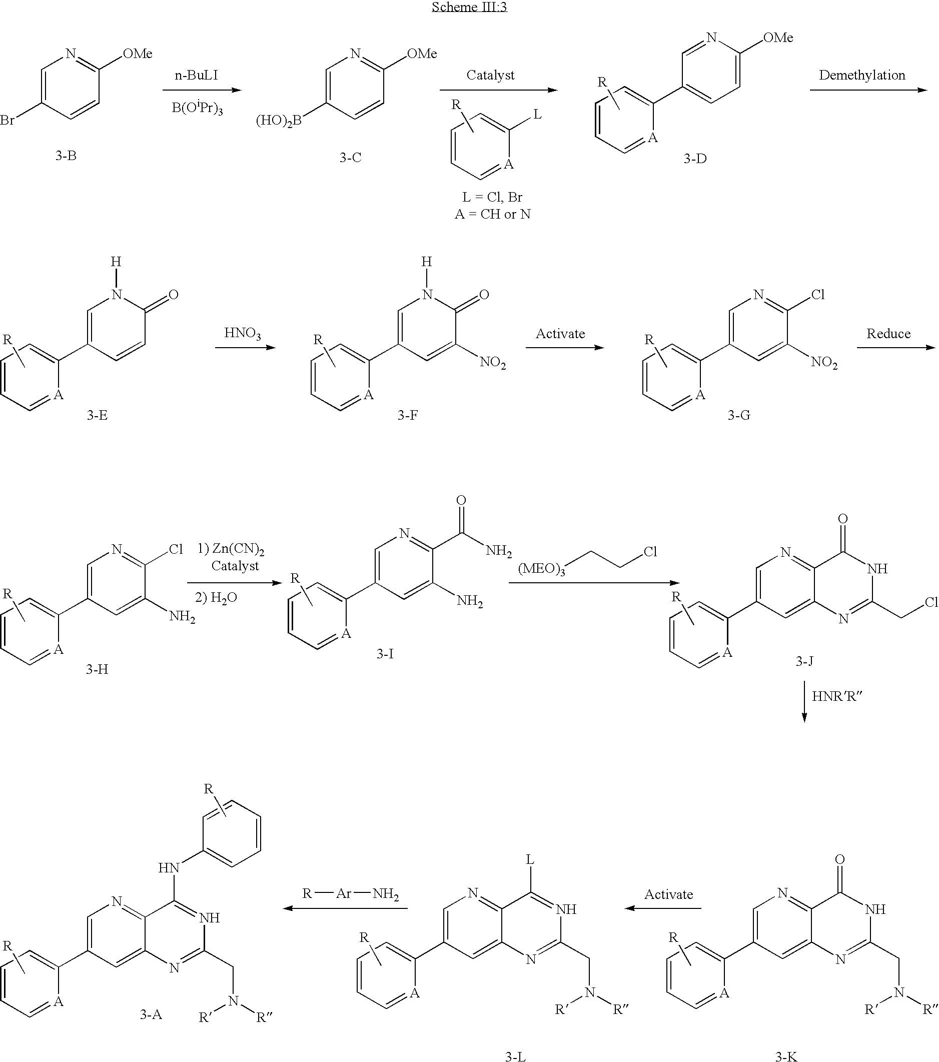

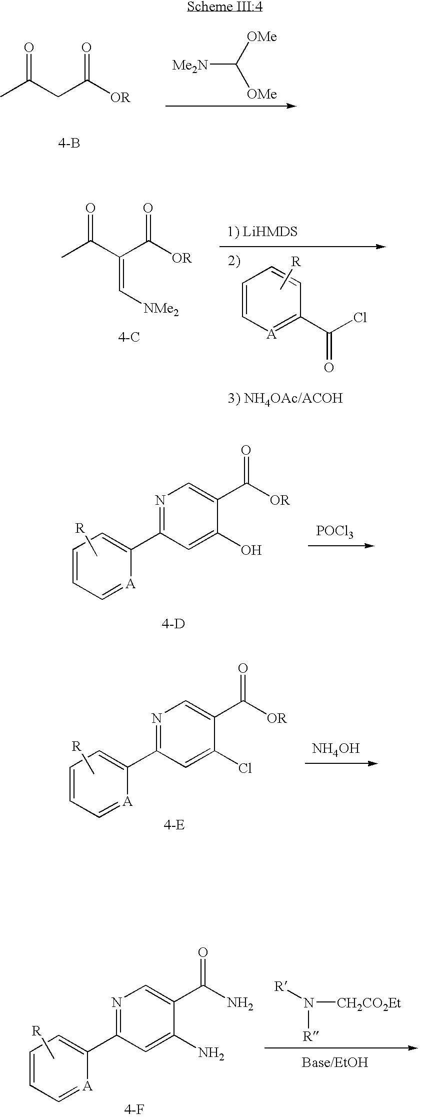

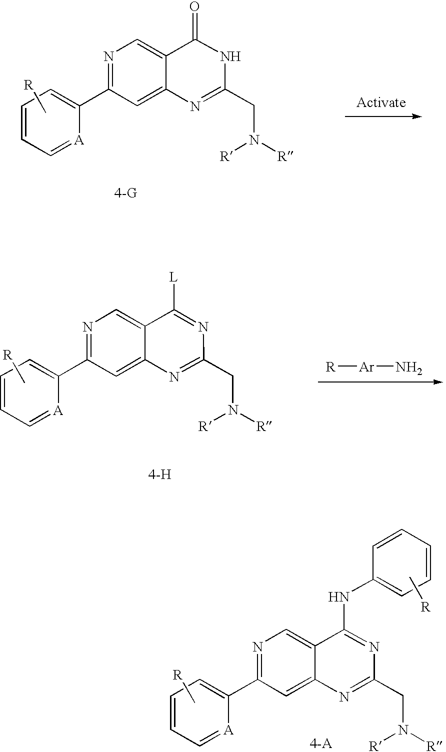

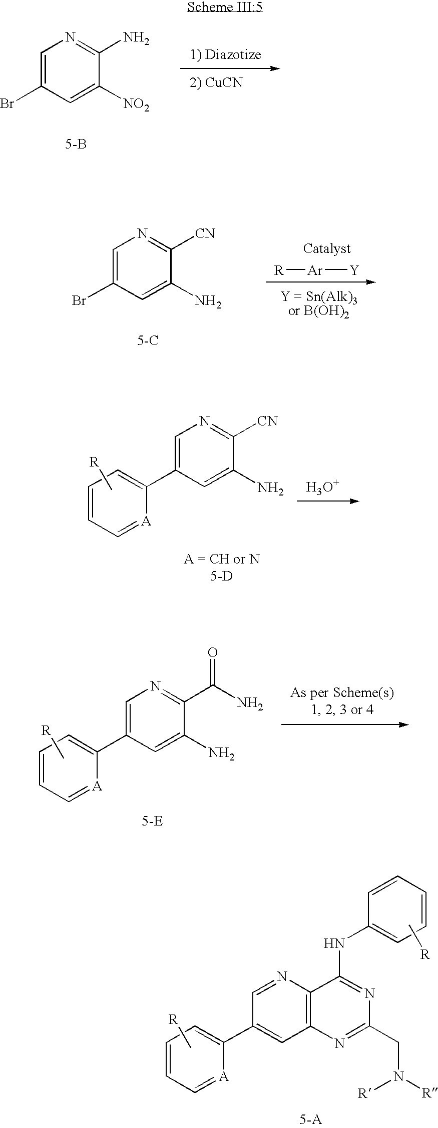

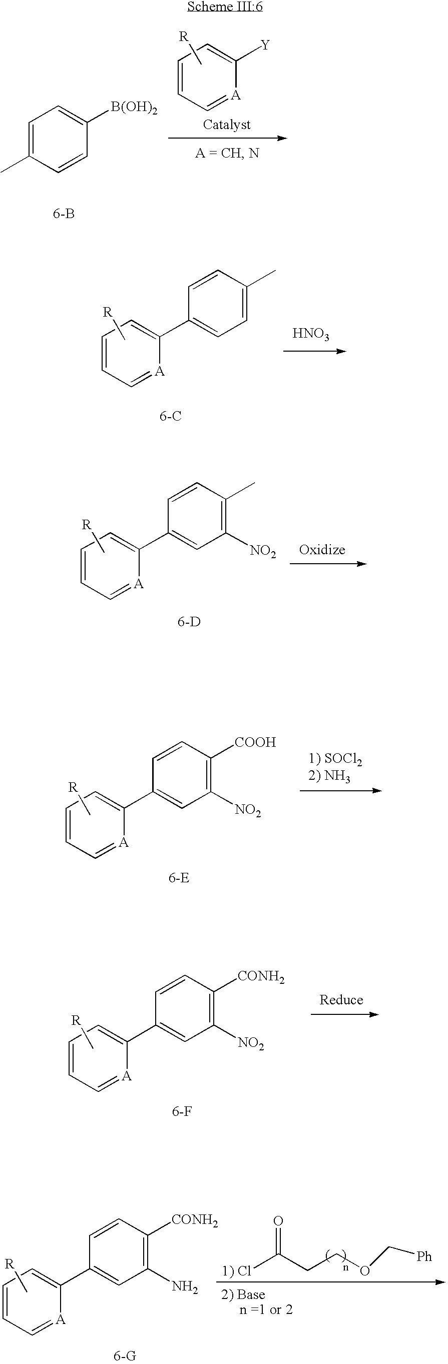

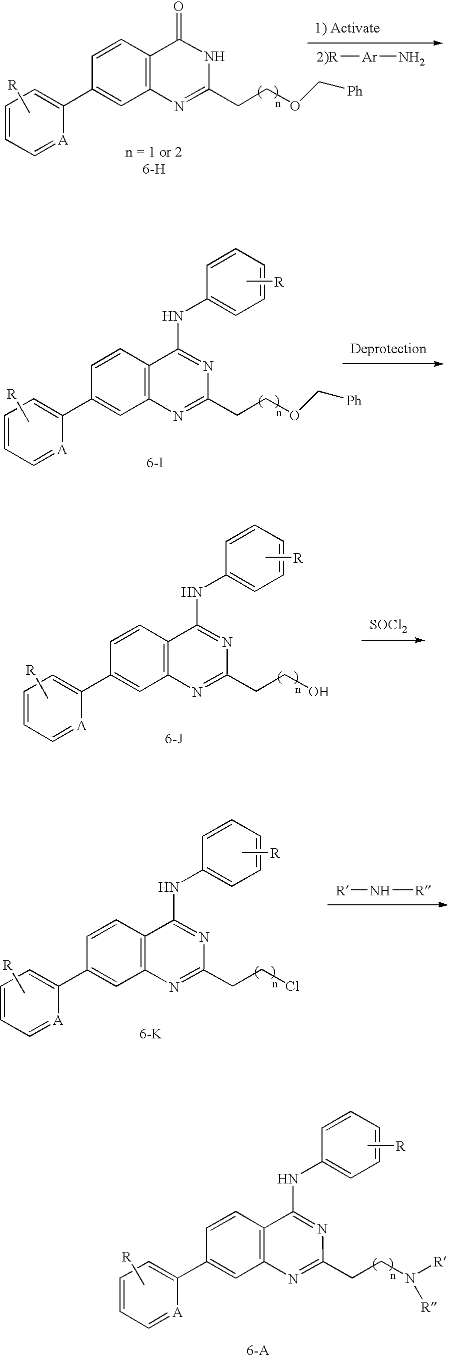

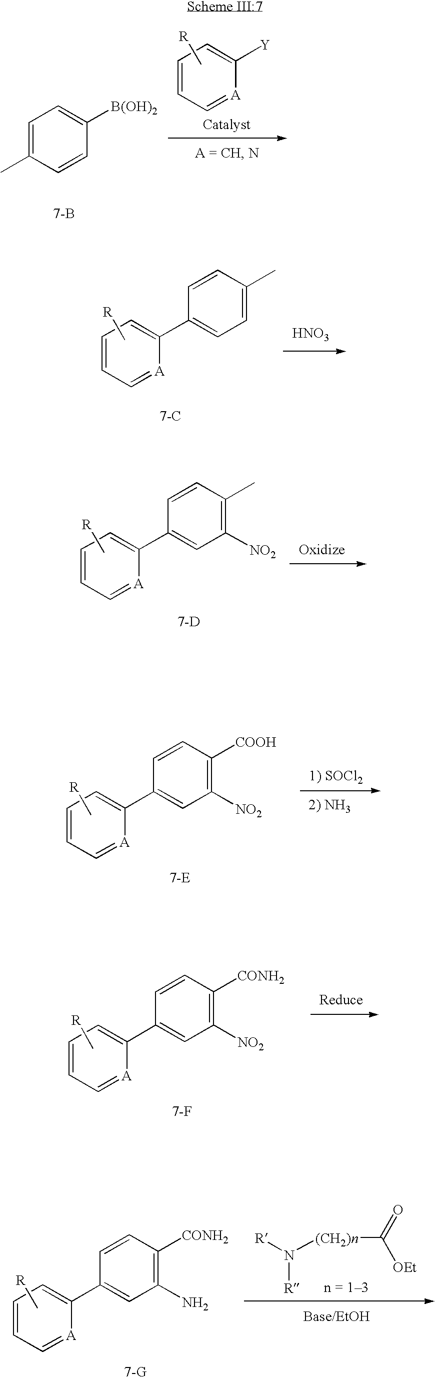

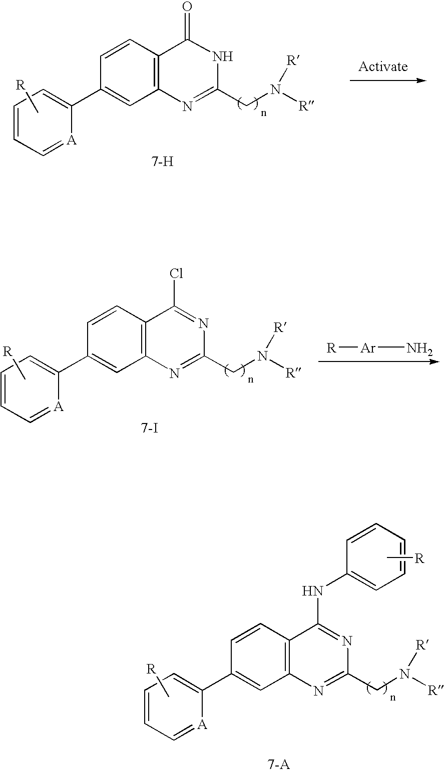

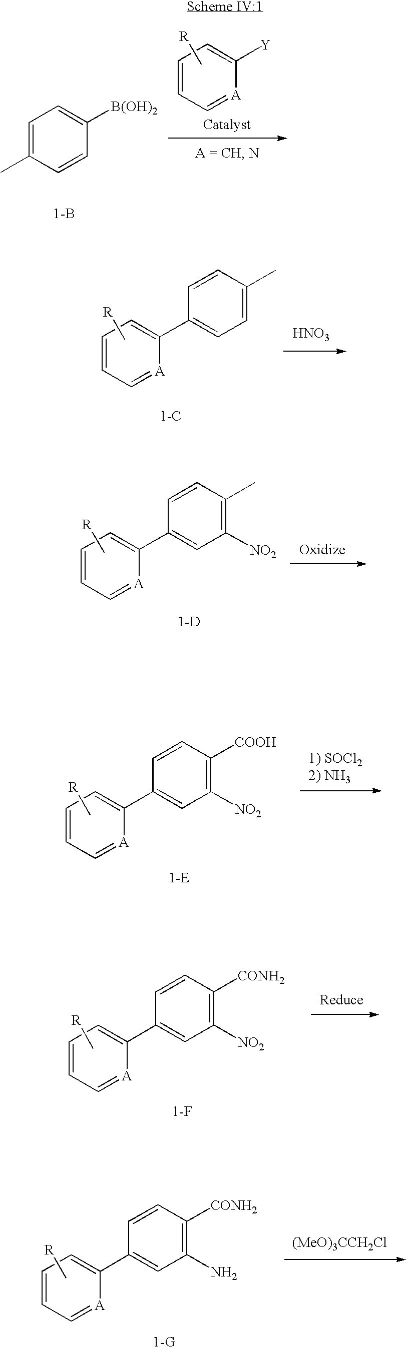

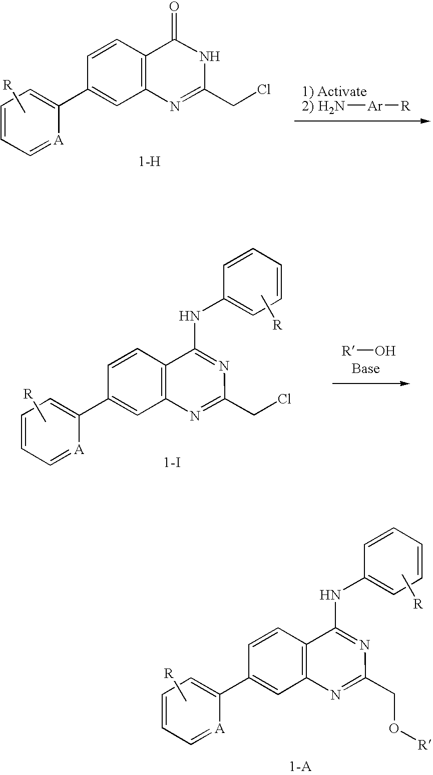

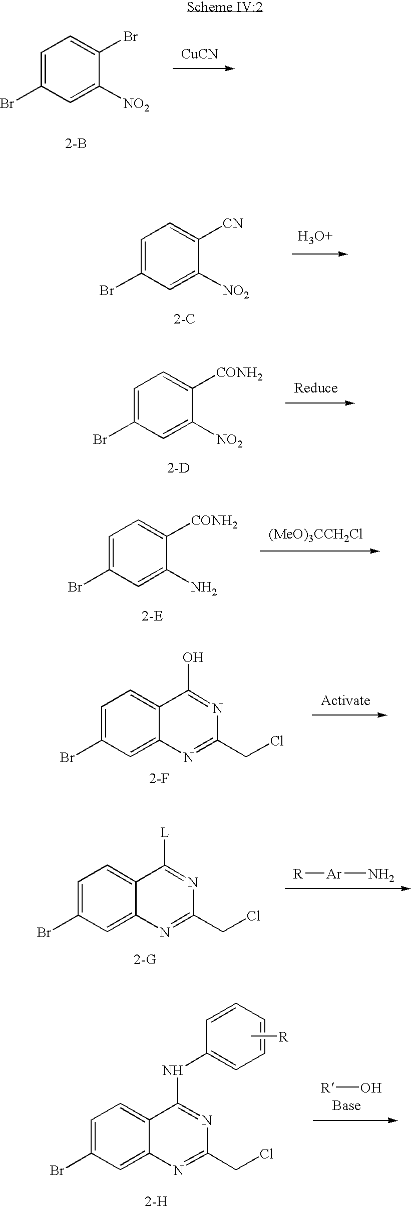

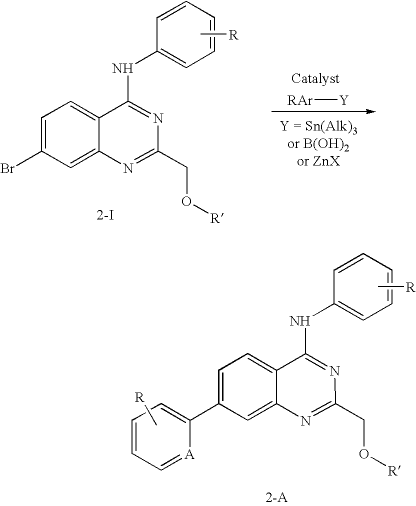

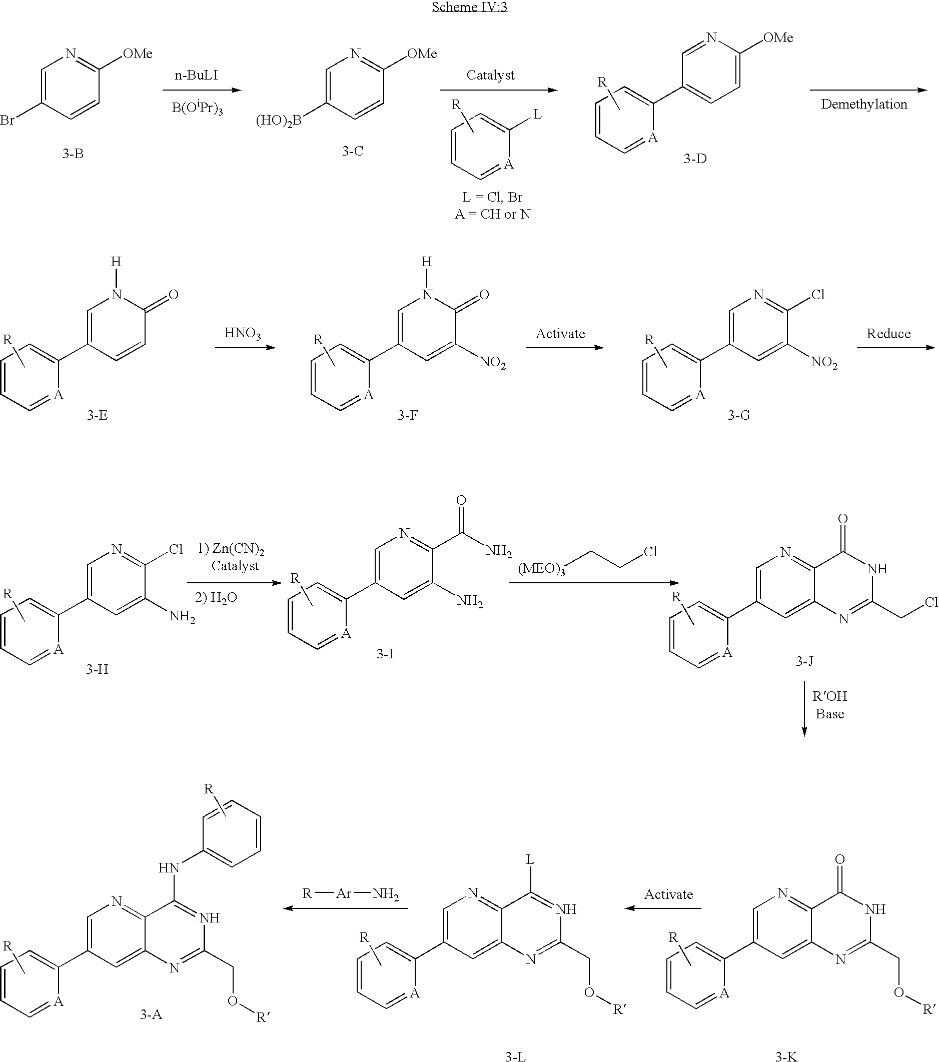

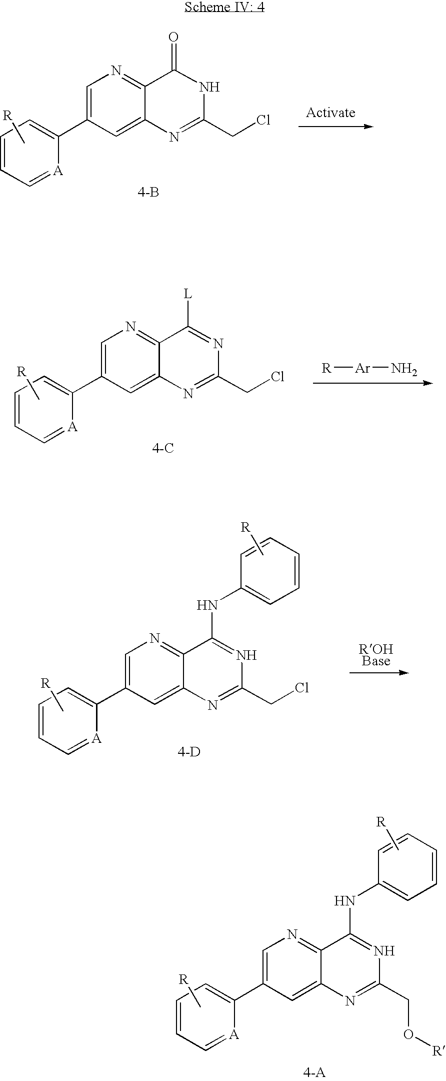

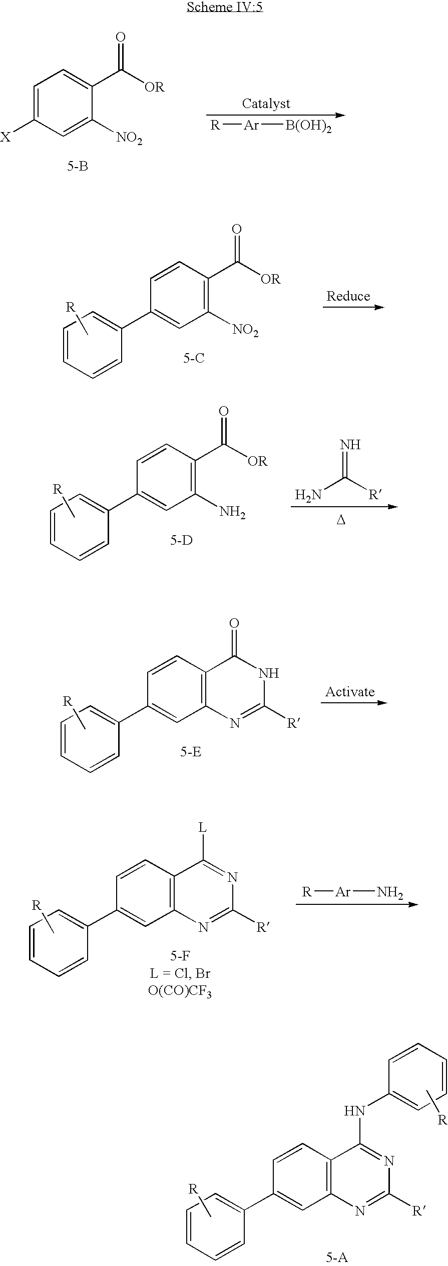

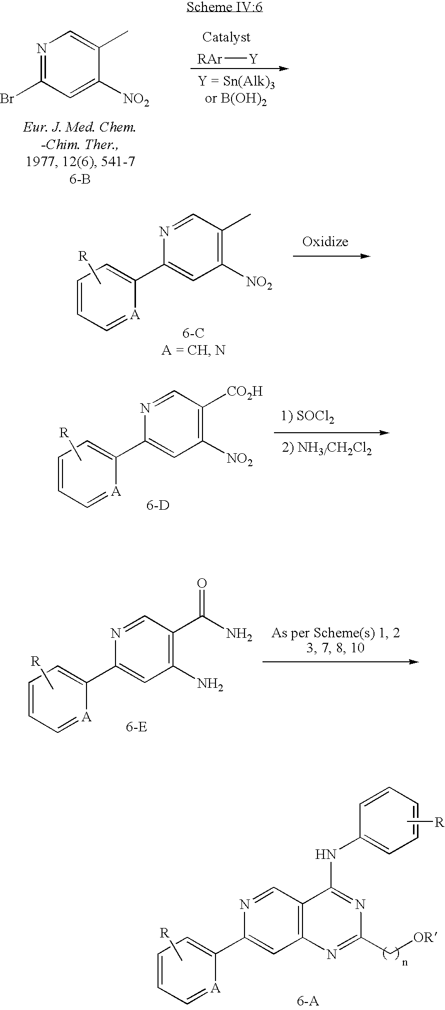

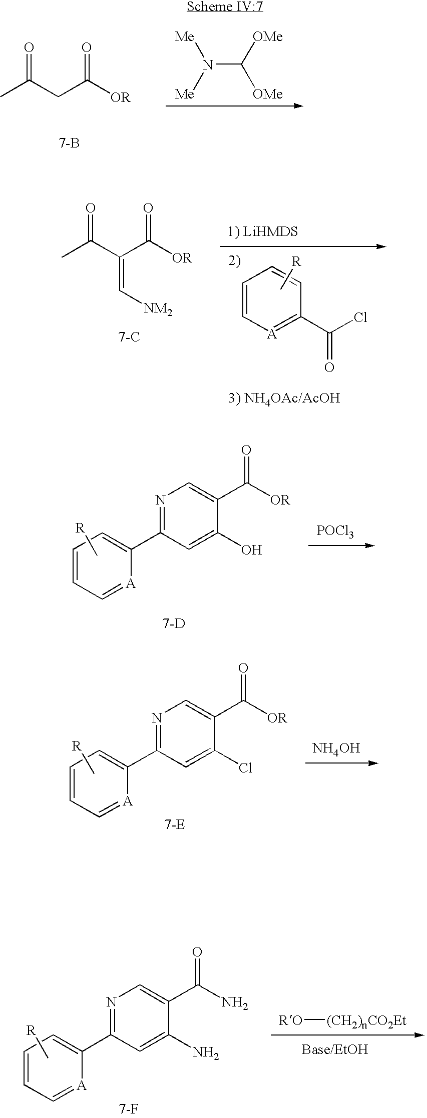

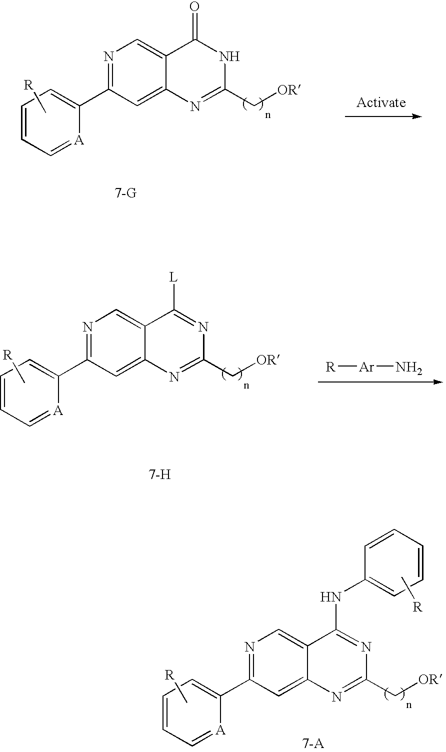

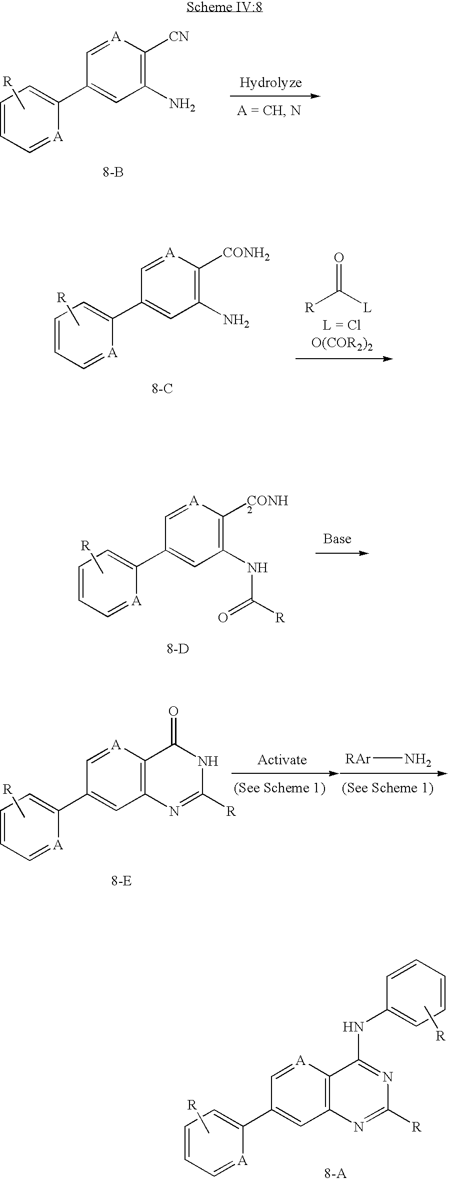

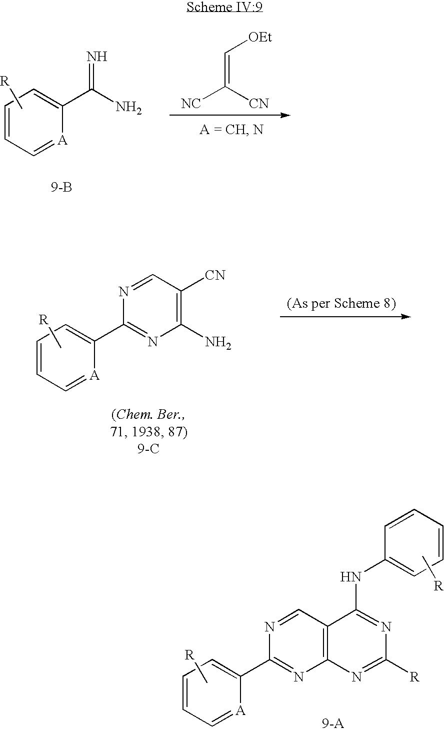

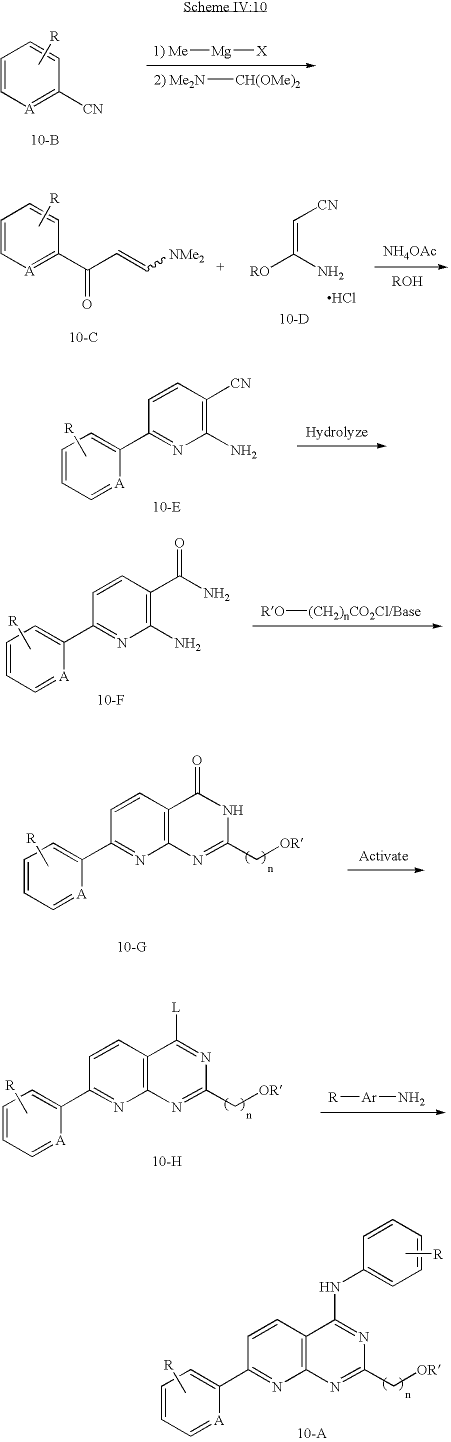

The following Examples are offered by way of illustration and not by way of limitation. Unless otherwise specified all reagents and solvent are of standard commercial grade and are used without further purification.

### EXAMPLES

### Example 1

### Preparation of Representative VR1 Receptor Antagonists

The following VR1 antagonists of Formula I are prepared as described in PCT International Application Publication Number WO 02/08221, which published on Jan. 31, 2002. Such VR1 antagonists may be used in the compositions and methods provided herein.

(R)-4-(3-Chloro-pyridin-2-yl)-2-methyl-piperazine-1-carboxylic acid (4-sec-butyl-phenyl)-amide;

(R)-(−)-4-(3-Chloro-pyridin-2-yl)-2-methyl-piperazine-1-carboxylic acid (4-trifluoromethyl-phenyl)-amide;

(R)-3-Chloro-pyridin-2-yl)-2-methyl-piperazine-1-carboxylic acid 4-tert-butyl-phenyl ester;

2-Methyl-4-(3-trifluoromethyl-pyridin-2-yl)-piperazine-1-carboxylic acid [4-(2,2,2-trifluoro-1-methyl-ethyl)-phenyl]-amide;

4-(3-Chloro-pyridin-2-yl)-2-methyl-piperazine-1-carboxylic acid [4-(2,2,2-trifluoro-1-methyl-ethyl)-phenyl]-amide;

(R)-4-(3-Chloro-pyridin-2-yl)-2-methyl-piperazine-1-carboxylic acid [4-(1,2,2,2-tetrafluoro-1-trifluoromethyl-ethyl)-phenyl]-amide;

4-(3-Chloro-pyridin-2-yl)-2-methyl-piperazine-1-carbothioic acid (4-isopropyl-phenyl)-amide;

4-(3-Trifluoromethyl-2-pyridinyl)-N-(3-methoxy-4-hydroxyphenylmethyl)-1-piperazine carboxamide;

4-(3-Nitro-2-pyridinyl)-N-[4-(n-butyl)phenyl]-1-piperazinecarboxamide;

4-(3-Trifluoromethyl-2-pyridinyl)-N-[4-(n-butyl)phenyl]-1-piperazinecarboxamide;

4-(3-Methyl-2-pyridinyl)-N-[4-(isopropyl)phenyl]-1-Piperazinecarboxamide;

4-(3-Methyl-2-pyridinyl)-N-[4-(n-butyl)phenyl]-1-piperazinecarboxamide;

4-(3-Trifluoromethyl-2-pyridinyl)-N-[4-(isopropyl)phenyl]-1-piperazinecarboxamide;

4-(3-Chloro-5-trifluoromethyl-2-pyridinyl)-N-[4-(isopropyl)phenyl]-1-piperazinecarboxamide;

4-(3-Chloro-2-pyridinyl)-N-[4-(isopropyl)phenyl]-1-piperazinecarboxamide;

4-(3,5-Dichloro-2-pyridinyl)-N-[4-(isopropyl)phenyl]-1-piperazinecarboxamide;

1-(3-Methyl-2-pyridinyl)-3-(4-trifluoromethyl phenyl)-prop-2-en-1-one;

1-(3-Trifluoromethyl-2-pyridinyl)-3-(4-isopropylphenyl)-prop-2-en-1-one;

4-(3-Cyano-2-pyridinyl)-N-[4-(isopropyl)phenyl]-1-piperazinecarboxamide;

4-(3-Chloro-2-pyridinyl)-N-[4-(isopropyl)phenyl]-2-methyl-1-piperazinecarboxamide;

4-(3-Chloro-2-pyridinyl)-N-[4-(isopropyl)phenyl]-1-piperazinecarboxamide;

4-(3-Chloro-2-pyridinyl)-N-[4-(isopropyl)phenyl]-2-methylthio-1-piperazinecarboxamide;

4-(3,5-Dichloro-2-pyridinyl)-N-[4-(isopropyl)phenyl]-1-piperazinecarboxamide;

N-(4-tert-butylphenyl)-4-(3-chloropyridin-2-yl)piperazine-1-carboxamide;

(2R)-4-(3-chloropyridin-2-yl)-N-(4-cyclohexylphenyl)-2-methylpiperazine-1-carboxamide;

(2R)-4-(3-chloropyridin-2-yl)-N-[2-chloro4-(trifluoromethyl)phenyl]-2-methylpiperazine-1-carboxamide;

(2R)-4-(3-chloropyridin-2-yl)-2-methyl-N-[4-(trifluoromethyl)phenyl]piperazine-1-carboxamide;

(2R)-N-(4-tert-butylphenyl)-4-(3-chloropyridin-2-yl)-2-methylpiperazine-1-carboxamide;

(2R)-4-(3-chloropyridin-2-yl)-N-(4-isopropylphenyl)-2-methylpiperazine-1-carboxamide;

(2S)-4-(3-chloropyridin-2-yl)-N-(4-trifluoromethylphenyl)-2-methylpiperazine-1-carboxamide;

(2S)-N-(4-tert-butylphenyl)4-(3-chloropyridin-2-yl)-2-methylpiperazine-1-carboxamide;

(2S)-4-(3-chloropyridin-2-yl)-N-(4-isopropylphenyl)-2-methylpiperazine-1-carboxamide;

(2R)-4-(3-chloropyridin-2-yl)-2-methyl-N-(4-piperidin-1-ylphenyl)piperazine-1-carboxamide;

(2R)-4-(3-chloropyridin-2-yl)-N-[2-fluoro-4-(trifluoromethyl)phenyl]-2-methylpiperazine-1-carboxamide;

(2R)-2-methyl-N-[4-(trifluoromethyl)phenyl]-4-[3-(trifluoromethyl)pyridin-2-yl]piperazine-1-carboxamide;

(2R)-N-(4-tert-butylphenyl)-2-methyl-4-[3-(trifluoromethyl)pyridin-2-yl]piperazine-1-carboxamide;

(2R)-N-(4-isopropylphenyl)-2-methyl-4-[3-(trifluoromethyl)pyridin-2-yl]piperazine-1-carboxamide;

4-(3-chloropyridin-2-yl)-2,6-dimethyl-N-[4-(trifluoromethyl)phenyl]piperazine-1-carboxamide;

N-(4-tert-butylphenyl)-4-(3-chloropyridin-2-yl)-2,6-dimethylpiperazine-1-carboxamide;

4-(3-chloropyridin-2-yl)-N-(4-isopropylphenyl)-2,6-dimethylpiperazine-1-carboxamide;

(2R)-N-(4-cyclohexylphenyl)-2-methyl4-[3-(trifluoromethyl)pyridin-2-yl]piperazine-1-carboxamide;

4-(3 chloropyridin-2-yl)-N-(4-cyclohexylphenyl)-2,6-dimethylpiperazine-1-carboxamide;

(2R)-4-(3-chloropyridin-2-yl)-N-(4-cyclopentylphenyl)-2-methylpiperazine-1-carboxamide;

(2R)-N-(4-cyclopentylphenyl)-2-methyl-4-[3-(trifluoromethyl)pyridin-2-yl]piperazine-1-carboxamide;

(2R)-4-isoquinolin-1-yl-2-methyl-N-[4-(trifluoromethyl)phenyl]piperazine-1-carboxamide;

(2R)-N-(4-tert-butylphenyl)-4-isoquinolin-1-yl-2-methylpiperazine-1-carboxamide;

(2R)-N-(4-isopropylphenyl)-4-isoquinolin-1-yl-2-methylpiperazine-1-carboxamide;

(2R)-N-(4-cyclopentylphenyl)-4-isoquinolin-1-yl-2-methylpiperazine-1-carboxamide;

(2R)-N-(4-cyclohexylphenyl)-4-isoquinolin-1-yl-2-methylpiperazine-1-carboxamide;

(2R)-N-(4-tert-butylphenyl)-4-[3-(dimethylamino)pyridin-2-yl]-2-methylpiperazine-1-carboxamide;

(2R)-4-[3-(dimethylamino)pyridin-2-yl]-2-methyl-N-[4-(trifluoromethyl)phenyl]piperazine-1-carboxamide;

(2R)-N-(4-tert-butylphenyl)-4-(3-methoxypyridin-2-yl)-2-methylpiperazine-1-carboxamide;

(2R)-4-(3-methoxypyridin-2-yl)-2-methyl-N-[4-(trifluoromethyl)phenyl]piperazine-1-carboxamide;

(2R)-N-(4-cyclohexylphenyl)-4-(3-methoxypyridin-2-yl)-2-methylpiperazine-1-carboxamide;

(2R)-4-(3-chloropyridin-2-yl)-N-[4-(3,6-dihydro-2H-pyran-4-yl)phenyl]-2-methylpiperazine-1-carboxamide;

(2R)-4-(3-chloropyridin-2-yl)-2-methyl-N-(4-tetrahydro-2H-pyran-4-ylphenyl)piperazine-1-carboxamide;

(2R)-4-(3-chloropyridin-2-yl)-N-[4-(4-hydroxytetrahydro-2H-pyran-4-yl)phenyl]-2-methylpiperazine-1-carboxamide;

(2R)-N-[4-(4-hydroxytetrahydro-2H-pyran4-yl)phenyl]-2-methyl-4-[3-(trifluoromethyl)pyridin-2-yl]piperazine-1-carboxamide;

(2R)-4-(3-chloropyridin-2-yl)-2-methyl-N-[4-(2-methyl-1,3-thiazol-4-yl)phenyl]piperazine-1-carboxamide;

(2R)-4-(3-chloropyridin-2-yl)-N-[4-(2-ethyl-1,3-thiazol-4-yl)phenyl]-2-methylpiperazine-1-carboxamide;

(2R)-4-(3-chloropyridin-2-yl)-N-[4-(2-methoxy-1,1-dimethylethyl)phenyl]-2-methylpiperazine-1-carboxamide;

(2R)-N-[4-(2-methoxy-1,1-dimethylethyl)phenyl]-2-methyl-4-[3-(trifluoromethyl)pyridin-2-yl]piperazine-1-carboxamide;

(2R)-4-(3-chloropyridin-2-yl)-N-[4-(1-cyano-1-methylethyl)phenyl]-2-methylpiperazine-1-carboxamide;

(2R)-N-[4-(1-cyano-1-methylethyl)phenyl]-2-methyl-4-[3-(trifluoromethyl)pyridin-2-yl]piperazine-1-carboxamide;

N-(4-tert-butylphenyl)-4-(3-chloropyridin-2-yl)-2-ethylpiperazine-1-carboxamide;

4-(3-chloropyridin-2-yl)-2-ethyl-N-[4-(trifluoromethyl)phenyl]piperazine-1-carboxamide;

4-(3-chloropyridin-2-yl)-2-ethyl-N-(4-isopropylphenyl)piperazine-1-carboxamide;

N-(4-tert-butylphenyl)-2-ethyl4-[3-(trifluoromethyl)pyridin-2-yl]piperazine-1-carboxamide;

2-ethyl-N-[4-(trifluoromethyl)phenyl]-4-[3-(trifluoromethyl)pyridin-2-yl]piperazine-1-carboxamide;

2-ethyl-N-(4-isopropylphenyl)-4-[3-(trifluoromethyl)pyridin-2-yl]piperazine-1-carboxamide;

2-tert-butyl-N-(4-tert-butylphenyl)-4-(3-chloropyridin-2-yl)piperazine-1-carboxamide;

2-tert-butyl-4-(3-chloropyridin-2-yl)-N-[4-(trifluoromethyl)phenyl]piperazine-1-carboxamide;

2-tert-butyl-4-(3-chloropyridin-2-yl)-N-(4-isopropylphenyl)piperazine-1-carboxamide;

2-tert-butyl-N-(4-tert-butylphenyl)-4-[3-(trifluoromethyl)pyridin-2-yl]piperazine-1-carboxamide;

2-tert-butyl-N-[4-(trifluoromethyl)phenyl]4-[3-(trifluoromethyl)pyridin-2-y]piperazine-carboxamide;

N-(4-tert-butylphenyl)-4-(3-chloropyridin-2-yl)-2-isopropylpiperazine-1-carboxamide;

4-(3-chloropyridin-2-yl)-2-isopropyl-N-[4-(trifluoromethyl)phenyl]piperazine-1-carboxamide;

4-(3-chloropyridin-2-yl)-2-isopropyl-N-(4-isopropylphenyl)piperazine-1-carboxamide;

N-(4-tert-butylphenyl)-2-isopropyl4-[3-(trifluoromethyl)pyridin-2-yl]piperazine-1-carboxamide;

2-isopropyl-N-[4-(trifluoromethyl)phenyl]-4-[3-(trifluoromethyl)pyridin-2-yl]piperazine-1-carboxamide;

2-isopropyl-N-(4-isopropylphenyl)-4-[3-(trifluoromethyl)pyridin-2-yl]piperazine-1-carboxamide;

(2R)-4-(3-fluoropyridin-2-yl)-2-methyl-N-[4-(trifluoromethyl)phenyl]piperazine-1-carboxamide;

(2R)-N-(4-tert-butylphenyl)-4-(3-fluoropyridin-2-yl)-2-methylpiperazine-1-carboxamide;

(2R)-4-(3-fluoropyridin-2-yl)-N-(4-isopropylphenyl)-2-methylpiperazine-1-carboxamide;

(2R)-N-(4-cyclohexylphenyl)-4-(3-fluoropyridin-2-yl)-2-methylpiperazine-1-carboxamide;

(2R)-N-(4-cyclopentylphenyl)-4-(3-fluoropyridin-2-yl)-2-methylpiperazine-1-carboxamide;

N-(4-chlorophenyl)-4-(6-chloropyridin-2-yl)piperazine-1-carboxamide;

4-(6-chloropyridin-2-yl)-N-phenylpiperazine-1-carboxamide;

(2R)-N-(4-tert-butylphenyl)-4-(3-cyanopyridin-2-yl)-2-methylpiperazine-1-carboxamide;

(2R)-4-(3-cyanopyridin-2-yl)-2-methyl-N-[4-(trifluoromethyl)phenyl]piperazine-1-carboxamide;

(2R)-2-methyl-4-(6-methylpyridin-2-yl)-N-[4-(trifluoromethyl)phenyl]piperazine-1-carboxamide;

(2R)-4-(6-methoxypyridin-2-yl)-2-methyl-N-[4-(trifluoromethyl)phenyl]piperazine-1-carboxamide;

(2R)-N-(4-tert-butylphenyl)-2-methyl-4-(6-methylpyridin-2-yl)piperazine-1-carboxamide;

(2R)-N-(4-tert-butylphenyl)-4-(6-methoxypyridin-2-yl)-2-methylpiperazine-1-carboxamide;

(2R)-N-(4-isopropylphenyl)-2-methyl-4-(6-methylpyridin-2-yl)piperazine-1-carboxamide;

(2R)-N-(4-isopropylphenyl)-4-(6-methoxypyridin-2-yl)-2-methylpiperazine-1-carboxamide;

(2R)-N-(4-cyclopentylphenyl)-2-methyl-4-(6-methylpyridin-2-yl)piperazine-1-carboxamide;

(2R)-N-(4-cyclopentylphenyl)-4-(6-methoxypyridin-2-yl)-2-methylpiperazine-1-carboxamide;

4-(3-chloropyridin-2-yl)-N-[5-(trifluoromethyl)pyridin-2-yl]piperazine-1-carboxamide (2R)-4-(3-chloropyridin-2-yl)-2-methyl-N-[5-(trifluoromethyl)pyridin-2-yl]piperazine-1-carboxamide;

(2R)-N-(4-tert-butylphenyl)-4-(3-chloropyrazin-2-yl)-2-methylpiperazine-1-carboxamide;

(2R)-4-(3-chloropyrazin-2-yl)-N-(4-isopropylphenyl)-2-methylpiperazine-1-carboxamide;

(2R)-4-(3-chloropyrazin-2-yl)-2-methyl-N-[4-(trifluoromethyl)phenyl]piperazine-1-carboxamide;

(2R)-4-(3-chloropyridin-2-yl)-2-methyl-N-[6-(trifluoromethyl)pyridin-3-yl]piperazine-1-carboxamide;

(2R)-N-(4-tert-butylcyclohexyl)-4-(3-chloropyridin-2-yl)-2-methylpiperazine-1-carboxamide;

(2R)-4-(3-chloropyridin-2-yl)-N-(4-isopropylcyclohexyl)-2-methylpiperazine-1-carboxamide;

(2R)-N-(4-isopropylcyclohexyl)-2-methyl-4-[3-(trifluoromethyl)pyridin-2-yl]piperazine-1-carboxamide;

(2R)-4-isoquinolin-1-yl-2-methyl-N-[4-(trifluoromethyl)phenyl]piperazine-1-carboxamide;

(2R)-N-(4-tert-butylphenyl)-4-isoquinolin-1-yl-2-methylpiperazine-1-carboxamide;

(2R)-N-(4-isopropylphenyl)-4-isoquinolin-1-yl-2-methylpiperazine-1-carboxamide;

(2R)-N-(4-cyclopentylphenyl)-4-isoquinolin-1-yl-2-methylpiperazine-1-carboxamide;

(2R)-N-(4-cyclohexylphenyl)-4-isoquinolin-1-yl-2-methylpiperazine-1-carboxamide;

N-(4-chlorophenyl)-4-[4-(trifluoromethyl)pyridin-2-yl]piperazine-1-carboxamide;

N-[4-(trifluoromethoxy)phenyl]-4-[4-(trifluoromethyl)pyridin-2-yl]piperazine-1-carboxamide;

N-(3-chlorophenyl)4-[4-(trifluoromethyl)pyridin-2-yl]piperazine-1-carboxamide;

N-[3-(trifluoromethyl)phenyl]-4-[3-(trifluoromethyl)pyridin-2-yl]piperazine-1-carboxamide;

N-(4-methylphenyl)-4-[3-(trifluoromethyl)pyridin-2-yl]piperazine-1-carboxamide;

N-(3-bromophenyl)-4-[3-(trifluoromethyl)pyridin-2-yl]piperazine-1-carboxamide;

N-(3-methoxyphenyl)-4-[3-(trifluoromethyl)pyridin-2-yl]piperazine-1-carboxamide;

4-(5-nitropyridin-2-yl)-N-[4-(trifluoromethoxy)phenyl]piperazine-1-carboxamide;

N-(1-naphthyl)-4-[3-(trifluoromethyl)pyridin-2-yl]piperazine-1-carboxamide;

N-(3-nitrophenyl)-4-[3-(trifluoromethyl)pyridin-2-yl]piperazine-1-carboxamide N-[4-(trifluoromethoxy)phenyl]-4-[3-(trifluoromethyl)pyridin-2-yl]piperazine-1-carboxamide;

N-(4-chloro-3-nitrophenyl)-4-[3-(trifluoromethyl)pyridin-2-yl]piperazine-1-carboxamide;

N-(3,5-dichlorophenyl)-4-[3-(trifluoromethyl)pyridin-2-yl]piperazine-1-carboxamide;

(2R)-4-(3-chloropyridin-2-yl)-N-{4-[cyano(phenyl)methyl]phenyl}-2-methylpiperazine-1-carboxamide;

(2R)-4-(3-chloropyridin-2-yl)-2-methyl-N-[3-methyl-4-(trifluoromethyl)phenyl]piperazine-1-carboxamide;

(2R)-4-(3-fluoropyridin-2-yl)-2-methyl-N-[3-methyl-4-(trifluoromethyl)phenyl]piperazine-1-carboxamide;

(2R)-4-{3-[bis(methylsulfonyl)amino]pyridin-2-yl}-N-(4-tert-butylphenyl)-2-methylpiperazine-1-carboxamide;

(2R)-2-methyl-N-[3-methyl-4-(trifluoromethyl)phenyl]-4-[3-(trifluoromethyl)pyridin-2-yl]piperazine-1-carboxamide;

(2R)-4-(3-chloropyridin-2-yl)-2-methyl-N-{4-[1-(trifluoromethyl)vinyl]phenyl}piperazine-1-carboxamide;

(2R)-2-methyl-4-[3-(trifluoromethyl)pyridin-2-yl]-N-{4-[1-(trifluoromethyl)vinyl]phenyl}piperazine-1-carboxamide;

(2R)-4-(3-fluoropyridin-2-yl)-2-methyl-N-{4-[1-(trifluoromethyl)vinyl]phenyl}piperazine-1-carboxamide;

(2R)-N-(4-sec-butylphenyl)-4-(3-fluoropyridin-2-yl)-2-methylpiperazine-1-carboxamide;

(2R)-2-methyl-N-[4-(2,2,2-trifluoro-1-methylethyl)phenyl]-4-[3-(trifluoromethyl)pyridin-2-yl]piperazine-1-carboxamide;

(2R)-4-(3-fluoropyridin-2-yl)-2-methyl-N-[4-(2,2,2-trifluoro-1-methylethyl)phenyl]piperazine-1-carboxamide;

(2R)-4-(3-chloro-5-nitropyridin-2-yl)-2-methyl-N-[4-(trifluoromethyl)phenyl]piperazine-1-carboxamide;

(2R)-4-(5-amino-3-chloropyridin-2-yl)-2-methyl-N-[4-(trifluoromethyl)phenyl]piperazine-1-carboxamide;

(2R)-4-(3-fluoropyridin-2-yl)-N-[3-fluoro-4-(trifluoromethyl)phenyl]-2-methylpiperazine-1-carboxamide;

(2R)-N-[3-fluoro-4-(trifluoromethyl)phenyl]-2-methyl-4-[3-(trifluoromethyl)pyridin-2-yl]piperazine-1-carboxamide;

(2R)-4-(3-chloropyridin-2-yl)-2-methyl-N-[4-(2,2,2-trifluoro-1-methylethyl)phenyl]piperazine-1-carboxamide;

(2R)-4-(3-chloropyridin-2-yl)-2-methyl-N-(2,2,4,4-tetrafluoro-4H-1,3-benzodioxin-6-yl)piperazine-1-carboxamide;

(2R)-4-(3-fluoropyridin-2-yl)-2-methyl-N-(2,2,4,4-tetrafluoro-4H-1,3-benzodioxin-6-yl)piperazine-1-carboxamide;

(2R)-2-methyl-N-(2,2,4,4-tetrafluoro-4H-1,3-benzodioxin-6-yl)-4-[3-(trifluoromethyl)pyridin-2-yl]piperazine-1-carboxamide;

(2R)-4-[3-(aminosulfonyl)pyridin-2-yl]-N-(4-tert-butylphenyl)-2-methylpiperazine-1-carboxamide;

(2R)-N-(4-benzoylphenyl)-4-(3-chloropyridin-2-yl)-2-methylpiperazine-1-carboxamide;

(2R)-4-(3-chloropyridin-2-yl)-N-(4-iodophenyl)-2-methylpiperazine-1-carboxamide;

(2R)-4-(3-chloropyridin-2-yl)-N-(9H-fluoren-2-yl)-2-methylpiperazine-1-carboxamide;

(2R)-N-(9H-fluoren-2-yl)-2-methyl-4-[3-(trifluoromethyl)pyridin-2-yl]piperazine-1-carboxamide;

(2R)-4-[3-cyano-6-(trifluoromethyl)pyridin-2-yl]-2-methyl-N-[4-(trifluoromethyl) phenyl]piperazine-1-carboxamide;

(2R)-N-(4-tert-butylphenyl)-4-[3-cyano-6-(trifluoromethyl)pyridin-2-yl]-2-methylpiperazine-1-carboxamide;

(2R)-4-[3-cyano-6-(trifluoromethyl)pyridin-2-yl]-N-(4-cyclopentylphenyl)-2-methylpiperazine-1-carboxamide;

(2R)-4-[3-cyano-6-(trifluoromethyl)pyridin-2-yl]-N-(4-cyclohexylphenyl)-2-methylpiperazine-1-carboxamide;

(2R)-4-(3-chloropyridin-2-yl)-2-methyl-N-{4-[2,2,2-trifluoro-1,1-bis(trifluoromethyl)ethyl]phenyl}piperazine-1-carboxamide;

(2R)-2-methyl-N-{4-[2,2,2-trifluoro-1,1-bis(trifluoromethyl)ethyl]phenyl}-4-[3-(trifluoromethyl)pyridin-2-yl]piperazine-1-carboxamide;

(2R)-4-(3-chloropyridin-2-yl)-N-(3-iodophenyl)-2-methylpiperazine-1-carboxamide;

(2R)-4-(3-fluoropyridin-2-yl)-N-(3-iodophenyl)-2-methylpiperazine-1-carboxamide;

(2R)-N-(4-butylphenyl)-4-(3-chloropyridin-2-yl)-2-methylpiperazine-1-carboxamide;

2-(fluoromethyl)-N-[4-(trifluoromethyl)phenyl]-4-[3-(trifluoromethyl)pyridin-2-yl]piperazine-1-carboxamide;

(2R)-4-(3-chloropyridin-2-yl)-2-methyl-N-[4-methyl-3-(trifluoromethyl)phenyl]piperazine-1-carboxamide;

(2R)-2-methyl-N-[4-methyl-3-(trifluoromethyl)phenyl]-4-[3-(trifluoromethyl)pyridin-2-yl]piperazine-1-carboxamide;

(2R)-N-[4-bromo-3-(trifluoromethyl)phenyl]-4-(3-chloropyridin-2-yl)-2-methylpiperazine-1-carboxamide;

(2R)-N-[4-bromo-3-(trifluoromethyl)phenyl]-2-methyl-4-[3-(trifluoromethyl)pyridin-2-yl]piperazine-1-carboxamide;

(2R)-4-(3-chloropyridin-2-yl)-N-[4-chloro-3-(trifluoromethyl)phenyl]-2-methylpiperazine-1-carboxamide;

(2R)-4-(3-chloropyridin-2-yl)-N-[4-fluoro-3-(trifluoromethyl)phenyl]-2-methylpiperazine-1-carboxamide;

(2R)-N-[4-chloro-3-(trifluoromethyl)phenyl]-2-methyl-4-[3-(trifluoromethyl)pyridin-2-yl]piperazine-1-carboxamide;

(2R)-N-[4-fluoro-3-(trifluoromethyl)phenyl]-2-methyl-4-[3-(trifluoromethyl)pyridin-2-yl]piperazine-1-carboxamide;

(2R)-4-(3-chloropyridin-2-yl)-2-methyl-N-{4-[1,2,2,2-tetrafluoro-1-(trifluoromethyl)ethyl]phenyl}piperazine-1-carboxamide;

(2R)-2-methyl-N-{4-[1,2,2,2-tetrafluoro-1-(trifluoromethyl)ethyl]phenyl}-4-[3-(trifluoromethyl)pyridin-2-yl]piperazine-1-carboxamide; (2R)-4-(3-chloropyrazin-2-yl)-2-methyl-N-{4-[1,2,2,2-tetrafluoro-1-(trifluoromethyl)ethyl]phenyl}piperazine-1-carboxamide;

(2R)-4-(3-chloropyrazin-2-yl)-2-methyl-N-{4-cyclopentyl-phenyl}piperazine-1-carboxamide; and

(2R)-4-(3-chloropyrazin-2-yl)-2-methyl-N-{4-cyclohexyl-phenyl}piperazine-1-carboxamide.

### Example 2

### Preparation of Representative VR1 Receptor Antagonists

This Example illustrates the preparation of representative substituted quinazolin-4-ylamine analogue VR1 antagonists, which may be used within the compositions and methods provided herein. Synthesis of the compounds provided in this Example is also described in PCT International Application Publication Number WO 03/062209, which published on Jul. 31, 2003.

### A. (4-Trifluoromethyl-phenyl)-[7-(2-trifluoromethyl-phenyl)-quinazolin-4-yl]-amine

### 1. 3-Nitro-2′-trifluoromethyl-biphenyl-4-carboxylic acid methyl ester

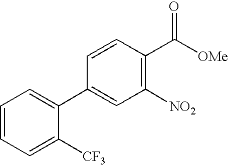

To a solution of 2-(trifluoromethyl)-phenylboronic acid (4.4 g, 0.0232 mol), 2-(dicyclohexylphosphino)biphenyl (111 mg, 0.318 mmol), and potassium phosphate (6.52 g, 0.031 mmol) in toluene, add palladium (II) acetate (36 mg, 0.160 mmol). Purge the reaction mixture for 10 minutes with dry nitrogen and then add 4-chloro-2-nitrobenzoic acid methyl ester. Heat the stirring reaction mixture overnight at 80° C., cool the mixture and filter through celite using ethyl acetate. Concentrate under reduced pressure, take up in fresh ethyl acetate and wash the solution with NaHCO₃ (saturated aqueous). Dry the solution (Na₂SO₄), concentrate under reduced pressure and then filter through a pad of silica gel using ethyl acetate as eluent. Removal of solvent under reduced pressure gives pure 3-nitro-2′-trifluoromethyl-biphenyl-4-carboxylic acid methyl ester as an oil.

### 2. 3-amino-2′-trifluoromethyl-biphenyl-4-carboxylic acid methyl ester

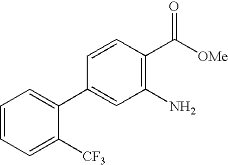

In a Parr apparatus, hydrogenate an ethanolic solution of 3-nitro-2′-trifluoromethyl-biphenyl-4-carboxylic acid methyl ester (5.54 g, 0.0169 mol) under 55 psi of hydrogen using tetrakis(triphenylphosphine)palladium (0) (300 mg). After 18 hours, filter the mixture through celite and concentrate under reduced pressure to give 3-amino-2′-trifluoromethyl-biphenyl-4-carboxylic acid methyl ester as a solid.

### 3. 7-(2-Trifluoromethyl-phenyl)-3H-quinazolin-4-one

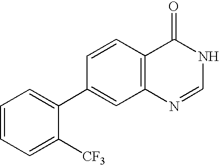

Heat a solution of 3-amino-2′-trifluoromethyl-biphenyl-4-carboxylic acid methyl ester (5.0 g, 0.0169 mol) and formamidine acetate (2.8 g, 0.0203 mol) in 2-methoxyethanol at reflux for 8 hours. Cool the mixture and concentrate under reduced pressure to give a dark oil. Dissolve the residue in 10% NaOH and wash the aqueous with ether (3×). Bring the aqueous layer to pH 4 using 12N HCl to produce a milky solution. Extract the solution with EtOAc, wash the EtOAc with brine, dry (Na₂SO₄) and concentrate under reduced pressure to give 7-(2-Trifluoromethyl-phenyl)-3H-quinazolin-4-one as a beige solid.

### 4. 4-chloro-7-(2-trifluoromethyl-phenyl)-quinazoline

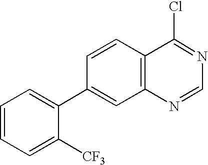

Reflux a solution of 7-(2-Trifluoromethyl-phenyl)-3H-quinazolin-4-one (1.12 g, 0.0039 mol) in POCl₃ for 16 hours. Cool the mixture and concentrate under reduced pressure. Partition the residue between saturated aqueous NaHCO₃ and EtOAc. Wash the EtOAc layer once with additional NaHCO₃, dry it (Na₂SO₄), and concentrate under reduced pressure to obtain the crude product as a solid. Filter the residue through a 2 inch pad of silica gel (50% EtOAc/Hexanes) and concentrate under reduce pressure to give 4-chloro-7-(2-trifluoromethyl-phenyl)-quinazoline as a pale yellow-brown solid.

### 5. (4-Trifluoromethyl-phenyl)-[7-(2-trifluoromethyl-phenyl)-quinazolin-4-yl]-amine

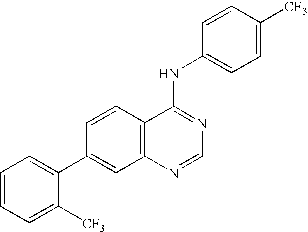

Reflux a solution of 4-chloro-7-(2-trifluoromethyl-phenyl)-quinazoline (258 mg, 0.836 mmol) and 4-(trifluoromethyl)-aniline (269 mg, 1.67 mmol) in isopropyl alcohol for 8 hours. Cool the solution, collect the precipitate via filtration and wash with dry ether (3×) to give pure (4-trifluoromethyl-phenyl)-[7-(2-trifluoromethyl-phenyl)-quinazolin-4-yl]-amine as the mono-HCl salt. Mass spec. 433.1.

### B. (4-tert-Butyl-phenyl)-[7-(2-trifluoromethyl-phenyl)-quinolin-4-yl]-amine

### 1. 7-(2-Trifluoromethyl-phenyl)-quinolin-4-ol

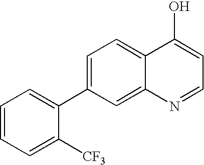

Combine 7-chloroquinolin-4-ol (1000 mg, 5.55 mmol,) 2-(trifluoromethyl)phenylboronic acid (1583 mg, 8.33 mmol) and toluene (50 mL), and bubble nitrogen into the solution for 10 minutes. Add palladium acetate (25 mg, 0.11 mmol), 2-(dicyclohexylphosphino)biphenyl (78 mg, 0.22 mmol), and K₃PO₄ (2353 mg, 11.1 mmol) and heat at 90° C. for 16 hours. Let cool, add water (25 mL) and EtOAc (50 mL), and remove any insoluble material by filtration. Separate the EtOAc layer, and extract the aqueous layer twice with EtOAc (25 mL each). Combine the EtOAc extracts, dry (Na₂SO₄), and evaporate. Purify by silica gel chromatography (94% CH₂Cl₂/5% MeOH/1% NH₄OH) to provide 110 mg of 7-(2-trifluoromethyl-phenyl)-quinolin-4-ol as a white solid.

### 2. 4-Chloro-7-(2-trifluoromethyl-phenyl)-quinoline

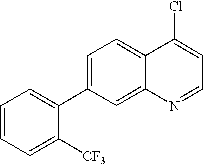

Heat a mixture of 7-(2-trifluoromethyl-phenyl)-quinolin-4-ol (50 mg, 0.17 mmol) in POCl₃ (10 mL) at 90° C. for 16 hours. Evaporate the POCl₃, and add ice (100 g) followed by careful addition of saturated NaHCO₃. Extract with EtOAc, dry (Na₂SO₄), and evaporate to provide 4-chloro-7-(2-trifluoromethyl-phenyl)-quinoline as a tan solid.

### 3. (4-tert-Butyl-phenyl)-[7-(2-trifluoromethyl-phenyl)-quinolin-4-yl]-amine

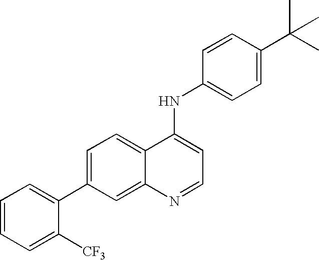

Heat a mixture of 4-chloro-7-(2-trifluoromethyl-phenyl)-quinoline (42 mg, 0.14 mmol) and 4-(tert-butyl)aniline (41 mg, 0.29 mmol) in 2-propanol (10 mL) at reflux for 3 hours. Evaporate the mixture, add 1M NaOH (10 mL), extract twice with EtOAc (10 mL each), dry (Na₂SO₄), and evaporate to provide the crude product. Purify by silica gel chromatography, eluting with 75% hexane-EtOAc to provide (4-tert-butyl-phenyl)-[7-(2-trifluoromethyl-phenyl)-quinolin-4-yl]-amine as a white solid. Mass spec. 420.2.

### C. (4-tert-Butylphenyl)-[7-(2-trifluoromethyl-phenyl)-pyrido[3,2-d]pyrimidin-4-yl]-amine

### 1. 5-bromo-3-nitropyridine-2-carbonitrile

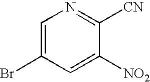

Heat a solution of 2,5-dibromo-3-nitropyridine (1.77 g, 6.3 mmol; Malinowski (1988) Bull. Soc. Chim. Belg. 97:51; see also U.S. Pat. No. 5,801,183) and cuprous cyanide (0.60 g, 6.69 mmol) in N,N-dimethylacetamide (25 mL) at 100° C. for 72 hours. After cooling, dilute the mixture with water (25 mL) and extract twice with EtOAc (25 mL each), then wash twice with water (25 mL each). The combined EtOAc extracts are dried (Na₂SO₄), evaporated, and purified by flash chromatography (50% EtOAc/hexane) to obtain 5-bromo-3-nitropyridine-2-carbonitrile as a pale solid.

### 2. 3-Amino-5-bromopyridine-2-carbonitrile

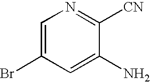

Mix 5-bromo-3-nitropyridine-2-carbonitrile (1.5 g, 5.3 mmol) and SnCl₂-dihydrate (5.00 g, 26.3 mmol) in concentrated HCl and stir at room temperature overnight. Add ice and carefully add 10 M NaOH until basic. Extract twice with Et₂O (200 mL), dry (Na₂SO₄) and evaporate. Purify by silica gel chromatography (75% hexane-EtOAc) to furnish 3-amino-5-bromopyridine-2-carbonitrile as a pale solid.

### 3. 7-Bromo-pyrido[3,2-d]pyrimidin-4-ol

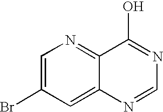

Reflux a mixture of 3-amino-5-bromopyridine-2-carbonitrile (504 mg, 2.00 mmol) and sodium acetate (312 mg, 3.81 mmol) in formic acid (20 mL) for 16 hours. Work up by evaporating to a white solid, and add 3N NaOH (50 mL). Filter off any undissolved material, then re-form the free pyrimidinol by adding concentrated HCl until a pH of 3 is achieved. Collect 7-bromo-pyrido[3,2-d]pyrimidin-4-ol and let dry overnight.

### 4. 7-Bromo-4-chloro-pyrido[3-2-d]pyrimidine

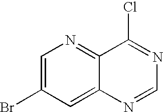

Heat a mixture of 7-bromo-pyrido[3,2-d]pyrimidin4-ol (35 mg, 0.15 mmol) and POCL₃ (10 mL) at 90° C. for 16 hours. Evaporate the POCl₃, and add ice (100 g) followed by careful addition of saturated NaHCO₃. Extract twice with EtOAc, dry (Na₂SO₄), and evaporate to provide 7-bromo4-chloro-pyrido[3-2-d]pyrimidine as a white solid.

### 5. (7-Bromo-pyrido[3, 2-d]pyrimidin-4-yl)-4-tert-butyl-phenyl)-amine

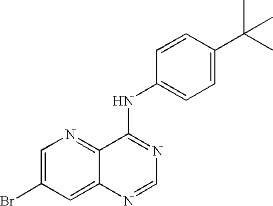

Heat a mixture of 7-bromo-4-chloro-pyrido[3-2-d]pyrimidine (35 mg, 0.14 mmol) and 4-(tert-butyl)aniline (43 mg, 0.29 mmol) in 2-propanol (10 mL) at reflux for 3 hours. Evaporate the mixture, add 1M NaOH (10 mL), extract twice with EtOAc (10 mL each), dry (Na₂SO₄), and evaporate to provide the crude product. Purify by silica gel chromatography, eluting with 75% hexane-EtOAc to provide (7-bromo-pyrido[3,2-d]pyrimidin4-yl)4-tert-butyl-phenyl)-amine as a white solid.

### 6. (4-tert-Butyl-phenyl)-[7-(2-trifluoromethyl-phenyl)-pyrido[3,2-d]pyrimidin-4-yl]-amine

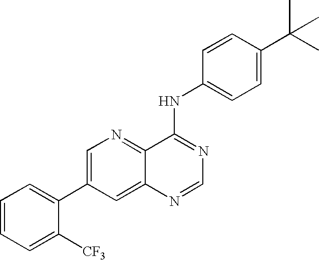

Combine (7-bromo-pyrido[3,2-d]pyrimidin-4-yl)-4-tert-butyl-phenyl)-amine (36 mg, 0.1 mmol), 2-(trifluoromethyl)phenyl-boronic acid (29 mg, 0.15 mmol) in 1,2-dimethoxyethane (10 mL) and bubble nitrogen into the mixture for 10 minutes. Add tetrakis(triphenylphosphine)palladium(0) (12 mg, 0.01 mmol) and 2M Na₂CO₃ (1 mL) and heat at 80° C. for 48 hours. Let the mixture cool to room temperature, dilute with water (10 mL), and extract twice with EtOAc (10 mL each). Dry (Na₂SO₄), evaporate, and purify on a preparative silica gel plate (2000 micron) eluting with 75% hexane-EtOAc to provide (4-tert-butyl-phenyl)-[7-(2-trifluoromethyl-phenyl)-pyrido[3,2-d]pyrimidin-4-yl]-amine as a light yellow solid. Mass spec. 422.2.

### D. (4-tert-Butyl-phenyl)-[6-(2-trifluoromethyl-phenyl)-phthalazin-1-yl]-amine

### 1. 4-Bromo-2-dibromomethyl-benzonitrile

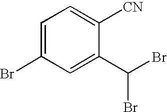

Reflux a mixture of 4-bromo-2-methyl-benzonitrile (19.6 g, 0.1 mol) and bromine (39.0 g, 0.22 mol) in carbon tetrachloride (500 mL) using a 500 watt sunlamp for 16 hours. Let cool to room temperature, and filter off succinimide. Evaporate the product fully to provide 4-bromo-2-dibromomethyl-benzonitrile as a yellow powder.

### 2. 5-Bromo-3-hydroxy-2,3-dihydro-isoindol-1-one

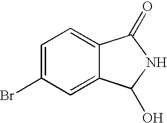

Combine 4-bromo-2-dibromomethyl-benzonitrile (7.0 g, 19.8 mmol) and acetonitrile (150 mL). Drip in a mixture of silver nitrate (7.0 g, 41.2 mmol) in water (40 mL) and reflux the resulting translucent yellow liquid for 72 hours. Evaporate the mixture, and add 1M NaOH (100 mL). Extract twice with EtOAc (100 mL each). Dry the solution (Na₂SO₄), evaporate, and purify by silica gel chromatography (80% hexanes-EtOAc) to obtain 600 mg of 4-bromo-2-formyl-benzonitrile and 1250 mg of 5-bromo-3-hydroxy-2,3-dihydro-isoindol-1-one as a white solid.

### 3. 6-Bromo-phthalazin-1-ol

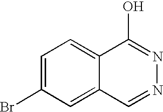

Combine 5-bromo-3-hydroxy-2,3-dihydro-isoindol-1-one (1.0 g, 4.39 mmol) and hydrazine hydrate (10 mL) and allow the suspension to stir at room temperature for 16 hours. Collect 6-bromo-phthalazin-1-ol as a white solid.

### 4. 6-Bromo-1-chloro-phthalazine

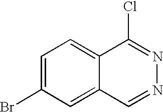

Heat a mixture of 6-Bromo-phthalazin-1-ol (300 mg, 1.33 mmol) in POCl₃ (10 mL) at 90° C. for 2 hours. Evaporate the POCl₃, and add ice (100 g) followed by careful addition of saturated NaHCO₃. Extract with EtOAc, dry (Na₂SO₄), and evaporate to provide 4-chloro-7-(2-trifluoromethyl-phenyl)-quinoline as a white solid.

### 5. (6-Bromo-phthalazin-1-yl)-(4-tert-butyl-phenyl)-amine

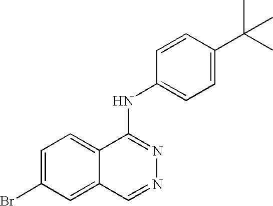

Heat a mixture of 6-bromo-1-chloro-phthalazine (500 mg, 2.05 mmol) and 4-(tert-butyl)aniline (611 mg, 4.10 mmol) in 2-propanol (10 mL) at reflux for 3 hours. Evaporate the mixture, add 1M NaOH (10 mL), extract twice with EtOAc (10 mL each), dry (Na₂SO₄), and evaporate to provide the crude product. Purify by silica gel chromatography, eluting with dichloromethane followed by 95% CH₂Cl₂-MeOH to provide (6-bromo-phthalazin-1-yl)-(4-tert-butyl-phenyl)-amine as a white solid.

### 6. (4-tert-Butyl-phenyl)-[6-(2-trifluoromethyl-phenyl)-phthalazin-1-yl]-amine

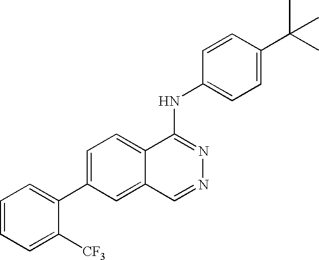

Combine (6-bromo-phthalazin-1-yl)-(4-tert-butyl-phenyl)-amine (60 mg, 0.19 mmol), 2-(trifluoromethyl)phenyl-boronic acid (50 mg, 0.26 mmol) in 1,2-dimethoxyethane (10 mL) and bubble nitrogen into the mixture for 10 minutes. Add tetrakis(triphenylphosphine)palladium(0) (12 mg, 0.01 mmol) and 2M Na₂CO₃ (1 mL) and heat at 80° C. for 48 hours. Let the mixture cool to room temperature, dilute with water (10 mL), and extract twice with EtOAc (10 mL each). Dry (Na₂SO₄), evaporate, and purify on a preparative silica gel plate (2000 micron) eluting with 75% hexane-EtOAc to provide (4-tert-butyl-phenyl)-[6-(2-trifluoromethyl-phenyl)-phthalazin-1-yl]-amine as a straw colored solid. Mass Spec. 421.2.

### E. (4-tert-Butyl-phenyl)-[7-(2-trifluoromethyl-phenyl)-pyrido[2,3-d]pyrimidin-4-yl]-amine

### 1. Oxo-3-phenyl-propionaldehyde

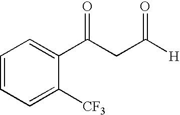

Heat a mixture of toluene and sodium ethoxide (40 mL of a 21% ethanolic solution) to 50° C. Add 2-trifluoromethylacetophenone (20.0 g, 0.11 mol) and ethyl formate (11.8 g, 0.16 mol), and let stir at 65° C. for 12 hours. Allow mixture to cool to room temperature and add 300 mL of diethyl ether. Collect the precipitate to obtain the sodium salt of 3-oxo-3-phenyl-propionaldehyde.

### 2. 7-(2-Trifluoromethyl-phenyl)-1H-pyrido[2,3-d]pyrimidine-2,4-dione

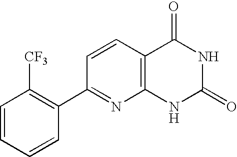

Finely divide the sodium salt of 3-oxo-3-phenyl-propionaldehyde (10.0 g, 0.043 mol) and add 50 mL of 90% phosphoric acid. Let stir until fully dissolved. Separately, similarly dissolve 6-amino-1H-pyrimidine-2,4-dione 5.7 g, 0.043 mol) in 50 mL of 90% phosphoric acid. Combine the 2 solutions and let stir for 12 hours at 100° C. Let the solution cool to room temperature, add 300 mL of water, and collect the product as a sticky solid. Triturate with ether to obtain 7-(2-trifluoromethyl-phenyl)-1H-pyrido[2,3-d]pyrimidine-2,4-dione as a white solid.

### 3. 2,4-Dichloro-7-(2-trifluoromethyl-phenyl)-pyrido[2,3-d]pyrimidine

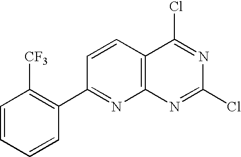

Heat a mixture of 7-(2-trifluoromethyl-phenyl)-1H-pyrido[2,3-d]pyrimidine-2,4-dione (5.0 g, 0.016 mol) and POCl₃ (100 mL) at 90° C. for 36 hours. Evaporate the POCl₃, and add ice (400 g) followed by careful addition of saturated NaHCO₃. Extract twice with EtOAc, dry (Na₂SO₄), and evaporate to provide 2,4-dichloro-7-(2-trifluoromethyl-phenyl)-pyrido[2,3-d]pyrimidine.

### 4. (4-tert-Butyl-phenyl)-[2-chloro-7-(2-trifluoromethyl-phenyl)-pyrido[2,3-d]pyrimidin-4-yl]-amine

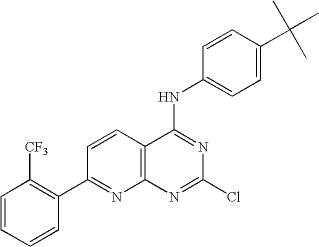

To a mixture of diisopropylethylamine (260 mg, 2.0 mmol) in acetonitrile (5 mL), add t-butylaniline (124 mg, 1.0 mmol) followed by (4-tert-butyl-phenyl)-[2-chloro-7-(2-trifluoromethyl-phenyl)-pyrido[2,3-d]pyrimidin-4-yl]-amine (310 mg, 1.0 mmol). Heat the mixture to 80° C. for six hours. Evaporate the solvent, and partition between 1M NaOH and EtOAc. Dry the solvent (Na₂SO₄) and evaporate. Purify by silica gel chromatography (1:1 hexanes/EtOAc to furnish the monoaniline (4-tert-butyl-phenyl)-[2-chloro-7-(2-trifluoromethyl-phenyl)-pyrido[2,3-d]pyrimidin-4-yl]-amine as a yellow solid.

### 5. (4-tert-Butyl-phenyl)-[7-(2-trifluoromethyl-phenyl)-pyrido[2,3-d]pyrimidin-4-yl-amine

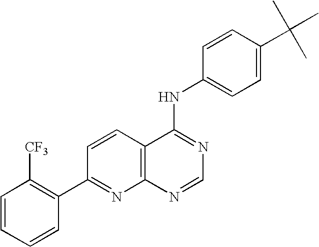

The 2-chloro substituent in (4-tert-butyl-phenyl)-[2-chloro-7-(2-trifluoromethyl-phenyl)-pyrido[2,3-d]pyrimidin-4-yl]-amine can be removed using a number of reducing conditions known to those skilled in the art of organic synthesis e.g. hydrogenolysis or treatment with aluminum hydride reducing agents (See, e.g., Hudlicky, M. Reductions in Organic Chemistry, ACS Monograph 188: 1996).

### F. [7-(3-fluoro-pyridin-2-yl)-quinazolin-4-yl]-(5-trifluoromethyl-pyridin-2-yl)-amine

### 1. 7-bromo-4-chloro-quinazoline

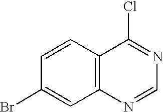

Reflux a solution of 7-bromo-3H-quinazolin-4-one (1.24 g, 0.0055 mol) in POCl₃ for 3.5 hours. Remove the excess POCl₃ under reduced pressure and partition the residue between EtOAc and saturated aqueous NaHCO₃. Dry the EtOAc layer and remove the solvent under reduced pressure to give 7-bromo-4-chloro-quinazoline as a yellow solid.

### 2. (7-bromo-quinazolin-4-yl)-(5-trifluoromethyl-pyridin-2-yl)-amine

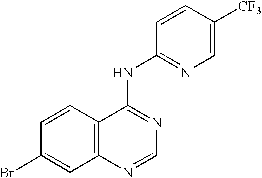

Heat a mixture of 7-bromo-4-chloro-quinazoline (200 mg, 0.821 mmol) and 2-amino-5-trifluoromethyl-pyridine (239 mg, 1.48 mmol) at 230° C. for 2 minutes. Cool and partition the solid residue between EtOAc and 10% NaOH. Dry the EtOAc layer (Na₂SO₄), remove the solvent under reduced pressure, and purify via flash chromatography to yield (7-bromo-quinazolin-4-yl)-(5-trifluoromethyl-pyridin-2-yl)-amine as a yellow solid.

### 3. 3-fluoro-2-tributylstannanyl-pyridine

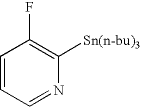

Cool a solution of 2-bromo-3-fluoro-pyridine (542 mg, 3.08 mmol) in dry THF to −78° C. using a dry ice acetone bath. Add n-butyl-lithium (1.6 M in THF, 2.0 mL) to the reaction mixture dropwise via syringe over a 20 minute period. After stirring for 1.5 hours at −78° C., add tributyltin chloride slowly via syringe and remove the cooling bath. After 2 hours, partition the reaction mixture between EtOAc and brine, dry the EtOAc layer (Na₂SO₄) and remove the solvents under reduced pressure. Flash chromatography (ether/hexanes) yields 3-fluoro-2-tributylstannanyl-pyridine as a colorless oil.

### 4. [7-(3-fluoro-pyridin-2-yl)-quinazolin-4-yl]-(5-trifluoromethyl-pyridin-2-yl)-amine

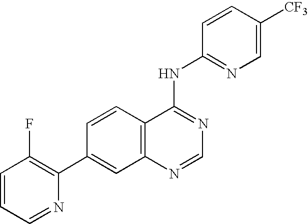

Using procedures analogous to those given above, [7-(3-fluoro-pyridin-2-yl)-quinazolin-4-yl]-(5-trifluoromethyl-pyridin-2-yl)-amine is prepared by coupling (7-bromo-quinazolin-4-yl)-(5-trifluoromethyl-pyridin-2-yl)-amine to 3-fluoro-2-tributylstannanyl-pyridine. Mass spec. 385.1.

### G. (4-tert-butyl-phenyl)-(7-pyridin-2-yl-quinazolin-4-yl)-amine

### 1. 4-bromo-2-nitro-benzonitrile

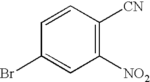

Stir the mixture of 1,4-dibromo-2-nitro-benzene (3.56 mmol)and CuCN (3.74 mmol) in DMA (4 ml) at 100° C. for 5 hours. Cool to room temperature, dilute with EtOAc, filter through celite, wash the organic layer with brine, dry over Na₂SO₄, and concentrate under vacuum. Purify the residue by flash chromatography (4:1 hexanes/EtOAc) to give 4-bromo-2-nitro-benzonitrile.

### 2. 2-amino-4-bromo-benzonitrile

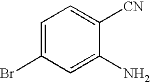

To a suspension of 4-bromo-2-nitro-benzonitrile (2.60 g, 0.0115 mol) in 12N HCl at 0° C., add SnCl₂-2H₂O portionwise. As the reaction is stirred vigorously, a white precipitate will form. After 1 h add ice to the reaction vessel followed by 10N NaOH until the solution is basic. Extract the aqueous mixture with ether (2×) and EtOAc (1×) and wash the combined organic layers with brine. Dry the solution (Na₂SO₄) and remove the solvents under reduced pressure to give 2-amino-4-bromo-benzonitrile as a beige solid.

### 3. 7-bromo-3H-quinazolin-4-one

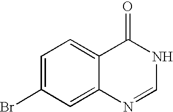

To a solution of 2-amino-4-bromo-benzonitrile (550 mg, 2.79 mmol) in formic acid, add sodium acetate (435 mg, 5.30 mmol) in one portion. Reflux the reaction mixture for 16 h then remove the formic acid under reduced pressure to give a solid. Add 20% aqueous NaOH and stir for 1 hour. Remove the undissolved solids via filtration and acidify the filtrate with 12N HCl to produce a white solid. Collect the solid via filtration and wash it with water (5×) and ether (1×) to give 7-bromo-3H-quinazolin-4-one as an off-white solid.

### 4. 7-pyridin-2-yl-3H-quinazolin-4-one

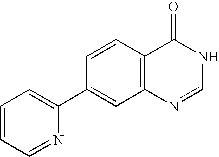

To a solution of 7-bromo-3H-quinazolin-4-one (100 mg, 0.444 mmol) in toluene/dioxane (3:1), add 2-tributylstannanyl-pyridine (162 mg, 0.444 mmol) followed by tetrakis-(triphenylphosphine)-palladium(0) (26 mg, 0.022 mmol). Bubble dry nitrogen through the solution for 10 minutes then heat the stirring solution to 115° C. under a nitrogen atmosphere. After several minutes the reaction mixture becomes homogeneous. After 16 hours, cool the reaction vessel and collect the precipitate via filtration. Wash the solid with 25% EtOAc/hexanes followed by hexanes to give 7-pyridin-2-yl-3H-quinazolin-4-one as a beige solid.

### 5. (4-tert-butyl-phenyl)-(7-pyridin-2-yl-quinazolin-4-yl)-amine

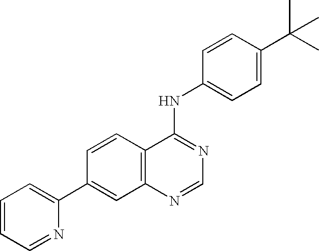

Using procedures analogous to those given above, (4-tert-butyl-phenyl)-(7-pyridin-2-yl-quinazolin-4-yl)-amine is prepared from 4-chloro-7-pyridin-2-yl-quinazoline and 4-tert-butylaniline. Mass spec. 354.2.

### H. (4-tert-Butyl-phenyl)-[7-(3-trifluoromethyl-pyridin-2-yl)-quinazolin-4-yl]-amine hydrochloride

### 1. 2-(4-bromo phenyl)-3-(trifluoromethyl)-pyridine

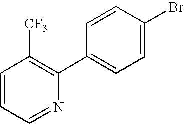

To a de-gassed mixture of 2-bromo-3-(trifluoromethyl)-pyridine (2.26 mmol), 4-bromo-phenylbronic acid (2.49 mmol), and 2M Na₂CO₃ (5.65 mmol), in DME (10 mL) under nitrogen add Pd(PPh₃)₄ (0.09 mmol). Stir the mixture at 80° C. overnight, concentrate, extract with EtOAc. fry over Na₂SO₄, concentrate under vacuum, and purify by flash chromatography (4:1 hexanes/EtOAc) to give 2-(4-bromo phenyl)-3-(trifluoromethyl)-pyridine.

### 2. 2-(4-bromo-3-nitro-phenyl)-3-(trifluoromethyl)-pyridine

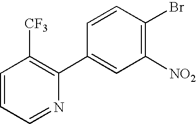

To a solution of 2-(4-bromophenyl)-3-(trifluoromethyl)-pyridine (0.93 mmol) in H₂SO₄ (4 mL) cautiously add fuming HNO₃ (2 ml). Stir the mixture 30 minutes at room temperature. Pour the mixture onto ice-water (20 mL) and collect the precipitate. Dissolve the precipitate in EtOAc and neutralize with saturated NaHCO₃, dry over Na₂SO₄, concentrate under vacuum to obtain 2-(4-bromo-3-nitro-phenyl)-3-(trifluoromethyl)-pyridine.

### 3. 2-nitro-4(3-trifluoromethyl-pyridin-2-yl)-benzonitrile

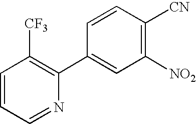

To a solution of 2-(4-bromo-3-nitro-phenyl)-3-(trifluoromethyl)-pyridine (0.55 mmol) in DMA (4 mL) add CuCN (0.60 mmol). Stir the mixture 4 hours at 110° C. Cool to room temperature, dilute with 20 ml of EtOAc, and filter through celite pad. Wash the filtrated with brine, dry over Na₂SO₄, concentrate under vacuum, and purify by flash chromatography (1:1 hexanes/EtOAc) to give 2-nitro-4(3-trifluoromethyl-pyridin-2-yl)-benzonitrile.

### 4. 2-amino-4-(3-trifluoromethyl-pyridin-2-yl)-benzo-nitrile

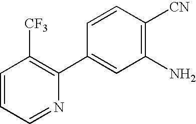

To an ice-water cooled solution of 2-nitro-4-(3-trifluoromethyl-pyridin-2-yl)-benzonitrile (0.44 mmol) in conc. HCl (6 mL) add SnCl₂ (1.457 mmol). Stir the mixture 2 hours at room temperature. Neutralize with NaOH, extract with EtOAc, dry over Na₂SO₄, and concentrate under vacuum. Purify the residue by flash chromatography (4:1 hexanes/EtOAc) to give 2-amino-4(3-trifluoromethyl-pyridin-2-yl)-benzo-nitrile.

### 5. 7-(3-trifluoromethyl-pyridin-2-yl)-quinazolin-4-ol

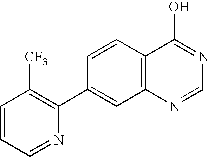

Reflux 2-amino-4-(3-trifluoromethyl-pyridin-2-yl)-benzo-nitrile (0.41 mmol) and NaOAc (1.23 mmol) for 16 hours in HCOOH (10 mL). Evaporate the solvent in vacuo, suspend the residue in 20 ml of 20% NaOH, stir for 30 minutes at room temperature. Filter, extract with EtOAc, dry over Na₂SO₄, and concentrate under vacuum to give 7-(3-trifluoromethyl-pyridin-2-yl)-quinazolin-4-ol.

### 6. 4-chloro-7-(3-trifluoromethyl-pyridin-2-yl)-quinazoline

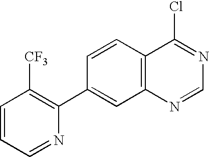

Reflux 7-(3-trifluoromethyl-pyridin-2-yl)-quinazolin-4-ol (0.38 mmol) for 18 hours in POCl₃ (5 mL). Evaporate the solvent in vacuo, then carefully neutralize with saturated NaHCO₃, and extract with EtOAc. Dry over Na₂SO₄, concentrate under vacuum to obtain 4-chloro-7-(3-trifluoromethyl-pyridin-2-yl)-quinazoline.

### 7. (4-tert-Butyl-phenyl)-[7-(3-trifluoromethyl-pyridin-2-yl)-quinazolin-4-yl]hydrochloride

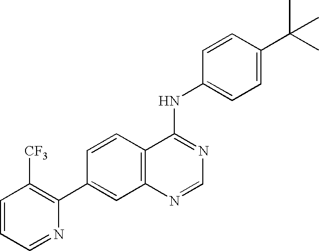

Stir 4-chloro-7-(3-trifluoromethyl-pyridin-2-yl)-quinazoline (0.16 mmol) and 4-tert-butyl-aniline (0.32 mmol) in IPA (4 mL) at 80° C. for 6 hours. Cool the mixture and collect the precipitate to obtain (4-tert-butyl-phenyl)-[7-(3-trifluoromethyl-pyridin-2-yl)-quinazolin-4-yl]hydrochloride. Mass spec. 422.2.

### I. (4-tert-Butyl-phenyl)-[2-methyl-7-(3-trifluoromethyl-pyridin-2-yl)-quinazolin-4-yl]-amine hydrochloride

### 1. 2-amino-4-(3-trifluoromethyl-pyridin-2-yl)-benzamide

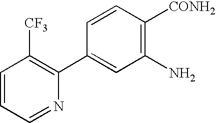

Stir a mixture of 2-amino-4(3-trifluoromethyl-pyridin-2-yl)-benzo-nitrile (0.50 mmol) in 70% H₂SO₄ (10 ml) at 110° C. for Ihour. Cool to room temperature, neutralize with NaOH, extract with EtOAc, dry over Na₂SO₄, and concentrate under vacuum. Purify the residue by flash chromatography (3:2 hexanes/EtOAc) to give 2-amino-4-(3-trifluoromethyl-pyridin-2-yl)-benzamide.

### 2. 2-acetylamino-4-(3-trifluoromethyl-pyridin-2-yl)-benzamide

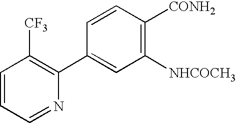

To a solution of 2-amino-4-(3-trifluoromethyl-pyridin-2-yl)-benzamide (0.5 mmol) and pyridine (0.55 mmol) in THF (5 ml) add acetyl chloride (0.55 mmol). Stir the mixture 10 minutes at room temperature. Concentrate under vacuum, extract with EtOAc, wash with brine, dry over Na₂SO₄, and concentrate under vacuum. Triturate with ether to give 2-acetylamino-4-(3-trifluoromethyl-pyridin-2-yl)-benzamide.

### 3. 2-methyl-7-(3-trifluoromethyl-pyridin-2-yl)-quinazolin-4-ol

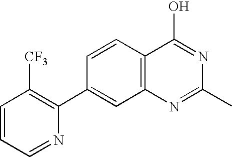

Suspend 2-acetylamino-4-(3-trifluoromethyl-pyridin-2-yl)-benzamide in 20 ml of 20% NaOH, stir for 30 minutes at room temperature. Filter, acidify to pH=6, extract with EtOAc, and concentrate under vacuum to give 2-methyl-7-(3-trifluoromethyl-pyridin-2-yl)-quinazolin-4-ol.

### 4. 4-chloro-2-methyl-7-(3-trifluoromethyl-pyridin-2-yl)-quinazoline

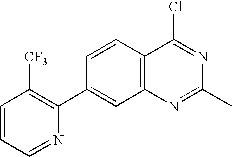

Using procedures analogous to those already described 4-chloro-2-methyl-7-(3-trifluoromethyl-pyridin-2-yl)-quinazoline is prepared from 2-methyl-7-(3-trifluoromethyl-pyridin-2-yl)-quinazolin-4-ol.

### 5. (4-tert-Butyl-phenyl)-[2-methyl-7-(3-trifluoromethyl-pyridin-2-yl)-quinazolin-4-yl]-amine

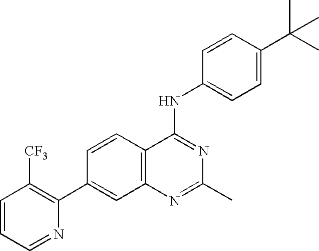

Using procedures analogous to those already described, (4-tert-Butyl-phenyl)-[2-methyl-7-(3-trifluoromethyl-pyridin-2-yl)-quinazolin-4-yl]-amine is prepared by condensing 4-chloro-2-methyl-7-(3-trifluoromethyl-pyridin-2-yl)-quinazoline with 4-tert-butylaniline. Mass spec. 436.2.

### J. [7-(3-Methyl-pyridin-2-yl)-quinazolin-4-yl]-(4-trifluoromethyl-phenyl)-amine

### 1. 7-[B(OH)₂]-3H-quinazolin-4-one

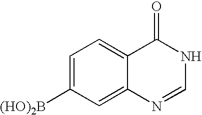

Reflux a mixture of 3-amino-4-carboethoxy-phenylboronic acid (1.46 g, 0.007 mol), prepared according to the procedure of Torssell et. al. (1957) Arkiv Kemi 10:497, and formamidine acetate (1.17 g, 0.008 mol) in methoxyethanol for 7 hours. Add an additional equivalent of formamidine acetate and continue to reflux for 16 hours. Cool the dark solution and remove the solvent under reduced pressure. Add ˜100 mL of water, stir for 10 minutes, and collect the light gray solid on a sintered glass funnel. Wash the solid with water (3×), dry, and recrystallize from methanol to give 7-[B(OH)₂]-3H-quinazolin-4-one as a white solid.

### 2. 7-(3-Methyl-pyridin-2-yl)-3H-quinazolin-4-one

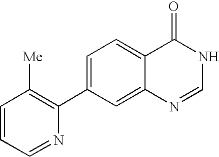

Purge a solution of 7-[B(OH)₂]-3H-quinazolin-4-one (115 mg, 0.605 mmol), 2-bromo-3-methyl-pyridine (103 mg, 0.605 mmol), Na₂CO₃ (0.757 mL, 1.51 mmol, 2M aqueous solution), and DMF (4 mL) with nitrogen for 10 minutes. Add a catalytic amount of tetrakis-(triphenylphosphine)-palladium(0) (35 mg, 0.03 mmol) and heat at 95° C. for 16 hours. Cool the reaction mixture, dilute with water and extract with ethyl acetate. Dry the combined organic layers (Na₂SO₄), concentrate under reduced pressure, and purify the crude product using silica gel chromatography (MeOH/CH₂Cl₂) to give 7-(3-Methyl-pyridin-2-yl)-3H-quinazolin-4-one.

### 3. [7-(3-Methyl-pyridin-2-yl)-quinazolin-4-yl]-(4-trifluoromethyl-phenyl)-amine

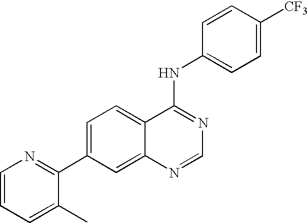

Using procedures analogous to those described above (see, for example, Schemes 1 and 2), [7-(3-Methyl-pyridin-2-yl)-quinazolin-4-yl]-(4-trifluoromethyl-phenyl)-amine is prepared from 7-(3-Methyl-pyridin-2-yl)-3H-quinazolin-4-one in two steps. Mass spec. 380.1.

### K. (4-tert-Butyl-phenyl)-[7-(3-trifluoromethyl-pyridin-2-yl)-quinazolin-4-yl]-amine hydrochloride

### 1. 2-p-tolyl-3-trifluoromethyl-pyridine

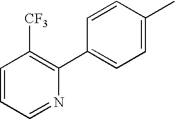

To a de-gassed mixture of 2-chloro-3-(trifluoromethyl)-pyridine (70.1 mmol), p-tolylboronic acid (70.6 mmol), and 2M Na₂CO₃ (175.0 mmol), in DME (200 mL) under nitrogen add Pd(PPh₃)₄ (2.8 mmol). Stir the mixture at 80° C. for overnight, concentrate, extract with EtOAc. Dry over Na₂SO₄, concentrate under vacuum, pass a silica gel pad to give 2-p-tolyl-3-trifluoromethyl-pyridine.

### 2. 2-(4-methyl-3-nitro-phenyl)-3-(trifluoromethyl)-pyridine

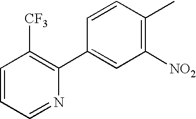

To a solution of 2-p-tolyl-3-trifluoromethyl-pyridine (8.4 mmol) in H₂SO₄ (6 mL) cautiously add fuming HNO₃ (2 ml). Stir the mixture 60 minutes at room temperature. Pour the mixture onto ice-water (30 mL), extract with EtOAc, neutralize with 1 N NaOH, dry over Na₂SO₄, and concentrate under vacuum to obtain 2-(4-methyl-3-nitro-phenyl)-3-(trifluoromethyl)-pyridine.

### 3. 2-nitro-4-(3-trifluoromethyl-pyridin-2-yl)-benzoic acid

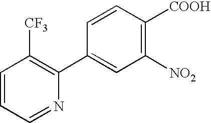

To a solution of 2-(4-methyl-3-nitro-phenyl)-3-(trifluoromethyl)-pyridine (7.1 mmol) in the mixture of pyridine (10 mL) and water (5 ml) add KMnO₄ (25.3 mmol) portionwise. Stir the mixture, 4 hours at 110° C. then add another 25.3 mmol of KMnO₄ with 10 ml of water. Stir the mixture at 110° C. for overnight. Cool to room temperature, filter through celite pad. Concentrate the filtrate under vacuum, dilute with water, and wash the aqueous with EtOAc. Neutralize the aqueous with 2 N HCl and collect the precipitate to give 2-nitro4(3-trifluoromethyl-pyridin-2-yl)-benzoic acid.

### 4. 2-amino-4-(3-trifluoromethyl-pyridin-2-yl)-benzoic acid

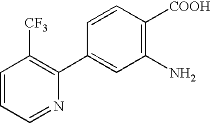

Hydrogenate the solution of 2-nitro4-(3-trifluoromethyl-pyridin-2-yl)-benzoic acid (3.84 mmol) in 95% EtOH (100 mL) with 10% Pd—C (150 mg) for over night. Filter through a celite pad and concentrate the filtrate to give 2-amino-4-(3-trifluoromethyl-pyridin-2-yl)-benzoic acid.

### 5. 7-(3-trifluoromethyl-pyridin-2-yl)-quinazolin-4-ol

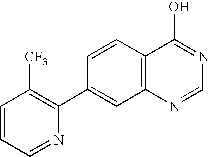

Stir the mixture of 2-amino-4-(3-trifluoromethyl-pyridin-2-yl)-benzoic acid (1.95 mmol) in HCONH₂ (10 mL) for 4 hours at 145° C. Cool to room temperature, dilute with 20 ml of water, and collect the precipitate to give 7-(3-trifluoromethyl-pyridin-2-yl)-quinazolin-4-ol.

### 6. (4-tert-Butyl-phenyl)-[7-(3-trifluoromethyl-pyridin-2-yl)-quinazolin-4-yl]-amine hydrochloride

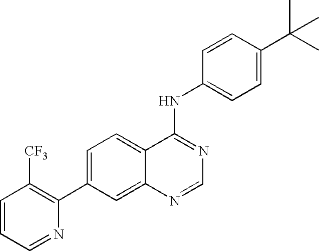

Using procedures analogous to those described above, (4-tert-Butyl-phenyl)-[7-(3-trifluoromethyl-pyridin-2-yl)-quinazolin-4-yl]-amide hydrochloride is prepared from 7-(3-trifluoromethyl-pyridin-2-yl)-quinazolin-4-ol in two steps. Mass spec. 422.2.

### L. [6-(proprane-2-sulfonyl)-pyridin-3-yl]-[7-(3-trifluoromethyl-pyridin-2-yl)-quinazolin-4-yl]-amine hydrochloride

### 1. 2-Isopropylsulfanyl-5-nitro-pyridine

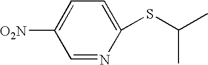

Stir the mixture of 2-mercapto-5-nitropyridine (10.0 mmol) and NaH (14.0 mmol) in DMA (10 ml) at room temperature for 30 minutes. Add 2-iodopropane (11.0 mmol) and stir overnight at room temperature. Dilute with H₂O, extract with EtOAc, wash with brine, dry over Na₂SO₄, and concentrate under vacuum. Purify the residue by flash chromatography (9:1 hexanes/EtOAc) to give 2-isopropyl-sulfanyl-5-nitro-pyridine.

### 2. 5-Nitro-2-(propane-2-sulfonyl)-pyridine

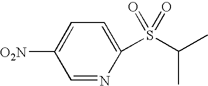

Heat the mixture of 2-isopropyl-sulfanyl-5-nitro-pyridine (3.5 mmol) and KMnO₄ (14.1 mmol) in HOAc (15 ml) at 110° C. for overnight. Filter, concentrate the filtrate, and neutralize with NaHCO₃. Extract with EtOAc, wash with brine, dry over Na₂SO₄, and concentrate under vacuum to give 2-(propyl-2-sulfonyl)-5-nitro-pyridine.

### 3. 6-(Propane-2-sulfonyl)-pyridin-3-ylamine

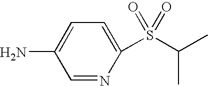

Suspend 2-(propyl-2-sulfonyl)-5-nitro-pyridine (0.44 mmol) in 10 ml of conc. HCl, add SnCl2 dihydrate (1.43 mmol), and stir for 2 hours at room temperature. Neutralize with NaOH. Extract with EtOAc, wash with brine, dry over Na₂SO₄, and concentrate under vacuum to give 6-(propane-2-sulfonyl)-pyridin-3-ylamine.

### 4. [6-(propane-2-sulfonyl)-pyridin-3-yl]-[7-(3-trifluoromethyl-pyridin-2-yl)-quinazolin-4-yl]-amine hydrochloride

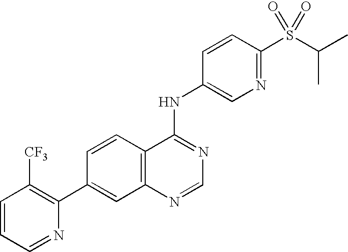

Use the method described in Example H.7 above to obtain [6-(propane-2-sulfonyl)-pyridin-3-yl]-[7-(3-trifluoromethyl-pyridin-2-yl)-quinazolin-4-yl]-amine hydrochloride. Mass spec. 473.1.

### M. Additional Representative Substituted Quinazolin-4-ylamine Analogues

Those having skill in the art will recognize that the starting materials may be varied and additional steps employed to produce other compounds encompassed by the present invention. The following compounds were prepared using the above methods, with readily apparent modifications, and may be used in the compositions and methods provided herein:

(5-trifluoromethyl-pyridin-2-yl)-[7-(3-trifluoromethyl-pyridin-2-yl)-quinazolin-4-yl]-amine;

(6-trifluoromethyl-pyridin-3-yl)-[7-(3-trifluoromethyl-pyridin-2-yl)-pyrido[3,2-d]pyrimidin-4-yl]-amine;

[2-methyl-7-(3-trifluoromethyl-pyridin-2-yl)-quinazolin-4-yl]-(6-trifluoromethyl-pyridin-3-yl)-amine;

(6-trifluoromethyl-pyridin-3-yl)-[7-(3-trifluoromethyl-pyridin-2-yl)-quinazolin-4-yl]-amine;

[2-chloro-7-(2-trifluoromethyl-phenyl)-pyrido[2,3-d]pyrimidin-4-yl]-(6-trifluoromethyl-pyridin-3-yl)-amine;

[2-chloro-7-(2-trifluoromethyl-phenyl)-pyrido[2,3-d]pyrimidin-4-yl]-(4-trifluoromethyl-phenyl)-amine;

[7-(2-trifluoromethyl-phenyl)-quinazolin-4-yl]-(5-trifluoromethyl-pyridin-2-yl)-amine;

(7-pyridin-2-yl-quinazolin-4-yl)-(5-trifluoromethyl-pyridin-2-yl)-amine;

(5-tert-butyl-isoxazol-3-yl)-(7-pyridin-2-yl-quinazolin-4-yl)-amine;

(4-trifluoromethyl-phenyl)-[6-(2-trifluoromethyl-phenyl)-phthalazin-1-yl]amine;

(4-tert-Butyl-phenyl)-(6-pyridin-2-yl-phthalazin-1-yl)-amine;

(4-tert-Butyl-phenyl)-[7-(3-trifluoromethyl-pyridin-2-yl)-quinolin-4-yl]-amine;

(4-trifluoromethoxy-phenyl)-7-(3-trifluoromethyl-pyridin-2-yl)-pyrido[3,2-d]pyrimidin-4-yl]-amine;

(4-tert-butyl-phenyl)-[7-(2-trifluoromethyl-phenyl)-quinazolin-4-yl]-amine;

(4-trifluoromethyl-phenyl)-[7-(2-trifluoromethyl-phenyl)-pyrido[3,2-d]pyrimidin-4-yl]amine;

[7-(1-Oxy-3-trifluoromethyl-pyridin-2-yl)-quinazolin-4-yl-(4-trifluoromethyl-phenyl)-amine;

[7-(1-Oxy-pyridin-2-yl)-quinazolin-4-yl]-(4-trifluoromethyl-phenyl)-amine;

(4-Trifluoromethyl-phenyl)-[7-(3-trifluoromethyl-pyridin-2-yl)-pyrido[3,2-d]pyrimidin-4-yl]-amine;

(4-tert-butyl-phenyl)-[2-methyl-7-(2-trifluoromethyl-phenyl)-quinazolin-4-yl]-amine;

[2-methyl-7-(2-trifluoromethyl-phenyl)-quinazolin-4-yl]-(4-trifluoromethyl-phenyl)-amine;

(4-tert-butyl-phenyl)-[2-isopropyl-7-(2-trifluoromethyl-phenyl)-quinazolin-4-yl]-amine;

N²-isobutyl-N⁴-(4-trifluoromethyl-phenyl)-7-(2-trifluoromethyl-phenyl)-pyrido[2,3-d]pyrimidine-2,4-diamine;

[4-(1,2,2,2-tetrafluoro-1-trifluoromethyl-ethyl)-phenyl-]-[7-(2-trifluoromethyl-phenyl)-quinazolin-4-yl]-amine;

(4-isopropyl-3-methyl-phenyl)-[7-(2-trifluoromethyl-phenyl)-pyrido[3,2-d]pyrimidin-4-yl]amine;

[2-Ethyl-7-(3-trifluoromethyl-pyridin-2-yl)-quinazolin-4-yl]-(1-methanesulfonyl-2,3-dihydro-1H-indol-5-yl)-amine;

(4-tert-butyl-phenyl)-[6-(2-trifluoromethyl-phenyl)-isoquinolin-1-yl]-amine;

(4-trifluoromethyl-phenyl)-[6-(2-trifluoromethyl-phenyl)-isoquinolin-1-yl]amine;

N,N-dimethyl4-[7-(3-trifluoromethyl-pyridin-2-yl)-quinazolin-4-ylamino]-benzenesulfonamide;

(4-trifluoromethanesulfonyl-phenyl)-[7-(3-trifluoromethyl-pyridin-2-yl)-quinazolin-4-yl]-amine;

(4-trifluoromethanesulfonyl-phenyl)-[7-(3-trifluoromethyl-pyridin-2-yl)-pyrido[3,2-d]pyrimidin4-yl]-amine;

[4-(pyrrolidine-1-sulfonyl)-phenyl]-[7-(3-trifluoromethyl-pyridin-2-yl)-quinazolin-4-yl]-amine;

[4-(3-Dimethylamino-pyrrolidine-1-sulfonyl)-phenyl]-[7-(3-trifluoromethyl-pyridin-2-yl)-quinazolin-4-yl]-amine;

[4-(piperdine-1-sulfonyl)-phenyl]-[7-(3-trifluoromethyl-pyridin-2-yl)-quinazolin-4-yl]-amine;

[4-(morpholine4-sulfonyl)-phenyl]-[7-(3-trifluoromethyl-pyridin-2-yl)-quinazolin-4-yl]-amine;

(4-(morpholine4-sulfonyl)-phenyl]-[7-(3-trifluoromethyl-pyridin-2-yl)-pyrido[3,2-d]pyrimidin-4-yl]-amine;

[4-(2-methyl-piperdine-1-sulfonyl)-phenyl]-[7-(3-trifluoromethyl-pyridin-2-yl)-quinazolin-4-yl]-amine;

[4-(2,6-Dimethyl-piperidine-1-sulfonyl)-phenyl]-[7-(3-trifluoromethyl-pyridin-2-yl)-quinazolin-4-yl]-amine (chiral);

[4-(2-methyl-pyrrolidine-1-sulfonyl)-phenyl]-[7-(3-trifluoromethyl-pyridin-2-yl)-quinazolin-4-yl]-amine;

[4-(2,5-dimethyl-pyrrolidine-1-sulfonyl)-phenyl]-[7-(3-trifluoromethyl-pyridin-2-yl)-quinazolin-4-yl]-amine;

[4-(2,6-dimethyl-morpholine-4-sulfonyl)-phenyl]-[7-(3-trifluoromethyl-pyridin-2-yl)-quinazolin-4-yl]-amine;

[4-(2-methoxymethyl-pyrrolidine-1-sulfonyl)-phenyl]-[7-(3-trifluoromethyl-pyridin-2-yl)-quinazolin-4-yl]-amine (chiral);

[4-(2-methoxymethyl-pyrrolidine-1-sulfonyl)-phenyl]-[7-(3-trifluoromethyl-pyridin-2-yl)-quinazolin-4-yl]-amine (chiral);

N,N-diisopropyl-4-[7-(3-trifluoromethyl-pyridin-2-yl)-quinazolin-4-ylamino]-benzenesulfonamide;

N-(2-Hydroxy-1,1-dimethyl-ethyl)4-[7-(3-trifluoromethyl-pyridin-2-yl)-quinazolin-4-ylamino]-benzenesulfonamide;

(1-{4-[7-(3-trifluoromethyl-pyridin-2-yl)-quinazolin-4-ylamino-benzenesulfonyl}-pyrrolidin-2-yl)-methanol (chiral);

(1-{4-[7-(3-trifluoromethyl-pyridin-2-yl)-quinazolin-4-ylamino]-benzenesulfonyl}-pyrrolidin-2-yl)-methanol (chiral);

1-{4-[7-(3-Trifluoromethyl-pyridin-2-yl)-quinazolin-4-ylamino]-benzenesulfonyl}-pyrrolidin-3-ol (chiral);

N²-isobutyl-N⁴-(4-trifluoromethyl-phenyl)-7-(3-trifluoromethyl-pyridin-2-yl)-quinazoline-2,4-diamine;

[6-Bromo-7-(3-trifluoromethyl-pyridin-2-yl)-quinazolin-4-yl]-(4-trifluoromethyl-phenyl)-amine;

4-(4-Trifluoromethyl-phenylamino)-7-(3-trifluoromethyl-pyridin-2-yl)-quinazoline-6-carbonitrile;

N²-(3-Morpholin4-yl-propyl)-N⁴-(4-trifluoromethyl-phenyl)-7-(2-trifluoromethyl-phenyl)-pyrido[2,3-d]pyrimidine-2,4-diamine;

[2-(2,6-Dimethyl-morpholin4-yl)-7-(2-trifluoromethyl-phenyl)-pyrido[2,3-d]pyrimidin-4-yl]-(4-trifluoromethyl-phenyl)-amine;

[2-(3-Methyl-piperidin-1-yl)-7-(2-trifluoromethyl-phenyl)-pyrido[2,3-d]pyrimidin-4-yl]-(4-trifluoromethyl-phenyl)-amine;

(6-Chloro-pyridin-3-yl)-[7-(2-trifluoromethyl-phenyl)-quinazolin-4-yl]-amine;

1,1,1,3,3,3-Hexafluoro-2-{4-[7-(3-trifluoromethyl-pyridin-2-yl)-quinazolin-4-ylamino]-phenyl}-propan-2-ol;

(4-Trifluoromethoxy-phenyl)-[7-(3-trifluoromethyl-pyridin-2-yl)-quinazolin-4-yl]-amine;

N-Isopropyl-4-[7-(3-trifluoromethyl-pyridin-2-yl)-quinazolin-4-ylamino]-benzenesulfonamide;

[4-(4-Methyl-piperazine-1-sulfonyl)-phenyl]-[7-(3-trifluoromethyl-pyridin-2-yl)-quinazolin-4-yl]-amine;

Pyrrolidin-1-yl-{4-[7-(3-trifluoromethyl-pyridin-2-yl)-quinazolin-4-ylamino]-phenyl}-methanone;

[4-(3-Dimethylamino-pyrrolidine-1-sulfonyl)-phenyl]-[7-(3-trifluoromethyl-pyridin-2-yl)-quinazolin-4-yl]-amine;

N,N-Bis-(2-methoxy-ethyl)-4-[7-(3-trifluoromethyl-pyridin-2-yl)-quinazolin-4-ylamino]-benzenesulfonamide;

N-(3-Chloro-propyl)-4-[7-(3-trifluoromethyl-pyridin-2-yl)-quinazolin-4-ylamino]-benzenesulfonamide;

(4-Methanesulfonyl-phenyl)-[7-(3-trifluoromethyl-pyridin-2-yl)-quinazolin-4-yl]-amine;

4 [4-(Azetidine-1-sulfonyl)-phenyl]-[7-(3-trifluoromethyl-pyridin-2-yl)-quinazolin-4-yl]-amine;

[4-(Propane-1-sulfonyl)-phenyl]-[7-(3-trifluoromethyl-pyridin-2-yl)-quinazolin-4-yl]-amine;

(6-Isobutyl-pyridin-3-yl)-[7-(3-trifluoromethyl-pyridin-2-yl)-quinazolin-4-yl]-amine;

N-tert-Butyl-4-[7-(3-trifluoromethyl-pyridin-2-yl)-quinazolin-4-ylamino]-benzenesulfonamide;

[4-(4-Fluoro-piperidine-1-sulfonyl)-phenyl]-[7-(3-trifluoromethyl-pyridin-2-yl)-quinazolin-4-yl]-amine;

N-tert-Butyl-N-methyl4-[7-(3-trifluoromethyl-pyridin-2-yl)-quinazolin-4-ylamino]-benzenesulfonamide;

2-Methyl-2-{4-[7-(3-trifluoromethyl-pyridin-2-yl)-quinazolin-4-ylamino]-phenyl}-propan-1-ol;

[4-(2,2,2-Trifluoro-1-methyl-ethyl)-phenyl]-[7-(3-trifluoromethyl-pyridin-2-yl)-quinazolin-4-yl]-amine;

[2-Chloromethyl-7-(3-trifluoromethyl-pyridin-2-yl)-quinazolin-4-yl]-[4-(2,2,2-trifluoro-1-methyl-ethyl)-phenyl]-amine;

(2-Ethyl-7-(3-trifluoromethyl-pyridin-2-yl)-quinazolin-4-yl]-[4-(2,2,2-trifluoro-1-methyl-ethyl)-phenyl]-amine;

2-[4-(4-Trifluoromethyl-phenylamino)-quinazolin-7-yl]-nicotinic acid ethyl ester;

2-{2-tert-Butyl-5-[7-(3-trifluoromethyl-pyridin-2-yl)-quinazolin-4-ylamino]-phenoxy}-ethanol;

[4-tert-Butyl-3-(2-methylamino-ethoxy)-phenyl]-[7-(3-trifluoromethyl-pyridin-2-yl)-quinazolin-4-yl]-amine;

[4-tert-Butyl-3-(2-ethylamino-ethoxy)-phenyl]-[7-(3-trifluoromethyl-pyridin-2-yl)-quinazolin-4-yl]-amine;

[4-tert-Butyl-3-(2-propylamino-ethoxy)-phenyl]-[7-(3-trifluoromethyl-pyridin-2-yl)-quinazolin-4-yl]-amine;

[4-tert-Butyl-3-(2-butylamino-ethoxy)-phenyl]-[7-(3-trifluoromethyl-pyridin-2-yl)-quinazolin-4-yl]-amine;

{4-tert-Butyl-3-[2-(2-methoxy-ethylamino)-ethoxy]-phenyl}-[7-(3-trifluoromethyl-pyridin-2-yl)-quinazolin-4-yl]-amine;

[4-tert-Butyl-3-(2-dimethylamino-ethoxy)-phenyl]-[7-(3-trifluoromethyl-pyridin-2-yl)-quinazolin-4-yl]-amine;

[4-tert-Butyl-3-(2-diethylamino-ethoxy)-phenyl]-[7-(3-trifluoromethyl-pyridin-2-yl)-quinazolin-4-yl]-amine;

[4-tert-Butyl-3-(2-pyrrolidin-1-ylethoxy)-phenyl]-[7-(3-trifluoromethyl-pyridin-2-yl)-quinazolin-4-yl]-amine;

[4-tert-Butyl-3-(2-piperidin-1-yl-ethoxy)-phenyl]-[7-(3-trifluoromethyl-pyridin-2-yl)-quinazolin-4-yl]-amine;

[4-tert-Butyl-3-(2-morpholin4-yl-ethoxy)-phenyl]-[7-(3-trifluoromethyl-pyridin-2-yl)-quinazolin-4-yl]-amine;

{4-tert-Butyl-3-[2-(4-methyl-piperazin-1-yl)-ethoxy]-phenyl}-[7-(3-trifluoromethyl-pyridin-2-yl)-quinazolin-4-yl]-amine;

1-{4-[2-Methyl-7-(3-methyl-pyridin-2-yl)-pyrido[2,3-d]pyrimidin-4-ylamino]-phenyl}-cyclobutanecarbonitrile;

1-{4-[2-Cyclobutyl-7-(3-methyl-pyridin-2-yl)-pyrido[2,3-d]pyrimidin-4-ylamino]-phenyl}-cyclobutane carbonitrile;

(4-tert-Butyl-3-vinyl-phenyl)-[7-(3-trifluoromethyl-pyridin-2-yl)-pyrido[3,2-d]pyrimidin-4-yl]-amine;

3-{2-tert-Butyl-5-[7-(3-trifluoromethyl-pyridin-2-yl)-pyrido[3,2-d]pyrimidin-4-ylamino]-phenoxy}-propan-1-ol;

[4-tert-Butyl-3-(3-methylamino-propoxy)-phenyl]-[7-(3-trifluoromethyl-pyridin-2-yl)-pyrido[3,2-d]pyrimidin-4-yl]-amine;

[4-tert-Butyl-3-(3-ethylamino-propoxy)-phenyl]-[7-(3-trifluoromethyl-pyridin-2-yl)-pyrido[3,2-d]pyrimidin-4-yl]-amine;

[4-tert-Butyl-3-(3-propylamino-propoxy)-phenyl]-[7-(3-trifluoromethyl-pyridin-2-yl)-pyrido[3,2-d]pyrimidin-4-yl]-amine;

{4-tert-Butyl-3-[3-(2-methoxy-ethylamino)-propoxy]-phenyl}-[7-(3-trifluoromethyl-pyridin-2-yl)-pyrido[3,2-d]pyrimidin-4-yl]-amine;

[4-tert-Butyl-3-(3-dimethylamino-propoxy)-phenyl]-[7-(3-trifluoromethyl-pyridin-2-yl)-pyrido[3,2-d]pyrimidin-4-yl]-amine;

[4-tert-Butyl-3-(3-diethylamino-propoxy)-phenyl]-[7-(3-trifluoromethyl-pyridin-2-yl)-pyrido[3,2-d]pyrimidin-4-yl]-amine;

[4-tert-Butyl-3-(3-pyrrolidin-1-yl-propoxy)-phenyl]-[7-(3-trifluoromethyl-pyridin-2-yl)-pyrido[3,2-d]pyrimidin-4-yl]-amine;

[4-tert-Butyl-3-(3-piperidin-1-yl-propoxy)-phenyl]-[7-(3-trifluoromethyl-pyridin-2-yl)-pyrido[3,2-d]pyrimidin-4-yl]-amine;

[4-tert-Butyl-3-(3-morpholin-4-yl-propoxy)-phenyl]-[7-(3-trifluoromethyl-pyridin-2-yl)-pyrido[3,2-d]pyrimidin-4-yl]-amine;

[4-tert-Butyl-3-(3-butylamino-propoxy)-phenyl]-[7-(3-trifluoromethyl-pyridin-2-yl)-pyrido[3,2-d]pyrimidin-4-yl]-amine;

2-{2-tert-Butyl-5-[7-(3-trifluoromethyl-pyridin-2-yl)-pyrido[3,2-d]pyrimidin-4-ylamino]-phenyl}-ethanol;

[4-tert-Butyl-3-(2-morpholin-4-yl-ethyl)-phenyl]-[7-(3-trifluoromethyl-pyridin-2-yl)-pyrido[3,2-d]pyrimidin-4-yl]-amine;

[4-tert-Butyl-3-(2-methylamino-ethyl)-phenyl]-[7-(3-trifluoromethyl-pyridin-2-yl)-pyrido[3,2-d]pyrimidin-4-yl]-amine;

[4-tert-Butyl-3-(2-piperidin-1-yl-ethyl)-phenyl]-[7-(3-trifluoromethyl-pyridin-2-yl)-pyrido[3,2-d]pyrimidin-4-yl]-amine;

{4-tert-Butyl-3-[2-(2,6-dimethyl-morpholin-4-yl)-ethyl]-phenyl}-[7-(3-trifluoromethyl-pyridin-2-yl)-pyrido[3,2-d]pyrimidin-4-yl]-amine (cis);

(S,S)-{4-tert-Butyl-3-[2-(2,6-dimethyl-morpholin-4-yl)-ethoxy]-phenyl}-[7-(3-trifluoromethyl-pyridin-2-yl)-quinazolin-4-yl]-amine;

(R,R)-{4-tert-Butyl-3-[2-(2,6-dimethyl-morpholin-4-yl)-ethoxy]-phenyl}-[7-(3-trifluoromethyl-pyridin-2-yl)-quinazolin-4-yl]-amine;

{4-tert-Butyl-3-[2-(2,6-dimethyl-morpholin-4-yl)-ethoxy]-phenyl}-[7-(3-trifluoromethyl-pyridin-2-yl)-quinazolin-4-yl]-amine (cis);

2-{4-[2-Cyclobutyl-7-(3-methyl-pyridin-2-yl)-pyrido[2,3-d]pyrimidin-4-ylamino]-phenyl}-2-methyl-propionitrile;

2-Methyl-2-{4-[2-methyl-7-(3-methyl-pyridin-2-yl)-pyrido[2,3-d]pyrimidin-4-ylamino]-phenyl}-propionitrile;

N,N-Diethyl-2-{4-[2-methyl-7-(3-methyl-pyridin-2-yl)-pyrido[2,3-d]pyrimidin-4-ylamino]-phenyl}-isobutyramide;

[4-(2-Diethylamino-1,1-dimethyl-ethyl)-phenyl]-[2-methyl-7-(3-methyl-pyridin-2-yl)-pyrido[2,3-d]pyrimidin-4-yl]-amine;

2-{3-[7-(3-Trifluoromethyl-pyridin-2-yl)-pyrido[3,2-d]pyrimidin-4-ylamino]-phenoxy}-ethanol;

[3-(2-Morpholin-4-yl-ethoxy)-phenyl]-[7-(3-trifluoromethyl-pyridin-2-yl)-pyrido[3,2-d]pyrimidin-4-yl]-amine;

{3-[2-(2,6-Dimethyl-morpholin-4-yl)-ethoxy]-phenyl}-[7-(3-trifluoromethyl-pyridin-2-yl)-pyrido[3,2-d]pyrimidin-4-yl]-amine (cis);

2-{2-tert-Butyl-5-[7-(3-trifluoromethyl-pyridin-2-yl)-pyrido[3,2-d]pyrimidin-4-ylamino]-phenoxy}-1-morpholin4-yl-ethanone;

2-{2-tert-Butyl-5-[7-(3-trifluoromethyl-pyridin-2-yl)-pyrido[3,2-d]pyrimidin-4-ylamino]-phenoxy}-1-(2,6-dimethyl-morpholin4-yl)-ethanone (cis);

[2-Methyl-7-(3-methyl-pyridin-2-yl)-pyrido[2,3-d]pyrimidin-4-yl]-(4-trifluoromethoxy-phenyl)-amine;

(6-tert-Butyl-pyridin-3-yl)-[2-methyl-7-(3-methyl-pyridin-2-yl)-pyrido[2,3-d]pyrimidin-4-yl]-amine;

2-Methyl-2-{4-[2-methyl-7-(3-trifluoromethyl-pyridin-2-yl)-pyrido[2,3-d]pyrimidin-4-ylamino]-phenyl}-propionitrile;

[4-(2-Methoxy-1,1-dimethyl-ethyl)-phenyl]-[2-methyl-7-(3-trifluoromethyl-pyridin-2-yl)-pyrido[2,3-d]pyrimidin-4-yl]-amine;

[2-Methyl-7-(3-trifluoromethyl-pyridin-2-yl)-pyrido[2,3-d]pyrimidin-4-yl]-(6-trifluoromethyl-pyridin-3-yl)-amine;

[2-Methyl-7-(3-trifluoromethyl-pyridin-2-yl)-pyrido[2,3-d]pyrimidin-4-yl]-(4-trifluoromethane sulfonyl-phenyl)-amine;

3-Methyl-3-{4-[2-methyl-7-(3-trifluoromethyl-pyridin-2-yl)-pyrido[2,3-d]pyrimidin-4-ylamino]-phenyl}-butan-2-one;

3-Methyl-3-{4-[2-methyl-7-(3-methyl-pyridin-2-yl)-pyrido[2,3-d]pyrimidin4-ylamino]-phenyl}-butan-2-one;

[4-(1-Methoxy-1-methyl-ethyl)-phenyl]-[2-methyl-7-(3-methyl-pyridin-2-yl)-pyrido[2,3-d]pyrimidin-4-yl]-amine; and

(4-Medianesulfonyl-phenyl)-[2-methyl-7-(3-trifluoromethyl-pyridin-2-yl)-pyrido[2,3-d]pyrimidin-4-yl]-amine.

### Example 3

### Preparation of Representative VR1 Receptor Antagonists

This Example illustrates the preparation of representative substituted 2-aminoalkyl-quinazolin-4-ylamine analogues. Synthesis of the compounds provided in this Example is also described in PCT International Application Publication Number WO 03/062209, which published on Jul. 31, 2003.

### A. [2-Pyrrolidin-1-ylmethyl-7-(3-trifluoromethyl-pyridin-2-yl)-quinazolin-4-yl]-(4-trifluoro methyl-phenyl)-amine

### 1. 2-p-tolyl-3-trifluoromethyl-pyridine

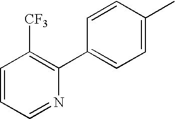

To a de-gassed mixture of 2-chloro-3-(trifluoromethyl)-pyridine (70.1 mmol), p-tolylboronic acid (70.6 mmol), and 2M Na₂CO₃ (175.0 mmol), in dimethyl ether (DME; 200 mL) under nitrogen, add Pd(PPh₃)₄ (2.8 mmol). Stir the mixture at 80° C. overnight, concentrate, and extract with EtOAc. Dry over Na₂SO₄, concentrate under vacuum, and pass through a silica gel pad to give 2-p-tolyl-3-trifluoromethyl-pyridine.

### 2. 2-(4-methyl-3-nitro-phenyl)-3-(trifluoromethyl)-pyridine

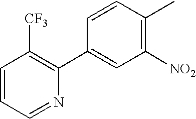

To a solution of 2-p-tolyl-3-trifluoromethyl-pyridine (8.4 mmol) in H₂SO₄ (6 mL) cautiously add fuming HNO₃ (2 ml). Stir the mixture for 60 minutes at room temperature. Pour the mixture onto ice-water (30 mL), extract with EtOAc, neutralize with 1 N NaOH, dry over Na₂SO₄, and concentrate under vacuum to obtain 2-(4-methyl-3-nitro-phenyl)-3-(trifluoromethyl)-pyridine.

### 3. 2-nitro-4-(3-trifluoromethyl-pyridin-2-yl)-benzoic acid

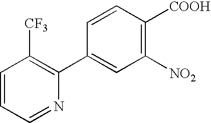

To a solution of 2-(4-methyl-3-nitro-phenyl)-3-(trifluoromethyl)-pyridine (7.1 mmol) in a mixture of pyridine (10 mL) and water (5 ml) add KMnO₄ (25.3 mmol) portionwise. Stir the mixture for 4 hours at 110° C. then add another 25.3 mmol of KMnO₄ with 10 ml of water. Stir the mixture at 110° C. overnight. Cool to room temperature, and filter through celite pad. Concentrate the filtrate under vacuum, dilute with water, and wash the aqueous solution with EtOAc. Neutralize the aqueous solution with 2 N HCl and collect the precipitate to give 2-nitro-4(3-trifluoromethyl-pyridin-2-yl)-benzoic acid.

### 4. 2-nitro-4-(3-trifluoromethyl-pyridin-2-yl)-benzamide

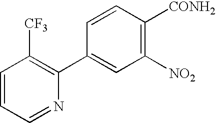

Reflux a mixture of 2-amino-4(3-trifluoromethyl-pyridin-2-yl)-benzoic acid (25 g) with SOCl₂ (50 ml) for 4 hours and concentrate. Dissolve the residue in dichloromethane (DCM), cool with ice-water bath, pass NH₃ gas through the solution for 30 minutes, and stir for 15 minutes at room temperature. Concentrate and wash with water to give 2-nitro-4-(3-trifluoromethyl-pyridin-2-yl)-benzamide.

### 5. 2-amino-4-(3-trifluoromethyl-pyridin-2-yl)-benzamide

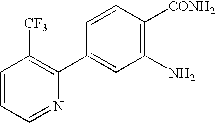

Hydrogenate 2-nitro-4-(3-trifluoromethyl-pyridin-2-yl)-benzamide (1.0 g, 0.0032 mol) with 50 psi of H₂ and 100 mg of 10% Pd/C in ethanol. After 16 hours, filter the mixture through celite and concentrate under reduced pressure to give 2-amino-4-(3-trifluoromethyl-pyridin-2-yl)-benzamide as a solid.

### 6. 2-chloromethyl-7-(3-trifluoromethyl-pyridin-2-yl)-3H-quinazolin-4-one

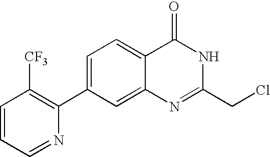

Heat a solution of 2-amino-4-(3-trifluoromethyl-pyridin-2-yl)-benzamide (100 mg, 0.356 mmol) in 2-chloro-1,1,1-trimethoxyethane (bp 138° C.) at 130° C. for 4 hours. Concentrate the mixture under reduced pressure to give 2-chloromethyl-7-(3-trifluoromethyl-pyridin-2-yl)-3H-quinazolin-4-one as an oil which crystallizes on standing.

### 7. 4-chloro-2-chloromethyl-7-(3-trifluoromethyl-pyridin-2-yl)-quinazoline

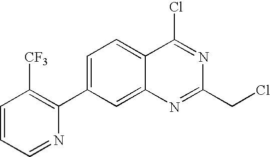

Reflux a mixture of 2-chloromethyl-7-(3-trifluoromethyl-pyridin-2-yl)-3H-quinazolin-4-one (obtained from the reaction above) and POCl₃ for 16 hours. Cool the mixture and concentrate under reduced pressure. Partition the residue between EtOAc and saturated NaHCO₃ solution. Wash the EtOAc portion with additional NaHCO₃ and then dry (Na₂SO₄) and concentrate under reduced pressure. Filter the brown residue through 2 inches of silica gel (1:1 EtOAc/hexanes eluent) and concentrate under reduced pressure to give 4-chloro-2-chloromethyl-7-(3-trifluoromethyl-pyridin-2-yl)-quinazoline.

### 8. [2-chloromethyl-7-(3-trifluoromethyl-pyridin-2-yl)-quinazolin-4-yl]-(4-trifluoro methyl-phenyl)-amine

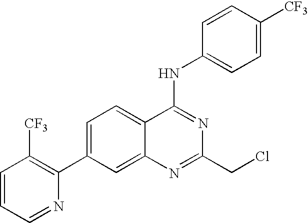

Heat a mixture of 4-chloro-2-chloromethyl-7-(3-trifluoromethyl-pyridin-2-yl)-quinazoline (42 mg, 0.117 mmol) and 4-trifluoromethyl-aniline (19 mg, 0.117 mmol) in isopropyl alcohol (1 mL) at 75° C. for 4 hours. Cool the mixture and wash the precipitate with isopropyl alcohol followed by ether to give [2-chloromethyl-7-(3-trifluoromethyl-pyridin-2-yl)-quinazolin-4-yl]-(4-trifluoromethyl-phenyl)-amine as the mono-HCl salt.

### 9. [2-Pyrrolidin-1-ylmethyl-7-(3-trifluoromethyl-pyridin-2-yl)-quinazolin-4-yl]-(4-trifluoromethyl-phenyl)-amine

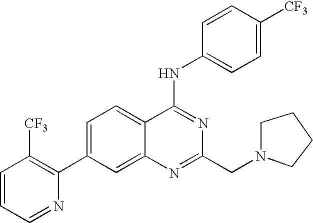

Heat a solution of [2-chloromethyl-7-(3-trifluoromethyl-pyridin-2-yl)-quinazolin-4-yl]-(4-trifluoromethyl-phenyl)-amine HCl (30 mg, 0.058 mmol) in pyrrolidine (1 mL) at 100° C. for 1 hour. Remove the excess pyrrolidine under reduced pressure and partition the residue between EtOAc and 10% NaOH solution. Dry the EtOAc layer (Na₂SO₄) and concentrate under reduced pressure to give [2-pyrrolidin-1-ylmethyl-7-(3-trifluoromethyl-pyridin-2-yl)-quinazolin-4-yl]-(4-trifluoromethyl-phenyl)-amine as a foam.

### B. [2-(2,6-Dimethyl-molpholin-4-ylmethyl)-7-(2-trifluoromethyl-phenyl)-pyrido[4,3-d]pyrimidin-4-yl]-(4-trifluoromethyl-phenyl)-amine

### 1. 4-Hydroxy-6-(2-trifluoromethyl-phenyl)-nicotinic acid ethyl ester

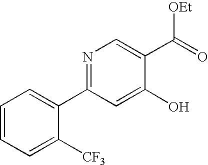

Dissolve lithium bis(trimethylsilyl)amide (LiHMDS) (34 g, 0.20 mol) in dry THF (150 mL) and cool to −70° C. under N₂ atm. Add 4-dimethylamino-3-ethoxy-but-3-en-2-one (15 g, 0.081 mol; see J. Heterocyclic Chem. (1987) 24:1669) and 2-(trifluoromethyl)benzoyl chloride (20.0 g, 0.097 mol) in THF (50 mL) into the solution for 10 minutes. Remove the cooling bath and stir for 10 minutes. Add ammonium acetate (10 g) and acetic acid (200 mL) to the reaction mixture and distil THF under reduced pressure. Heat the mixture at 60-65° C. for 18 hours, cool and add water (250 mL) and CH₂Cl₂ (250 mL). Separate the CH₂Cl₂ layer, and extract the aqueous layer twice with CH₂Cl₂ (2×250 mL each). Combine the CH₂Cl₂ extracts, dry (MgSO₄), and evaporate. Purify by silica gel chromatography to provide 4-hydroxy-6-(2-trifluoromethyl-phenyl)-nicotinic acid ethyl ester as a yellow solid.

### 2. 4-Chloro-6-(2-trifluoromethyl-phenyl)-nicotinic acid ethyl ester

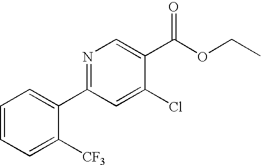

Heat a mixture of 4-hydroxy-6-(2-trifluoromethyl-phenyl)-nicotinic acid ethyl ester (9.0 g, 0.029 mol) in POCl₃ (22 g) at 110° C. for 2 hours. Evaporate the POCl₃, and add ice (100 g) followed by careful addition of saturated NaHCO₃. Extract with EtOAc, dry (MgSO₄), and evaporate to provide 4-chloro-6-(2-trifluoromethyl-phenyl)-nicotinic acid ethyl ester as a brown oil.

### 3. 4-Amino-6-(2-trifluoromethyl-phenyl)-nicotinamide

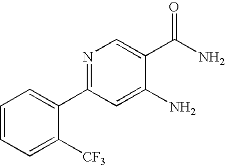

Heat a mixture of 4-chloro-6-(2-trifluoromethyl-phenyl)-nicotinic acid ethyl ester (5.2 g) and 28% aq. NH₄OH (100 mL) in a 350 ml resealable pressure vessel for 60 hours. Cool, extract with EtOAc (3×100 mL each), dry (MgSO₄), and evaporate to provide the crude product. Purify by silica gel chromatography to provide 4-amino-6-(2-trifluoromethyl-phenyl)-nicotinamide as a solid.

### 4. 2-(2, 6-Dimethyl-morpholin-4-ylmethyl)-7-(2-trifluoromethyl-phenyl)-pyrido[4,3-d]pyrimidin-4-ol

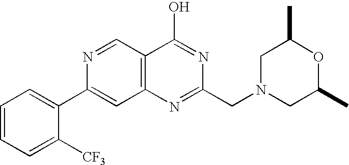

Heat a solution of 4-amino-6-(2-trifluoromethyl-phenyl)-nicotinamide (1 g, 3.5 mmol), 2,6-dimethyl-morpholin-4-yl)-acetic acid ethyl ester (2.85 g, 14 mmol), NaOEt (5.0 eq.) in EtOH (10 mL) for 20 hours. After cooling, concentrate the reaction mixture under reduced pressure, dilute the mixture with water (25 mL) and extract with EtOAc (3×25 mL 15 each), then wash twice with water (25 mL each) and dry with MgSO₄. Evaporate, and purify by flash chromatography to obtain 2-(2,6-dimethyl-morpholin4-ylmethyl)-7-(2-trifluoromethyl-phenyl)-pyrido[4,3-d]-pyrimidin-4-ol.

### 5. 4-Chloro-2-(2,6-dimethyl-morpholin-4-ylmethyl)-7-(2-trifluoromethyl-phenyl)-pyrido[4,3-d]pyrimidine

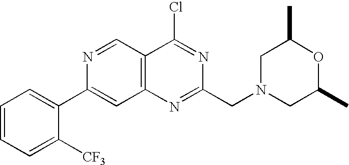

Reflux a mixture of 2-(2,6-dimethyl-morpholin-4-ylmethyl)-7-(2-trifluoromethyl-phenyl)-pyrido[4,3-d]-pyrimidin-4-ol (0.6 g), 2,6-lutidine (0.62 g), and POCl₃ (1.1 g) in CHCl₃ (15 mL) for 20 hours. Cool the mixture and concentrate under reduced pressure. Partition the residue between EtOAc and saturated NaHCO₃ solution. Wash the EtOAc portion with additional NaHCO₃ and then dry (Na₂SO₄) and concentrate under reduced pressure. Filter the brown residue through 2 inches of silica gel (1:1 EtOAc/hexanes eluent) and concentrate under reduced pressure to give 4-chloro-2-(2,6-dimethyl-morpholin-4-ylmethyl)-7-(2-trifluoromethyl-phenyl)-pyrido[4,3-d]pyrimidine.

### 6. [2-(2, 6-Dimethyl-morpholin-4-ylmethyl)-7-(2-trifluoromethyl-phenyl)-pyrido[4,3-d]pyrimidin-4-yl]-(4-trifluoromethyl-phenyl)-amine

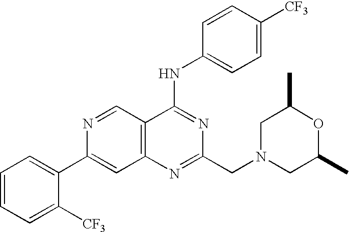

Heat a mixture of 4-chloro-2-(2,6-dimethyl-morpholin4-ylmethyl)-7-(2-trifluoromethyl-phenyl)-pyrido[4,3-d]pyrimidine (43.7 mg, 0.1 mmol) and 4-trifluoromethyl-aniline (16.1 mg, 0.1 mmol) in AcCN (1 mL) at 80° C. for 24 hours. Cool the mixture and wash the precipitate with ether to give 4-chloro-2-(2,6-dimethyl-morpholin-4-ylmethyl)-7-(2-trifluoromethyl-phenyl)-pyrido[4,3-d]pyrimidine as the mono-HCl salt.

### C. [2-Morpholin-4-ylmethyl-7(3-trifluoromethyl-pyridin-2-yl)-pyrido[3,2-a]pyrimidin-4-yl]-(4-trifluoromethyl-phenyl)-amine

### 1. 6′-Methoxy-3-trifluoromethyl-[2,3′bipyridinyl

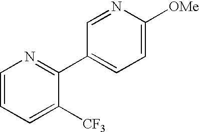

Heat a mixture of 2-chloro-3-trifluoromethylpyridine (37 g, 0.2 mol), 2-methoxypyridine-5-boronic acid (32 g, 0.21 mol), tetrakis(triphenylphosphine)palladium(0) (9 g, 7 mmol) and 2M potassium carbonate (150 mL) in toluene (500 mL) under a nitrogen atmosphere, at 90° C. for 8 hours. Cool the reaction mixture and separate the layers. Extract the aqueous layer with ethyl acetate (2×250 mL) and wash the combined organics with 4M sodium hydroxide (250 mL), water (250 mL), and brine (250 mL). Dry (MgSO₄) and concentrate under reduced pressure. Purify the oil by flash chromatography on silica gel (50% ether/50% hexane) to give the title compound as a colorless oil.

### 2. 3-Trifluoromethyl-1′H-[2,3′bipyridinyl-6′-one

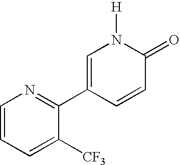

Heat 6′-methoxy-3-trifluoromethyl-[2,3′]bipyridinyl (41 g, 0.16 mol) in 30% HBr/AcOH (100 mL) to reflux for 1 hour. Cool the mixture and filter, and wash the precipitate with ether (100 mL). Transfer the precipitate into 10M sodium hydroxide (500 mL) and stir for 1 hour, and treat the solution with hydrochloric acid until the solution is pH 7. Collect the white solid by filtration and air dry to give the title compound as a white solid.

### 3. 5′-Nitro-3-trifluoromethyl-1′H-[2, 3′bipyridinyl-6′-one

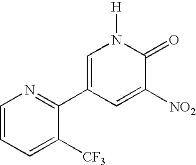

To a solution of 3-trifluoromethyl-1′H-[2,3′]bipyridinyl-6′-one (25 g, 0.1 mol) in concentrated sulfuric acid (100 mL) at 0° C., add dropwise a solution of fuming nitric acid (35 mL) and concentrated sulfuric acid (10 mL). Heat the reaction mixture to 70° C. for 1 hour, cool and pour onto ice (500 mL). Filter the mixture and treat the filtrate with 10 M sodium hydroxide until the solution is at pH 4-5. Collect the precipitate by filtration and air dry to give the title compound as a white solid.

### 4. 6′-Chloro-5′-nitro-3-trifluoromethyl-[2,3′bipyridinyl

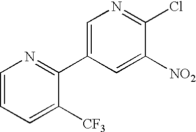

Heat a solution of 5′-nitro-3-trifluoromethyl-1′H-[2,3′]bipyridinyl-6′-one (25 g, 0.088 mol), thionyl chloride (300 mL) and DMF (3 mL) to reflux for 4 hours. Remove the volatiles by rotary evaporation and partition the residue between ethyl acetate (350 mL) and saturated sodium bicarbonate solution (250 mL). Extract the aqueous layer with further ethyl acetate (250 mL) and wash the combined organics with brine (250 mL). Dry (MgSO₄) and concentrate under reduced pressure to give the title compound as a yellow oil.

### 5. 6′-Chloro-3-trifluoromethyl-[2,3′bipyridinyl-5′-ylamine

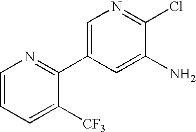

To a solution of 6′-chloro-5′-nitro-3-trifluoromethyl-[2,3′]bipyridinyl (25 g, 0.082 mol) and calcium chloride (11 g, 0.1 mol) in ethanol (300 mL) and water (50 mL), add iron powder (45 g, 0.82 mol). Heat the solution to reflux for 1.5 hours, cool and filter through Celite. Concentrate the mixture under reduced pressure, re-dissolve in ethyl acetate (300 mL) and wash with brine (200 mL). Concentrate the solution under reduced pressure and purify by flash chromatography on silica gel (50% ether/50% hexane) to give the title compound as a pale yellow solid.

### 6. 3-Amino-5-[3-(trifluoromethyl)(2-pyridyl)]pyridine-2-carboxamide

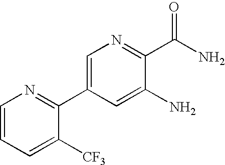

Heat a solution of 6′-chloro-3-trifluoromethyl-[2,3′]bipyridinyl-5′-ylamine (25 g, 0.091 mol), zinc cyanide (6.75 g, 0.058 mol), tris[dibenzylidineacetone]di-palladium (also referred to as “pd₂(dba)₃”;2.63 g, 2.86 mmol), 1,1′-bis(diphenylphosphino)ferrocene (also referred to as “DPPF”; 3.16 g, 5.72 mmol) in DMF (250 mL) and water (2.5 mL), under a nitrogen atmosphere, at 120° C. for 1 hour. Add water (30 mL) and heat the solution at 120° C. for a further 4 hours to complete the hydrolysis. Cool the reaction to 0° C. and add a solution of saturated ammonium chloride (200 ml), water (200 mL) and concentrated ammonium hydroxide (50 mL). After stirring at 0° C. for 1 hour, filter the yellow precipitate, and wash with water (200 mL) and a 1:1 mixture of ether-hexane (200 mL). Dry the solid in air and then in a vacuum oven to give the title compound.

### 7. 2-(Chloromethyl)-7-[3-(trifluoromethyl)(2-pyridyl)]-3-hydropyridino[3,2-d]pyrimidin-4-one

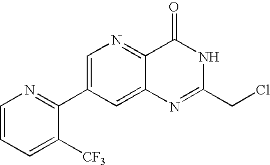

Heat a solution of 3-amino-5-[3-(trifluoromethyl)(2-pyridyl)]pyridine-2-carboxamide (23 g, 81.5 mmol) and 2-chloro-1,1,1-trimethoxyethane (250 mL) at 130° C. for 1 hour. Remove the volatiles by evaporation and triturate the solid (50% ether/50% hexane) to give the title compound as a light brown solid.

### 8. 2-(Morpholin-4-ylmethyl)-7-[3-(trifluoromethyl)(2-pyridyl)]-3-hydropyridino[3,2-d]pyrimidin-4-one

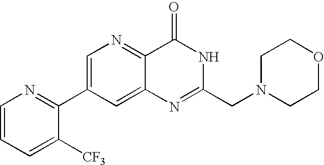

Heat a solution of 2-(chloromethyl)-7-[3-(trifluoromethyl)(2-pyridyl)]-3-hydropyridino[3,2-d]pyrimidin-4-one (20 g, 0.058 mol), morpholine (15.66 g, 0.18 mol) in acetonitrile (500 mL) at 80° C. for 12 hours. Evaporate the solution and partition the residue between ethyl acetate (500 mL) and saturated sodium bicarbonate solution (500 mL). Extract the aqueous layer with further ethyl acetate (250 mL) and wash the combined organics with brine (500 mL). Dry (MgSO₄) and concentrate under reduced pressure to give the title compound as a brown solid.

### 9. 4-({4-Chloro-7-[3-(trifluoromethyl)(2-pyridyl)]pyridino[3,2-d]pyrimidin-2-yl}methyl)-methylmorpholine

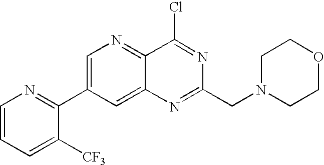

Heat a solution of 2-(morpholin-4-ylmethyl)-7-[3-(trifluoromethyl)(2-pyridyl)]-3-hydropyridino[3,2-d]pyrimidin-4-one (11.73 g, 0.03 mol), POCl₃ (13.8 g, 0.09 mol) and 2,6-lutidine (9.63 g, 0.09 mol) in chloroform (500 mL) at 60° C. for 12 hours. Evaporate the solution and partition the residue between ethyl acetate (500 mL) and saturated sodium bicarbonate solution (500 mL). Extract the aqueous layer with further ethyl acetate (250 mL) and wash the combined organics with brine (500 mL). Dry (MgSO₄) and concentrate under reduced pressure to give the title compound as a brown solid.

### 10. [2-Morpholin-4-ylmethyl-7(3-trifluoromethyl-pyridin-2-yl)-pyrido[3,2-a]pyrimidin-4-yl]-(4-trifluoromethyl-phenyl)-amine

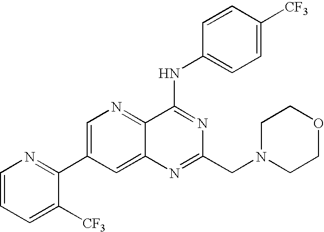

Heat a solution of 4-({4-chloro-7-[3-(trifluoromethyl)(2-pyridyl)]pyridino[3,2-d]pyrimidin-2-yl}methyl)-methylmorpholine (12.2 g, 0.03 mol), 4-(trifluoromethyl)aniline (4.8 g, 0.03 mol) in acetonitrile (500 mL) at 80° C. for 12 hours. Evaporate the solution and partition the residue between ethyl acetate (500 mL) and saturated sodium bicarbonate solution (500 mL). Extract the aqueous layer with further ethyl acetate (2×250 mL) and wash the combined organics with brine (500 mL). Dry (MgSO₄) and concentrate under reduced pressure. Purify the residue by flash chromatography on silica gel (90% ether/10% hexane then 100% ether) to give the title compound.

### D. [2-(2-Pyrrolidin-1-yl-ethyl)-7-(3-trifluoromethyl-pyridiny-2-yl)-quinazolin-4-yl]-(4-trifluoromethyl-phenyl)-amine

### 1. 3-Benzyloxy-propionic acid

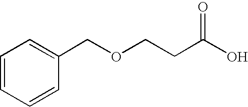

In small portions, add sodium hydride (2.22 g, 60% dispersion in mineral oil, 55.4 mmol) to a cold (0° C.) solution of benzyl alcohol (4.0 g, 37 mmol) in toluene (100 mL). Add ethyl 3-bromopropionate (8.0 g, 44 mmol) dropwise to the mixture, allow the resulting solution to warm to room temperature and stir for 1 hour. Quench the reaction with the addition of water until all bubbling ceases. Dilute the mixture with ethyl acetate (100 mL) and extract with water (100 mL) and brine (100 mL). Dry the organic extract over Na₂SO₄ and remove the solvent under reduced pressure to yield the crude ester as a clear oil. Dissolve the oil in methanol (20 mL) and 6 N NaOH (20 mL), and stir for 1 hour. Concentrate the mixture (approximately 20 mL) and dilute with water (20 mL). Extract the aqueous mixture once with CH₂Cl₂ (40 mL). Acidify the aqueous phase with conc. HCl and extract with EtOAc (3×50 mL). Dry the combined EtOAc extracts over Na₂SO₄. Remove the solvent under reduced pressure to yield the title compound as a clear oil that solidifies upon standing.

### 2. 2-(2-Benzyloxy-ethyl)-7-(3-trifluoromethyl-pyridiny-2-yl)-3H-quinazolin-4-one

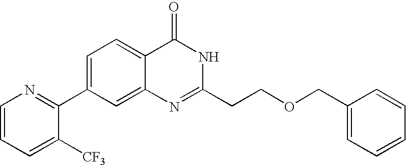

Cool a solution of 3-benzyloxy-propionic acid (1.66 g, 9.19 mmol) in hexanes (40 mL) to 0° C. and add oxalyl chloride (3.50 g, 27.6 mmol) dropwise. After the addition is completed, add DMF (2 drops), and stir the resulting mixture for 1 hour. Remove the solvent under reduced pressure and dissolve the crude acid chloride in dry THF (20 mL). In a separate flask, dissolve 2-amino-4-(3-trifluoromethyl-pyridin-2-yl)-benzamide (2.35 g, 8.37 mmol) in dry THF (40 mL) and pyridine (0.727 g, 9.19 mmol) and cool to 0° C. Add the solution containing the crude acid chloride dropwise to the second solution. Allow the mixture to warm to room temperature and stir for 1 hour. Add a solution of 10% NaOH_{(aq)} (20 mL) to the mixture and stir the solution for 1 hour. Concentrate the mixture (˜20 mL), dilute with water (20 mL), and acidify with conc. HCl. Extract the resulting solution with EtOAC (3×50 mL). Wash the combined organic extracts with brine and dry over Na₂SO₄. Remove the solvent under reduced pressure to yield the title compound as a white solid.

### 3. 2-(2-Benzyloxy-ethyl)-4-chloro-7-(3-trifluoromethyl-pyridiny-2-yl)-quinazoline

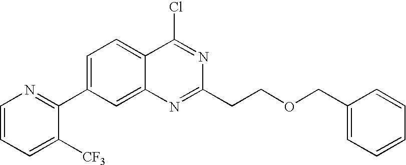

Dissolve 2-(2-benzyloxy-ethyl)-7-(3-trifluoromethyl-pyridiny-2-yl)-3H-quinazolin-4-one (3.24 g, 7.62 mmol) in CHCl₃ (40 mL) and 2,6-lutidine (2.45 g, 22.9 mmol). Add phosphorous oxychloride (1.77 mL, 19.0 mmol) dropwise and heat the resulting solution to reflux for 18 hours. Cool the solution and remove the solvent under reduced pressure. Partition the crude residue between EtOAc (200 mL) and saturated NaHCO₃ (aq) (200 mL). Remove the organic phase and extract the aqueous phase with EtOAc (200 mL). Combine the two organic extracts, wash with brine (200 mL), and dry over Na₂SO₄. Remove the solvent to yield the title compound as a light brown solid.

### 4. [2-(2-Benzyloxy-ethyl)-7-(3-trifluoromethyl-pyridiny-2-yl)-quinazolin-4-yl]-(4-trifluoromethyl-phenyl)-amine

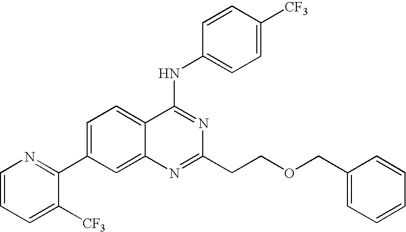

Dissolve 2-(2-benzyloxy-ethyl)-4-chloro-7-(3-trifluoromethyl-pyridiny-2-yl)-quinazoline (2.47 g, 5.57 mmol) into a solution of acetonitrile (50 mL) and 4-trifluoromethyl-aniline (0.986 g, 6.12 mmol). Heat the mixture to 80° C. for 2 hours, to form a white precipitate. Cool the solution in an ice bath and add diethyl ether (25 mL). Filter off the white precipitate and dry in a vacuum oven to yield the title compound as the mono-hydrochloride salt (2.96 g, 87.8 %).

### 5. 2-[4-(4-Trifluoromethyl-phenylamino)-7-(3-trifluoromethyl-pyridin-2-yl)-quinazolin-2-yl]-ethanol

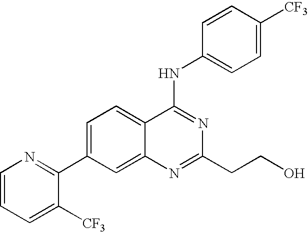

Dissolve [2-(2-benzyloxy-ethyl)-7-(3-trifluoromethyl-pyridiny-2-yl)-quinazolin4-yl]-(4-trifluoromethyl-phenyl)-amine hydrochloride (2.96 g, 4.89 mmol) in MeOH (150 mL) and add 10% Pd/C (200 mg). Hydrogenate the mixture at 50 p.s.i. at 60° C. for 8 hours. Quickly filter the mixture through Celite and wash the Celite filter cake with hot MeOH (200 mL). Remove the solvent under reduced pressure to yield the mono-hydrochloride salt of title compound as a white solid.

### 6. [2-(2-Chloro-ethyl)-7-(3-trifluoromethyl-pyridiny-2-yl)-quinazolin-4-yl]-(4-trifluoromethyl-phenyl)-amine

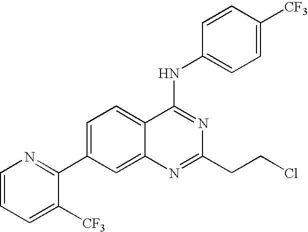

Dissolve 2-[4-(4-trifluoromethyl-phenylamino)-7-(3-trifluoromethyl-pyridin-2-yl)-quinazolin-2-yl]-ethanol hydrochloride (1.54 g, 2.99 mmol) in thionyl chloride (20 mL) and heat to 60° C. for 1 hour. Remove the excess thionyl chloride under reduced pressure and triturate the residue with diethyl ether to yield the mono-hydrochloride salt of the title compound as a light brown solid.

### 7. [2-(2-Pyrrolidin-1-yl-ethyl)-7-(3-trifluoromethyl-pyridiny-2-yl)-quinazolin-4-yl]-(4-trifluoromethyl-phenyl)-amine

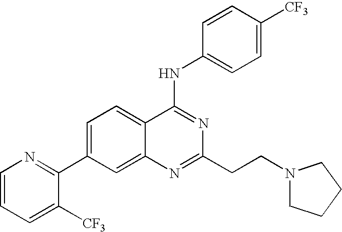

Dissolve [2-(2-chloro-ethyl)-7-(3-trifluoromethyl-pyridiny-2-yl)-quinazolin-4-yl]-(4-trifluoromethyl-phenyl)-amine hydrochloride (20 mg, 0.0375 mmol) in CH₃CN/10% diisopropylethylamine (0.187 mL) and add a 0.2 N solution of pyrrolidine in acetonitrile (0.281 mL). Heat the mixture at 70° C. for 18 hours. Remove the solvent under reduced pressure and partition the crude reaction mixture between EtOAc (1 mL) and 1 N (NaOH). Remove the organic extract and extract the aqueous phase again with EtOAc (1 mL). Chromatograph the combined organic extracts through a small pad of silica gel, eluting with acetone to yield the title compound as a light brown solid.

E. [2-(3-morpholin4-yl-propyl)7-(3-trifluoromethyl-pyridin-2-yl)-quinazolin-4-yl]-(4-trifluoromethyl-phenyl)-amine hydrochloride

### 1. 3-[4-hydroxy-7-(3-trifluoromethyl-pyridin-2-yl)-quinazolin-2-yl]-propionic acid ethyl ester

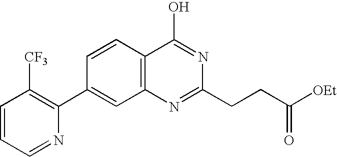

To a solution of 2-amino-4-(3-trifluoromethyl-pyridin-2-yl)-benzamide (0.5 mmol) and pyridine (0.55 mmol) in THF (5 ml), add 3-chlorocarbonyl-propionic acid ethyl ester chloride (0.55 mmol). Stir the mixture for 20 minutes at room temperature, add 20 ml of 21% NaOEt in EtOH, and stir for 30 minutes at 50° C. Concentrate, add water, filter, acidify to pH 6, and collect the precipitate to give 3-[4-hydroxy-7-(3-trifluoromethyl-pyridin-2-yl)-quinazolin-2-yl]-propionic acid ethyl ester.

### 2. 3-[4-chloro-7-(3-trifluoromethyl-pyridin-2-yl)-quinazolin-2-yl]-propionic acid ethyl ester

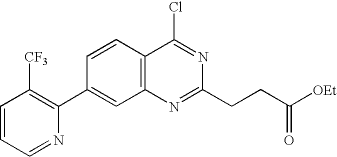

Using procedures analogous to those already described, 3-[4-chloro-7-(3-trifluoromethyl-pyridin-2-yl)-quinazolin-2-yl]-propionic acid ethyl ester is prepared from 3-[4-hydroxy-7-(3-trifluoromethyl-pyridin-2-yl)-quinazolin-2-yl]-propionic acid ethyl ester.

### 3. 3-[4-(4-trifluoromethyl-phenylamino)-7-(3-trifluoromethyl-pyridin-2-yl)-quinazolin-2-yl]-propionic acid ethyl ester

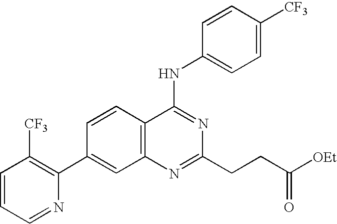

Using procedures analogous to those already described, 3-[4-(4-trifluoromethyl-phenylamino)-7-(3-trifluoromethyl-pyridin-2-yl)-quinazolin-2-yl]-propionic acid ethyl ester is prepared from 3-[4-chloro-7-(3-trifluoromethyl-pyridin-2-yl)-quinazolin-2-yl]-propionic acid ethyl ester.

### 4. 3-[4-(4-trifluoromethyl-phenylamino)-7-(3-trifluoromethyl-pyridin-2-yl)-quinazolin-2-yl]-propionic acid

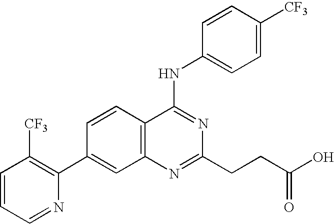

To a mixture of 3-[4-(4-trifluoromethyl-phenylamino)-7-(3-trifluoro-methyl-pyridin-2-yl)-quinazolin-2-yl]-propionic acid ethyl ester (0.5 mmol) in THF (20 ml) and H₂O (20 ml), add LiOH (1.5 mmol). Stir the mixture for 2 hours at 60° C. Concentrate, add water, extract with ether, acidify the aqueous layer to pH 4-5, extract with EtOAc, and concentrate to give 3-[4-(4-trifluoromethyl-phenylamino)-7-(3-trifluoro-methyl-pyridin-2-yl)-quinazolin-2-yl]-propionic acid.

### 5. 1-morpholin-4-yl-3-[4-(4-trifluoromethyl-phenylamino)-7-(3-trifluoromethyl-pyridin-2-yl)-quinazolin-2-yl]-propan-1-one

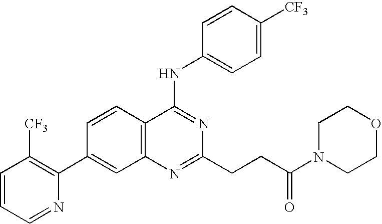

To a solution of 3-[4-(4-trifluoromethyl-phenylamino)-7-(3-trifluoro-methyl-pyridin-2-yl)-quinazolin-2-yl]-propionic acid (0.5 mmol) and triethylamine (0.5 mmol) in DMF (10 ml), add benzotriazol-1-yl-oxy-tris(dimethylamino)phosphonium hexafluorophosphate (BOP; 0.5 mmol). Stir the mixture for 18 hours at room temperature, dilute with water, extract with EtOAc, and wash with brine. Concentrate to give 1-morpholin-4-yl-3-[4-(4-trifluoromethyl-phenylamino)-7-(3-trifluoromethyl-pyridin-2-yl)-quinazolin-2-yl]-propan-1-one.

### 6. [2-(3-morpholin-4-yl-propyl)-7-(3-trifluoromethyl-pyridin-2-yl)-quinazolin-4-yl]-(4-trifluoromethyl-phenyl)-amine hydrochloride

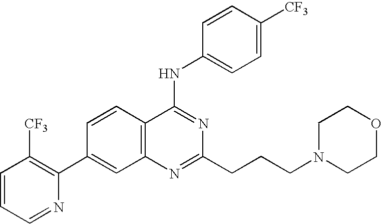

To a solution of 1-morpholin-4-yl-3-[4-(4-trifluoromethyl-phenylamino)-7-(3-trifluoromethyl-pyridin-2-yl)-quinazolin-2-yl]-propan-1-one (0.14 mmol) in THF (20 ml), add LAH (0.67 mmol). Stir the mixture for 6 hours at room temperature, quench with 10% NaOH, extract with EtOAc, dry over Na₂SO₄, and add HCl-EtOAc. Collect the precipitate to give [2-(3-morpholin-4-yl-propyl)-7-(3-trifluoromethyl-pyridin-2-yl)-quinazolin-4-yl]-(4-trifluoromethyl-phenyl)-amine hydrochloride.

### F. 4-trifluoromethylphenyl-[2-(2,6-dimethylmorphonli-4-ylmethyl)-7-(2-trifluoromethyl phenyl)-quinazolin-4-yl]-amine

### 1. 7-Bromo-2-chloromethyl-3H-quinazolin-4-one

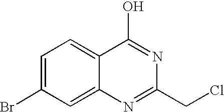

Reflux a solution of 2-amino-4-bromobenzamide (27 g, 0.13 mol; see Joshi and Chaudhari, (1987) Indian J. Chem., Sect. B, 26B(6):6024) in 2-chloro-1,1,1-trimethoxyethane (50 mL) for 30 minutes, during which time a large precipitate appears. Evaporate the mixture fully and triturate with ether to collect 7-bromo-2-chloromethyl-3H-quinazolin-4-one as a white solid.

### 2. 7-Bromo-4-chloro-2-chloromethylquinazoline

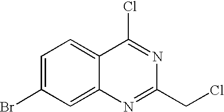

Heat a mixture of 7-bromo-2-chloromethyl-3H-quinazolin-4-one (5 g, 18.2 mmol), 2,6-lutidine (5 g), and phosphorus oxychloride (5 mL) in 1,2-dimethoxyethane (500 mL) at 80° C. for 16 hours. Cool the mixture to room temperature and fully evaporate the mixture, then dilute with ether and wash with water. Dry the solvent (Na2SO₄) and evaporate the ether to obtain 7-bromo4-chloro-2-chloromethylquinazoline as a yellow solid.

### 3. 7-Bromo-2-chloromethylquinazolin-4-yl)-(4-trifluoromethylphenyl)-amine

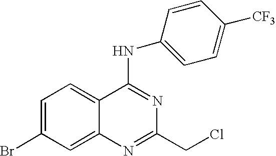

Heat a mixture of 7-bromo-4-chloro-2-chloromethylquinazoline (1168 mg, 4.0 mmol) and 4-(trifluoromethyl)aniline (644 mg, 4.0 mmol) in chloroform (50 mL) at 60° C. for 16 hours. Cool and collect the precipitated product 7-bromo-2-chloromethylquinazolin-4-yl)-(4-trifluoromethylphenyl)-amine as the HCL salt.

### 4. [7-Bromo-2-(cis-2,6-dimethylmorpholin-4-ylmethyl)-quinazolin-4-yl]-4-(trifluoro methylphenyl)-amine

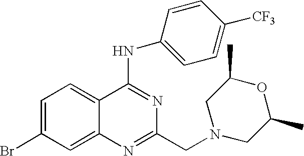

Heat a mixture of 7-bromo-2-chloromethylquinazolin-4-yl)-(4-trifluoromethylphenyl)-amine (416 mg, 1.0 mmol), cis-2,6-dimethylmorpholine (150 mg, 1.3 mmol), and triethylamine (202 mg, 2.0 mmol) in N,N-dimethylacetamide (7 mL) for 1 hour. Cool to room temperature, dilute with EtOAc (50 mL), and wash four times with water (25 mL each). Dry (Na₂SO₄) and evaporate. Triturate with ether to give [7-bromo-2-(cis-2,6-dimethylmorpholin-4-ylmethyl)-quinazolin-4-yl]-4-(trifluoromethylphenyl)-amine as a yellow solid.

### 5. [2-(cis-2, 6-dimethylmorpholin-4-yloxymethyl)-7-(2-trifluoromethylphenyl)-quinazolin-4-yl]-(4-trifluoromethylphenyl)-amine

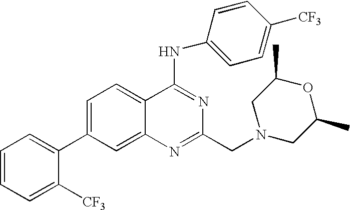

Under nitrogen, heat a mixture of [7-bromo-2-(cis-2,6-dimethylmorpholin-4-ylmethyl)-quinazolin-4-yl]4-(trifluoromethylphenyl)-amine (75 mg, 0.15 mmol), 2-(trifluoromethyl phenyl)boronic acid (45 mg, 0.23 mmol), tetrakis(triphenylphosphine)palladium(0) (21 mg, 0.018 mmol), 2M Na₂CO₃ in water (1 mL), and 1,2-dimethoxyethane (5 mL) at 60° C. for 16 hours. Cool the mixture to room temperature, dilute with EtOAc, and wash twice with water (10 mL each). Dry the organic layer (Na₂SO₄) and evaporate. Purify by preparative TLC (9:1 CH₂CL₂:MeOH) to obtain [2-(cis-2,6-dimethylmorpholin-4-yloxymethyl)-7-(2-trifluoro methylphenyl)-quinazolin-4-yl]-(4-trifluoromethylphenyl)-amine as a yellow solid.

### G. [7-(3-Methyl-pyridin-2-yl)-2-pyrrolidin-1-ylmethyl-pyrido[3,2-d]pyrimidin-4-yl]-(4-trifluoromethyl-phenyl)-amine

### 1. 5-Bromo-3-nitropyridine-2-carbonitrile

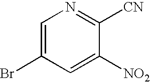

Heat a solution of 2-amino-5-bromo-3-nitropyridine (2.18 g, 10 mmol), cuprous cyanide (1.33 g, 15 mmol) and tert-butylnitrite (2.0 mL, 15 mmol) in acetonitrile (50 mL) to 60° C. for 2 hours. Cool the solution and partition between ethyl acetate (100 mL) and saturated aqueous NaHCO₃ (100 mL). Extract the aqueous solution with ethyl acetate (2×50 mL), wash with water (100 mL), brine (100 mL), dry (MgSO₄) and evaporate. Purify the solid by flash chromatography on silica gel (25% ether/75% hexane) to obtain the title compound as a pale yellow solid.

### 2. 5-(3-Methyl(2-pyridyl))-3-nitropyridine-2-carbonitrile

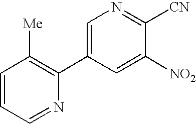

Heat a solution of 5-bromo-3-nitropyridine-2-carbonitrile (228 mg, 1.0 mmol), tetrakis(triphenylphosphine)palladium(0) (15 mg), 3-methyl-2-pyridylzinc bromide (0.5 M in THF, 3 mL, 1.5 mmol) in THF (5 mL) to 60° C. for 2 hours. Cool the solution and partition between ethyl acetate (10 mL) and saturated aqueous NaHCO₃ (10 mL). Extract the aqueous solution with ethyl acetate (2×15 mL), wash with water (10 mL), brine (10 mL), dry (MgSO₄) and evaporate to obtain the title compound as a pale yellow solid. 3. 3-Amino-5-(3-methyl(2-pyridyl))pyridine-2-carboxamide

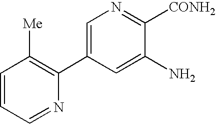

Heat a solution of 5-(3-methyl(2-pyridyl))-3-nitropyridine-2-carbonitrile (1 g, 4.1 mmol), iron (2.3 g, 40 mmol) and calcium chloride (560 mg, 5 mmol) in ethanol (15 mL) and water (4 mL) to reflux for 1 hour. Cool the mixture, filter through Celite and wash with ethyl acetate. Evaporate the filtrate and re-dissolve the residue in ethyl acetate, wash with water and then with brine, dry (MgSO₄) and evaporate to obtain the title compound as a pale yellow solid.

### 4. [7-(3-Methyl-pyridin-2-yl)-2-pyrrolidin-1-ylmethyl-pyrido[3,2-d]pyrimidin-4-yl]-(4-trifluoromethyl-phenyl)-amine

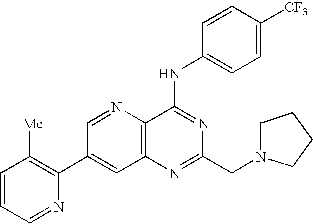

The title compound is prepared from 3-amino-5-(3-methyl(2-pyridyl))pyridine-2-carboxamide in a manner analogous to that used for the preparation of [2-pyrrolidin-1-ylmethyl-7-(3-trifluoromethyl-pyridin-2-yl)-quinazolin-4-yl]-(4-trifluoromethyl-phenyl)-amine (Example 1.A, steps 6 to 9).

### H. Additional Representative Substituted 2-Aminoalkyl-Quinazolin-4-ylamine Analogues

Those having skill in the art will recognize that the starting materials may be varied and additional steps employed to produce other compounds encompassed by the present invention. The following compounds were prepared using the above methods, with readily apparent modifications, and may be used in the compositions and methods provided herein:

(1-{3-[4-(4-Trifluoromethyl-phenylamino)-7-(3-trifluoromethyl-pyridin-2-yl)-quinazolin-2-yl]-propyl}-piperidin-4-yl)-methanol;

(2,6-Dimethyl-morpholin-4-yl)-[4-(4-trifluoromethyl-phenylamino)-7-(3-trifluoromethyl-pyridin-2-yl)-quinazolin-2-yl]-methanone (cis);

(4-Cyclopropyl-phenyl)-[2-(2,6-dimethyl-morpholin-4-ylmethyl)-7-(3-trifluoromethyl-pyridin-2-yl)-quinazolin-4-yl]-amine (cis);

(4-sec-Butyl-phenyl)-[7-(3-chloro-pyridin-2-yl)-2-(2,6-dimethyl-morpholin-4-ylmethyl)-quinazolin-4-yl]-amine (cis);

(4-tert-Butyl-phenyl)-[2-(1,1-dioxo-1λ⁶-isothiazolidin-2-ylmethyl)-7-(3-trifluoromethyl-pyridin-2-yl)-quinazolin-4-yl]-amine;

(4-tert-Butyl-phenyl)-[2-(2,6-dimethyl-morpholin-4-ylmethyl)-7-(2-trifluoromethyl-phenyl)-pyrido[4,3-d]pyrimidin-4-yl]-amine (cis);

(4-tert-Butyl-phenyl)-[2-(2,6-dimethyl-morpholin-4-ylmethyl)-7-(3-trifluoromethyl-pyridin-2-yl)-pyrido[2,3-d]pyrimidin-4-yl]-amine (cis);

(4-tert-Butyl-phenyl)-[2-(2,6-dimethyl-morpholin-4-ylmethyl)-7-(3-trifluoromethyl-pyridin-2-yl)-pyrido[3,2-d]pyrimidin-4-yl]-amine (cis);

(4-tert-Butyl-phenyl)-[2-(2,6-dimethyl-morpholin-4-ylmethyl)-7-(3-methyl-pyridin-2-yl)-quinazolin-4-yl]-amine (cis);

(4-tert-Butyl-phenyl)-[2-(2,6-dimethyl-morpholin-4-ylmethyl)-7-(6-methyl-pyridin-2-yl)-quinazolin-4-yl]-amine (cis);

(4-tert-Butyl-phenyl)-[2-(2,6-dimethyl-morpholin-4-ylmethyl)-7-pyridin-2-yl-quinazolin-4-yl]-amine (cis);

(4-tert-Butyl-phenyl)-[2-piperidin-1-ylmethyl-7-(3-trifluoromethyl-pyridin-2-yl)-quinazolin-4-yl]-amine;

(4-tert-Butyl-phenyl)-[2-pyrrolidin-1-ylmethyl-7-(3-trifluoromethyl-pyridin-2-yl)-quinazolin-4-yl]-amine;

(4-tert-Butyl-phenyl)-[7-(3-chloro-pyridin-2-yl)-2-(2,6-dimethyl-morpholin-4-ylmethyl)-pyrido[3,2-d]pyrimidin-4-yl]-amine (cis);

(4-Trifluoromethyl-phenyl)-[7-(3-trifluoromethyl-pyridin-2-yl)-2-(3,3,5-trimethyl-azepan-1-ylmethyl)-quinazolin-4-yl]-amine;

(4-Trifluoromethyl-phenyl)-{7-(3-trifluoromethyl-pyridin-2-yl)-2-[2-(3,3,5-trimethyl-azepan-1-yl)-ethyl]-quinazolin-4-yl}-amine;

(6-tert-Butyl-pyridin-3-yl)-[7-(3-chloro-pyridin-2-yl)-2-(2,6-dimethyl-morpholin-4-ylmethyl)-pyrido[3,2-d]pyrimidin-4-yl]-amine (cis);

(R)-(4-Isopropyl-phenyl)-[2-(2-methyl-morpholin-4-ylmethyl)-7-(3-trifluoromethyl-pyridin-2-yl)-quinazolin-4-yl]-amine;

(R)-(4-tert-Butyl-phenyl)-[2-(2-methyl-morpholin-4-ylmethyl)-7-(3-trifluoromethyl-pyridin-2-yl)-quinazolin-4-yl]-amine;

(R)-(4-tert-Butyl-phenyl)-[7-(3-chloro-pyridin-2-yl)-2-(2-methyl-morpholin-4-ylmethyl)-quinazolin-4-yl]-amine;

(R)-[2-(2-Methyl-morpholin-4-ylmethyl)-7-(3-trifluoromethyl-pyridin-2-yl)-quinazolin-4-yl]-(4-trifluoromethyl-phenyl)-amine;

(R)-[7-(3-Chloro-pyridin-2-yl)-2-(2-methyl-morpholin-4-ylmethyl)-quinazolin-4-yl]-(4-isopropyl-phenyl)-amine;

(R)-[7-(3-Chloro-pyridin-2-yl)-2-(2-methyl-morpholin-4-ylmethyl)-quinazolin-4-yl]-(4-trifluoromethyl-phenyl)-amine;

(R,R)-(4-Chloro-phenyl)-[2-(2,6-dimethyl-morpholin-4-ylmethyl)-7-(3-trifluoromethyl-pyridin-2-yl)-quinazolin-4-yl]-amine;

(R,R)-(4-Chloro-phenyl)-[7-(3-chloro-pyridin-2-yl)-2-(2,6-dimethyl-morpholin-4-ylmethyl)-quinazolin-4-yl]-amine;

(R,R)-(4-Ethyl-phenyl)-[7-(3-chloro-pyridin-2-yl)-2-(2,6-dimethyl-morpholin-4-ylmethyl)-quinazolin-4-yl]-amine;

(R,R)-(4-Fluoro-phenyl)-[7-(3-chloro-pyridin-2-yl)-2-(2,6-dimethyl-morpholin-4-ylmethyl)-quinazolin-4-yl]-amine;

(R,R)-(4-Isopropyl-phenyl)-[7-(3-chloro-pyridin-2-yl)-2-(2,6-dimethyl-morpholin-4-ylmethyl)-quinazolin-4-yl]-amine;

(R,R)-(4-tert-Butyl-phenyl)-[7-(3-chloro-pyridin-2-yl)-2-(2,6-dimethyl-morpholin-4-ylmethyl)-quinazolin-4-yl]-amine;

(R,R)-(6-tert-Butyl-pyridin-3-yl)-[2-(2,6-dimethyl-morpholin-4-ylmethyl)-7-(3-trifluoromethyl-pyridin-2-yl)-quinazolin-4-yl]-amine;

(R,R)-(6-tert-Butyl-pyridin-3-yl)-[7-(3-chloro-pyridin-2-yl)-2-(2,6-dimethyl-morpholin-4-ylmethyl)-quinazolin-4-yl]-amine;

(R,R)-[2-(2,6-Dimethyl-morpholin-4-ylmethyl)-7-(3-trifluoromethyl-pyridin-2-yl)-quinazolin-4-yl]-(4-trifluoromethyl-phenyl)-amine;

(R,R)-[2-(2,6-Dimethyl-morpholin-4-ylmethyl)-7-(3-trifluoromethyl-pyridin-2-yl)-quinazolin-4-yl]-(6-trifluoromethyl-pyridin-3-yl)-amine;

(R,R)-[2-(2,6-Dimethyl-morpholin-4-ylmethyl)-7-(3-trifluoromethyl-pyridin-2-yl)-quinazolin-4-yl]-(4-isopropyl-phenyl)-amine;

(R,R)-[2-(2,6-Dimethyl-morpholin-4-ylmethyl)-7-(3-trifluoromethyl-pyridin-2-yl)-quinazolin-4-yl]-(4-ethyl-phenyl)-amine;

(R,R)-[2-(2,6-Dimethyl-morpholin-4-ylmethyl)-7-(3-trifluoromethyl-pyridin-2-yl)-quinazolin-4-yl]-[4-(propane-2-sulfonyl)-phenyl]-amine;

(R,R)-[2-(2,6-Dimethyl-morpholin-4-ylmethyl)-7-(3-trifluoromethyl-pyridin-2-yl)-pyrido[3,2-d]pyrimidin-4-yl]-(4-trifluoromethyl-phenyl)-amine;

(R,R)-[7-(3-Chloro-pyridin-2-yl)-2-(2,6-dimethyl-morpholin-4-ylmethyl)-quinazolin-4-yl]-(4-trifluoromethyl-phenyl)-amine;

(R,R)-[7-(3-Chloro-pyridin-2-yl)-2-(2,6-dimethyl-morpholin-4-ylmethyl)-quinazolin-4-yl]-(6-trifluoromethyl-pyridin-3-yl)-amine;

(R,R)-[7-(3-Chloro-pyridin-2-yl)-2-(2,6-dimethyl-morpholin-4-ylmethyl)-quinazolin-4-yl]-(6-isopropoxy-pyridin-3-yl)-amine;

(R,R)-[7-(3-Chloro-pyridin-2-yl)-2-(2,6-dimethyl-morpholin-4-ylmethyl)-quinazolin-4-yl]-[4-(propane-2-sulfonyl)-phenyl]-amine;

(R,R)-1-{4-[7-(3-Chloro-pyridin-2-yl)-2-(2,6-dimethyl-morpholin-4-ylmethyl)-quinazolin-4-ylamino]-phenyl}-ethanone;

(R,R)4-[7-(3-Chloro-pyridin-2-yl)-2-(2,6-dimethyl-morpholin-4-ylmethyl)-quinazolin-4-ylamino]-benzonitrile;

(S)-(4-Isopropyl-phenyl)-[2-(2-methyl-morpholin-4-ylmethyl)-7-(3-trifluoromethyl-pyridin-2-yl)-quinazolin-4-yl]-amine;

(S)-(4-tert-Butyl-phenyl)-[2-(2-methyl-morpholin-4-ylmethyl)-7-(3-trifluoromethyl-pyridin-2-yl)-quinazolin-4-yl]-amine;

(S)-(4-tert-Butyl-phenyl)-[7-(3-chloro-pyridin-2-yl)-2-(2-methyl-morpholin-4-ylmethyl)-quinazolin-4-yl]-amine;

(S)-[2-(1-Propyl-pyrrolidin-2-yl)-7-(3-trifluoromethyl-pyridin-2-yl)-quinazolin-4-yl]-(4-trifluoromethyl-phenyl)-amine;

(S)-[2-(2-Methyl-morpholin-4-ylmethyl)-7-(3-trifluoromethyl-pyridin-2-yl)-quinazolin-4-yl]-(4-trifluoromethyl-phenyl)-amine;

(S)-[2-Pyrrolidin-2-yl-7-(3-trifluoromethyl-pyridin-2-yl)-quinazolin-4-yl]-(4-trifluoromethyl-phenyl)-amine;

(S)-[7-(3-Chloro-pyridin-2-yl)-2-(2-methyl-morpholin-4-ylmethyl)-quinazolin-4-yl]-[4-(propane-2-sulfonyl)-phenyl]-amine;

(S)-[7-(3-Chloro-pyridin-2-yl)-2-(2-methyl-morpholin-4-ylmethyl)-quinazolin-4-yl]-(4-isopropyl-phenyl)-amine;

(S,S)-(4-Chloro-phenyl)-[2-(2,6-dimethyl-morpholin-4-ylmethyl)-7-(3-trifluoromethyl-pyridin-2-yl)-quinazolin-4-yl]-amine;

(S,S)-(4-Chloro-phenyl)-[7-(3-chloro-pyridin-2-yl)-2-(2,6-dimethyl-morpholin-4-ylmethyl)-quinazolin-4-yl]-amine;

(S,S)-(4-tert-Butyl-phenyl)-[7-(3-chloro-pyridin-2-yl)-2-(2,6-dimethyl-morpholin-4-ylmethyl)-quinazolin-4-yl]-amine;

(S,S)-(6-tert-Butyl-pyridin-3-yl)-[2-(2,6-dimethyl-morpholin-4-ylmethyl)-7-(3-trifluoromethyl-pyridin-2-yl)-quinazolin-4-yl]-amine;

(S,S)-(6-tert-Butyl-pyridin-3-yl)-[7-(3-chloro-pyridin-2-yl)-2-(2,6-dimethyl-morpholin-4-ylmethyl)-quinazolin-4-yl]-amine;

(S,S)-[2-(2,6-Dimethyl-morpholin-4-ylmethyl)-7-(3-trifluoromethyl-pyridin-2-yl)-quinazolin-4-yl]-(4-trifluoromethyl-phenyl)-amine;

(S,S)-[2-(2,6-Dimethyl-morpholin-4-ylmethyl)-7-(3-trifluoromethyl-pyridin-2-yl)-quinazolin-4-yl]-(6-trifluoromethyl-pyridin-3-yl)-amine;

(S,S)-[2-(2,6-Dimethyl-morpholin-4-ylmethyl)-7-(3-trifluoromethyl-pyridin-2-yl)-quinazolin-4-yl]-(4-isopropyl-phenyl)-amine;

(S,S)-[2-(2,6-Dimethyl-morpholin-4-ylmethyl)-7-(3-trifluoromethyl-pyridin-2-yl)-quinazolin-4-yl]-(4-ethyl-phenyl)-amine;

(S,S)-[2-(2,6-Dimethyl-morpholin-4-ylmethyl)-7-(3-trifluoromethyl-pyridin-2-yl)-quinazolin-4-yl]-[4-(propane-2-sulfonyl)-phenyl]-amine;

(S,S)-[2-(2,6-Dimethyl-morpholin-4-ylmethyl)-7-(3-trifluoromethyl-pyridin-2-yl)-pyrido[3,2-d]pyrimidin-4-yl]-(4-trifluoromethyl-phenyl)-amine;

(S,S)-[7-(3-Chloro-pyridin-2-yl)-2-(2,6-dimethyl-morpholin-4-ylmethyl)-quinazolin-4-yl]-(4-trifluoromethyl-phenyl)-amine;

(S,S)-[7-(3-Chloro-pyridin-2-yl)-2-(2,6-dimethyl-morpholin-4-ylmethyl)-quinazolin-4-yl]-(6-trifluoromethyl-pyridin-3-yl)-amine;

(S,S)-[7-(3-Chloro-pyridin-2-yl)-2-(2,6-dimethyl-morpholin-4-ylmethyl)-quinazolin-4-yl]-[4-(propane-2-sulfonyl)-phenyl]-amine;

(S,S)-[7-(3-Chloro-pyridin-2-yl)-2-(2,6-dimethyl-morpholin-4-ylmethyl)-quinazolin-4-yl]-(4-isopropyl-phenyl)-amine;

(S,S)-[7-(3-Chloro-pyridin-2-yl)-2-(2,6-dimethyl-morpholin-4-ylmethyl)-quinazolin-4-yl]-(4-ethyl-phenyl)-amine;

[2-(1,1-Dioxo-1λ⁶-thiomorpholin-4-ylmethyl)-7-(3-trifluoromethyl-pyridin-2-yl)-quinazolin-4-yl]-(6-trifluoromethyl-pyridin-3-yl)-amine;

[2-(1,4-Dioxa-8-aza-spiro[4.5]dec-8-ylmethyl)-7-(3-trifluoromethyl-pyridin-2-yl)-quinazolin-4-yl]-(4-trifluoromethyl-phenyl)-amine;

[2-(1-Ethyl-piperidin4-yl)-7-(3-trifluoromethyl-pyridin-2-yl)-quinazolin-4-yl]-(4-trifluoromethyl-phenyl)-amine;

[2-(1-Methanesulfonyl-piperidin4-yl)-7-(3-trifluoromethyl-pyridin-2-yl)-quinazolin-4-yl]-(4-trifluoromethyl-phenyl)-amine;

[2-(1-Methyl-3,4-dihydro-1H-isoquinolin-2-ylmethyl)-7-(3-trifluoromethyl-pyridin-2-yl)-quinazolin-4-yl]-(4-trifluoromethyl-phenyl)-amine;

[2-( -Propyl-piperidin-4-yl)-7-(3-trifluoromethyl-pyridin-2-yl)-quinazolin-4-yl]-(4-trifluoromethyl-phenyl)-amine;

[2-(1-Pyridin4-ylmethyl-piperidin4-yl)-7-(3-trifluoromethyl-pyridin-2-yl)-quinazolin-4-yl]-(4-trifluoromethyl-phenyl)-amine;

[2-(2,6-Dimethyl-morpholin-4-ylmethyl)-7-(2-methoxy-phenyl)-quinazolin-4-yl]-(4-trifluoromethyl-phenyl)-amine (cis);

[2-(2,6-Dimethyl-morpholin-4-ylmethyl)-7-(3-methyl-pyridin-2-yl)-pyrido[3,2-d]pyrimidin-4-yl]-(4-trifluoromethyl-phenyl)-amine (cis);

[2-(2,6-Dimethyl-morpholin-4-ylmethyl)-7-(3-methyl-pyridin-2-yl)-quinazolin-4-yl]-(4-trifluoromethyl-phenyl)-amine (cis);

[2-(2,6-Dimethyl-morpholin-4-ylmethyl)-7-(3-trifluoromethyl-pyridin-2-yl)-quinazolin-4-yl]-(6-trifluoromethyl-pyridin-3-yl)-amine;

[2-(2,6-Dimethyl-morpholin-4-ylmethyl)-7-(3-trifluoromethyl-pyridin-2-yl)-quinazolin-4-yl]-(4-trifluoromethyl-phenyl)-amine;

[2-(2,6-Dimethyl-morpholin-4-ylmethyl)-7-(3-trifluoromethyl-pyridin-2-yl)-quinazolin-4-yl]-(4-trifluoromethyl-phenyl)-amine (cis);

[2-(2,6-Dimethyl-morpholin-4-ylmethyl)-7-(3-trifluoromethyl-pyridin-2-yl)-quinazolin-4-yl]-(5-trifluoromethyl-pyridin-2-yl)-amine;

[2-(2,6-Dimethyl-morpholin-4-ylmethyl)-7-(3-trifluoromethyl-pyridin-2-yl)quinazolin-4-yl]-[4-(2,2,2-trifluoro-1-methyl-ethyl)-phenyl]-amine;

[2-(2,6-Dimediyl-morpholin-4-ylmethyl)-7-(3-trifluoromethyl-pyridin-2-yl)-quinazolin-4-yl]-(6-trifluoromethyl-pyridin-3-yl)-amine (cis);

[2-(2,6-Dimethyl-morpholin-4-ylmethyl)-7-(3-trifluoromethyl-pyridin-2-yl)-quinazolin-4-yl]-(5-trifluoromethyl-pyridin-2-yl)-amine (cis);

[2-(2,6-Dimethyl-morpholin-4-ylmethyl)-7-(3-trifluoromethyl-pyridin-2-yl)-pyrido[3,2-d]pyrimidin-4-yl]-(4-trifluoromethyl-phenyl)-amine (cis);

[2-(2,6-Dimethyl-morpholin-4-ylmethyl)-7-(3-trifluoromethyl-pyridin-2-yl)-pyrido[3,2-d]pyrimidin-4-yl]-(6-trifluoromethyl-pyridin-3-yl)-amine (cis);

[2-(2,6-Dimethyl-morpholin-4-ylmethyl)-7-(3-trifluoromethyl-pyridin-2-yl)-quinazolin-4-yl]-(4-trifluoromethanesulfonyl-phenyl)-amine;

[2-(2,6-Dimethyl-morpholin-4-ylmethyl)-7-(3-trifluoromethyl-pyridin-2-yl)-quinazolin-4-yl]-(4-trifluoromethanesulfonyl-phenyl)-amine (cis);

[2-(2,6-Dimethyl-morpholin-4-ylmethyl)-7-(3-trifluoromethyl-pyridin-2-yl)-pyrido[3,2-d]pyrimidin-4-yl]-(4-methanesulfonyl-phenyl)-amine (cis);

[2-(2,6-Dimethyl-morpholin-4-ylmethyl)-7-(3-trifluoromethyl-pyridin-2-yl)-pyrido[3,2-d]pyrimidin-4-yl]-(4-trifluoromethanesulfonyl-phenyl)-amine (cis);

[2-(2,6-Dimethyl-morpholin-4-ylmethyl)-7-(3-trifluoromethyl-pyridin-2-yl)-pyrido[3,2-d]pyrimidin-4-yl]-[4-(propane-1-sulfonyl)-phenyl]-amine (cis);

[2-(2,6-Dimethyl-morpholin-4-ylmethyl)-7-(3-trifluoromethyl-pyridin-2-yl)-pyrido[3,2-d]pyrimidin-4-yl]-[4-(propane-2-sulfonyl)-phenyl]-amine (cis);

[2-(2,6-Dimethyl-morpholin-4-ylmethyl)-7-(3-trifluoromethyl-pyridin-2-yl)-pyrido[3,2-d]pyrimidin-4-yl]-(4-isopropyl-phenyl)-amine (cis);

[2-(2,6-Dimethyl-morpholin-4-ylmethyl)-7-(3-trifluoromethyl-pyridin-2-yl)-quinazolin-4-yl]-(4-isopropyl-phenyl)-amine (cis);

[2-(2,6-Dimethyl-morpholin-4-ylmethyl)-7-pyridin-2-yl-quinazolin-4-yl]-(4-trifluoromethyl-phenyl)-amine (cis);

[2-(2-Ethyl-piperidin-1-ylethyl)-7-(3-trifluoromethyl-pyridin-2-yl)-quinazolin-4-yl]-(4-trifluoromethyl-phenyl)-amine;

[2-(2-Ethyl-piperidin-1-ylmethyl)-7-(3-trifluoromethyl-pyridin-2-yl)-quinazolin-4-yl]-(4-trifluoromethyl-phenyl)-amine;

[2-(2-Methyl-piperidin-1-ylethyl)-7-(3-trifluoromethyl-pyridin-2-yl)-quinazolin4-yl]-(4-trifluoromethyl-phenyl)-amine;

[2-(2-Methyl-piperidin-1-ylmethyl)-7-(3-trifluoromethyl-pyridin-2-yl)-quinazolin-4-yl]-(4-trifluoromethyl-phenyl)-amine;

[2-(3,3-Dimethyl-piperidin-1-ylethyl)-7-(3-trifluoromethyl-pyridin-2-yl)-quinazolin-4-yl]-(4-trifluoromethyl-phenyl)-amine;

[2-(3,3-Dimethyl-piperidin-1-ylmethyl)-7-(3-trifluoromethyl-pyridin-2-yl)-quinazolin-4-yl]-(4-trifluoromethyl-phenyl)-amine;

[2-(3,3-Dimethyl-piperidin-1-ylmethyl)-7-(3-trifluoromethyl-pyridin-2-yl)-quinazolin-4-yl]-(6-trifluoromethyl-pyridin-3-yl)-amine;

[2-(3,3-Dimethyl-piperidin-1-ylmethyl)-7-(3-trifluoromethyl-pyridin-2-yl)-quinazolin-4-yl]-(4-trifluoromethyl-phenyl)-amine;

[2-(3,3-Dimethyl-piperidin-1-ylmethyl)-7-(3-trifluoromethyl-pyridin-2-yl)-pyrido[3,2-d]pyrimidin-4-yl]-(4-trifluoromethyl-phenyl)-amine;

[2-(3,4-Dihydro-1H-isoquinolin-2-ylmethyl)-7-(3-trifluoromethyl-pyridin-2-yl)-quinazolin-4-yl]-(4-trifluoromethyl-phenyl)-amine;

[2-(3,5-Dimethyl-piperidin-1-ylethyl)-7-(3-trifluoromethyl-pyridin-2-yl)-quinazolin-4-yl]-(4-trifluoromethyl-phenyl)-amine;

[2-(3,5-Dimethyl-piperidin-1-ylmethyl)-7-(3-trifluoromethyl-pyridin-2-yl)-quinazolin-4-yl]-(4-trifluoromethyl-phenyl)-amine;

[2-(3,5-Dimethyl-piperidin-1-ylmethyl)-7-(3-trifluoromethyl-pyridin-2-yl)-quinazolin-4-yl]-(6-trifluoromethyl-pyridin-3-yl)-amine;

[2-(3,5-Dimethyl-piperidin-1-ylmethyl)-7-(3-trifluoromethyl-pyridin-2-yl)-quinazolin-4-yl]-(4-trifluoromethyl-phenyl)-amine;

[2-(3,5-Dimethyl-piperidin-1-ylmethyl)-7-(3-trifluoromethyl-pyridin-2-yl)-pyrido[3,2-d]pyrimidin-4-yl]-(4-trifluoromethyl-phenyl)-amine (cis);

[2-(3-Hydroxy-piperidin-1-ylethyl)-7-(3-trifluoromethyl-pyridin-2-yl)-quinazolin-4-yl]-(4-trifluoromethyl-phenyl)-amine;

[2-(3-Methoxy-piperidin-1-ylethyl)-7-(3-trifluoromethyl-pyridin-2-yl)-quinazolin-4-yl]-(4-trifluoromethyl-phenyl)-amine;

[2-(3-Methyl-piperidin-1-ylethyl)-7-(3-trifluoromethyl-pyridin-2-yl)-quinazolin-4-yl]-(4-trifluoromethyl-phenyl)-amine;

[2-(3-Methyl-piperidin-1-ylmethyl)-7-(3-trifluoromethyl-pyridin-2-yl)-quinazolin-4-yl]-(6-trifluoromethyl-pyridin-3-yl)-amine;

[2-(3-Methyl-piperidin-1-ylmethyl)-7-(3-trifluoromethyl-pyridin-2-yl)-quinazolin-4-yl]-(4-trifluoromethyl-phenyl)-amine;

[2-(3-Methyl-piperidin-1-ylmethyl)-7-(3-trifluoromethyl-pyridin-2-yl)-pyrido[3,2-d]pyrimidin-4-yl]-(4-trifluoromethyl-phenyl)-amine;

[2-(3-Pyrrolidin-1-yl-propyl)-7-(3-trifluoromethyl-pyridin-2-yl)-quinazolin-4-yl]-(4-trifluoromethyl-phenyl)-amine;

[2-(4-Cyclopentyl-piperazin-1-ylmethyl)-7-(3-trifluoromethyl-pyridin-2-yl)-quinazolin-4-yl]-(4-trifluoromethyl-phenyl)-amine;

[2-(4-Ethoxy-piperidin-1-ylethyl)-7-(3-trifluoromethyl-pyridin-2-yl)-quinazolin-4-yl]-(4-trifluoromethyl-phenyl)-amine;

[2-(4-Ethyl-piperazin-1-ylmethyl)-7-(3-trifluoromethyl-pyridin-2-yl)-quinazolin-4-yl]-4-trifluoromethyl-phenyl)-amine;

[2-(4-Hydroxy-piperidin-1-ylethyl)-7-(3-trifluoromethyl-pyridin-2-yl)-quinazolin-4-yl]-(4-trifluoromethyl-phenyl)-amine;

[2-(4-Isopropyl-piperazin-1-ylmethyl)-7-(3-trifluoromethyl-pyridin-2-yl)-quinazolin-4-yl]-(4-trifluoromethyl-phenyl)-amine;

[2-(4-Methoxy-piperidin-1-ylethyl)-7-(3-trifluoromethyl-pyridin-2-yl)-quinazolin-4-yl]-(4-trifluoromethyl-phenyl)-amine;

[2-(4-Methyl-[1,4]diazepan-1-ylmethyl)-7-(3-trifluoromethyl-pyridin-2-yl)-quinazolin-4-yl]-(4-trifluoromethyl-phenyl)-amine;

[2-(4-Methyl-piperazin-1-ylmethyl)-7-(3-trifluoromethyl-pyridin-2-yl)-quinazolin-4-yl]-(5-trifluoromethyl-pyridin-2-yl)-amine;

[2-(4-Methyl-piperazin-1-ylmethyl)-7-(3-trifluoromethyl-pyridin-2-yl)-quinazolin-4-yl]-[4-(2,2,2-trifluoro-1-methyl-ethyl)-phenyl]-amine;

[2-(4-Methyl-piperazin-1-ylmethyl)-7-(3-trifluoromethyl-pyridin-2-yl)-quinazolin-4-yl]-(4-trifluoromethyl-phenyl)-amine;

[2-(4-Methyl-piperidin-1-ylethyl)-7-(3-trifluoromethyl-pyridin-2-yl)-quinazolin-4-yl]-(4-trifluoromethyl-phenyl)-amine;

[2-(4-Methyl-piperidin-1-ylmethyl)-7-(3-trifluoromethyl-pyridin-2-yl)-quinazolin-4-yl]-(4-trifluoromethyl-phenyl)-amine;

[2-(5,6-Dihydro4H-pyrimidin-1-ylmethyl)-7-(3-trifluoromethyl-pyridin-2-yl)-quinazolin-4-yl]-(4-trifluoromethyl-phenyl)-amine;

[2-(5H-Tetrazol-5-yl)-7-(3-trifluoromethyl-pyridin-2-yl)-quinazolin-4-yl]-(4-trifluoromethyl-phenyl)-amine;

[2-(Benzylamino-methyl)-7-(3-trifluoromethyl-pyridin-2-yl)-quinazolin-4-yl]-(5-trifluoromethyl-pyridin-2-yl)-amine;

[2-(Benzylamino-methyl)-7-(3-trifluoromethyl-pyridin-2-yl)-quinazolin-4-yl]-(6-trifluoromethyl-pyridin-3-yl)-amine;

[2-(Isobutylamino-methyl)-7-(3-trifluoromethyl-pyridin-2-yl)-quinazolin-4-yl]-(4-trifluoromethyl-phenyl)-amine;

[2-(Isopropylamino-methyl)-7-(3-trifluoromethyl-pyridin-2-yl)-quinazolin-4-yl]-(4-trifluoromethyl-phenyl)-amine;

[2-(Octahydro-quinolin-1-ylethyl)-7-(3-trifluoromethyl-pyridin-2-yl)-quinazolin-4-yl]-(4-trifluoromethyl-phenyl)-amine;

[2-(Octahydro-quinolin-1-ylmethyl)-7-(3-trifluoromethyl-pyridin-2-yl)-quinazolin-4-yl]-(4-trifluoromethyl-phenyl)-amine;

[2-(tert-Butylamino-methyl)-7-(3-trifluoromethyl-pyridin-2-yl)-quinazolin-4-yl]-(4-trifluoromethyl-phenyl)-amine;

[2-[(2-Methoxy-benzylamino)-methyl]-7-(3-trifluoromethyl-pyridin-2-yl)-quinazolin-4-yl]-(6-trifluoromethyl-pyridin-3-yl)-amine;

[2-[(2-Methoxy-ethylamino)-methyl]-7-(3-trifluoromethyl-pyridin-2-yl)-quinazolin-4-yl]-(4-trifluoromethyl-phenyl)-amine;

[2-[(3-Methyl-butylamino)-methyl]-7-(3-trifluoromethyl-pyridin-2-yl)-quinazolin-4-yl]-(4-trifluoromethyl-phenyl)-amine;

[2-[(4-Methoxy-benzylamino)-methyl]-7-(3-trifluoromethyl-pyridin-2-yl)-quinazolin-4-yl]-(6-trifluoromethyl-pyridin-3-yl)-amine;

[2-[(Allyl-methyl-amino)-ethyl]-7-(3-trifluoromethyl-pyridin-2-yl)-quinazolin-4-yl]-(4-trifluoromethyl-phenyl)-amine;

[2-[(Allyl-methyl-amino)-methyl]-7-(3-trifluoromethyl-pyridin-2-yl)-quinazolin-4-yl]-(4-trifluoromethyl-phenyl)-amine;

[2-[(Ally,-methyl-amino)-methyl]-7-(3-trifluoromethyl-pyridin-2-yl)-pyrido[3,2-d]pyrimidin-4-yl]-(4-trifluoromethyl-phenyl)-amine;

[2-[(Benzyl-cyclopropyl-amino)-methyl]-7-(3-trifluoromethyl-pyridin-2-yl)-quinazolin-4-yl]-(4-trifluoromethyl-phenyl)-amine;

[2-[(Benzyl-methyl-amino)-methyl]-7-(3-trifluoromethyl-pyridin-2-yl)-quinazolin-4-yl]-(6-trifluoromethyl-pyridin-3-yl)-amine;

[2-[(Benzyl-methyl-amino)-methyl]-7-(3-trifluoromethyl-pyridin-2-yl)-quinazolin-4-yl]-(4-trifluoromethyl-phenyl)-amine;

[2-[(Butyl-ethyl-amino)-ethyl]-7-(3-trifluoromethyl-pyridin-2-yl)-quinazolin-4-yl]-(4-trifluoromethyl-phenyl)-amine;

[2-[(Butyl-ethyl-amino)-methyl]-7-(3-trifluoromethyl-pyridin-2-yl)-quinazolin-4-yl]-(4-trifluoromethyl-phenyl)-amine;

[2-[(Butyl-methyl-amino)-ethyl]-7-(3-trifluoromethyl-pyridin-2-yl)-quinazolin-4-yl]-(4-trifluoromethyl-phenyl)-amine;

[2-[(Butyl-methyl-amino)-methyl]-7-(3-trifluoromethyl-pyridin-2-yl)-quinazolin-4-yl]-(4-trifluoromethyl-phenyl)-amine;

[2-[(Cyclohexyl-ethyl-amino)-ethyl]-7-(3-trifluoromethyl-pyridin-2-yl)-quinazolin-4-yl]-(4-trifluoromethyl-phenyl)-amine;

[2-[(Cyclohexyl-ethyl-amino)-methyl]-7-(3-trifluoromethyl-pyridin-2-yl)-quinazolin-4-yl]-(4-trifluoromethyl-phenyl)-amine;

[2-[(Cyclohexyl-methyl-amino)-ethyl]-7-(3-trifluoromethyl-pyridin-2-yl)-quinazolin-4-yl]-(4-trifluoromethyl-phenyl)-amine;

[2-[(Cyclohexyl-methyl-amino)-methyl]-7-(3-trifluoromethyl-pyridin-2-yl)-quinazolin-4-yl]-(4-trifluoromethyl-phenyl)-amine;

[2-[(Cyclopropylmethyl-propyl-amino)-ethyl]-7-(3-trifluoromethyl-pyridin-2-yl)-quinazolin-4-yl]-(4-trifluoromethyl-phenyl)-amine;

[2-[(Cyclopropylmethyl-propyl-amino)-methyl]-7-(3-trifluoromethyl-pyridin-2-yl)-quinazolin-4-yl]-(4-trifluoromethyl-phenyl)-amine;

[2-[(Cyclopropylmethyl-propyl-amino)-methyl]-7-(3-trifluoromethyl-pyridin-2-yl)-quinazolin-4-yl]-(6-trifluoromethyl-pyridin-3-yl)-amine;

[2-[(Cyclopropylmethyl-propyl-amino)-methyl]-7-(3-trifluoromethyl-pyridin-2-yl)-quinazolin-4-yl]-(4-trifluoromethyl-phenyl)-amine;

[2-[(Cyclopropylmethyl-propyl-amino)-methyl]-7-(3-trifluoromethyl-pyridin-2-yl)-pyrido[3,2-d]pyrimidin-4-yl]-(4-trifluoromethyl-phenyl)-amine;

[2-[(Ethyl-isopropyl-amino)-methyl]-7-(3-trifluoromethyl-pyridin-2-yl)-quinazolin-4-yl]-(4-trifluoromethyl-phenyl)-amine;

[2-[(Hexyl-methyl-amino)-ethyl]-7-(3-trifluoromethyl-pyridin-2-yl)-quinazolin-4-yl]-(4-trifluoromethyl-phenyl)-amine;

[2-[(Hexyl-methyl-amino)-methyl]-7-(3-trifluoromethyl-pyridin-2-yl)-quinazolin-4-yl]-(4-trifluoromethyl-phenyl)-amine;

[2-[(Indan-1-yl-methyl-amino)-methyl]-7-(3-trifluoromethyl-pyridin-2-yl)-quinazolin-4-yl]-(4-trifluoromethyl-phenyl)-amine;

[2-[(Isopropyl-ethyl-amino)-ethyl]-7-(3-trifluoromethyl-pyridin-2-yl)-quinazolin-4-yl]-(4-trifluoromethyl-phenyl)-amine;

[2-[(Isopropyl-methyl-amino)-ethyl]-7-(3-trifluoromethyl-pyridin-2-yl)-quinazolin-4-yl]-(4-trifluoromethyl-phenyl)-amine;

[2-[(Isopropyl-methyl-amino)-methyl]-7-(3-trifluoromethyl-pyridin-2-yl)-quinazolin-4-yl]-(4-trifluoromethyl-phenyl)-amine;

[2-[(Methyl-propyl-amino)-methyl]-7-(3-trifluoromethyl-pyridin-2-yl)-quinazolin-4-yl]-(4-trifluoromethyl-phenyl)-amine;

[2-[(Propyl-methyl-amino)-ethyl]-7-(3-trifluoromethyl-pyridin-2-yl)-quinazolin-4-yl]-(4-trifluoromethyl-phenyl)-amine;

[2-[(Tetrahydro-thiopyran-4-ylamino)-methyl]-7-(3-trifluoromethyl-pyridin-2-yl)-quinazolin-4-yl]-(6-trifluoromethyl-pyridin-3-yl)-amine;

[2-[1-(1-Methyl-1H-imidazol-2-ylmethyl)-piperidin-4-yl]-7-(3-trifluoromethyl-pyridin-2-yl)-quinazolin-4-yl]-(4-trifluoromethyl-phenyl)-amine;

[2-[2-(1,4-Dioxa-8-aza-spiro[4.5]dec-8-yl)-ethyl]-7-(3-trifluoromethyl-pyridin-2-yl)-quinazolin-4-yl]-(4-trifluoromethyl-phenyl)-amine;

[2-[2-(1-Methyl-3,4-dihydro-1H-isoquinolin-2-yl)-ethyl]-7-(3-trifluoromethyl-pyridin-2-yl)-quinazolin-4-yl]-(4-trifluoromethyl-phenyl)-amine;

[2-[2-(2,6-Dimethyl-morpholin-4-yl)-ethyl]-7-(3-trifluoromethyl-pyridin-2-yl)-quinazolin-4-yl]-(4-trifluoromethyl-phenyl)-amine;

[2-[2-(3,4-Dihydro-1H-isoquinolin-2-yl)-ethyl]-7-(3-trifluoromethyl-pyridin-2-yl)-quinazolin-4-yl]-(4-trifluoromethyl-phenyl)-amine;

[2-[2-(4-Methyl-piperazin-1-yl)-ethyl]-7-(3-trifluoromethyl-pyridin-2-yl)-quinazolin-4-yl]-(4-trifluoromethyl-phenyl)-amine;

[2-[2-(Benzyl-cyclopropyl-amino)-ethyl]-7-(3-trifluoromethyl-pyridin-2-yl)-quinazolin-4-yl]-(4-trifluoromethyl-phenyl)-amine;

[2-[2-(Benzyl-methyl-amino)-ethyl]-7-(3-trifluoromethyl-pyridin-2-yl)-quinazolin-4-yl]-(4-trifluoromethyl-phenyl)-amine;

[2-[2-(Indan-1-yl-methyl-amino)-ethyl]-7-(3-trifluoromethyl-pyridin-2-yl)-quinazolin-4-yl]-(4-trifluoromethyl-phenyl)-amine;

[2-[2-(Methyl-phenethyl-amino)-ethyl]-7-(3-trifluoromethyl-pyridin-2-yl)-quinazolin-4-yl]-(4-trifluoromethyl-phenyl)-amine;

[2-[3-(2,6-Dimethyl-morpholin-4-yl)-propyl]-7-(3-trifluoromethyl-pyridin-2-yl)-quinazolin-4-yl]-(6-trifluoromethyl-pyridin-3-yl)-amine;

[2-[3-(2,6-Dimethyl-morpholin-4-yl)-propyl]-7-(3-trifluoromethyl-pyridin-2-yl)-quinazolin-4-yl]-(4-trifluoromethyl-phenyl)-amine;

[2-[3-(2,6-Dimethyl-morpholin-4-yl)-propyl]-7-(3-trifluoromethyl-pyridin-2-yl)-quinazolin-4-yl]-(4-trifluoromethyl-phenyl)-amine (cis);

[2-[3-(3-Methyl-piperidin-1-yl)-propyl]-7-(3-trifluoromethyl-pyridin-2-yl)-quinazolin-4-yl]-(4-trifluoromethyl-phenyl)-amine;

[2-[3-(4-Methyl-piperazin-1-yl)-propyl]-7-(3-trifluoromethyl-pyridin-2-yl)-quinazolin-4-yl]-(4-trifluoromethyl-phenyl)-amine;

[2-[4-(2-Diethylamino-ethyl)-piperazin-1-ylmethyl]-7-(3-trifluoromethyl-pyridin-2-yl)-quinazolin-4-yl]-(4-trifluoromethyl-phenyl)-amine;

[2-[4-(2-Dimethylamino-ethyl)-piperazin-1-ylmethyl]-7-(3-trifluoromethyl-pyridin-2-yl)-quinazolin-4-yl]-(4-trifluoromethyl-phenyl)-amine;

[2-[4-(2-Methoxy-ethyl)-piperazin-1-ylmethyl]-7-(3-trifluoromethyl-pyridin-2-yl)-quinazolin-4-yl]-(4-trifluoromethyl-phenyl)-amine;

[2-[4-(2-Morpholin4-yl-ethyl)-piperazin-1-ylmethyl]-7-(3-trifluoromethyl-pyridin-2-yl)-quinazolin-4-yl]-(4-trifluoromethyl-phenyl)-amine;

[2-[4-(2-Pyrrolidin-1-yl-ethyl)-piperazin-1-ylmethyl]-7-(3-trifluoromethyl-pyridin-2-yl)-quinazolin-4-yl]-(4-trifluoromethyl-phenyl)-amine;

[2-[4-(3-Dimethylamino-propyl)-piperazin-1-ylmethyl]-7-(3-trifluoromethyl-pyridin-2-yl)-quinazolin-4-yl]-(4-trifluoromethyl-phenyl)-amine;

[2-{[(2-Fluoro-benzyl)-methyl-amino]-methyl}-7-(3-trifluoromethyl-pyridin-2-yl)-quinazolin-4-yl]-(4-trifluoromethyl-phenyl)-amine;

[2-{[(3-Fluoro-benzyl)-methyl-amino]-methyl}-7-(3-trifluoromethyl-pyridin-2-yl)-quinazolin-4-yl]-(4-trifluoromethyl-phenyl)-amine;

[2-{[(Pyridin-2-ylmethyl)-amino]-methyl}-7-(3-trifluoromethyl-pyridin-2-yl)-quinazolin-4-yl]-(6-trifluoromethyl-pyridin-3-yl)-amine;

[2-{[Bis-(2-methoxy-ethyl)-amino]-methyl}-7-(3-trifluoromethyl-pyridin-2-yl)-quinazolin-4-yl]-(5-trifluoromethyl-pyridin-2-yl)-amine;

[2-{[Bis-(2-methoxy-ethyl)-amino]-methyl}-7-(3-trifluoromethyl-pyridin-2-yl)-quinazolin-4-yl]-[4-(2,2,2-trifluoro-1-methyl-ethyl)-phenyl]-amine;

[2-{[Bis-(2-methoxy-ethyl)-amino]-methyl}-7-(3-trifluoromethyl-pyridin-2-yl)-quinazolin-4-yl]-(4-trifluoromethyl-phenyl)-amine;

[2-{[Bis-(2-methoxy-ethyl)-amino]-methyl}-7-(3-trifluoromethyl-pyridin-2-yl)-pyrido[3,2-d]pyrimidin-4-yl]-(4-trifluoromethyl-phenyl)-amine;

[2-{[Ethyl-(2-methyl-allyl)-amino]-methyl}-7-(3-trifluoromethyl-pyridin-2-yl)-quinazolin-4-yl]-(4-trifluoromethyl-phenyl)-amine;

[2-{[Ethyl-(2-methyl-allyl)-amino]-methyl}-7-(3-trifluoromethyl-pyridin-2-yl)-pyrido[3,2-d]pyrimidin-4-yl]-(4-trifluoromethyl-phenyl)-amine;

[2-{[Methyl-(1-phenyl-ethyl)-amino]-methyl}-7-(3-trifluoromethyl-pyridin-2-yl)-quinazolin-4-yl]-(4-trifluoromethyl-phenyl)-amine;

[2-{[Methyl-(1-phenyl-propyl)-amino]-methyl}-7-(3-trifluoromethyl-pyridin-2-yl)-quinazolin-4-yl]-(4-trifluoromethyl-phenyl)-amine;

[2-{[Methyl-(2-methyl-benzyl)-amino]-methyl}-7-(3-trifluoromethyl-pyridin-2-yl)-quinazolin-4-yl]-(4-trifluoromethyl-phenyl)-amine;

[2-{[Methyl-(2-phenyl-ethyl)-amino]-methyl}-7-(3-trifluoromethyl-pyridin-2-yl)-quinazolin-4-yl]-(4-trifluoromethyl-phenyl)-amine;

[2-{2-[(2-Fluoro-benzyl)-methyl-amino]-ethyl}-7-(3-trifluoromethyl-pyridin-2-yl)-quinazolin-4-yl]-(4-trifluoromethyl-phenyl)-amine;

[2-{2-[(3-Fluoro-benzyl)-methyl-amino]-ethyl}-7-(3-trifluoromethyl-pyridin-2-yl)-quinazolin-4-yl]-(4-trifluoromethyl-phenyl)-amine;

[2-{2-[Bis-(2-methoxy-ethyl)-amino]-ethyl}-7-(3-trifluoromethyl-pyridin-2-yl)-quinazolin-4-yl]-(4-trifluoromethyl-phenyl)-amine;

[2-{2-[Ethyl-(2-methyl-allyl)-amino]-ethyl}-7-(3-trifluoromethyl-pyridin-2-yl)-quinazolin-4-yl]-(4-trifluoromethyl-phenyl)-amine;

[2-{2-[Methyl-(1-phenyl-ethyl)-amino]-ethyl}-7-(3-trifluoromethyl-pyridin-2-yl)-quinazolin-4-yl]-(4-trifluoromethyl-phenyl)-amine;

[2-{2-[Methyl-(1-phenyl-propyl)-amino]-ethyl}-7-(3-trifluoromethyl-pyridin-2-yl)-quinazolin-4-yl]-(4-trifluoromethyl-phenyl)-amine;

[2-{2-[Methyl-(2-methyl-benzyl)-amino]-ethyl}-7-(3-trifluoromethyl-pyridin-2-yl)-quinazolin-4-yl]-(4-trifluoromethyl-phenyl)-amine;

[2-Azepan-1-ylethyl-7-(3-trifluoromethyl-pyridin-2-yl)-quinazolin-4-yl]-(4-trifluoromethyl-phenyl)-amine;

[2-Azepan-1-ylmethyl-7-(3-trifluoromethyl-pyridin-2-yl)-pyrido[3,2-d]pyrimidin-4-yl]-(4-trifluoromethyl-phenyl)-amine;

[2-Azepan-1-ylmethyl-7-(3-trifluoromethyl-pyridin-2-yl)-quinazolin-4-yl]-(4-trifluoromethyl-phenyl)-amine;

[2-Azocan-1-ylethyl-7-(3-trifluoromethyl-pyridin-2-yl)-quinazoelin-4-yl]-(4-trifluoromethyl-phenyl)-amine;

[2-Azocan-1-ylmethyl-7-(3-trifluoromethyl-pyridin-2-yl)-quinazolin-4-yl]-(4-trifluoromethyl-phenyl)-amine;

[2-Cyclohexylaminomethyl-7-(3-trifluoromethyl-pyridin-2-yl)-quinazolin-4-yl]-(4-trifluoromethyl-phenyl)-amine;

[2-Diallylaminoethyl-7-(3-trifluoromethyl-pyridin-2-yl)-quinazolin-4-yl]-(4-trifluoromethyl-phenyl)-amine;

[2-Diallylaminomethyl-7-(3-trifluoromethyl-pyridin-2-yl)-pyrido[3,2-d]pyrimidin-4-yl]-(4-trifluoromethyl-phenyl)-amine;

[2-Diallylaminomethyl-7-(3-trifluoromethyl-pyridin-2-yl)-quinazolin-4-yl]-(4-trifluoromethyl-phenyl)-amine;

[2-Dibutylaminoethyl-7-(3-trifluoromethyl-pyridin-2-yl)-quinazolin-4-yl]-(4-trifluoromethyl-phenyl)-amine;

[2-Dibutylaminomethyl-7-(3-trifluoromethyl-pyridin-2-yl)-pyrido[3,2-d]pyrimidin-4-yl]-(4-trifluoromethyl-phenyl)-amine;

[2-Dibutylaminomethyl-7-(3-trifluoromethyl-pyridin-2-yl)-quinazolin-4-yl]-(4-trifluoromethyl-phenyl)-amine;

[2-Diethylaminoethyl-7-(3-trifluoromethyl-pyridin-2-yl)-quinazolin-4-yl]-(4-trifluoromethyl-phenyl)-amine;

[2-Diethylaminomethyl-7-(3-trifluoromethyl-pyridin-2-yl)-quinazolin-4-yl]-(4-trifluoromethyl-phenyl)-amine;

[2-Dihexylaminoethyl-7-(3-trifluoromethyl-pyridin-2-yl)-quinazolin-4-yl]-(4-trifluoromethyl-phenyl)-amine;

[2-Dihexylaminomethyl-7-(3-trifluoromethyl-pyridin-2-yl)-quinazolin-4-yl]-(4-trifluoromethyl-phenyl)-amine;

[2-Dimethylaminoethyl-7-(3-trifluoromethyl-pyridin-2-yl)-quinazolin-4-yl]-(4-trifluoromethyl-phenyl)-amine;

[2-Dimethylaminomethyl-7-(3-trifluoromethyl-pyridin-2-yl)-quinazolin-4-yl]-(4-trifluoromethyl-phenyl)-amine;

[2-Dipentylaminoethyl-7-(3-trifluoromethyl-pyridin-2-yl)-quinazolin-4-yl]-(4-trifluoromethyl-phenyl)-amine;

[2-Dipentylaminomethyl-7-(3-trifluoromethyl-pyridin-2-yl)-quinazolin-4-yl]-(4-trifluoromethyl-phenyl)-amine;

[2-Dipropylaminoethyl-7-(3-trifluoromethyl-pyridin-2-y)-quinazolin-4-y]-(4-trifluoromethyl-phenyl)-amine;

[2-Dipropylaminomethyl-7-(3-trifluoromethyl-pyridin-2-yl)-pyrido[3,2-d]pyrimidin-4-yl]-(4-trifluoromethyl-phenyl)-amine;

[2-Dipropylaminomethyl-7-(3-trifluoromethyl-pyridin-2-yl)-quinazolin-4-yl]-(6-trifluoromethyl-pyridin-3-yl)-amine;

[2-Dipropylaminomethyl-7-(3-trifluoromethyl-pyridin-2-yl)-quinazolin-4-yl]-(4-trifluoromethyl-phenyl)-amine;

[2-Ethylaminomethyl-7-(3-trifluoromethyl-pyridin-2-yl)-quinazolin-4-yl]-(4-trifluoromethyl-phenyl)-amine;

[2-Hexylaminomethyl-7-(3-trifluoromethyl-pyridin-2-yl)-quinazolin-4-yl]-(4-trifluoromethyl-phenyl)-amine;

[2-Imidazol-1-ylmethyl-7-(3-trifluoromethyl-pyridin-2-yl)-pyrido[3,2-d]pyrimidin-4-yl]-(4-trifluoromethyl-phenyl)-amine;

[2-Imidazol-1-ylmethyl-7-(3-trifluoromethyl-pyridin-2-yl)-quinazolin-4-yl]-(6-trifluoromethyl-pyridin-3-yl)-amine;

[2-Imidazol-1-ylmethyl-7-(3-trifluoromethyl-pyridin-2-yl)-quinazolin-4-yl]-(4-trifluoromethyl-phenyl)-amine;

[2-Methylaminomethyl-7-(3-trifluoromethyl-pyridin-2-yl)-quinazolin-4-yl]-(4-trifluoromethyl-phenyl)-amine;

[2-Morpholin-4-ylethyl-7-(3-trifluoromethyl-pyridin-2-yl)-quinazolin-4-yl]-(4-trifluoromethyl-phenyl)-amine;

[2-Morpholin-4-ylmethyl-7-(3-trifluoromethyl-pyridin-2-yl)-pyrido[3,2-d]pyrimidin-4-yl]-(6-trifluoromethyl-pyridin-3-yl)-amine;

[2-Morpholin-4-ylmethyl-7-(3-trifluoromethyl-pyridin-2-yl)-pyrido[3,2-d]pyrimidin-4-yl]-(6-trifluoromethyl-pyridin-2-yl)-amine;

[2-Morpholin-4-ylmethyl-7-(3-trifluoromethyl-pyridin-2-yl)-quinazolin-4-yl]-(5-trifluoromethyl-pyridin-2-yl)-amine;

[2-Morpholin-4-ylmethyl-7-(3-trifluoromethyl-pyridin-2-yl)-quinazolin-4-yl]-[4-(2,2,2-trifluoro-1-methyl-ethyl)-phenyl]-amine;

[2-Morpholin-4-ylmethyl-7-(3-trifluoromethyl-pyridin-2-yl)-quinazolin-4-yl]-(6-trifluoromethyl-pyridin-3-yl)-amine;

[2-Morpholin-4-ylmethyl-7-(3-trifluoromethyl-pyridin-2-yl)-quinazolin-4-yl]-(4-trifluoromethanesulfonyl-phenyl)-amine;

[2-Morpholin-4-ylmethyl-7-(3-trifluoromethyl-pyridin-2-yl)-quinazolin-4-yl]-(4-trifluoromethyl-phenyl)-amine;

[2-Octylaminomethyl-7-(3-trifluoromethyl-pyridin-2-yl)-quinazolin-4-yl]-(4-trifluoromethyl-phenyl)-amine;

[2-Piperidin-1-ylethyl-7-(3-trifluoromethyl-pyridin-2-yl)-quinazolin-4-yl]-(4-trifluoromethyl-phenyl)-amine;

[2-Piperidin-1-ylmethyl-7-(3-trifluoromethyl-pyridin-2-yl)-quinazolin-4-yl]-[4-(2,2,2-trifluoro-1-methyl-ethyl)-phenyl]-amine;

[2-Piperidin-1-ylmethyl-7-(3-trifluoromethyl-pyridin-2-yl)-quinazolin-4-yl]-(4-trifluoromethyl-phenyl)-amine;

[2-Piperidin4-yl-7-(3-trifluoromethyl-pyridin-2-yl)-quinazolin-4-yl]-(4-trifluoromethyl-phenyl)-amine;

[2-Thiomorpholin-4-ylethyl-7-(3-trifluoromethyl-pyridin-2-yl)-quinazolin-4-yl]-(4-trifluoromethyl-phenyl)-amine;

[2-Thiomorpholin-4-ylmethyl-7-(3-trifluoromethyl-pyridin-2-yl)-pyrido[3,2-d]pyrimidin-4-yl]-(4-trifluoromethyl-phenyl)-amine;

[2-Thiomorpholin-4-ylmethyl-7-(3-trifluoromethyl-pyridin-2-yl)-quinazolin-4-yl]-(6-trifluoromethyl-pyridin-3-yl)-amine;

[2-Thiomorpholin-4-ylmethyl-7-(3-trifluoromethyl-pyridin-2-yl)-quinazolin-4-yl]-(4-trifluoromethyl-phenyl)-amine;

[7-(3-Chloro-pyridin-2-yl)-2-(2,6-dimethyl-morpholin-4-ylmethyl)-quinazolin-4-yl]-(4-trifluoromethyl-phenyl)-amine (cis);

[7-(3-Chloro-pyridin-2-yl)-2-(2,6-dimethyl-morpholin-4-ylmethyl)-quinazolin-4-yl]-(6-trifluoromethyl-pyridin-3-yl)-amine (cis);

[7-(3-Chloro-pyridin-2-yl)-2-(2,6-dimethyl-morpholin-4-ylmethyl)-quinazolin-4-yl]-(4-trifluoromethanesulfonyl-phenyl)-amine (cis);

[7-(3-Chloro-pyridin-2-yl)-2-(2,6-dimethyl-morpholin-4-ylmethyl)-pyrido[3,2-d]pyrimidin-4-yl]-(4-trifluoromethyl-phenyl)-amine (cis);

[7-(3-Chloro-pyridin-2-yl)-2-(2,6-dimethyl-morpholin-4-ylmethyl)-pyrido[3,2-d]pyrimidin-4-yl]-(6-trifluoromethyl-pyridin-3-yl)-amine (cis);

[7-(3-Chloro-pyridin-2-yl)-2-(2,6-dimethyl-morpholin-4-ylmethyl)-pyrido[3,2-d]pyrimidin-4-yl]-(4-trifluoromethanesulfonyl-phenyl)-amine (cis);

[7-(3-Chloro-pyridin-2-yl)-2-(2,6-dimethyl-morpholin-4-ylmethyl)-quinazolin-4-yl]-(4-isopropyl-phenyl)-amine (cis);

[7-(3-Chloro-pyridin-2-yl)-2-(3,5-dimethyl-piperazin-1-ylmethyl)-quinazolin-4-yl]-(4-trifluoromethyl-phenyl)-amine;

[7-(3-Chloro-pyridin-2-yl)-2-imidazol-1-ylmethyl-pyrido[3,2-d]pyrimidin-4-yl]-(4-trifluoromethyl-phenyl)-amine;

{1-[4-(4-Trifluoromethyl-phenylamino)-7-(3-trifluoromethyl-pyridin-2-yl)-quinazolin-2-ylmethyl]-piperidin-4-yl}-methanol;

{1-[4-(4-Trifluoromethyl-phenylamino)-7-(3-trifluoromethyl-pyridin-2-yl)-quinazolin-2-ylmethyl]-piperidin-2-yl}-methanol;

{1-[4-(4-Trifluoromethyl-phenylamino)-7-(3-trifluoromethyl-pyridin-2-yl)-quinazolin-2-ylmethyl]-piperidin-3-yl}-methanol;

{1-[7-(3-Trifluoromethyl-pyridin-2-yl)4-(6-trifluoromethyl-pyridin-3-ylamino)-quinazolin-2-ylmethyl]-piperidin-4-yl}-methanol;

{1-[7-(3-Trifluoromethyl-pyridin-2-yl)-4-(6-trifluoromethyl-pyridin-3-ylamino)-quinazolin-2-ylmethyl]-piperidin-3-yl }-methanol;

1-[4-(4-Trifluoromethyl-phenylamino)-7-(3-trifluoromethyl-pyridin-2-yl)-quinazolin-2-ylmethyl]-piperidin-4-ol;

1-[4-(4-Trifluoromethyl-phenylamino)-7-(3-trifluoromethyl-pyridin-2-yl)-quinazolin-2-ylmethyl]-piperidin-3-ol;

1-[4-(4-Trifluoromethyl-phenylamino)-7-(3-trifluoromethyl-pyridin-2-yl)-quinazolin-2-ylmethyl]-piperidine-4-carboxylic acid amide;

1-[7-(3-Trifluoromethyl-pyridin-2-yl)4-(6-trifluoromethyl-pyridin-3-ylamino)-quinazolin-2-ylmethyl]-piperidin4-ol;

1-[7-(3-Trifluoromethyl-pyridin-2-yl)-4-(6-trifluoromethyl-pyridin-3-ylamino)-quinazolin-2-ylmethyl]-piperidin-3-ol;

1-[7-(3-Trifluoromethyl-pyridin-2-yl)4-(6-trifluoromethyl-pyridin-3-ylamino)-quinazolin-2-ylmethyl]-piperidine-4-carboxylic acid amide;

1-{2-[2-(2,6-Dimethyl-morpholin-4-ylmethyl)-4-(4-trifluoromethyl-phenylamino)-quinazolin-7-yl]-phenyl}-ethanone (cis);

1-{2-[4-(4-Trifluoromethyl-phenylamino)-7-(3-trifluoromethyl-pyridin-2-yl)-quinazolin-2-yl]-ethyl}-piperidine-4-carboxylic acid amide;

1-{3-[4-(4-Trifluoromethyl-phenylamino)-7-(3-trifluoromethyl-pyridin-2-yl)-quinazolin-2-yl]-propyl-piperidin-4-ol;

1-{3-[4-(4-Trifluoromethyl-phenylamino)-7-(3-trifluoromethyl-pyridin-2-yl)-quinazolin-2-yl]-propyl}piperidin-3-ol;

1-{4-[2-(2,6-Dimethyl-morpholin-4-ylmethyl)-7-(3-trifluoromethyl-pyridin-2-yl)-pyrido[3,2-d]pyrimidin-4-ylamino]-phenyl}-ethanone (cis);

1-{4-[4-(4-Trifluoromethyl-phenylamino)-7-(3-trifluoromethyl-pyridin-2-yl)-quinazolin-2-yl]-piperidin-1-yl}-ethanone;

1-{4-[4-(4-Trifluoromethyl-phenylamino)-7-(3-trifluoromethyl-pyridin-2-yl)-quinazolin-2-yl]-piperidin-1-yl}-propan-1-one;

1-{4-[7-(3-Trifluoromethyl-pyridin-2-yl)4-(6-trifluoromethyl-pyridin-3-ylamino)-quinazolin-2-ylmethyl]-piperazin-1-yl}-ethanone;

1-Pyrrolidin-1-yl-3-[4-(4-trifluoromethyl-phenylamino)-7-(3-trifluoromethyl-pyridin-2-yl)-quinazolin-2-yl]-propan-1-one;

2-(1-{3-[4-(4-Trifluoromethyl-phenylamino)-7-(3-trifluoromethyl-pyridin-2-yl)-quinazolin-2-yl]-propyl}-piperidin-4-yl)-ethanol;

2-{[4-(4-Trifluoromethyl-phenylamino)-7-(3-trifluoromethyl-pyridin-2-yl)-quinazolin-2-ylmethyl]-amino}-ethanol;

2-{1-[4-(4-Trifluoromethyl-phenylamino)-7-(3-trifluoromethyl-pyridin-2-yl)-quinazolin-2-ylmethyl]-piperidin-4-yl}-ethanol;

2-{1-[7-(3-Trifluoromethyl-pyridin-2-yl)4-(6-trifluoromethyl-pyridin-3-ylamino)-quinazolin-2-ylmethyl]-piperidin-4-yl }-ethanol;

2-{4-[4-(4-Trifluoromethyl-phenylamino)-7-(3-trifluoromethyl-pyridin-2-yl)-quinazolin-2-ylmethyl]-piperazin-1-yl }-ethanol;

2-{4-[7-(3-Trifluoromethyl-pyridin-2-yl)-4-(6-trifluoromethyl-pyridin-3-ylamino)-quinazolin-2-ylmethyl]-piperazin-1-yl}-ethanol;

2-Methyl-2-{[4-(4-trifluoromethyl-phenylamino)-7-(3-trifluoromethyl-pyridin-2-yl)-quinazolin-2-ylmethyl]-amino}-propan-1-ol;

4-(4-Trifluoromethyl-phenylamino)-7-(3-trifluoromethyl-pyridin-2-yl)-quinazoline-2-carboxylic acid dimethylamide;

4-(4-Trifluoromethyl-phenylamino)-7-(3-trifluoromethyl-pyridin-2-yl)-quinazoline-2-carboxylic acid methylamide;

4-(4-Trifluoromethyl-phenylamino)-7-(3-trifluoromethyl-pyridin-2-yl)-quinazoline-2-carboxylic acid (2-dimethylamino-ethyl)-amide;

4-(4-Trifluoromethyl-phenylamino)-7-(3-trifluoromethyl-pyridin-2-yl)-quinazoline-2-carboxylic acid (2-morpholin-4-yl-ethyl)-amide;

4-{2-[4-(4-Trifluoromethyl-phenylamino)-7-(3-trifluoromethyl-pyridin-2-yl)-quinazolin-2-yl]-ethyl}-piperazine-1-carbaldehyde;

N,N,N′-Trimethyl-N′-[4-(4-trifluoromethyl-phenylamino)-7-(3-trifluoromethyl-pyridin-2-yl)-quinazolin-2-ylmethyl]-propane-1,3-diamine;

N-[4-(4-Trifluoromethyl-phenylamino)-7-(3-trifluoromethyl-pyridin-2-yl)-quinazolin-2-ylmethyl]-methanesulfonamide;

[2-Dimethylaminomethyl-7-(3-methyl-pyridin-2-yl)-quinazolin-4-yl]-(4-isopropyl-phenyl)-amine;

(4-Isopropyl-phenyl)-[7-(3-methyl-pyridin-2-yl)-2-morpholin-4-ylmethyl-quinazolin-4-yl]-amine

(4-Isopropyl-phenyl)-[7-(3-methyl-pyridin-2-yl)-2-thiomorpholin-4-ylmethyl-quinazolin-4-yl]-amine;

[2-(3,3-Dimethyl-piperidin-1-ylmethyl)-7-(3-methyl-pyridin-2-yl)-quinazolin-4-yl]-(4-isopropyl-phenyl)-amine;

[2-[(Ethyl-propyl-amino)-methyl]-7-(3-methyl-pyridin-2-yl)-quinazolin-4-yl]-(4-isopropyl-phenyl)-amine;

(4-Isopropyl-phenyl)-[2-[(methyl-propyl-amino)-methyl]-7-(3-methyl-pyridin-2-yl)-quinazolin-4-yl]-amine;

[2-[(Ethyl-isopropyl-amino)-methyl]-7-(3-methyl-pyridin-2-yl)-quinazolin-4-yl]-(4-isopropyl-phenyl)-amine;

[2-[(Isopropyl-methyl-amino)-methyl]-7-(3-methyl-pyridin-2-yl)-quinazolin-4-yl]-(4-isopropyl-phenyl)-amine;

[2-{[Bis-(2-methoxy-ethyl)-amino]-methyl}-7-(3-methyl-pyridin-2-yl)-quinazolin-4-yl]-(4-isopropyl-phenyl)-amine;

[2-Pyridin4-yl-7-(3-trifluoromethyl-pyridin-2-yl)-quinazolin-4-yl]-(4-trifluoromethyl-phenyl)-amine;

[2-Pyridin-3-yl-7-(3-trifluoromethyl-pyridin-2-yl)-quinazolin-4-yl]-(4-trifluoromethyl-phenyl)-amine;

[2-(6-Methoxy-pyridin-3-yl)-7-(3-trifluoromethyl-pyridin-2-yl)-quinazolin-4-yl]-(4-trifluoromethyl-phenyl)-amine;

[2-(6-Pyrrolidin-1-yl-pyridin-3-yl)-7-(3-trifluoromethyl-pyridin-2-yl)-quinazolin-4-yl]-(4-trifluoromethyl-phenyl)-amine;

(4-tert-Butyl-phenyl)-[2-(2,6-dimethyl-morpholin-4-ylmethyl)-7-pyridin-4-yl-quinazolin-4-yl]-amine (cis);

(4-tert-Butyl-phenyl)-[2-(2,6-dimethyl-morpholin-4-ylmethyl)-7-pyridin-3-yl-quinazolin-4-yl]-amine (cis);

(4-tert-Butyl-phenyl)-[2-(2,6-dimethyl-morpholin-4-ylmethyl)-7-pyrimidin-5-yl-quinazolin-4-yl]-amine (cis);

(4-tert-Butyl-phenyl)-[7-(2,4-dimethoxy-pyrimidin-5-yl)-2-(2,6-dimethyl-morpholin-4-ylmethyl)-quinazolin-4-yl]-amine (cis);

[7-(3-Chloro-pyridin-2-yl)-2-(2,6-dimethyl-morpholin-4-ylmethyl)-pyrido[3,2-d]pyrimidin-4-yl]-[4-(;

morpholine-4-sulfonyl)-phenyl]-amine;

[2-Dimethylaminomethyl-7-(3-trifluoromethyl-pyridin-2-yl)-quinazolin-4-yl]-[4-(morpholine-4-sulfonyl)-phenyl]-amine;

[2-[(Methyl-propyl-amino)-methyl]-7-(3-trifluoromethyl-pyridin-2-yl)-quinazolin-4-yl]-[4-(morpholine-4-sulfonyl)-phenyl]-amine;

[2-[(Isopropyl-methyl-amino)-methyl]-7-(3-trifluoromethyl-pyridin-2-yl)-quinazolin-4-yl]-[4-(morpholine-4-sulfonyl)-phenyl]-amine;

[2-[(Ethyl-propyl-amino)-methyl]-7-(3-trifluoromethyl-pyridin-2-yl)-quinazolin-4-yl]-[4-(morpholine-4-sulfonyl)-phenyl]-amine;

[2-[(Bis-ethoxymethyl-amino)-methyl]-7-(3-methyl-pyridin-2-yl)-quinazolin-4-yl]-(4-trifluoromethyl-phenyl)-amine;

[2-Dipropylaminomethyl-7-(3-methyl-pyridin-2-yl)-quinazolin-4-yl]-(4-trifluoromethyl-phenyl)-amine;

[2-(3,3-Dimethyl-piperidin-1-ylmethyl)-7-(3-methyl-pyridin-2-yl)-quinazolin-4-yl]-(4-trifluoromethyl-phenyl)-amine;

1-[7-(3-Methyl-pyridin-2-yl)-4-(4-trifluoromethyl-phenylamino)-quinazolin-2-ylmethyl]-pyrrolidin-3-ol (chiral);

[2-{[Methyl-(1-phenyl-ethyl)-amino]-methyl}-7-(3-trifluoromethyl-pyridin-2-yl)-quinazolin-4-yl]-(6-trifluoromethyl-pyridin-3-yl)-amine;

[2-[(Indan-1-yl-methyl-amino)-methyl]-7-(3-trifluoromethyl-pyridin-2-yl)-quinazolin-4-yl]-(6-trifluoromethyl-pyridin-3-yl)-amine;

[2-{[Methyl-(1-phenyl-propyl)-amino]-methyl}-7-(3-trifluoromethyl-pyridin-2-yl)-quinazolin-4-yl]-(6-trifluoromethyl-pyridin-3-yl)-amine;

[2-( l-Methyl-3,4-dihydro-1H-isoquinolin-2-ylmethyl)-7-(3-trifluoromethyl-pyridin-2-yl)-quinazolin-4-yl]-(6-trifluoromethyl-pyridin-3-yl)-amine;

[2-[(Benzyl-methyl-amino)-methyl]-7-(3-trifluoromethyl-pyridin-2-yl)-quinazolin-4-yl]-(6-trifluoromethyl-pyridin-3-yl)-amine;

[2-(3,4-Dihydro-1H-isoquinolin-2-ylmethyl)-7-(3-trifluoromethyl-pyridin-2-yl)-quinazolin-4-yl]-(6-trifluoromethyl-pyridin-3-yl)-amine;

[2-{[(3-Fluoro-benzyl)-methyl-amino]-methyl}-7-(3-trifluoromethyl-pyridin-2-yl)-quinazolin-4-yl]-(6-trifluoromethyl-pyridin-3-yl)-amine;

[2-{[Methyl-(2-methyl-benzyl)-amino]-methyl}-7-(3-trifluoromethyl-pyridin-2-yl)-quinazolin-4-yl]-(6-trifluoromethyl-pyridin-3-yl)-amine;

[2-{[(2-Fluoro-benzyl)-methyl-amino]-methyl}-7-(3-trifluoromethyl-pyridin-2-yl)-quinazolin-4-yl]-(6-trifluoromethyl-pyridin-3-yl)-amine;

[2-[(Benzyl-cyclopropyl-amino)-methyl]-7-(3-trifluoromethyl-pyridin-2-yl)-quinazolin-4-yl]-(6-trifluoromethyl-pyridin-3-yl)-amine;

[2-[(Methyl-phenethyl-amino)-methyl]-7-(3-trifluoromethyl-pyridin-2-yl)-quinazolin-4-yl]-(6-trifluoromethyl-pyridin-3-yl)-amine;

[2-Pyrrolidin-1-ylmethyl-7-(3-trifluoromethyl-pyridin-2-yl)-quinazolin-4-yl]-(6-trifluoromethyl-pyridin-3-yl)-amine;

[2-Piperidin-1-ylmethyl-7-(3-trifluoromethyl-pyridin-2-yl)-quinazolin-4-yl]-(6-trifluoromethyl-pyridin-3-yl)-amine;

[2-(4-Methyl-piperidin-1-ylmethyl)-7-(3-trifluoromethyl-pyridin-2-yl)-quinazolin-4-yl]-(6-trifluoromethyl-pyridin-3-yl)-amine;

[2-Azepan-1-ylmethyl-7-(3-trifluoromethyl-pyridin-2-yl)-quinazolin-4-yl]-(6-trifluoromethyl-pyridin-3-yl)-amine;

[2-Azocan-1-ylmethyl-7-(3-trifluoromethyl-pyridin-2-yl)-quinazolin-4-yl]-(6-trifluoromethyl-pyridin-3-yl)-amine;

(6-Trifluoromethyl-pyridin-3-yl)-[7-(3-trifluoromethyl-pyridin-2-yl)-2-(3,3,5-trimethyl-azepan-1-ylmethyl)-quinazolin-4-yl]-amine;

[2-(1,4-Dioxa-8-aza-spiro[4.5]dec-8-ylmethyl)-7-(3-trifluoromethyl-pyridin-2-yl)-quinazolin-4-yl]-(6-trifluoromethyl-pyridin-3-yl)-amine;

[2-(Octahydro-quinolin-1-ylmethyl)-7-(3-trifluoromethyl-pyridin-2-yl)-quinazolin-4-yl]-(6-trifluoromethyl-pyridin-3-yl)-amine;

[2-Dimethylaminomethyl-7-(3-trifluoromethyl-pyridin-2-yl)-quinazolin-4-yl]-(6-trifluoromethyl-pyridin-3-yl)-amine;

[2-[(Allyl-methyl-amino)-methyl]-7-(3-trifluoromethyl-pyridin-2-yl)-quinazolin-4-yl]-(6-trifluoromethyl-pyridin-3-yl)-amine;

[2-Diethylaminomethyl-7-(3-trifluoromethyl-pyridin-2-yl)-quinazolin-4-yl]-(6-trifluoromethyl-pyridin-3-yl)-amine;

[2-[(Methyl-propyl-amino)-methyl]-7-(3-trifluoromethyl-pyridin-2-yl)-quinazolin-4-yl]-(6-trifluoromethyl-pyridin-3-yl)-amine;

[2-[(Butyl-methyl-amino)-methyl]-7-(3-trifluoromethyl-pyridin-2-yl)-quinazolin-4-yl]-(6-trifluoromethyl-pyridin-3-yl)-amine;

[2-[(Ethyl-isopropyl-amino)-methyl]-7-(3-trifluoromethyl-pyridin-2-yl)-quinazolin-4-yl]-(6-trifluoromethyl-pyridin-3-yl)-amine;

[2-Diallylaminomethyl-7-(3-trifluoromethyl-pyridin-2-yl)-quinazolin-4-yl]-(6-trifluoromethyl-pyridin-3-yl)-amine;

[2-Diproplaminomethyl-7-(3-trifluoromethyl-pyridin-2-yl)-quinazolin-4-yl]-(6-trifluoromethyl-pyridin-3-yl)-amine;

[2-[(Butyl-ethyl-amino)-methyl]-7-(3-trifluoromethyl-pyridin-2-yl)-quinazolin-4-yl]-(6-trifluoromethyl-pyridin-3-yl)-amine;

[2-[(Cyclopropylmethyl-propyl-amino)-methyl]-7-(3-trifluoromethyl-pyridin-2-yl)-quinazolin-4-yl]-(6-trifluoromethyl-pyridin-3-yl)-amine;

[2-[(Hexyl-methyl-amino)-methyl]-7-(3-trifluoromethyl-pyridin-2-yl)-quinazolin-4-yl]-(6-trifluoromethyl-pyridin-3-yl)-amine;

[2-Dibutylaminomethyl-7-(3-trifluoromethyl-pyridin-2-yl)-quinazolin-4-yl]-(6-trifluoromethyl-pyridin-3-yl)-amine;

[2-[(Isopropyl-methyl-amino)-methyl]-7-(3-trifluoromethyl-pyridin-2-yl)-quinazolin-4-yl]-(6-trifluoromethyl-pyridin-3-yl)-amine;

[2-(2-Methyl-piperidin-1-ylmethyl)-7-(3-trifluoromethyl-pyridin-2-yl)-quinazolin-4-yl]-(6-trifluoromethyl-pyridin-3-yl)-amine;

[2-{[Ethyl-(2-methyl-allyl)-amino]-methyl}-7-(3-trifluoromethyl-pyridin-2-yl)-quinazolin-4-yl]-(6-trifluoromethyl-pyridin-3-yl)-amine;

[2-[(Cyclohexyl-methyl-amino)-methyl]-7-(3-trifluoromethyl-pyridin-2-yl)-quinazolin-4-yl]-(6-trifluoromethyl-pyridin-3-yl)-amine;

[2-(2-Ethyl-piperidin-1-ylmethyl)-7-(3-trifluoromethyl-pyridin-2-yl)-quinazolin-4-yl]-(6-trifluoromethyl-pyridin-3-yl)-amine;

[2-[(Cyclohexyl-ethyl-amino)-methyl]-7-(3-trifluoromethyl-pyridin-2-yl)-quinazolin-4-yl]-(6-trifluoromethyl-pyridin-3-yl)-amine;

[2-{[Bis-(2-methoxy-ethyl)-amino]-methyl}-7-(3-trifluoromethyl-pyridin-2-yl)-quinazolin-4-yl]-(6-trifluoromethyl-pyridin-3-yl)-amine;

[2-Dipentylaminomethyl-7-(3-trifluoromethyl-pyridin-2-yl)-quinazolin-4-yl]-(6-trifluoromethyl-pyridin-3-yl)-amine;

[2-Dihexylaminomethyl-7-(3-trifluoromethyl-pyridin-2-yl)-quinazolin-4-yl]-(6-trifluoromethyl-pyridin-3-yl)-amine;

[2-(3,5-Dimethyl-piperidin-1-ylmethyl)-7-(3-trifluoromethyl-pyridin-2-yl)-quinazolin-4-yl]-(6-trifluoromethyl-pyridin-3-yl)-amine;

{1-[7-(3-Methyl-pyridin-2-yl)4-(4-trifluoromethyl-phenylamino)-quinazolin-2-ylmethyl]-pyrrolidin-3-yl}-methanol (chiral);

{1-[7-(3-Methyl-pyridin-2-yl)4-(4-trifluoromethyl-phenylamino)-quinazolin-2-ylmethyl]-pyrrolidin-3-yl}-methanol (chiral);

[2-(2,6-Dimethyl-morpholin-4-ylmethyl)-7-(3-methyl-pyridin-2-yl)-pyrido[2,3-d]pyrimidin-4-yl]-(4-trifluoromethyl-phenyl)-amine;

[2-Azetidin-3-yl-7-(3-methyl-pyridin-2-yl)-pyrido[2,3-d]pyrimidin-4-yl]-(4-trifluoromethyl-phenyl)-amine;

[2-(2,2-Dimethyl-morpholin-4-ylmethyl)-7-(3-trifluoromethyl-pyridin-2-yl)-quinazolin-4-yl]-(4-trifluoromethyl-phenyl)-amine;

[7-(3-Chloro-pyridin-2-yl)-2-(2,2-dimethyl-morpholin-4-ylmethyl)-quinazolin-4-yl]-(4-trifluoromethyl-phenyl)-amine;

(4-Cyclopropyl-phenyl)-[2-(2,2-dimethyl-morpholin4-ylmethyl)-7-(3-trifluoromethyl-pyridin-2-yl)-quinazolin-4-yl]-amine;

[2-(2,2-Dimethyl-morpholin-4-ylmethyl)-7-(3-trifluoromethyl-pyridin-2-yl)-pyrido[3,2-d]pyrimidin-4-yl]-(4-trifluoromethyl-phenyl)-amine;

[2-{[Bis-(2-methoxy-ethyl)-amino]-methyl}-7-(3-methyl-pyridin-2-yl)-pyrido[2,3-d]pyrimidin-4-yl]-(4-trifluoromethyl-phenyl)-amine;

[2-(3,3-Dimethyl-piperidin-1-ylmethyl)-7-(3-methyl-pyridin-2-yl)-pyrido[2,3-d]pyrimidin-4-yl]-(4-trifluoromethyl-phenyl)-amine;

[2-(2,6-Dimethyl-morpholin-4-ylmethyl)-7-(3-trifluoromethyl-pyridin-2-yl)-pyrido[2,3-d]pyrimidin-4-yl]-(4-trifluoromethyl-phenyl)-amine (cis);

[2-{[Bis-(2-methoxy-ethyl)-amino]-methyl}-7-(3-trifluoromethyl-pyridin-2-yl)-pyrido[2,3-d]pyrimidin-4-yl]-(4-trifluoromethyl-phenyl)-amine;

[2-(3,3-Dimethyl-piperidin-1-ylmethyl)-7-(3-trifluoromethyl-pyridin-2-yl)-pyrido[2,3-d]pyrimidin-4-yl]-(4-trifluoromethyl-phenyl)-amine;

2-{[4-(4-tert-Butyl-phenylamino)-7-(3-trifluoromethyl-pyridin-2-yl)-quinazolin-2-ylmethyl]-propyl-amino}-ethanol;

{1-[4-(4-tert-Butyl-phenylamino)-7-(3-trifluoromethyl-pyridin-2-yl)-quinazolin-2-ylmethyl]-pyrrolidin-2-yl}-methanol; and

[2-( 1,1-Dioxo-1λ⁶-[1,2]thiazinan-2-ylmethyl)-7-(3-trifluoromethyl-pyridin-2-yl)-quinazolin-4-yl]-(4-trifluoromethyl-phenyl)-amine.

### Example 4

### Preparation of Representative VR1 Receptor Antagonists

This Example illustrates the preparation of representative substituted 2-hydroxyalkyl-quinazolin-4-ylamine analogues. Synthesis of the compounds provided in this Example is also described in PCT International Application Publication Number WO 03/062209, which published on Jul. 31, 2003.

### A. [2-Isopropoxymethyl-7-(3-trifluoromethylpyridin-2-yl)-quinazolin-4-yl]-(4-trifluoromethyl-phenyl)-amine ester

### 1. 2-p-tolyl-3-trifluoromethyl-pyridine

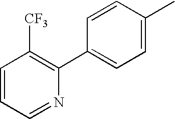

To a de-gassed mixture of 2-chloro-3-(trifluoromethyl)-pyridine (70.1 mmol), p-tolylboronic acid (70.6 mmol), and 2M Na₂CO₃ (175.0 mmol), in dimethyl ether (DME; 200 mL) under nitrogen add Pd(PPh₃)₄ (2.8 mmol). Stir the mixture at 80° C. overnight, concentrate, and extract with EtOAc. Dry over Na₂SO₄, concentrate under vacuum, and pass through a silica gel pad to give 2-p-tolyl-3-trifluoromethyl-pyridine.

### 2. 2-(4-methyl-3-nitro-phenyl)-3-(trifluoromethyl)-pyridine

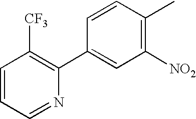

To a solution of 2-p-tolyl-3-trifluoromethyl-pyridine (8.4 mmol) in H₂SO₄ (6 mL) cautiously add fuming HNO₃ (2 ml). Stir the mixture for 60 minutes at room temperature. Pour the mixture onto ice-water (30 mL), extract with EtOAc, neutralize with 1 N NaOH, dry over Na₂SO₄, and concentrate under vacuum to obtain 2-(4-methyl-3-nitro-phenyl)-3-(trifluoromethyl)-pyridine.

### 3. 2-nitro-4-(3-trifluoromethyl-pyridin-2-yl)-benzoic acid

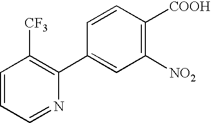

To a solution of 2-(4-methyl-3-nitro-phenyl)-3-(trifluoromethyl)-pyridine (7.1 mmol) in a mixture of pyridine (10 mL) and water (5 ml), add KMnO₄ (25.3 mmol) portionwise. Stir the mixture for 4 hours at 110° C., and then add another 25.3 mmol of KMnO4 with 10 ml of water. Stir the mixture at 110° C. over night. Cool to room temperature, and filter through celite pad. Concentrate the filtrate under vacuum, dilute with water, and wash the aqueous solution with EtOAc. Neutralize the aqueous solution with 2 N HCl and collect the precipitate to give 2-nitro-4(3-trifluoromethyl-pyridin-2-yl)-benzoic acid.

### 4. 2-nitro-4-(3-trifluoromethyl-pyridin-2-yl)-benzamide

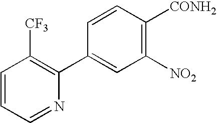

Reflux a mixture of 2-amino-4(3-trifluoromethyl-pyridin-2-yl)-benzoic acid (25 g) with SOCl₂ (50 ml) for 4 hours and concentrate. Dissolve the residue in dichloromethane (DCM), cool with ice-water bath, pass NH₃ gas through the solution for 30 minutes, and stir for 15 minutes at room temperature. Concentrate and wash with water to give 2-nitro4-(3-trifluoromethyl-pyridin-2-yl)-benzamide.

### 5. 2-amino-4-(3-trifluoromethyl-pyridin-2-yl)-benzamide

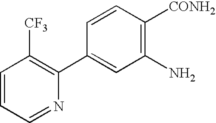

Hydrogenate 2-nitro4-(3-trifluoromethyl-pyridin-2-yl)-benzamide (1.0 g, 0.0032 mol) with 50 psi of H₂ and 100 mg of 10% Pd/C in ethanol. After 16 hours, filter the mixture through celite and concentrate under reduced pressure to give 2-amino-4-(3-trifluoromethyl-pyridin-2-yl)-benzamide as a solid.

### 6. 2-chloromethyl-7-(3-trifluoromethyl-pyridin-2-yl)-3H-quinazolin-4-one

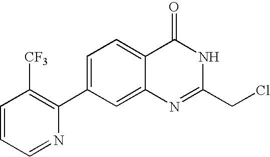

Heat a solution of 2-amino-4-(3-trifluoromethyl-pyridin-2-yl)-benzamide (100 mg, 0.356 mmol) in 2-chloro-1,1,1-trimethoxyethane (bp 138° C.) at 130° C. for 4 hours. Concentrate the mixture under reduced pressure to give 2-chloromethyl-7-(3-trifluoromethyl-pyridin-2-yl)-3H-quinazolin-4-one as an oil which crystallizes on standing.

### 7. 4-chloro-2-chloromethyl-7-(3-trifluoromethyl-pyridin-2-yl)-quinazoline

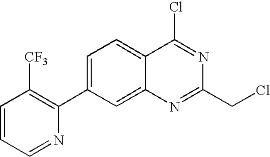

Reflux a mixture of 2-chloromethyl-7-(3-trifluoromethyl-pyridin-2-yl)-3H-quinazolin-4-one (obtained from the reaction above) and POCl₃ for 16 hours. Cool the mixture and concentrate under reduced pressure. Partition the residue between EtOAc and saturated NaHCO₃ solution. Wash the EtOAc portion with additional NaHCO₃ and then dry (Na₂SO₄) and concentrate under reduced pressure. Filter the brown residue through 2 inches of silica gel (1:1 EtOAc/hexanes eluent) and concentrate under reduced pressure to give 4-chloro-2-chloromethyl-7-(3-trifluoromethyl-pyridin-2-yl)-quinazoline.

### 8. [2-chloromethyl-7-(3-trifluoromethyl-pyridin-2-yl)-quinazolin-4-yl]-(4-trifluoro methyl-phenyl)-amine

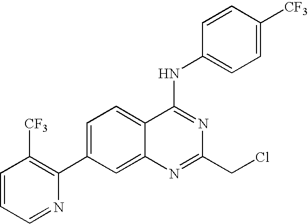

Heat a mixture of 4-chloro-2-chloromethyl-7-(3-trifluoromethyl-pyridin-2-yl)-quinazoline (42 mg, 0.117 mmol) and 4-trifluoromethyl-aniline (19 mg, 0.117 mmol) in isopropyl alcohol (1 mL) at 75° C. for 4 hours. Cool the mixture and wash the precipitate with isopropyl alcohol followed by ether to give [2-chloromethyl-7-(3-trifluoromethyl-pyridin-2-yl)-quinazolin-4-yl]-(4-trifluoromethyl-phenyl)-amine as the mono-HCl salt.

### 9. [2-Isopropoxymethyl-7-(3-trifluoromethyl-pyridin-2-yl)-quinazolin-4-yl]-(4-trifluoromethyl-phenyl)-amine

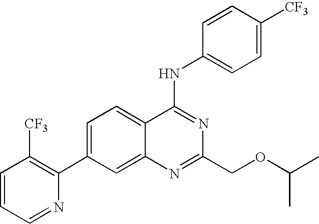

To a suspension of [2-chloromethyl-7-(3-trifluoromethyl-pyridin-2-yl)-quinazolin-4-yl]-(4-trifluoromethyl-phenyl)-amine hydrochloride (1.9 g, 0.0037 mol) in dry isopropanol (100 mL), add 20 equivalents of NaO-i-Pr (prepared from Na and isopropanol). Stir the pale yellow mixture at 60° C. for 5 hours, cool and evaporate the solvent under reduced pressure. Partition the residue between ethyl acetate and water and wash the organic layer with water (1×). Dry the organic layer (Na₂SO₄) and concentrate to give [2-isopropoxymethyl-7-(3-trifluoromethyl-pyridin-2-yl)-quinazolin-4-yl]-(4-trifluoromethyl-phenyl)-amine as a foam.

### B. 2-[4-(4-Trifluoromethylphenylamino)-7-(3-trifluoromethyl-pyridin-2-yl)-quinazolin-2-yl]-ethanol

### 1. 3-Benzyloxy-propionic acid

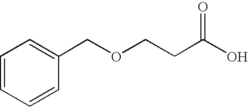

Add sodium hydride (2.22 g, 60% dispersion in mineral oil, 55.4 mmol) in small portions to a cold (0° C.) solution of benzyl alcohol (4.0 g, 37 mmol) in toluene (100 mL). Add ethyl 3-bromopropionate (8.0 g, 44 mmol) dropwise to the mixture, allow the resulting solution to warm to room temperature and stir for 1 hour. Quench the reaction with the addition of water until all bubbling ceases. Dilute the mixture with ethyl acetate (100 mL) and extract with water (100 mL) and brine (100 mL). Dry the organic extract over Na₂SO₄ and remove the solvent under reduced pressure to yield the crude ester as a clear oil. Dissolve the oil in methanol (20 mL) and 6 N NaOH (20 mL), stir for 1 hour, concentrate the mixture ( 20 mL) and dilute with water (20 mL). Extract the aqueous mixture once with CH₂Cl₂ (40 mL). Acidify the aqueous phase with conc. HCl, extract with EtOAc (3×50 mL), and dry the combined EtOAc extracts over Na₂SO₄. Remove the solvent under reduced pressure to yield the title compound as a clear oil that solidifies upon standing.

### 2. 2-(2-Benzyloxy-ethyl)-7-(3-trifluoromethyl-pyridiny-2-yl)-3H-quinazolin-4-one

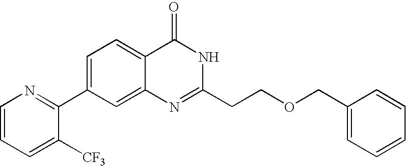

Cool a solution of 3-benzyloxy-propionic acid (1.66 g, 9.19 mmol) in hexanes (40 mL) to 0° C. and add oxalyl chloride (3.50 g, 27.6 mmol) dropwise. After the addition is completed, add DMF (2 drops) and stir the resulting mixture for 1 hour. Remove the solvent under reduced pressure and dissolve the crude acid chloride in dry THF (20 mL). In a separate flask, dissolve 2-amino-4-(3-trifluoromethyl-pyridin-2-yl)-benzamide (2.35 g, 8.37 mmol) in dry THF (40 mL) and pyridine (0.727 g, 9.19 mmol) and cool to 0° C. Add the solution containing the crude acid chloride dropwise to the second solution. Allow the mixture to warm to room temperature and stir for 1 hour. Add a solution of 10% NaOH(aq) (20 mL) to the mixture and stir the solution for 1 hour. Concentrate the mixture (20 mL), dilute with water (20 mL), and acidify with conc. HCl. Extract the resulting solution with EtOAC (3×50 mL). Wash the combined organic extracts with brine and dry over Na₂SO₄. Remove the solvent under reduced pressure to yield the title compound as a white solid.

### 3. 2-(2-Benzyloxy-ethyl)4-chloro-7-(3-trifluoromethyl-pyridiny-2-yl)-quinazoline

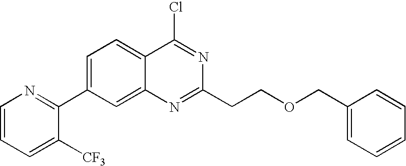

Dissolve 2-(2-Benzyloxy-ethyl)-7-(3-trifluoromethyl-pyridiny-2-yl)-3H-quinazolin-4-one (3.24 g, 7.62 mmol) in CHCl₃ (40 mL) and 2,6-lutidine (2.45 g, 22.9 mmol). Add phosphorous oxychloride (1.77 mL, 19.0 mmol) dropwise and heat the resulting solution to reflux for 18 hours. Cool the solution and remove the solvent under reduced pressure. Partition the crude residue between EtOAc (200 mL) and saturated NaHCO₃ (aq) (200 mL). Remove the organic phase and extract the aqueous phase with EtOAc (200 mL). Combine the two organic extracts, wash with brine (200 mL), and dry over Na₂SO₄. Remove the solvent to yield the title compound as a light brown solid.

### 4. [2-(2-Benzyloxy-ethyl)-7-(3-trifluoromethyl-pyridiny-2-yl)-quinazolin-4-yl]-(4-trifluoromethyl-phenyl)-amine

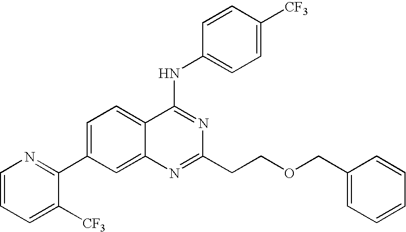

Dissolve 2-(2-Benzyloxy-ethyl)-4-chloro-7-(3-trifluoromethyl-pyridiny-2-yl)-quinazoline (2.47 g, 5.57 mmol) in a solution of acetonitrile (50 mL) and 4-trifluoromethyl-aniline (0.986 g, 6.12 mmol). Heat the mixture to 80° C. for 2 hours. A white precipitate forms. Cool the solution in an ice bath and add diethyl ether (25 mL). Filter off the white precipitate and dry in a vacuum oven to yield the title compound as the mono-hydrochloride salt.

### 5. 2-[4-(4-Trifluoromethyl-phenylamino)-7-(3-trifluoromethyl-pyridin-2-yl)-quinazolin-2-yl]-ethanol

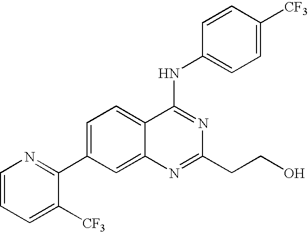

Dissolve [2-(2-Benzyloxy-ethyl)-7-(3-trifluoromethyl-pyridiny-2-yl)-quinazolin-4-yl]-(4-trifluoromethyl-phenyl)-amine hydrochloride (2.96 g, 4.89 mmol) in MeOH (150 mL) and add 10% Pd/C (200 mg). Hydrogenate the mixture at 50 p.s.i. at 60° C. for 8 hours. Quickly filter the mixture through Celite and wash the Celite filter cake with hot MeOH (200 mL). Remove the solvent under reduced pressure to yield the mono-hydrochloride salt of title compound as a white solid.

### C. [2-(2-methoxy-ethyl)-7-(3-trifluoromethylpyridin-2-yl)-quinazolin-4-yl]-(4-trifluoromethyl-phenyl)-amine

### 1. 2-(2-methoxy-ethyl)-7-(3-trifluoromethyl-pyridin-2-yl)-quinazolin-4-ol

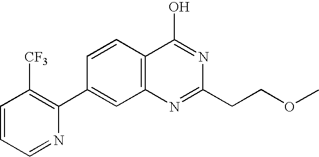

To a solution of 2-amino-4-(3-trifluoromethyl-pyridin-2-yl)-benzamide (3.56 mmol) and pyridine (3.91 mmol) in THF (20 ml), add 4-methoxy-butyryl chloride (3.91 mmol). Stir the mixture 20 minutes at room temperature, add 20 ml of 20% NaOH, stir for 60 minutes at 50° C. Concentrate, add water, filter, acidify to pH=6, collect the precipitate to obtain 2-(3-benzyloxy-propyl)7-(3-trifluoromethyl-pyridin-2-yl)-quinazolin-4-ol.

### 2. 2-(2-methoxy-ethyl)-4-chloro-7-(3-trifluoromethyl-pyridin-2-yl)-quinazoline

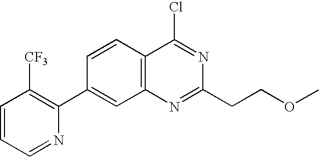

Using procedures analogous to those already described, 2-(2-methoxy-ethyl)-4-chloro-7-(3-trifluoromethyl-pyridin-2-yl)-quinazoline is prepared from 2-(2-methoxy-ethyl)-7-(3-trifluoromethyl-pyridin-2-yl)-quinazolin-4-ol.

### 3. [2-(2-methoxy-ethyl)-7-(3-trifluoromethyl-pyridin-2-yl)-quinazolin-4-yl]-(4-trifluoromethyl-phenyl)-amine

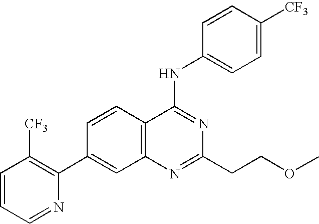

Using procedures analogous to those already described, [2-(2-methoxy-ethyl)-7-(3-trifluoromethyl-pyridin-2-yl)-quinazolin-4-yl]-(4-trifluoromethyl-phenyl)-amine is prepared from 2-(2-methoxy-ethyl)4-chloro-7-(3-trifluoro-methyl-pyridin-2-yl)-quinazoline.

### D. 3-[4-(4-trifluoromethyl-phenylamino)-7-(3-trifluoro-methyl-pyridin-2-yl)-quinazolin-2-yl]-propan-1-ol

### 1. 2-(3-benzyloxy-propyl)-7-(3-trifluoromethyl-pyridin-2-yl)-quinazolin-4-ol

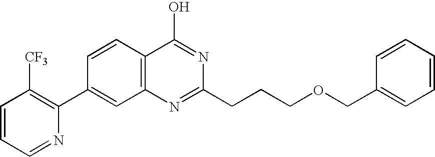

To a solution of 2-amino-4-(3-trifluoromethyl-pyridin-2-yl)-benzamide (3.56 mmol) and pyridine (3.91 mmol) in THF (20 ml) add 4-benzyloxy-butyryl chloride (3.91 mmol). Stir the mixture 20 minutes at room temperature, add 20 ml of 20% NaOH, stir for 60 minutes at 50° C. Concentrate, add water, filter, acidify to pH=6, collect the precipitate to obtain 2-(3-benzyloxy-propyl)-7-(3-trifluoromethyl-pyridin-2-yl)-quinazolin-4-ol.

### 2. 2-(3-benzyloxy-propyl)-4-chloro-7-(3-trifluoromethyl-pyridin-2-yl)-quinazoline

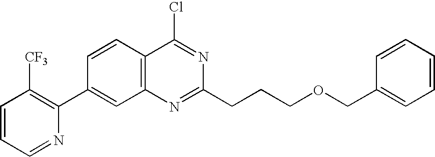

Using procedures analogous to those already described 2-(3-benzyloxy-propyl)-4-chloro-7-(3-trifluoromethyl-pyridin-2-yl)-quinazoline can be prepared from 2-(3-benzyloxy-propyl)-7-(3-trifluoromethyl-pyridin-2-yl)-quinazolin-4-ol.

### 3. [2-(3-benzyloxy-propyl)-7-(3-trifluoromethyl-pyridin-2-yl)-quinazolin-4-yl]-(4-trifluoromethyl-phenyl)-amine

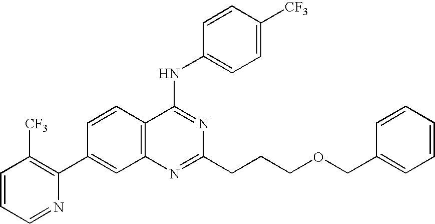

Using procedures analogous to those already described, [2-(3-benzyloxy-propyl)-7-(3-trifluoromethyl-pyridin-2-yl)-quinazolin-4-yl]-(4-trifluoromethyl-phenyl)-amine is prepared from 2-(3-benzyloxy-propyl)4-chloro-7-(3-trifluoromethyl-pyridin-2-yl)-quinazoline.

### 4. 3-[4-(4-trifluoromethyl-phenylamino)-7-(3-trifluoro-methyl-pyridin-2-yl)-quinazolin-2-yl]-propan-1-ol

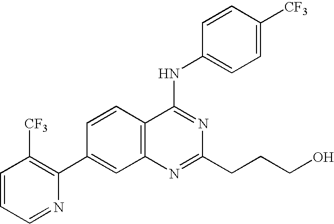

Hydrogenate the mixture of 2-(3-benzyloxy-propyl)-4-chloro-7-(3-trifluoromethyl-pyridin-2-yl)-quinazoline (0.5 mmol) and 10% Pd—C in EtOH (100 ml) at 50 psi for 30 hours. Filter, concentrate, and chromatograph to give 3-[4-(4-trifluoromethyl-phenylamino)-7-(3-trifluoro-methyl-pyridin-2-yl)-quinazolin-2-yl]-propan-1-ol.

### E. 7-(3-trifluoromethyl-pyridin-2-yl)-2-methoxymethyl-pyrido[3,2-d]pyrimidin-4-yl]-(4-trifluoromethyl-phenyl)-amine

### 1. 6′-Methoxy-3-trifluoromethyl-[2,3′]bipyridinyl

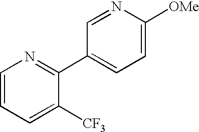

Heat a mixture of 2-chloro-3-trifluoromethylpyridine (37 g, 0.2 mol), 2-methoxypyridine-5-boronic acid (32 g, 0.21 mol), tetrakis(triphenylphosphine)palladium(0) (9 g, 7 mmol) and 2M potassium carbonate (150 mL) in toluene (500 mL) under a nitrogen atmosphere at 90° C. for 8 hours. Cool the reaction mixture and separate the layers. Extract the aqueous layer with ethyl acetate (2×250 mL) and wash the combined organics with 4M sodium hydroxide (250 mL), water (250 mL), and brine (250 mL). Dry (MgSO₄) and concentrate under reduced pressure. Purify the resulting oil by flash chromatography on silica gel (50% ether/50% hexane) to give the title compound as a colorless oil. 2. 3-Trifluoromethyl-1′H-[2,3′bipyridinyl-6′-one

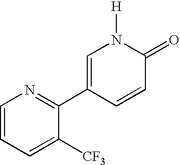

Heat 6′-Methoxy-3-trifluoromethyl-[2,3′]bipyridinyl (41 g, 0.16 mol) in 30% HBr/AcOH (100 mL) to reflux for 1 hour. Cool the mixture, filter and wash the precipitate with ether (100 mL). Transfer the precipitate into 10M sodium hydroxide (500 mL) and stir for 1 hour. Treat the solution with hydrochloric acid until the solution is pH 7. Collect the white solid by filtration and air dry to give the title compound as a white solid.

### 3. 5 ′-Nitro-3-trifluoromethyl-1′H-[2,3′bipyridinyl-6′-one

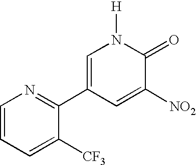

To a solution of 3-trifluoromethyl-1′H-[2,3′]bipyridinyl-6′-one (25 g, 0.1 mol) in concentrated sulfuric acid (100 mL) at 0° C., add dropwise a solution of fuming nitric acid (35 mL) and concentrated sulfuric acid (10 mL). Heat the reaction mixture to 70° C. for 1 hour, cool and pour onto ice (500 mL). Filter the mixture and treat the filtrate with 10 M sodium hydroxide until the solution is at pH 4-5. Collect the precipitate by filtration and air dry to give the title compound as a white solid.

### 4.6 ′-Chloro-5 ′-nitro-3-trifluoromethyl-[2,3′bipyridinyl

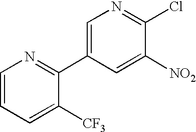

Heat a solution of 5′-nitro-3-trifluoromethyl-1′H-[2,3′]bipyridinyl-6′-one (25 g, 0.088 mol), thionyl chloride (300 mL) and DMF (3 mL) to reflux for 4 hours. Remove the volatiles by rotary evaporation and partition the residue between ethyl acetate (350 mL) and saturated sodium bicarbonate solution (250 mL). Extract the aqueous layer with further ethyl acetate (250 mL) and wash the combined organics with brine (250 mL). Dry (MgSO₄) and concentrate under reduced pressure to give the title compound as a yellow oil.

### 5. 6′-Chloro-3-trifluoromethyl-[2,3]′bipyridinyl-5′-ylamine

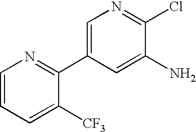

To a solution of 6′-chloro-5′-nitro-3-trifluoromethyl-[2,3′]bipyridinyl (25 g, 0.082 mol) and calcium chloride (11 g, 0.1 mol) in ethanol (300 mL) and water (50 mL), add iron powder (45 g, 0.82 mol). Heat the solution to reflux for 1.5 hours, cool and filter through Celite. Concentrate the mixture under reduced pressure, re-dissolve in ethyl acetate (300 mL) and wash with brine (200 mL). Concentrate the solution under reduced pressure and purify by flash chromatography on silica gel (50% ether/50% hexane) to give the title compound as a pale yellow solid.

### 6. 3-Amino-5-[3-(trifluoromethyl)(2-pyridyl)]pyridine-2-carboxamide

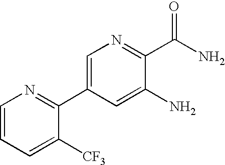

Heat a solution of 6′-chloro-3-trifluoromethyl-[2,3′]bipyridinyl-5′-ylamine (25 g, 0.091 mol), zinc cyanide (6.75 g, 0.058 mol), tris[dibenzylidineacetone]di-palladium (pd₂(dba)₃; 2.63 g, 2.86 mmol), and 1,1′-bis(diphenylphosphino)ferrocene (DPPF; 3.16 g, 5.72 mmol) in DMF (250 mL) and water (2.5 mL), under a nitrogen atmosphere, at 120° C. for 1 hour. Add water (30 mL) and heat the solution at 120° C. for a further 4 hours to complete the hydrolysis. Cool the reaction to 0° C. and add a solution of saturated ammonium chloride (200 ml), water (200 mL) and concentrated ammonium hydroxide (50 mL). After stirring at 0° C. for 1 hour, filter the yellow precipitate, and wash with water (200 mL) and a 1:1 mixture of ether-hexane (200 mL). Air dry the solid, and then dry in a vacuum oven to give of the title compound.

### 7. 2-(Chloromethyl)-7-[3-(trifluoromethyl)(2-pyridyl)]-3-hydropyridino[3,2-d]pyrimidin-4-one

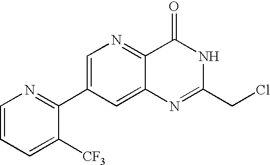

Heat a solution of 3-amino-5-[3-(trifluoromethyl)(2-pyridyl)]pyridine-2-carboxamide (23 g, 81.5 mmol) and 2-chlorol,1,1-trimethoxyethane (250 mL) at 130° C. for 1 hour. Remove the volatiles by evaporation and triturate the solid (50% ether/50% hexane) to give the title compound as a light brown solid.

### 8. 4-Chloro-2-chloromethyl-7-(3-chloro-pyridin-2-yl) -pyrido[3,2-d]pyrimidine

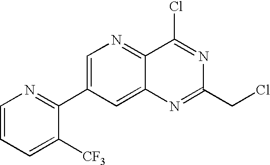

Heat a solution of 2-(chloromethyl)-7-[3-(trifluoromethyl)(2-pyridyl)]-3-hydropyridino[3,2-d]pyrimidin-4-one (2.49 g, 7.31 mmol), phosphorous oxychloride (10 mL), 2,6-lutidine (2.13 mL, 18.3 mmol) and toluene to reflux for 8 hours. Remove the solvent and partition the crude residue between. EtOAc (150 mL) and H₂O (150 mL). Remove the organic phase and extract the aqueous phase with EtOAc (150 mL). Combine the organic extractions, wash with saturated NaHCO₃(aq) (150 mL) and brine (150 mL), and dry over Na₂SO₄. Remove the solvent to yield the title compound as a light brown solid.

### 9. [2-(2-Chloromethyl)-7-(3-trifluoromethyl-pyridiny-2-yl)-quinazolin-4-yl]-(4-trifluoromethyl-phenyl)-amine

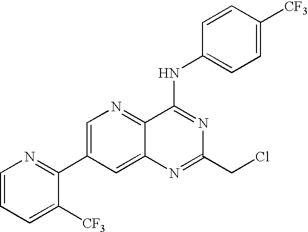

Dissolve 4-Chloro-2-chloro-methyl-7-(3-chloro-pyridin-2-yl)-pyrido[3,2-d]pyrimidine (2.30 g, 6.40 mmol) in a solution of acetonitrile (20 mL) and 4-trifluoromethyl aniline (1.13 g, 7.04 mmol). Heat the mixture at 80° C. for 18 hours. Cool the mixture to 0° C. and dilute with diethyl ether (20 mL). The mono-hydrochloride salt of the title compound forms a light brown precipitate (2.85 g 85.6%), which is removed by filtration and dried in a vacuum oven.

### 10. 7-(3-trifluoromethyl-pyridin-2-yl)-2-methoxymethyl-pyrido[3,2-d]pyrimidin-4-yl]-(4-trifluoromethyl-phenyl)-amine

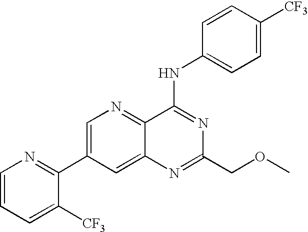

Treat [2-(2-Chloromethyl)-7-(3-trifluoromethyl-pyridiny-2-yl)-quinazolin-4-yl]-(4-trifluoromethyl-phenyl)-amine with NaOMe as described in Example 1.A-9 above. This affords 7-(3-trifluoromethyl-pyridin-2-yl)-2-methoxymethyl-pyrido[3,2-d]pyrimidin-4-yl]-(4-trifluoromethyl-phenyl)-amine as a solid.

### F. 7-(3-methyl-pyridin-2-yl)-2-methoxymethyl-pyrido[3,2-d]pyrimidin-4-yl]-(4-trifluoromethyl-phenyl)-amine

### 1. 5-Bromo-3-nitropyridine-2-carbonitrile

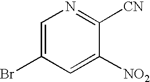

Heat a solution of 2-amino-5-bromo-3-nitropyridine (2.18 g, 10 mmol), cuprous cyanide (1.33 g, 15 mmol) and tert-butylnitrite (2.0 mL, 15 mmol) in acetonitrile (50 mL) at 60° C. for 2 hours. Cool the solution and partitioned between ethyl acetate (100 mL) and saturated aqueous NaHCO₃ (100 mL). Extract the aqueous layer with ethyl acetate (2×50 mL), wash with water (100 mL) and brine (100 mL), dry (MgSO₄) and evaporate. Purify the solid by flash chromatography on silica gel (25% ether/75% hexane) to give the title compound as a pale yellow solid.

### 2. 5-(3-Methyl(2-pyridyl))-3-nitropyridine-2-carbonitrile

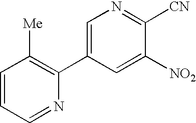

Heat a solution of 5-bromo-3-nitropyridine-2-carbonitrile (228 mg, 1.0 mmol), tetrakis(triphenylphosphine)palladium(0) (15 mg), 3-methyl-2-pyridylzinc bromide (0.5 M in THF, 3 mL, 1.5 mmol) in THF (5 mL) at 60° C. for 2 hours. Cool the solution and partition between ethyl acetate (10 mL) and saturated aqueous NaHCO₃ (10 mL). Extract the aqueous layer with ethyl acetate (2×15 mL), wash with water (10 mL) and brine (10 mL), dry (MgSO₄) and evaporate to give the title compound as a pale yellow solid.

### 3. 3-Amino-5-(3-methyl(2-pyridyl))pyridine-2-carboxamide

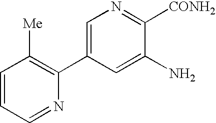

Heat a solution of 5-(3-methyl(2-pyridyl))-3-nitropyridine-2-carbonitrile (1 g, 4.1 mmol), iron (2.3 g, 40 mmol) and calcium chloride (560 mg, 5 mmol) in ethanol (15 mL) and water (4 mL) to reflux for 1 hour. Cool the mixture, filter through Celite and wash with ethyl acetate. Evaporate the filtrate and re-dissolve the residue in ethyl acetate. Wash with water and brine, dry (MgSO₄) and evaporate to give the title compound as a pale yellow solid.

### 4. 7-(3-methyl-pyridin-2-yl)-2-methoxymethyl-pyrido[3,2-d]pyrimidin-4-yl(-(4-trifluoromethyl-phenyl)-amine

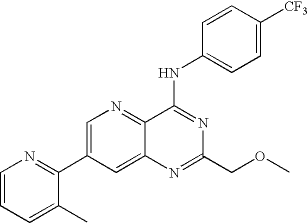

### G. 7-(3-trifluoromethyl-pyridin-2-yl)-2-methoxymethyl-pyrido[3,2-d]pyrimidin-4-yl]-(4-trifluoromethyl-phenyl)-amine

### 1. 7-(3-trifluoromethyl-pyridin-2-yl)-2-methoxymethyl-3H-pyrido[3,2-d]pyrimidin-4-one

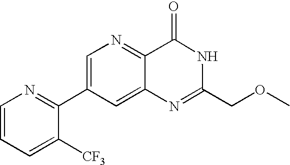

Treat a solution of 3-amino-5-[3-(chloro-pyridin-2-yl)]pyridine-2-carboxamide (340 mg, 1.21 mmol) in THF (5 mL) and pyridine (0.11 mL) with methoxy-acetyl chloride (0.11 mL, 144 mg, 1.33 mmol). Stir the mixture for 3 hours at room temperature. Then, add 5 N NaOH (10 mL) and stir the solution for an additional 18 hours. Concentrate the solution (˜5 mL) and acidify with conc. HCl. Extract the aqueous mixture with EtOAc (3×25 mL), and dry the combined organic extracts over Na₂SO₄. Remove the solvent under reduced pressure to yield the title compound as a white solid.

### 2. 4-Chloro-7-(3-trifluoromethyl-pyridin-2-yl)-2-methoxymethyl-pyrido[3,2-d]pyrimidine

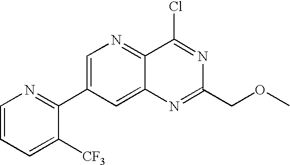

Dissolve 7-(3-trifluoromethyl-pyridin-2-yl)-2-methoxymethyl-3H-pyrido[3,2-d]pyrimidin-4-one (276 mg, 0.822 mmol) in CHCl₃ (25 mL) and 2,6-lutidine (294 mg, 2.74 mmol). Add phosphorous oxycloride (0.255 mL, 2.74 mmol) dropwise and heat the resulting solution to reflux for 24 hours. Cool the solution and remove the solvent under reduced pressure. Partition the crude residue between EtOAc (50 mL) and saturated NaHCO_{3 (aq)} (50 mL). Remove the organic phase and extract the aqueous phase with additional EtOAc (50 mL). Combine the two organic extracts, wash with brine (100 mL), and dry over Na₂SO₄. Remove the solvent to yield the title compound as a light brown solid.

### 3. 7-(3-trifluoromethyl-pyridin-2-yl)-2-methoxymethyl-pyrido[3, 2-d]pyrimidin-4-yl]-(4-trifluoromethyl-phenyl)-amine

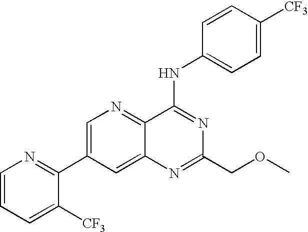

Dissolve 4-Chloro-7-(3-trifluoromethyl-pyridin-2-yl)-2-methoxymethyl-pyrido[3,2-d]pyrimidine (30 mg, 0.0934 mmol) into a solution of acetonitrile (3 mL) and 4-trifluoromethyl-aniline (18.0 mg, 0.112 mmol). Heat the mixture to 80° C. for 16 hours. Cool the reaction mixture in an ice bath and add diethyl ether (3 mL). Filter off the off-white precipitate and dry in a vacuum oven to yield the title compound as the mono-hydrochloride salt.

### H. [7-(3-Chloro-pyridin-2-yl)-2-methoxymethyl-pyrido[2,3-d]pyrimidin-4-yl]-(4-trifluoromethyl-phenyl)-amine

### 1. 2-Acetyl-3-chloropyridine

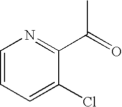

Dissolve 3-chloro-2-cyanopyridine (10.0 g, 0.072 mol, Chem. Pharm. Bull. (1985) 33:565-571) in anhydrous THF (200 mL) under N₂ atmosphere and coolin an ice bath. Add drop wise 3.0 M MeMgI in diethyl ether (48 ml, 0.14 mol) to the reaction mixture and stir in an ice bath for 2 hours. Pour the reaction mixture over ice cold water, acidify the mixture with 2.0 N aq. HCl to pH 2 to 3. Extract the reaction mixture with EtOAc (3×100 mL) and dry over anhydrous MgSO₄. Filter, concentrate under vacuum and then filter through a pad of silica gel using 20% ethyl acetate/hexane as eluent. Removal of solvent under reduced pressure gives pure 2-acetyl-3-chloropyridine as oil.

### 2. 1-(3-Chloro-pyridin-2-yl)-3-dimethylaminopropenone

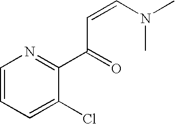

Heat 2-acetyl-3-chloropyridine (0.77 g, 5.0 mmol) with N,N-dimethylformamide dimethylacetal (3.0 g) at 105° C. for 20 hours. Concentrate under reduced pressure to give 1-(3-chloro-pyridin-2-yl)-3-dimethylaminopropenone as oil.

### 3. 2-Amino-4-(3-chloro-pyridin-2-yl)-benzonitrile

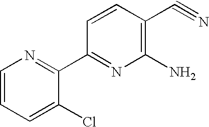

Heat a solution of 1-(3-chloro-pyridin-2-yl)-3-dimethylaminopropenone (1.05 g, 5 mmol), 3-amino-3-methoxy-acrylonitrile hydrochloride (1.35 g, 10 mmol) and ammonium acetate (2.2 g, 15.0 mmol) in ethanol (25 mL) at reflux for 20 hours. Cool the mixture and concentrate under reduced pressure to give dark oil. Dissolve the residue in EtOAc/water (100 mL). Extract the aqueous solution with EtOAc, wash the EtOAc with brine, dry (MgSO₄) and concentrate under reduced pressure to give 2-amino-4-(3-chloro-pyridin-2-yl)-benzonitrile as a brown solid.

### 4. 6-Amino-3′-chloro-[2,2′bipyridinyl-5-carboxylic acid amide

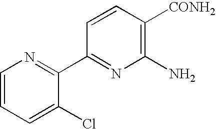

Cool concentrated sulfuric acid (10 mL) in an ice bath under nitrogen atmosphere. Add in portions 2-amino-4-(3-chloro-pyridin-2-yl)-benzonitrile (1.0 g, 4.3 mmol) over a period of 15 minutes. Stir at room temperature overnight. Pour the reaction mixture over ice, adjust the pH to 10 using 10 N aq. NaOH, filter the solid, wash the solid with water and dry under vacuum to give 6-amino-3′-chloro-[2,2′]bipyridinyl-5-carboxylic acid amide as a yellow solid.

### 5. 7-(3-Chloro-pyridin-2-yl)-2-methoxymethyl-3H-pyrido[2,3-d]pyrimidin-4-one

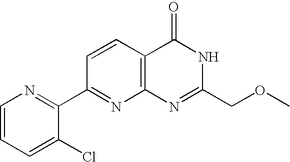

Dissolve 6-amino-3′-chloro-[2,2′]bipyridinyl-5-carboxylic acid amide (0.5 g, 2.02 mmol) in anhydrous THF (10 mL) under N₂ atmosphere. Add drop wise pyridine (0.36 g, 4.04 mmol) and methoxyacetyl chloride (0.48 g, 4.04 mmol) to the reaction mixture and stir at room temperature overnight. Add 10 % aq. NaOH (10 mL) and reflux for 4 hours. Concentrate in vacuum, adjust the pH to 6.0 using AcOH, collect the solid by filtration and dry under vacuum to give 7-(3-chloro-pyridin-2-yl)-2-methoxymethyl-3H-pyrido[2,3-d]pyrimidin-4-one as a white solid.

### 6. 4-Chloro-7-(3-chloro-pyridin-2-yl)-2-methoxymethyl-pyrido[2,3-d]pyrimidine

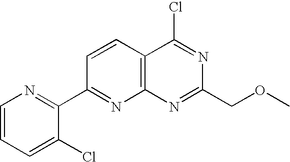

Reflux a mixture of 7-(3-chloro-pyridin-2-yl)-2-methoxymethyl-3H-pyrido[2,3-d]pyrimidin-4-one (0.25 g), 2,6-lutidine (0.44 g), and POCl₃ (0.51 g) in CHCl₃ (5 mL) for 20 hours. Cool the mixture and concentrate under reduced pressure. Partition the residue between EtOAc and saturated NaHCO₃ solution. Wash the EtOAc portion with additional NaHCO₃ and then dry (Na₂SO₄) and concentrate under reduced pressure. Filter the brown residue through 2 inches of silica gel (1:1 EtOAc/hexanes eluent) and concentrate under reduced pressure to give 4-chloro-7-(3-chloro-pyridin-2-yl)-2-methoxymethyl-pyrido[2,3-d]pyrimidine.

### 7. [7-(3-Chloro-pyridin-2-yl)-2-methoxymethyl-pyrido[2,3-d]pyrimidin-4-yl]-(4-trifluoromethyl-phenyl)-amine

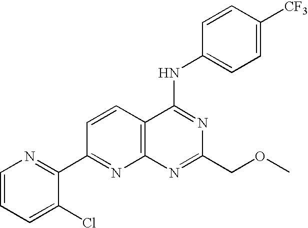

Heat a mixture of 4-chloro-7-(3-chloro-pyridin-2-yl)-2-methoxymethyl-pyrido[2,3-d]pyrimidine (0.1 mmol) and 4-trifluoromethyl-aniline (16.1 mg, 0.1 mmol) in AcCN (1 mL) at 80° C. for 24 hours. Cool the mixture and wash the precipitate with ether to give [7-(3-chloro-pyridin-2-yl)-2-methoxymethyl-pyrido[2,3-d]pyrimidin-4-yl]-(4-trifluoromethyl-phenyl)-amine as the mono-HCl salt.

### I. [2-Methoxymethyl-7-(3-methylpyridin-2-yl)-quinazolin-4-yl)-(4-trifluoromethylphenyl)-amine

### 1. 7-bromo-2-methoxymethylquinazolin-4-yl)-(4-trifluoromethylphenyl)-amine

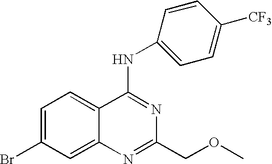

Heat a mixture of 7-bromo-2-chloromethylquinazolin-4-yl)-(4-trifluoromethylphenyl)-amine (from Example C, 200 mg, 0.48 mmol), 4.4M sodium methoxide in methanol (2.4 mL), and methanol (1 mL) to 60° C. for 4 hours. Cool to room temperature and evaporate the mixture. Dilute with EtOAc (10 mL) and wash 2× with water (10 mL each). Dry the organic layer (Na₂SO₄) and evaporate. Purify by preparative TLC (3:1 hexanes:EtOAc) to obtain 2-methoxymethyl-7-pyridin4-yl-quinazolin-4-yl)-(4-trifluoromethylphenyl)-amine as a yellow solid.

### 2. [2-Methoxymethyl-7-(3-methylpyridin-2-yl)-quinazolin-4-yl)-(4-trifluoromethyl phenyl)-amine

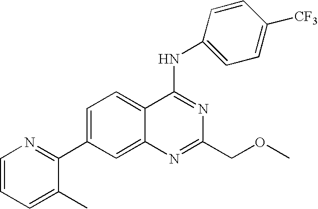

Heat a mixture of 2-methoxymethyl-7-pyridin-4-yl-quinazolin-4-yl)-(4-trifluoromethylphenyl)-amine (100 mg, 0.243 mmol), 3-methyl-2-pyridylzinc bromide (1 mL of a 0.5M THF solution), tetrakis(triphenylphosphinepalladium(0) (50 mg, 0.043 mmol) in 1,2-dimethoxymethane (5 mL) for 3 hours at 80° C. under nitrogen. Cool to room temperature and dilute with EtOAc (10 mL). Wash with water (2×10 mL) and dry the organic layer (Na₂SO₄) and evaporate. Purify by preparative TLC to obtain 12-methoxymethyl-7-(3-methylpyridin-2-yl)-quinazolin-4-yl)-(4-trifluoromethylphenyl)-amine as an off-white solid.

### J. Additional Representative Substituted 2-Hydroxyalkyl-Quinazolin-4-ylamine Analogues

Those having skill in the art will recognize that the starting materials may be varied and additional steps employed to produce other compounds encompassed by the present invention. The following compounds were prepared using the above methods, with readily apparent modifications, and may be used in the compositions and methods provided herein:

(1-Methanesulfonyl-2,3-dihydro-1H-indol-5-yl)-[2-methoxymethyl-7-(3-trifluoromethyl-pyridin-2-yl)-quinazolin-4-yl]-amine;

(2,6-Dimethyl-morpholin-4-yl)-(1-{4-[2-methoxymethyl-7-(3-trifluoromethyl-pyridin-2-yl)-pyrido[2,3-d]pyrimidin-4-ylamino]-phenyl}-cyclobutyl)-methanone;

(4-Cyclohexyl-phenyl)-[2-methoxymethyl-7-(3-trifluoromethyl-pyridin-2-yl)-pyrido[3,2-d]pyrimidin-4-yl]-amine;

(4-Cyclopentyl-phenyl)-[2-methoxymethyl-7-(3-trifluoromethyl-pyridin-2-yl)-pyrido[2,3-d]pyrimidin-4-yl]-amine;

(4-Cyclopropyl-phenyl)-[2-methoxymethyl-7-(3-trifluoromethyl-pyridin-2-yl)-quinazolin-4-yl]-amine;

(4-Ethyl-phenyl)-[2-methoxymethyl-7-(3-trifluoromethyl-pyridin-2-yl)-pyrido[3,2-d]pyrimidin-4-yl]-amine;

(4-Isopropyl-phenyl)-[2-(2-methoxy-ethyl)-7-(3-trifluoromethyl-pyridin-2-yl)-quinazolin-4-yl]-amine;

(4-Isopropyl-phenyl)-[2-(tetrahydro-pyran-4-yloxymethyl)-7-(3-trifluoromethyl-pyridin-2-yl)-quinazolin-4-yl]-amine;

(4-Isopropyl-phenyl)-[2-methoxymethyl-7-(3-methyl-pyridin-2-yl)-pyrido[2,3-d]pyrimidin-4-yl]-amine;

(4-Isopropyl-phenyl)-[2-methoxymethyl-7-(3-methyl-pyridin-2-yl)-quinazolin-4yl]-amine;

(4-Isopropyl-phenyl)-[2-methoxymethyl-7-(3-trifluoromethyl-pyridin-2-yl)-pyrido[3,2-d]pyrimidin-4-yl]-amine;

(4-Isopropyl-phenyl)-[2-methoxymethyl-7-(3-trifluoromethyl-pyridin-2-yl)-quinazolin-4-yl]-amine;

(4-Isopropyl-phenyl)-[2-methoxymethyl-7-(3-trifluoromethyl-pyridin-2-yl)-pyrido[2,3-d]pyrimidin-4-yl]-amine;

(4-Isopropyl-phenyl)-[7-(3-methyl-pyridin-2-yl)-2-(tetrahydro-pyran-4-yloxymethyl)-quinazolin-4-yl]-amine;

(4-Methanesulfonyl-phenyl)-[2-methoxymethyl-7-(3-trifluoromethyl-pyridin-2-yl)-quinazolin-4-yl]-amine;

(4-Methanesulfonyl-phenyl)-[2-methoxymethyl-7-(3-trifluoromethyl-pyridin-2-y)-pyrido[2,3-d]pyrimidin-4-yl]-amine;

(4-sec-Butyl-phenyl)-[2-methoxymethyl-7-(3-methyl-pyridin-2-yl)-pyrido[2,3-d]pyrimidin-4-yl]-amine;

(4-sec-Butyl-phenyl)-[2-methoxymethyl-7-(3-trifluoromethyl-pyridin-2-yl)-pyrido[2,3-d]pyrimidin-4-yl]-amine;

(4-sec-Butyl-phenyl)-[7-(3-chloro-pyridin-2-y)-2-(2-methoxy-ethoxymethyl)-pyrido[2,3-d]pyrimidin-4-yl]-amine;

(4-sec-Butyl-phenyl)-[7-(3-chloro-pyridin-2-yl)-2-methoxymethyl-pyrido[2,3-d]pyrimidin-4-yl]-amine;

(4-tert-Butyl-phenyl)-[2-(2-methoxy-ethyl)-7-(3-trifluoromethyl-pyridin-2-yl)-quinazolin-4-yl]-amine;

(4-tert-Butyl-phenyl)-[2-(3-diethylamino-1-methyl-propoxymethyl)-7-(3-trifluoromethyl-pyridin-2-yl)-quinazolin-4-yl]-amine;

(4-tert-Butyl-phenyl)-[2-(3-diethylamino-1-methyl-propoxymethyl)-7-(3-trifluoromethyl-pyridin-2-yl)-quinazolin-4-yl]-amine;

(4-tert-Butyl-phenyl)-[2-(3-diethylamino-propoxymethyl)-7-(3-trifluoromethyl-pyridin-2-yl)-quinazolin-4-yl]-amine;

(4-tert-Butyl-phenyl)-[2-(3-dimethylamino-propoxymethyl)-7-(3-trifluoromethyl-pyridin-2-yl)-quinazolin-4-yl]-amine;

(4-tert-Butyl-phenyl)-[2-(3-morpholin-4-yl-propoxymethyl)-7-(3-trifluoromethyl-pyridin-2-yl)-quinazolin-4-yl]-amine;

(4-tert-Butyl-phenyl)-[2-(4-dimethylamino-butoxymethyl)-7-(3-trifluoromethyl-pyridin-2-yl)-quinazolin-4-yl]-amine;

(4-tert-Butyl-phenyl)-[2-(4-morpholin-4-yl-butoxymethyl)-7-(3-trifluoromethyl-pyridin-2-yl)-quinazolin-4-yl]-amine;

(4-tert-Butyl-phenyl)-[2-isobutoxymethyl-7-(3-methyl-pyridin-2-yl)-pyrido[2,3-d]pyrimidin-4-yl]-amine;

(4-tert-Butyl-phenyl)-[2-isobutoxymethyl-7-(3-trifluoromethyl-pyridin-2-yl)-pyrido[2,3-d]pyrimidin-4-yl]-amine;

(4-tert-Butyl-phenyl)-[2-methoxymethyl-7-(3-methyl-pyridin-2-yl)-pyrido[2,3-d]pyrimidin-4-yl]-amine;

(4-tert-Butyl-phenyl)-[2-methoxymethyl-7-(3-trifluoromethyl-pyridin-2-yl)-pyrido[3,2-d]pyrimidin-4-yl]-amine;

(4-tert-Butyl-phenyl)-[2-methoxymethyl-7-(3-trifluoromethyl-pyridin-2-yl)-quinazolin-4-yl]-amine;

(4-tert-Butyl-phenyl)-[2-methoxymethyl-7-(3-trifluoromethyl-pyridin-2-yl)-pyrido[2,3-d]pyrimidin-4-yl]-amine;

(4-tert-Butyl-phenyl)-[7-(3-chloro-pyridin-2-yl)-2-(2-methoxy-ethoxymethyl)-pyrido[2,3-d]pyrimidin-4-yl]-amine;

(4-tert-Butyl-phenyl)-[7-(3-chloro-pyridin-2-yl)-2-ethoxymethyl-pyrido[2,3-d]pyrimidin-4-yl]-amine;

(4-tert-Butyl-phenyl)-[7-(3-chloro-pyridin-2-yl)-2-ethoxymethyl-quinazolin-4-yl]-amine;

(4-tert-Butyl-phenyl)-[7-(3-chloro-pyridin-2-yl)-2-methoxymethyl-pyrido[2,3-d]pyrimidin-4-yl]-amine;

(4-tert-Butyl-phenyl)-[7-(3-chloro-pyridin-2-yl)-2-methoxymethyl-pyrido[3,2-d]pyrimidin-4-yl]-amine;

(4-tert-Butyl-phenyl)-[7-(3-chloro-pyridin-2-yl)-2-methoxymethyl-quinazolin-4-yl]-amine;

(6-tert-Butyl-pyridin-3-yl)-[2-(2-methoxy-ethyl)-7-(3-trifluoromethyl-pyridin-2-yl)-quinazolin-4-yl]-amine;

(6-tert-Butyl-pyridin-3-yl)-[2-isobutoxymethyl-7-(3-methyl-pyridin-2-yl)-pyrido[2,3-d]pyrimidin-4-yl]-amine;

(6-tert-Butyl-pyridin-3-yl)-[2-isobutoxymethyl-7-(3-trifluoromethyl-pyridin-2-yl)-pyrido[2,3-d]pyrimidin-4-yl]-amine;

(6-tert-Butyl-pyridin-3-yl)-[2-methoxymethyl-7-(3-methyl-pyridin-2-yl)-pyrido[2,3-d]pyrimidin-4-yl]-amine;

(6-tert-Butyl-pyridin-3-yl)-[2-methoxymethyl-7-(3-trifluoromethyl-pyridin-2-yl)-quinazolin-4-yl]-amine;

(6-tert-Butyl-pyridin-3-yl)-[2-methoxymethyl-7-(3-trifluoromethyl-pyridin-2-yl)-pyrido[2,3-d]pyrimidin-4-yl]-amine;

(6-tert-Butyl-pyridin-3-yl)-[7-(3-chloro-pyridin-2-yl)-2-ethoxymethyl-quinazolin-4-yl]-amine;

(6-tert-Butyl-pyridin-3-yl)-[7-(3-chloro-pyridin-2-yl)-2-isobutoxymethyl-pyrido[2,3-d]pyrimidin-4-yl]-amine;

(6-tert-Butyl-pyridin-3-yl)-[7-(3-chloro-pyridin-2-yl)-2-methoxymethyl-quinazolin-4-yl]-amine;

(6-tert-Butyl-pyridin-3-yl)-[7-(3-chloro-pyridin-2-yl)-2-methoxymethyl-pyrido[2,3-d]pyrimidin-4-yl]-amine;

[2-(1-Methyl-piperidin-4-yloxymethyl)-7-(3-trifluoromethyl-pyridin-2-yl)-quinazolin-4-yl]-(6-trifluoromethyl-pyridin-3-yl)-amine;

[2-(2-Diethylamino-ethoxymethyl)-7-(3-trifluoromethyl-pyridin-2-yl)-quinazolin-4-yl]-(6-trifluoromethyl-pyridin-3-yl)-amine;

[2-(2-Dimethylamino-ethoxymethyl)-7-(3-trifluoromethyl-pyridin-2-yl)-quinazolin-4-yl]-(4-trifluoromethyl-phenyl)-amine;

[2-(2-Piperidin-1-y-ethoxymethyl)-7-(3-trifluoromethyl-pyridin-2-yl)-quinazolin-4-yl]-(6-trifluoromethyl-pyridin-3-yl)-amine;

[2-(3-Benzyloxy-propyl)-7-(3-trifluoromethyl-pyridin-2-yl)-quinazolin-4-yl]-(4-trifluoromethyl-phenyl)-amine;

[2-(3-Benzyloxy-propyl)-7-(3-trifluoromethyl-pyridin-2-yl)-quinazolin-4-yl]-(6-trifluoromethyl-pyridin-3-yl)-amine;

[2-(3-Benzyloxy-propyl)-7-(3-trifluoromethyl-pyridin-2-yl)-quinazolin-4-yl]-(6-tert-butyl-pyridin-3-yl)-amine;

[2-(3-Diethylamino-propoxymethyl)-7-(3-trifluoromethyl-pyridin-2-yl)-quinazolin-4-yl]-(4-trifluoromethyl-phenyl)-amine;

[2-(3-Dimethylamino-2,2-dimethyl-propoxymethyl)-7-(3-trifluoromethyl-pyridin-2-yl)-quinazolin-4-yl]-(4-trifluoromethyl-phenyl)-amine;

[2-(3-Dimethylamino-propoxymethyl)-7-(3-trifluoromethyl-pyridin-2-yl)-quinazolin-4-yl]-(4-trifluoromethyl-phenyl)-amine;

[2-(Pyridin-3-ylmethoxymethyl)-7-(3-trifluoromethyl-pyridin-2-yl)-quinazolin-4-yl]-(6-trifluoromethyl-pyridin-3-yl)-amine;

[2-(Pyridin-4-ylmethoxymethyl)-7-(3-trifluoromethyl-pyridin-2-yl)-quinazolin-4-yl]-(6-trifluoromethyl-pyridin-3-yl)-amine;

[2-(Tetrahydro-pyran4-yloxymethyl)-7-(3-trifluoromethyl-pyridin-2-yl)-quinazolin-4-yl]-(4-trifluoromethyl-phenyl)-amine;

[2-(Tetrahydro-pyran4-yloxymethyl)-7-(3-trifluoromethyl-pyridin-2-yl)-quinazolin-4-yl]-(6-trifluoromethyl-pyridin-3-yl)-amine;

[2-Benzyloxymethyl-7-(3-trifluoromethyl-pyridin-2-yl)-quinazolin-4-yl]-[4-(2,2,2-trifluoro-1-methyl-ethyl)-phenyl]-amine;

[2-Benzyloxymethyl-7-(3-trifluoromethyl-pyridin-2-yl)-quinazolin-4-yl]-(4-trifluoromethyl-phenyl)-amine;

[2-Benzyloxymethyl-7-(3-trifluoromethyl-pyridin-2-yl)-quinazolin-4-yl]-(6-trifluoromethyl-pyridin-3-yl)-amine;

[2-Benzyloxymethyl-7-(3-trifluoromethyl-pyridin-2-yl)-quinazolin-4-yl]-(5-trifluoromethyl-pyridin-2-yl)-amine;

[2-Benzyloxymethyl-7-(3-trifluoromethyl-pyridin-2-yl)-quinazolin-4-yl]-(4-trifluoromethoxy-phenyl)-amine;

[2-Benzyloxymethyl-7-(3-trifluoromethyl-pyridin-2-yl)-quinazolin-4-yl]-(4-trifluoromethanesulfonyl-phenyl)-amine;

[2-Benzyloxymethyl-7-(3-trifluoromethyl-pyridin-2-yl)-quinazolin-4-yl]-(4-methanesulfonyl-phenyl)-amine;

[2-Benzyloxymethyl-7-(3-trifluoromethyl-pyridin-2-yl)-quinazolin-4-yl]-[4-(2-methoxy-1,1-dimethyl-ethyl)-phenyl]-amine;

[2-Benzyloxymethyl-7-(3-trifluoromethyl-pyridin-2-yl)-quinazolin-4-yl]-(4-tert-butyl-phenyl)-amine;

[2-Benzyloxymethyl-7-(3-trifluoromethyl-pyridin-2-yl)-quinazolin-4-yl]-(1-methanesulfonyl-2,3-dihydro-1H-indol-5-yl)-amine;

[2-Cyclopentyloxymethyl-7-(3-trifluoromethyl-pyridin-2-yl)-quinazolin-4-yl]-[4-(morpholine-4-sulfonyl)-phenyl]-amine;

[2-Cyclopropylmetboxymethyl-7-(3-trifluoromethyl-pyridin-2-yl)-quinazolin-4-yl]-[4-(morpholine-4-sulfonyl)-phenyl]-amine;

[2-Ethoxymethyl-7-(3-methyl-pyridin-2-yl)-pyrido[2,3-d]pyrimidin-4-yl]-(4-trifluoromethyl-phenyl)-amine;

[2-Ethoxymethyl-7-(3-methyl-pyridin-2-yl)-pyrido[2,3-d]pyrimidin-4-yl]-(4-isopropyl-phenyl)-amine;

[2-Ethoxymethyl-7-(3-methyl-pyridin-2-yl)-pyrido[2,3-d]pyrimidin-4-yl]-(4-trifluoromethanesulfonyl-phenyl)-amine;

[2-Ethoxymethyl-7-(3-methyl-pyridin-2-yl)-quinazolin-4-yl]-(4-isopropyl-phenyl)-amine;

[2-Ethoxymethyl-7-(3-methyl-pyridin-2-yl)-quinazolin-4-yl]-(4-trifluoromethyl-phenyl)-amine;

[2-Ethoxymethyl-7-(3-trifluoromethyl-pyridin-2-yl)-pyrido[2,3-d]pyrimidin-4-yl]-(4-trifluoromethyl-phenyl)-amine;

[2-Ethoxymethyl-7-(3-trifluoromethyl-pyridin-2-yl)-pyrido[2,3-d]pyrimidin-4-yl]-(6-trifluoromethyl-pyridin-3-yl)-amine;

[2-Ethoxymethyl-7-(3-trifluoromethyl-pyridin-2-yl)-quinazolin-4-yl]-(4-trifluoromethyl-phenyl)-amine;

[2-Ethoxymethyl-7-(3-trifluoromethyl-pyridin-2-yl)-quinazolin-4-yl]-[4-(morpholine-4-sulfonyl)-phenyl]-amine;

[2-Ethoxymethyl-7-(3-trifluoromethyl-pyridin-2-yl)-quinazolin-4-yl]-(6-trifluoromethyl-pyridin-3-yl)-amine;

[2-Isobutoxymethyl-7-(3-methyl-pyridin-2-yl)-pyrido[2,3-d]pyrimidin-4-yl]-(4-isopropyl-phenyl)-amine;

[2-Isobutoxymethyl-7-(3-methyl-pyridin-2-yl)-pyrido[2,3-d]pyrimidin-4-yl]-(4-trifluoromethyl-phenyl)-amine;

[2-Isobutoxymethyl-7-(3-methyl-pyridin-2-yl)-pyrido[2,3-d]pyrimidin-4-yl]-(3-methyl-4-trifluoromethyl-phenyl)-amine;

[2-Isobutoxymethyl-7-(3-methyl-pyridin-2-yl)-pyrido[2,3-d]pyrimidin-4-yl]-(4-trifluoromethoxy-phenyl)-amine;

[2-Isobutoxymethyl-7-(3-methyl-pyridin-2-yl)-pyrido[2,3-d]pyrimidin-4-yl]-[4-(2-methoxy-1,1-dimethyl-ethyl)-phenyl]-amine;

[2-Isobutoxymethyl-7-(3-methyl-pyridin-2-yl)-pyrido[2,3-d]pyrimidin-4-yl]-(4-methanesulfonyl-phenyl)-amine;

[2-Isobutoxymethyl-7-(3-trifluoromethyl-pyridin-2-yl)-pyrido[2,3-d]pyrimidin-4-yl]-(4-isopropyl-phenyl)-amine;

[2-Isobutoxymethyl-7-(3-trifluoromethyl-pyridin-2-yl)-pyrido[2,3-d]pyrimidin-4-yl]-(4-trifluoromethyl-phenyl)-amine;

[2-Isobutoxymethyl-7-(3-trifluoromethyl-pyridin-2-yl)-pyrido[2,3-d]pyrimidin-4-yl]-(6-trifluoromethyl-pyridin-3-yl)-amine;

[2-Isobutoxymethyl-7-(3-trifluoromethyl-pyridin-2-yl)-pyrido[2,3-d]pyrimidin-4-yl]-(3-methyl-4-trifluoromethyl-phenyl)-amine;

[2-Isobutoxymethyl-7-(3-(trifluoromethyl-pyridin-2-yl)-pyrido[2,3-d]pyrimidin-4-yl]-(4-trifluoromethanesulfonyl-phenyl)-amine;

[2-Isobutoxymethyl-7-(3-trifluoromethyl-pyridin-2-yl)-pyrido[2,3-d]pyrimidin-4-yl]-(4-trifluoromethoxy-phenyl)-amine;

[2-Isobutoxymethyl-7-(3-trifluoromethyl-pyridin-2-yl)-pyrido[2,3-d]pyrimidin-4-yl]-(4-methanesulfonyl-phenyl)-amine;

[2-Isopropoxymethyl-7-(3-methyl-pyridin-2-yl)-quinazolin-4-yl]-(4-trifluoromethyl-phenyl)-amine;

[2-Isopropoxymethyl-7-(3-methyl-pyridin-2-yl)-quinazolin-4-yl]-(4-isopropyl-phenyl)-amine;

[2-Isopropoxymethyl-7-(3-trifluoromethyl-pyridin-2-yl)-quinazolin-4-yl]-[4-(morpholine-4-sulfonyl)-phenyl]-amine;

[2-Isopropoxymethyl-7-(3-trifluoromethyl-pyridin-2-yl)-quinazolin-4-yl]-(6-trifluoromethyl-pyridin-3-yl)-amine;

[2-Methoxymethyl-7-(3-methyl-pyridin-2-yl)-pyrido[2,3-d]pyrimidin-4-yl]-(4-trifluoromethyl-phenyl)-amine;

[2-Methoxymethyl-7-(3-methyl-pyridin-2-yl)-pyrido[2,3-d]pyrimidin-4-yl]-(6-trifluoromethyl-pyridin-3-yl)-amine;

[2-Methoxymethyl-7-(3-methyl-pyridin-2-yl)-pyrido[2,3-d]pyrimidin-4-yl]-(4-trifluoromethanesulfonyl-phenyl)-amine;

[2-Methoxymethyl-7-(3-methyl-pyridin-2-yl)-pyrido[2,3-d]pyrimidin-4-yl]-(4-trifluoromethoxy-phenyl)-amine;

[2-Methoxymethyl-7-(3-methyl-pyridin-2-yl)-pyrido[2,3-d]pyrimidin-4-yl]-(3-methyl-4-trifluoromethyl-phenyl)-amine;

[2-Methoxymethyl-7-(3-trifluoromethyl-pyridin-2-yl)-pyrido[2,3-d]pyrimidin-4-yl]-[4-(2,2,2-trifluoro-1-methyl-ethyl)-phenyl]-amine;

[2-Methoxymethyl-7-(3-trifluoromethyl-pyridin-2-yl)-pyrido[2,3-d]pyrimidin-4-yl]-(4-trifluoromethyl-phenyl)-amine;

[2-Methoxymethyl-7-(3-trifluoromethyl-pyridin-2-yl)-pyrido[2,3-d]pyrimidin-4-yl]-(4-trifluoromethanesulfonyl-phenyl)-amine;

[2-Methoxymethyl-7-(3-trifluoromethyl-pyridin-2-yl)-pyrido[2,3-d]pyrimidin-4-yl]-[4-(morpholine-4-sulfonyl)-phenyl]-amine;

[2-Methoxymethyl-7-(3-trifluoromethyl-pyridin-2-yl)-pyrido[2,3-d]pyrimidin-4-yl]-(6-trifluoromethyl-pyridin-3-yl)-amine;

[2-Methoxymethyl-7-(3-trifluoromethyl-pyridin-2-yl)-pyrido[2,3-d]pyrimidin-4-yl]-(3-methyl-4-trifluoromethyl-phenyl)-amine;

[2-Methoxymethyl-7-(3-trifluoromethyl-pyridin-2-yl)-pyrido[2,3-d]pyrimidin-4-yl]-(4-trifluoromethoxy-phenyl)-amine;

[2-Methoxymethyl-7-(3-trifluoromethyl-pyridin-2-yl)-pyrido[3,2-d]pyrimidin-4-yl]-(6-trifluoromethyl-pyridin-3-yl)-amine;

[2-Methoxymethyl-7-(3-trifluoromethyl-pyridin-2-yl)-pyrido[3,2-d]pyrimidin-4-yl]-(4-morpholin-4-yl-phenyl)-amine;

[2-Methoxymethyl-7-(3-trifluoromethyl-pyridin-2-yl)-quinazolin-4-yl]-(4-trifluoromethyl-phenyl)-amine;

[2-Methoxymethyl-7-(3-trifluoromethyl-pyridin-2-yl)-quinazolin-4-yl]-(6-trifluoromethyl-pyridin-3-yl)-amine;

[2-Methoxymethyl-7-(3-trifluoromethyl-pyridin-2-yl)-quinazolin-4-yl]-(4-trifluoromethanesulfonyl-phenyl)-amine;

[2-Methoxymethyl-7-(3-trifluoromethyl-pyridin-2-yl)-quinazolin-4-yl]-(5-trifluoromethyl-pyridin-2-yl)-amine;

[2-Methoxyethyl-7-(3-trifluoromethyl-pyridin-2-yl)-quinazolin-4-yl]-[4-(morpholine-4-sulfonyl)-phenyl]-amine;

[4-(2-Diethylamino-1,1-dimethyl-ethyl)-phenyl]-[2-methoxymethyl-7-(3-methyl-pyridin-2-yl)-pyrido[2,3-d]pyrimidin-4-yl]-amine;

[4-(2-Methoxy-1,1-dimethyl-ethyl)-phenyl]-[2-methoxymethyl-7-(3-trifluoromethyl-pyridin-2-yl)-quinazolin-4-yl]-amine;

[4-(2-Methoxy-1,1-dimethyl-ethyl)-phenyl]-[2-methoxymethyl-7-(3-trifluoromethyl-pyridin-2-yl)-pyrido [3,2-d]pyrimidin-4-yl]-amine;

[4-(4-Isopropyl-phenylamino)-7-(3-trifluoromethyl-pyridin-2-yl)-quinazolin-2-yl]-methanol;

[4-(4-tert-Butyl-phenylamino)-7-(3-chloro-pyridin-2-yl)-quinazolin-2-yl]-methanol;

[4-(4-tert-Butyl-phenylamino)-7-(3-methyl-pyridin-2-yl)-pyrido[2,3d]pyrimidin-2-yl]-methanol;

[4-(4-tert-Butyl-phenylamino)-7-(3-trifluoromethyl-pyridin-2-yl)-quinazolin-2-yl]-methanol;

[4-(4-tert-Butyl-phenylamino)-7-(3-trifluoromethyl-pyridin-2-yl)-pyrido[3,2-d]pyrimidin-2-yl]-methanol;

[4-(4-Trifluoromethyl-phenylamino)-7-(3-trifluoromethyl-pyridin-2-y)-quinazolin-2-yl]-methanol;

[4-(4-Trifluoromethyl-phenylamino)-7-(3-trifluoromethyl-pyridin-2-yl)-pyrido[3,2-d]pyrimidin-2-yl]-methanol;

[4-(4-Trifluoromethyl-phenylamino)-7-(3-trifluoromethyl-pyridin-2-yl)-pyrido[2,3-d]pyrimidin-2-yl]-methanol;

[4-(Morpholine4-sulfonyl)-phenyl]-[2-(tetrahydro-pyran4-yloxymethyl)-7-(3-trifluoromethyl-pyridin-2-yl)-quinazolin-4-yl]-amine;

[4-[4-(Piperidine-1-sulfonyl)-phenylamino]-7-(3-trifluoromethyl-pyridin-2-yl)-quinazolin-2-yl]-methanol;

[4-[4-(Piperidine-1-sulfonyl)-phenylamino]-7-(3-trifluoromethyl-pyridin-2-yl)-pyrido[3,2-d]pyrimidin-2-yl]-methanol;

[7-(3-Chloro-pyridin-2-yl)-2-(2-methoxy-ethoxymethyl)-pyrido[2,3-d]pyrimidin-4-yl]-(4-trifluoromethyl-phenyl)-amine;

[7-(3-Chloro-pyridin-2-yl)-2-(2-methoxy-ethoxymethyl)-pyrido[2,3-d]pyrimidin-4-yl]-(4-isopropyl-phenyl)-amine;

[7-(3-Chloro-pyridin-2-yl)-2-(2-methoxy-ethyl)-pyrido[2,3-d]pyrimidin-4-yl]-(4-trifluoromethyl-phenyl)-amine;

[7-(3-Chloro-pyridin-2-yl)-2-(tetrahydro-pyran4-yloxymethyl)-quinazolin-4-yl]-(4-trifluoromethyl-phenyl)-amine;

[7-(3-Chloro-pyridin-2-yl)-2-(tetrahydro-pyran4-yloxymethyl)-quinazolin-4-yl]-(4-isopropyl-phenyl)-amine;

[7-(3-Chloro-pyridin-2-yl)-2-ethoxymethyl-pyrido[2,3-d]pyrimidin-4-yl]-(4-isopropyl-phenyl)-amine;

[7-(3-Chloro-pyridin-2-yl)-2-ethoxymethyl-pyrido[2,3-d]pyrimidin-4-yl]-(4-trifluoromethyl-phenyl)-amine;

[7-(3-Chloro-pyridin-2-yl)-2-ethoxymethyl-pyrido[2,3-d]pyrimidin-4-yl]-(4-trifluoromethanesulfonyl-phenyl)-amine;

[7-(3-Chloro-pyridin-2-yl)-2-ethoxymethyl-pyrido[2,3-d]pyrimidin-4-yl]-[4-(2,2,2-trifluoro-1-methyl-ethyl)-phenyl]-amine;

[7-(3-Chloro-pyridin-2-yl)-2-ethoxymethyl-pyrido[2,3-d]pyrimidin-4-yl]-(4-cyclopentyl-phenyl)-amine;

[7-(3-Chloro-pyridin-2-yl)-2-ethoxymethyl-pyrido[2,3-d]pyrimidin-4-yl]-(6-trifluoromethyl-pyridin-3-yl)-amine;

[7-(3-Chloro-pyridin-2-yl)-2-ethoxymethyl-pyrido[2,3-d]pyrimidin-4-yl]-(3-methyl-4-trifluoromethyl-phenyl)-amine;

[7-(3-Chloro-pyridin-2-yl)-2-ethoxymethyl-quinazolin-4-yl]-(4-isopropyl-phenyl)-amine;

[7-(3-Chloro-pyridin-2-yl)-2-ethoxymethyl-quinazolin-4-yl]-(4-trifluoromethyl-phenyl)-amine;

[7-(3-Chloro-pyridin-2-yl)-2-isobutoxymethyl-pyrido[2,3-d]pyrimidin-4-yl]-(4-trifluoromethyl-phenyl)-amine;

[7-(3-Chloro-pyridin-2-yl)-2-isobutoxymethyl-pyrido[2,3-d]pyrimidin-4-yl]-(6-trifluoromethyl-pyridin-3-yl)-amine;

[7-(3-Chloro-pyridin-2-yl)-2-isobutoxymethyl-pyrido[2,3-d]pyrimidin-4-yl]-(3-methyl-4-trifluoromethyl-phenyl)-amine;

[7-(3-Chloro-pyridin-2-yl)-2-isobutoxymethyl-pyrido[2,3-d]pyrimidin-4-yl]-(4-trifluoromethoxy-phen; yl)-amine;

[7-(3-Chloro-pyridin-2-yl)-2-isobutoxymethyl-pyrido[2,3-d]pyrimidin-4-yl]-(4-trifluoromethanesulfonyl-phenyl)-amine;

[7-(3-Chloro-pyridin-2-yl)-2-isobutoxymethyl-pyrido[2,3-d]pyrimidin-4-yl]-(4-methanesulfonyl-phenyl)-amine;

[7-(3-Chloro-pyridin-2-yl)-2-methoxymethyl-pyrido[2,3-d]pyrimidin-4-yl]-(4-isopropyl-phenyl)-amine;

[7-(3-Chloro-pyridin-2-yl)-2-methoxymethyl-pyrido[2,3-d]pyrimidin-4-yl]-[4-(2-methoxy-1,1-dimethyl-ethyl)-phenyl]-amine;

[7-(3-Chloro-pyridin-2-yl)-2-methoxymethyl-pyrido[2,3-d]pyrimidin-4-yl]-(4-trifluoromethoxy-phenyl)-amine;

[7-(3-Chloro-pyridin-2-yl)-2-methoxymethyl-pyrido[2,3-d]pyrimidin-4-yl]-(3-methyl-4-trifluoromethyl-phenyl)-amine;

[7-(3-Chloro-pyridin-2-yl)-2-methoxymethyl-pyrido[3,2-d]pyrimidin-4-yl]-(4-trifluoromethyl-phenyl)-amine;

[7-(3-Chloro-pyridin-2-yl)-2-methoxymethyl-pyrido[3,2-d]pyrimidin-4-yl]-(4-isopropyl-phenyl)-amine;

[7-(3-Chloro-pyridin-2-yl)-2-methoxymethyl-pyrido[3,2-d]pyrimidin-4-yl]-[4-(morpholine-4-sulfonyl)-phenyl]-amine;

[7-(3-Chloro-pyridin-2-yl)-2-methoxymethyl-quinazolin-4-yl]-(4-isopropyl-phenyl)-amine;

[7-(3-Chloro-pyridin-2-yl)-2-methoxymethyl-quinazolin-4-yl]-(4-trifluoromethyl-phenyl)-amine;

[7-(3-Chloro-pyridin-2-yl)4-(4-isopropyl-phenylamino)-quinazolin-2-yl]-methanol;

[7-(3-Chloro-pyridin-2-yl)4-(4-trifluoromethyl-phenylamino)-pyrido[3,2-d]pyrimidin-2-yl]-methanol;

[7-(3-Methyl-pyridin-2-yl)-2-(tetrahydro-furan-3-yl)-pyrido[2,3-d]pyrimidin-4-yl]-(4-trifluoromethyl-phenyl)-amine;

[7-(3-Methyl-pyridin-2-yl)-2-(tetrahydro-pyran4-yloxymethyl)-quinazolin-4-yl]-(4-trifluoromethyl-phenyl)-amine;

[7-(3-Methyl-pyridin-2-yl)-4-(4-trifluoromethyl-phenylamino)-pyrido[2,3-d]pyrimidin-2-yl]-methanol;

[7-(3-Trifluoromethyl-pyridin-2-yl)4-(6-trifluoromethyl-pyridin-3-ylamino)-quinazolin-2-yl]-methanol;

1-{4-[2-Isobutoxymethyl-7-(3-methyl-pyridin-2-yl)-pyrido[2,3-d]pyrimidin-4-ylamino]-phenyl}-cyclobutanecarbonitrile;

1-{4-[2-Methoxymethyl-7-(3-methyl-pyridin-2-yl)-pyrido[2,3-d]pyrimidin-4-ylamino]-phenyl }-cyclobutanecarbonitrile;

1-{4-[2-Methoxymethyl-7-(3-trifluoromethyl-pyridin-2-yl)-pyrido[3,2-d]pyrimidin-4-ylamino]-phenyl}-ethanone;

1-{4-[2-Methoxymethyl-7-(3-trifluoromethyl-pyridin-2-yl)-pyrido[3,2-d]pyrimidin-4-ylamino]-phenyl}-butan-1-one;

1-{4-[7-(3-Chloro-pyridin-2-yl)-2-ethoxymethyl-pyrido[2,3-d]pyrimidin-4-ylamino]-phenyl}-cyclobutanecarbonitrile;

1-Dimethylamino-3-[4-(4-trifluoromethyl-phenylamino)-7-(3-trifluoromethyl-pyridin-2-yl)-quinazolin-2-ylmethoxy]-propan-2-ol;

2-[4-(4-tert-Butyl-phenylamino)-7-(3-trifluoromethyl-pyridin-2-yl)-quinazolin-2-yl]-2-methyl-propan-1-ol;

2-{4-[2-Benzyloxymethyl-7-(3-trifluoromethyl-pyridin-2-yl)-quinazolin-4-ylamino]-phenyl}-2-methyl-propionitrile;

2-{4-[2-Ethoxymethyl-7-(3-trifluoromethyl-pyridin-2-y)-pyrido[2,3-d]pyrimidin-4-ylamino]-phenyl}-2-methyl-propionitrile;

2-{4-[2-Isobutoxymethyl-7-(3-methyl-pyridin-2-yl)-pyrido[2,3-d]pyrimidin-4-ylamino]-phenyl}-2-methyl-propionitrile;

2-{4-[2-Isobutoxymethyl-7-(3-trifluoromethyl-pyridin-2-yl)-pyrido[2,3-d]pyrimidin-4-ylamino]-phenyl}-2-methyl-propionitrile;

2-{4-[2-Methoxymethyl-7-(3-methyl-pyridin-2-yl)-pyrido[2,3-d]pyrimidin-4-ylamino]-phenyl}-2-methyl-propionitrile;

2-{4-[2-Methoxymethyl-7-(3-trifluoromethyl-pyridin-2-yl)-quinazolin-4-ylamino]-phenyl}-2-methyl-propionitrile;

2-{4-[7-(3-Chloro-pyridin-2-yl)-2-(2-methoxy-ethoxymethyl)-pyrido[2,3-d]pyrimidin-4-ylamino]-phenyl}-2-methyl-propionitrile;

2-{4-[7-(3-Chloro-pyridin-2-yl)-2-isobutoxymethyl-pyrido[2,3-d]pyrimidin-4-ylamino]-phenyl}-2-methyl-propionitrile;

2-{4-[7-(3-Chloro-pyridin-2-yl)-2-methoxymethyl-pyrido[2,3-d]pyrimidin-4-ylamino]-phenyl}-2-methyl-propionitrile;

2-Methyl-2-[4-(4-trifluoromethyl-phenylamino)-7-(3-trifluoromethyl-pyridin-2-yl)-quinazolin-2-yl]-propan-1-ol;

2-Methyl-2-{4-[7-(3-methyl-pyridin-2-yl)-2-(tetrahydro-furan-3-yl)-pyrido[2,3-d]pyrimidin-4-ylamino]-phenyl}-propionitrile;

3-[4-(6-tert-Butyl-pyridin-3-ylamino)-7-(3-trifluoromethyl-pyridin-2-yl)-quinazolin-2-yl]-propan-1-ol;

3-[7-(3-Trifluoromethyl-pyridin-2-yl)4-(6-trifluoromethyl-pyridin-3-ylamino)-quinazolin-2-yl]-propan-1-ol;

3-{4-[2-Isobutoxymethyl-7-(3-methyl-pyridin-2-yl)-pyrido[2,3-d]pyrimidin-4-ylamino]-phenyl}-3-methyl-butan-2-one;

3-{4-[2-Isobutoxymethyl-7-(3-trifluoromethyl-pyridin-2-yl)-pyrido[2,3-d]pyrimidin-4-ylamino]-phenyl}-3-methyl-butan-2-one;

3-{4-[2-Methoxymethyl-7-(3-methyl-pyridin-2-yl)-pyrido[2,3-d]pyrimidin-4-ylamino]-phenyl}-3-methyl-butan-2-one;

3-{4-[2-Methoxymethyl-7-(3-trifluoromethyl-pyridin-2-yl)-pyrido[2,3-d]pyrimidin-4-ylamino]-phenyl}-3-methyl-butan-2-one;

3-{4-[7-(3-Chloro-pyridin-2-yl)-2-isobutoxymethyl-pyrido[2,3-d]pyrimidin-4-ylamino]-phenyl}-3-methyl-butan-2-one;

3-{4-[7-(3-Chloro-pyridin-2-yl)-2-methoxymethyl-pyrido[2,3-d]pyrimidin-4-ylamino]-phenyl}-3-methyl-butan-2-one;

4-[2-Benzyloxymethyl-7-(3-trifluoromethyl-pyridin-2-yl)-quinazolin-4-ylamino]-N-tert-butyl-benzenesulfonamide;

4-[2-Methoxymethyl-7-(3-trifluoromethyl-pyridin-2-yl)-pyrido[3,2-d]pyrimidin-4-ylamino]-benzonitrile;

N,N-Diethyl-2-{4-[2-isobutoxymethyl-7-(3-methyl-pyridin-2-yl)-pyrido[2,3-d]pyrimidin-4-ylamino]-phenyl}-isobutyramide;

N,N-Diethyl-2-{4-[2-methoxymethyl-7-(3-trifluoromethyl-pyridin-2-yl)-pyrido[2,3-d]pyrimidin-4-ylamino]-phenyl}-isobutyramide;

N-tert-Butyl-4-[2-hydroxymethyl-7-(3-trifluoromethyl-pyridin-2-yl)-quinazolin-4-ylamino]-N-methyl-benzenesulfonamide;

N-tert-Butyl-4-[2-hydroxymethyl-7-(3-trifluoromethyl-pyridin-2-yl)-pyrido[3,2-d]pyrimidin-4-ylamino]-N-methyl-benzenesulfonamide; and

N-tert-Butyl4-[2-methoxymethyl-7-(3-trifluoromethyl-pyridin-2-yl)-quinazolin-4-ylamino]-benzene sulfonamide.

### Example 5

### VR1-Transfected Cells and Membrane Preparations

This Example illustrates the preparation of VR1-transfected cells and membrane preparations for use in binding assays (Example 6) and functional assays (Example 7).

A cDNA encoding full length human capsaicin receptor (SEQ ID NO: 1, 2 or 3 of U.S. Pat. No. 6,482,611) was subcloned in the plasmid pBK-CMV (Stratagene, La Jolla, Calif.) for recombinant expression in mammalian cells.

Human embryonic kidney (HEK293) cells were transfected with the pBK-CMV expression construct encoding the full length human capsaicin receptor using standard methods. The transfected cells were selected for two weeks in media containing G418 (400 μg/ml) to obtain a pool of stably transfected cells. Independent clones were isolated from this pool by limiting dilution to obtain clonal stable cell lines for use in subsequent experiments.

For radioligand binding experiments, cells were seeded in T175 cell culture flasks in media without antibiotics and grown to approximately 90% confluency. The flasks were then washed with PBS and harvested in PBS containing 5 mM EDTA. The cells were pelleted by gentle centrifugation and stored at −80° C. until assayed.

Previously frozen cells were disrupted with the aid of a tissue homogenizer in ice-cold HEPES homogenization buffer (5mM KCl 5, 5.8 mM NaCl, 0.75 mM CaCl₂, 2 mM MgCl₂, 320 mM sucrose, and 10 mM HEPES pH 7.4). Tissue homogenates were first centrifuged for 10 minutes at 1000×g (4° C.) to remove the nuclear fraction and debris, and then the supernatant from the first centrifugation is further centrifuged for 30 minutes at 35,000×g (4° C.) to obtain a partially purified membrane fraction. Membranes were resuspended in the HEPES homogenization buffer prior to the assay. An aliquot of this membrane homogenate is used to determine protein concentration via the Bradford method (BIO-RAD Protein Assay Kit, #500-0001, BIO-RAD, Hercules, Calif.).

### Example 6

### Capsaicin Receptor Binding Assay

This Example illustrates a representative assay of capsaicin receptor binding that may be used to determine the binding affinity of compounds for the capsaicin (VR1) receptor.

Binding studies with [³H] Resiniferatoxin (RTX) are carried out essentially as described by Szallasi and Blumberg (1992) J. Pharmacol. Exp. Ter. 262:883-888. In this protocol, non-specific RTX binding is reduced by adding bovine alpha₁ acid glycoprotein (100 μg per tube) after the binding reaction has been terminated.

[³H] RTX (37 Ci/mmol) is synthesized by and obtained from the Chemical Synthesis and Analysis Laboratory, National Cancer Institute-Frederick Cancer Research and Development Center, Frederick, Md.. [³H] RTX may also be obtained from commercial vendors (e.g., Amersham Pharmacia Biotech, Inc.; Piscataway, N.J.).

The membrane homogenate of Example 5 is centrifuged as before and resuspended to a protein concentration of 333 μg/ml in homogenization buffer. Binding assay mixtures are set up on ice and contain [³H]RTX (specific activity 2200 mCi/ml), 2 μl non-radioactive test compound, 0.25 mg/ml bovine serum albumin (Cohn fraction V), and 5×10⁴−1×10⁵ VR1-transfected cells. The final volume is adjusted to 500 μl (for competition binding assays) or 1,000 μl (for saturation binding assays) with the ice-cold HEPES homogenization buffer solution (pH 7.4) described above. Non-specific binding is defined as that occurring in the presence of 1 μM non-radioactive RTX (Alexis Corp.; San Diego, Calif.). For saturation binding, [³H]RTX is added in the concentration range of 7-1,000 pM, using 1 to 2 dilutions. Typically 11 concentration points are collected per saturation binding curve.

Competition binding assays are performed in the presence of 60 pM [³H]RTX and various concentrations of test compound. The binding reactions are initiated by transferring the assay mixtures into a 37° C. water bath and are terminated following a 60 minute incubation period by cooling the tubes on ice. Membrane-bound RTX is separated from free, as well as any alpha,-acid glycoprotein-bound RTX, by filtration onto WALLAC glass fiber filters (PERKIN-ELMER, Gaithersburg, Md.) which were pre-soaked with 1.0% PEI (polyethyleneimine) for 2 hours prior to use. Filters are allowed to dry overnight then counted in a WALLAC 1205 BETA PLATE counter after addition of WALLAC BETA SCINT scintillation fluid.

Equilibrium binding parameters are determined by fitting the allosteric Hill equation to the measured values with the aid of the computer program FIT P (Biosoft, Ferguson, Mo.) as described by Szallasi, et al. (1993) J Pharmacol. Exp. Ther. 266:678-683. Compounds provided herein generally exhibit Kᵢ values for capsaicin receptor of less than 1 μM, 100 nM, 50 nM, 25 nM, 10 nM, or nM in this assay.

### Example 7

### Calcium Mobilization Assay

This Example illustrates representative calcium mobilization assays for use in evaluating test compounds for agonist and antagonist activity.

Cells transfected with expression plasmids (as described in Example 4) and thereby expressing human capsaicin receptor are seeded and grown to 70-90% confluency in FALCON black-walled, clear-bottomed 96-well plates (#3904, BECTON-DICKINSON, Franklin Lakes, N.J.). The culture medium is emptied from the 96 well plates and FLUO-3 AM calcium sensitive dye (Molecular Probes, Eugene, Oreg.) is added to each well (dye solution: 1 mg FLUO-3 AM, 440 μL DMSO and 440 μl 20% pluronic acid in DMSO, diluted 1:250 in Krebs-Ringer HEPES (KRH) buffer (25 mM HEPES, 5 mM KCl, 0.96 mM NaH₂PO₄, 1 mM MgSO₄, 2 mM CaCl₂, 5 mM glucose, 1 mM probenecid, pH 7.4), 50 μl diluted solution per well). Plates are covered with aluminum foil and incubated at 37° C. for 1-2 hours in an environment containing 5% CO₂. After the incubation, the dye is emptied from the plates, and the cells are washed once with KRH buffer, and resuspended in KRH buffer.

### Determination Capsicin EC₅₀

To measure the ability of a test compound to agonize or antagonize a calcium mobilization response in cells expressing capsaicin receptors to capsaicin or other vanilloid agonist, the EC₅₀ of the agonist capsaicin is first determined. An additional 20 μl of KRH buffer and 1 μl DMSO is added to each well of cells, prepared as described above. 100 μl capsaicin in KRH buffer is automatically transferred by the FLIPR instrument to each well. Capsaicin-induced calcium mobilization is monitored using either FLUOROSKAN ASCENT (Labsystems; Franklin, Mass.) or FLIPR (fluorometric imaging plate reader system; Molecular Devices, Sunnyvale, Calif.) instruments. Data obtained between 30 and 60 seconds after agonist application are used to generate an 8-point concentration response curve, with final capsaicin concentrations of 1 nM to 3 μM. KALEIDAGRAPH software (Synergy Software, Reading, Pa.) is used to fit the data to the equation:

y=a*(1/(1+(b/x)^{c}))

to determine the 50% excitatory concentration (EC₅₀) for the response. In this equation, y is the maximum fluorescence signal, x is the concentration of the agonist or antagonist (in this case, capsaicin), a is the Eₘₐₓ, b corresponds to the EC₅₀ value and c is the Hill coefficient.

### Determination of Agonist Activity

Test compounds are dissolved in DMSO, diluted in KRH buffer, and immediately added to cells prepared as described above. 100 nM capsaicin (an approximate EC₉₀ concentration) is also added to cells in the same 96-well plate as a positive control. The final concentration of test compounds in the assay wells is between 0.1 nM and 5 μM.

The ability of a test compound to act as an agonist of the capsaicin receptor is determined by measuring the fluorescence response of cells expressing capsaicin receptors elicited by the compound as function of compound concentration. This data is fit as described above to obtain the EC₅₀, which is generally less than 1 micromolar, preferably less than 100 nM, and more preferably less than 10 nM. The extent of efficacy of each test compound is also determined by calculating the response elicited by a concentration of test compound (typically 1 μM) relative to the response elicited by 100 nM capsaicin. This value, called Percent of Signal (POS), is calculated by the following equation:

POS=100*test compound response/100 nM capsaicin response

This analysis provides quantitative assessment of both the potency and efficacy of test compounds as human capsaicin receptor agonists. Agonists of the human capsaicin receptor generally elicit detectable responses at concentrations less than 100 μM, or preferably at concentrations less than 1 μM, or most preferably at concentrations less than 10 nM. Extent of efficacy at human capsaicin receptor is preferably greater than 30 POS, more preferably greater than 80 POS at a concentration of 1 μM. Certain agonists are essentially free of antagonist activity as demonstrated by the absence of detectable antagonist activity in the assay described below at compound concentrations below 4 nM, more preferably at concentrations below 10 μM and most preferably at concentrations less than or equal to 100 μM.

### Determination of Antagonist Activity

Test compounds are dissolved in DMSO, diluted in 20 μl KRH buffer so that the final concentration of test compounds in the assay well is between 1 μM and 5 μM, and added to cells prepared as described above. The 96 well plates containing prepared cells and test compounds are incubated in the dark, at room temperature for 0.5 to 6 hours. It is important that the incubation not continue beyond 6 hours. Just prior to determining the fluorescence response, 100 μl capsaicin in KRH buffer at twice the EC₅₀ concentration determined as described above is automatically added by the FLIPR instrument to each well of the 96 well plate for a final sample volume of 200 μl and a final capsaicin concentration equal to the EC₅₀. The final concentration of test compounds in the assay wells is between 1 μM and 5 μM. Antagonists of the capsaicin receptor decrease this response by at least about 20%, preferably by at least about 50%, and most preferably by at least 80%, as compared to matched control (i.e., cells treated with capsaicin at twice the EC₅₀ concentration in the absence of test compound), at a concentration of 10 micromolar or less, preferably 1 micromolar or less. The concentration of antagonist required to provide a 50% decrease, relative to the response observed in the presence of capsaicin and without antagonist, is the IC₅₀ for the antagonist, and is preferably below 1 micromolar, 100 nanomolar, 10 nanomolar or 1 nanomolar.

Certain preferred VR1 antagonists are essentially free of agonist activity as demonstrated by the absence of detectable agonist activity in the assay described above at compound concentrations below 4 nM, more preferably at concentrations below 10 μM and most preferably at concentrations less than or equal to 100 μM.

### Example 8

### Microsomal In Vitro Half-Life

This Example illustrates the evaluation of compound half-life values (t_{1/2} values) using a representative liver microsomal half-life assay.

Pooled human liver microsomes are obtained from XenoTech LLC (Kansas City, Kans.). Such liver microsomes may also be obtained from In Vitro Technologies (Baltimore, Md.) or Tissue Transformation Technologies (Edison, N.J.). Six test reactions are prepared, each containing 25 μl microsomes, 5 μl of a 100 μM solution of test compound, and 399 μl 0.1 M phosphate buffer (19 mL 0.1 M NaH₂PO₄, 81 mL 0.1 M Na₂HPO₄, adjusted to pH 7.4 with H₃PO₄). A seventh reaction is prepared as a positive control containing 25 μl microsomes, 399 μl 0.1 M phosphate buffer, and 5 μl of a 100 μM solution of a compound with known metabolic properties (e.g., DIAZEPAM or CLOZAPINE). Reactions are preincubated at 39° C. for 10 minutes.

CoFactor Mixture is prepared by diluting 16.2 mg NADP and 45.4 mg Glucose-6-phosphate in 4 mL 100 mM MgCl₂. Glucose-6-phosphate dehydrogenase solution is prepared by diluting 214.3 μl glucose-6-phosphate dehydrogenase suspension (Roche Molecular Biochemicals; Indianapolis, Ind.) into 1285.7 μl distilled water. 71 μl Starting Reaction Mixture (3.mL CoFactor Mixture; 1.2 mL Glucose-6-phosphate dehydrogenase solution) is added to 5 of the 6 test reactions and to the positive control. 71 μl 100 mM MgCl₂ is added to the sixth test reaction, which is used as a negative control. At each time point (0, 1, 3, 5, and 10 minutes), 75 μl of each reaction mix is pipetted into a well of a 96-well deep-well plate containing 75 μl ice-cold acetonitrile. Samples are vortexed and centrifuged 10 minutes at 3500 rpm (Sorval T 6000D centrifuge, H1000B rotor). 75 μl of supernatant from each reaction is transferred to a well of a 96-well plate containing 150 μl of a 0.5 μM solution of a compound with a known LCMS profile (internal standard) per well. LCMS analysis of each sample is carried out and the amount of unmetabolized test compound is measured as AUC, compound concentration vs. time is plotted, and the t_{1/2} value of the test compound is extrapolated.

Preferred compounds provided herein exhibit in vitro t_{1/2} values of greater than 10 minutes and less than 4 hours, preferably between 30 minutes and 1 hour, in human liver microsomes.

### Example 9

### MDCK Toxicity Assay

This Example illustrates the evaluation of compound toxicity using a Madin Darby canine kidney (MDCK) cell cytotoxicity assay.

1 μL of test compound is added to each well of a clear bottom 96-well plate (PACKARD, Meriden, Conn.) to give final concentration of compound in the assay of 10 micromolar, 100 micromolar or 200 micromolar. Solvent without test compound is added to control wells.

MDCK cells, ATCC no. CCL-34 (American Type Culture Collection, Manassas, Va.), are maintained in sterile conditions following the instructions in the ATCC production information sheet. Confluent MDCK cells are trypsinized, harvested, and diluted to a concentration of 0.1×10⁶ cells/ml with warm (37° C.) medium (VITACELL Minimum Essential Medium Eagle, ATCC catalog # 30-2003). 100 μL of diluted cells is added to each well, except for five standard curve control wells that contain 100 μL of warm medium without cells. The plate is then incubated at 37° C. under 95% 02, 5% CO₂ for 2 hours with constant shaking. After incubation, 50 μL of mammalian cell lysis solution (from the PACKARD (Meriden, Conn.) ATP-LITE-M Luminescent ATP detection kit) is added per well, the wells are covered with PACKARD TOPSEAL stickers, and plates are shaken at approximately 700 rpm on a suitable shaker for 2 minutes.

Compounds causing toxicity will decrease ATP production, relative to untreated cells. The ATP-LITE-M Luminescent ATP detection kit is generally used according to the manufacturer's instructions to measure ATP production in treated and untreated MDCK cells. PACKARD ATP LITE-M reagents are allowed to equilibrate to room temperature. Once equilibrated, the lyophilized substrate solution is reconstituted in 5.5 mL of substrate buffer solution (from kit). Lyophilized ATP standard solution is reconstituted in deionized water to give a 10 mM stock. For the five control wells, 10 μL of serially diluted PACKARD standard is added to each of the standard curve control wells to yield a final concentration in each subsequent well of 200 nM, 100 nM, 50 nM, 25 nM and 12.5 nM. PACKARD substrate solution (50 μL) is added to all wells, which are then covered, and the plates are shaken at approximately 700 rpm on a suitable shaker for 2 minutes. A white PACKARD sticker is attached to the bottom of each plate and samples are dark adapted by wrapping plates in foil and placing in the dark for 10 minutes. Luminescence is then measured at 22° C. using a luminescence counter (e.g., PACKARD TOPCOUNT Microplate Scintillation and Luminescence Counter or TECAN SPECTRAFLUOR PLUS), and ATP levels calculated from the standard curve. ATP levels in cells treated with test compound(s) are compared to the levels determined for untreated cells. Cells treated with 10 μM of a preferred test compound exhibit ATP levels that are at least 80%, preferably at least 90%, of the untreated cells. When a 100 μM concentration of the test compound is used, cells treated with preferred test compounds exhibit ATP levels that are at least 50%, preferably at least 80%, of the ATP levels detected in untreated cells.

### EXAMPLE 10

### Dorsal Root Ganglion Cell Assay

This Example illustrates a representative dorsal root ganglian cell assay for evaluating VR1 antagonist or agonist activity of a compound.

DRG are dissected from neonatal rats, dissociated and cultured using standard methods (Aguayo and White (1992) Brain Research 570:61-67). After 48 hour incubation, cells are washed once and incubated for 30-60 minutes with the calcium sensitive dye Fluo 4 AM (2.5-10 ug/ml; TefLabs, Austin, Tex.). Cells are then washed once. Addition of capsaicin to the cells results in a VR1-dependent increase in intracellular calcium levels which is monitored by a change in Fluo-4 fluorescence with a fluorometer. Data are collected for 60-180 seconds to determine the maximum fluorescent signal.

For antagonist assays, various concentrations of compound are added to the cells. Fluorescent signal is then plotted as a function of compound concentration to identify the concentration required to achieve a 50% inhibition of the capsaicin-activated response, or IC₅₀. Antagonists of the capsaicin receptor preferably have an IC₅₀ below 1 micromolar, 100 nanomolar, 10 nanomolar or 1 nanomolar.

For agonist assays, various concentrations of compound are added to the cells without the addition of capsaicin. Compounds that are capsaicin receptor agonists result in a VR1-dependent increase in intracellular calcium levels which is monitored by a change in Fluo-4 fluorescence with a fluorometer. The EC₅₀, or concentration required to achieve 50% of the maximum signal for a capsaicin-activated response, is preferably below 1 micromolar, below 100 nanomolar or below 10 nanomolar.

### Example 11

### Animal Models for Determining Pain Relief

This Example illustrates representative methods for assessing the degree of pain relief provided by a compound.

### A. Pain Relief Testing

The following methods may be used to assess pain relief.

### Mechanical Allodynia

Mechanical allodynia (an abnormal response to an innocuous stimulus) is assessed essentially as described by Chaplan et al. (1994) J. Neurosci. Methods 53:55-63 and Tal and Eliav (1998) Pain 64(3):511-518. A series of von Frey filaments of varying rigidity (typically 8-14 filaments in a series) are applied to the plantar surface of the hind paw with just enough force to bend the filament. The filaments are held in this position for no more than three seconds or until a positive allodynic response is displayed by the rat. A positive allodynic response consists of lifting the affected paw followed immediately by licking or shaking of the paw. The order and frequency with which the individual filaments are applied are determined by using Dixon up-down method. Testing is initiated with the middle hair of the series with subsequent filaments being applied in consecutive fashion, ascending or descending, depending on whether a negative or positive response, respectively, is obtained with the initial filament.

Compounds are effective in reversing or preventing mechanical allodynia-like symptoms if rats treated with such compounds require stimulation with a Von Frey filament of higher rigidity strength to provoke a positive allodynic response as compared to control untreated or vehicle treated rats. Alternatively, or in addition, testing of an animal in chronic pain may be done before and after compound administration. In such an assay, an effective compound results in an increase in the rigidity of the filament needed to induce a response after treatment, as compared to the filament that induces a response before treatment or in an animal that is also in chronic pain but is left untreated or is treated with vehicle. Test compounds are administered before or after onset of pain. When a test compound is administered after pain onset, testing is performed 10 minutes to three hours after administration.

### Mechanical Hyperalgesia

Mechanical hyperalgesia (an exaggerated response to painful stimulus) is tested essentially as described by Koch et al. (1996) Analgesia 2(3):157-164. Rats are placed in individual compartments of a cage with a warmed, perforated metal floor. Hind paw withdrawal duration (i.e., the amount of time for which the animal holds its paw up before placing it back on the floor) is measured after a mild pinprick to the plantar surface of either hind paw.

Compounds produce a reduction in mechanical hyperalgesia if there is a statistically significant decrease in the duration of hindpaw withdrawal. Test compound may be administered before or after onset of pain. For compounds administered after pain onset, testing is performed 10 minutes to three hours after administration.

### Thermal Hyperalgesia

Thermal hyperalgesia (an exaggerated response to noxious thermal stimulus) is measured essentially as described by Hargreaves et al. (1988) Pain. 32(1):77-88. Briefly, a constant radiant heat source is applied the animals' plantar surface of either hind paw. The time to withdrawal (ie., the amount of time that heat is applied before the animal moves its paw), otherwise described as thermal threshold or latency, determines the animal's hind paw sensitivity to heat.

Compounds produce a reduction in thermal hyperalgesia if there is a statistically significant increase in the time to hindpaw withdrawal (i.e., the thermal threshold to response or latency is increased). Test compound may be administered before or after onset of pain. For compounds administered after pain onset, testing is performed 10 minutes to three hours after administration.

### B. Pain Models

Pain may be induced using any of the following methods, to allow testing of analgesic efficacy of a compound. In general, compounds provided herein result in a statistically significant reduction in pain as determined by at least one of the previously described testing methods, using male SD rats and at least one of the following models.

### Acute Inflammatory Pain Model

Acute inflammatory pain is induced using the carrageenan model essentially as described by Field et al. (1997) Br. J. Pharmacol. 121(8):1513-1522. 100-200 μl of 1-2% carrageenan solution is injected into the rats' hind paw. Three to four hours following injection, the animals' sensitivity to thermal and mechanical stimuli is tested using the methods described above. A test compound (0.01 to 50 mg/kg) is administered to the animal, prior to testing, or prior to injection of carrageenan. The compound can be administered orally or through any parenteral route, or topically on the paw. Compounds that relieve pain in this model result in a statistically significant reduction in mechanical allodynia and/or thermal hyperalgesia.

### Chronic Inflammatory Pain Model

Chronic inflammatory pain is induced using one of the following protocols:

1. Essentially as described by Bertorelli et al. (1999) Br. J. Pharmacol. 128(6):1252-1258, and Stein et al. (1998) Pharmacol. Biochem. Behav. 31(2):455-51, 200 μl Complete Freund's Adjuvant (0.1 mg heat killed and dried M. Tuberculosis) is injected to the rats' hind paw: 100 μl into the dorsal surface and 100 μl into the plantar surface.

2. Essentially as described by Abbadie et al. (1994) J. Neurosci. 14(10):5865-5871 rats are injected with 150 μl of CFA (1.5 mg) in the tibio-tarsal joint.

Prior to injection with CFA in either protocol, an individual baseline sensitivity to mechanical and thermal stimulation of the animals' hind paws is obtained for each experimental animal.

Following injection of CFA, rats are tested for thermal hyperalgesia, mechanical allodynia and mechanical hyperalgesia as described above. To verify the development of symptoms, rats are tested on days 5, 6, and 7 following CFA injection. On day 7, animals are treated with a test compound, morphine or vehicle. An oral dose of morphine of 1-5 mg/kg is suitable as positive control. Typically, a dose of 0.01-50 mg/kg of test compound is used. Compounds can be administered as a single bolus prior to testing or once or twice or three times daily, for several days prior to testing. Drugs are administered orally or through any parenteral route, or applied topically to the animal.

Results are expressed as Percent Maximum Potential Efficacy (MPE). 0% MPE is defined as analgesic effect of vehicle, 100% MPE is defined as an animal's return to pre-CFA baseline sensitivity. Compounds that relieve pain in this model result in a MPE of at least 30%.

### Chronic Neuropathic Pain Model

Chronic neuropathic pain is induced using the chronic constriction injury (CCI) to the rat's sciatic nerve essentially as described by Bennett and Xie (1988) Pain 33:87-107. Rats are anesthetized (e.g. with an intraperitoneal dose of 50-65 mg/kg pentobarbital with additional doses administered as needed). The lateral aspect of each hind limb is shaved and disinfected. Using aseptic technique, an incision is made on the lateral aspect of the hind limb at the mid thigh level. The biceps femoris is bluntly dissected and the sciatic nerve is exposed. On one hind limb of each animal, four loosely tied ligatures are made around the sciatic nerve approximately 1-2 mm apart. On the other side the sciatic nerve is not ligated and is not manipulated. The muscle is closed with continuous pattern and the skin is closed with wound clips or sutures. Rats are assessed for mechanical allodynia, mechanical hyperalgesia and thermal hyperalgesia as described above.

Compounds that relieve pain in this model result in a statistically significant reduction in mechanical allodynia, mechanical hyperalgesia and/or thermal hyperalgesia when administered (0.01-50 mg/kg, orally, parenterally or topically) immediately prior to testing as a single bolus, or for several days: once or twice or three times daily prior to testing.

### Example 12

### Inhibition of Tolerance to Morphine

This Example illustrates the use of representative VR1 antagonists to inhibit the development of tolerance to morphine analgesia in rats.

CFA-induced chronic inflammatory pain was induced in rats by injection with 150 μl of 10 mg/mL CFA in the left ankle. One week after CFA injection animals were tested for mechanical allodynia. Animals were then treated with one of the following:

Vehicle: subcutaneous saline and oral 0.5% methylcellulose/0.1% triacetin (MCTA)

VR1 antagonist ((6-trifluoromethyl-pyridin-3-yl)-[7-(3-trifluoromethyl-pyridin-2-yl)-quinazolin-4-yl]-amine; 20 mg/kg): twice a day, orally in MCTA

Morphine: 3 mg/kg subcutaneous (s.c.) once daily

Morphine: 3 mg/kg s.c. (once daily) with VR1 antagonist (10 mg/kg: twice daily, orally in MCTA).

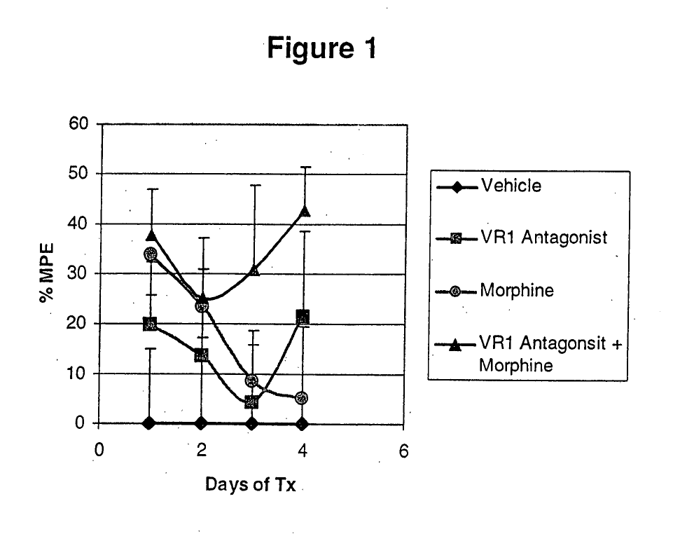

Animals were again tested for mechanical allodynia. The results are shown in FIG. 1, expressed as % MPE (% of maximum potential efficacy), where 100% is full analgesia and 0% indicates no detectable difference from vehicle alone.

In a similar experiment, CFA-induced chronic inflammatory pain was induced in rats as described above, and the animals were tested for mechanical allodynia 11 days after CFA injection. Animals were then treated with one of the following:

Vehicle: subcutaneous saline and oral MCTA

VR1 antagonist ([2-methyl-7-(3-trifluoromethyl-pyridin-2-yl)-quinazolin-4-yl]-(5-trifluoromethyl-pyridin-2-yl)-amine; 0.5 mg/kg): once a day, orally in MCTA, and saline injection s.c.

Morphine: 3 mg/kg s.c. once daily, and oral MCTA

Morphine (3 mg/kg s.c.; once daily) and VR1 antagonist (0.5 mg/kg: twice daily, orally).

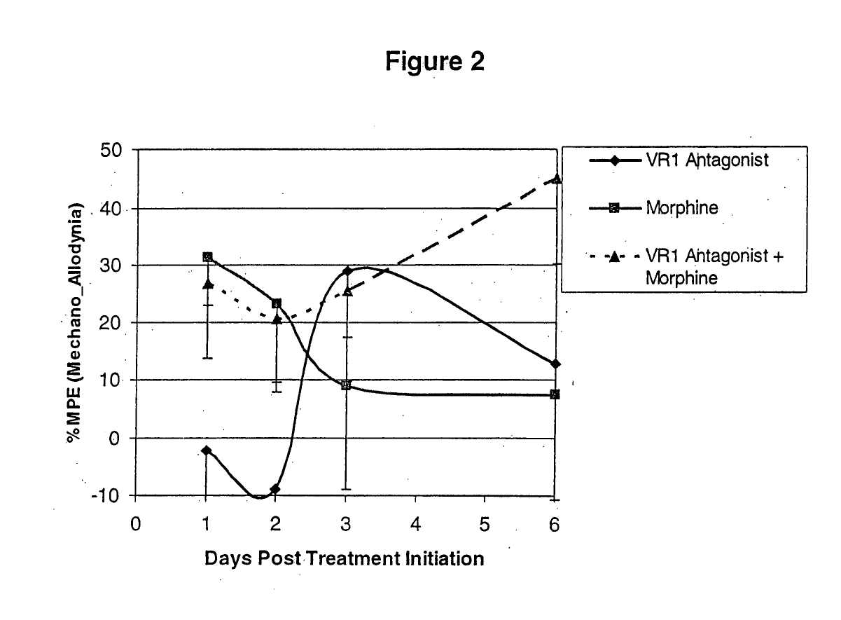

Animals were again tested for mechanical allodynia. The results are shown in FIG. 2, expressed as % MPE (maximum potential efficacy), where 100% is full analgesia and 0% indicates no detectable difference from vehicle alone. These data indicate that a VR1 antagonist can be used to inhibit the development of tolerance to morphine.

In a further experiment, chronic inflammatory pain was induced in rats by CFA injection in the left ankle as described above, and the animals were tested for mechanical allodynia 7 days later. The animals were then treated with one of the following, once per day for four days:

oral methylcellulose/triacetin vehicle (MC) and subcutaneous saline vehicle (saline);

oral MC and subcutaneous morphine sulfate (3 mg/kg);

oral VR1 antagonist (0.3 mg/kg) and subcutaneous saline; or

oral VR1 antagonist (0.3 mg/kg) and subcutaneous morphine sulfate (3 mg/kg).

The VR1 antagonist used in this experiment was [2-(2,6-dimethyl-morpholin-4-ylmethyl)-7-(3-trifluoromethyl-pyridin-2-yl)-pyrido[3,2-d]pyrimidin-4-yl]-(4-trifluoromethyl-phenyl)-amine (cis), which has the structure:

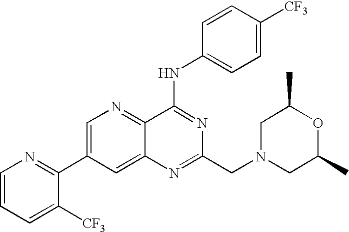

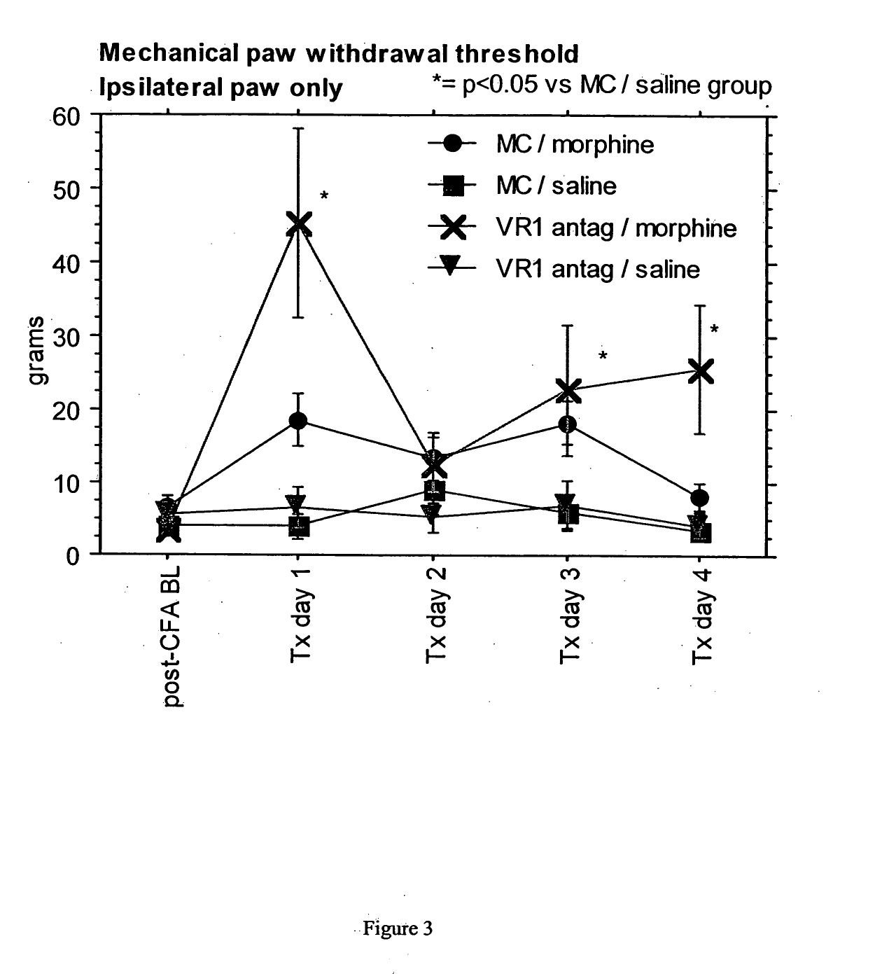

The withdrawal threshold was determined each day 1 hour after treatment, and the results are presented in FIG. 3 (which is a plot of withdrawal threshold from a von Frey filament (in gram force) as a function of treatment over the 5 day period). In FIG. 3, “Post CFA BL” is the von Frey filament threshold 7 days after injection of CFA in the left ankle. The data collected on Treatment Day 4 was analyzed by ANOVA and Fisher's PLSD post-hoc testing. This analysis shows that animals receiving oral vehicle and subcutaneous morphine are not significantly different from oral vehicle and subcutaneous vehicle controls. However, rats receiving morphine plus VR1 antagonist exhibit statistically significantly higher withdrawal thresholds than any other treatment group, indicating that VR1 antagonist prevents tolerance to repeated morphine dosing.

### Example 13

### Pain Relief Upon Administration of VR1 Antagonist and Morphine

This experiment illustrates the enhanced pain relief that is achieved upon administration of VR1 antagonist and morphine, in combination.

Rats received oral administration of 0.1 mg/kg VR1 antagonist ([2-(2,6-dimethyl-morpholin-4-ylmethyl)-7-(3-trifluoromethyl-pyridin-2-yl)-pyrido[3,2-d]pyrimidin-4-yl]-(4-trifluoromethyl-phenyl)-amine (cis)) or vehicle alone in a solution of 2% vitamin E (d-alpha tocopheryl polyethylene glycol 1000 succinate) in distilled water. One hour later, 100 μL of 1% carrageenan was injected in the intraplantar aspect of the left paw. 2 hours after carrageenan injection, the rats were treated subcutaneously with saline or 1 mg/kg of morphine sulfate in saline. One hour after subcutaneous treatment, each rat's thermal withdrawal latency (i.e., the amount of time that heat is applied before the animal moves its paw) was determined. This latency was compared with the latency determined for each rat on the day prior to the experiment (baseline day).

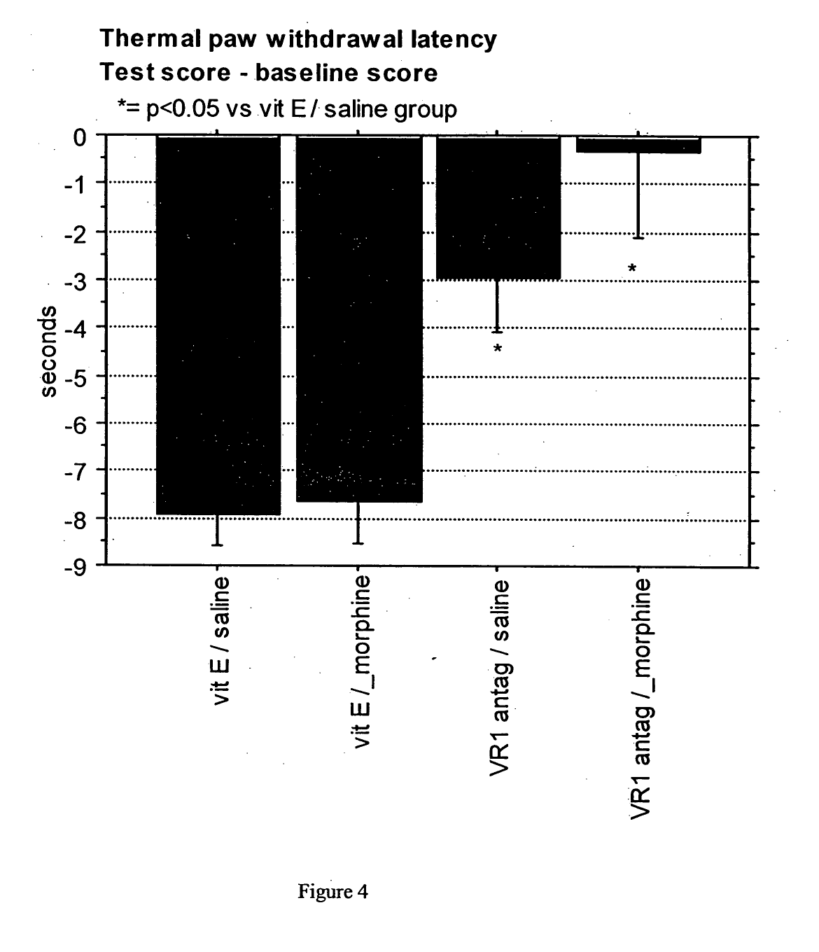

The data are presented in FIG. 4, expressed as a decrease in paw withdrawal latency (latency on test day—latency on baseline day). Statistical analysis by ANOVA followed by Fisher's post-hoc PLSD test indicates that 1 mg/kg morphine with oral vehicle was ineffective in reversing carrageenan-induced thermal hyperalgesia. VR1 antagonist alone had a statistically significant effect. However, coadministration of 1 mg/kg morphine with VR1 antagonist resulted in the greatest effect. These data indicate that VR1 antagonist coadministration results in efficacy of morphine doses that are ineffective without VR1 antagonist, and VR1 antagonists may be useful in reducing the side effects observed in patients treated with opioids by decreasing the dose needed to achieve effective analgesia.

From the foregoing it will be appreciated that, although specific embodiments of the invention have been described herein for purposes of illustration, various modifications may be made without deviating from the spirit and scope of the invention.

What is claimed is:

1. A composition comprising a narcotic analgesic and a nontoxic VR1 antagonist.

2. The composition of claim 1 wherein the narcotic analgesic is selected from alfentanyl, alphaprodine, anileridine, bezitramide, codeine, dihydrocodeine, diphenoxylate, ethylmorphine, fentanyl, heroin, hydrocodone, hydromorphone, isomethadone, levomethorphan, levorphanol, meperidine, metazocine, methadone, metopon, morphine, opium, oxycodone, oxymorphone, pethidine, phenazocine, piminodine, racemethorphan, racemorphan, thebaine, their mixtures and their pharmaceutically acceptable salts and hydrates.

3. The composition of claim 1, wherein the narcotic analgesic is selected from codeine, fentanyl, hydrocodone, meperidine, morphine, oxycodone, their mixtures and their pharmaceutically acceptable salts and hydrates.

4. The composition of claim 1, wherein the VR1 antagonist is not a vanilloid compound.

5. The composition of claim 1, wherein the VR1 antagonist exhibits a Kᵢ value of 1 micromolar or less in a capsaicin receptor binding assay.

6. The composition of claim 1, wherein the VR1 antagonist exhibits a Kᵢ value of 100 nanomolar or less in a capsaicin receptor binding assay.

7. The composition of claim 1 in sustained release dosage form.

8. The composition of claim 1 formulated for oral administration.

9. The composition of claim 1, wherein the VR1 antagonist is present in a tolerance-reducing amount.

10. The composition of claim 1, wherein the VR1 antagonist is present in a dependence-reducing amount.

11. The composition of claim 1, wherein the VR1 antagonist is present in a pain relief-enhancing amount.

12. A packaged pharmaceutical composition, comprising:
(i) a container holding a composition comprising a nontoxic VR1 antagonist; and
(ii) instructions indicating that the VR1 antagonist is to be administered to a patient contemporaneously with administration of an addictive substance.

13. The packaged pharmaceutical composition of claim 12, wherein the addictive substance is a narcotic analgesic.

14. The packaged pharmaceutical composition of claim 12, further comprising a narcotic analgesic.

15. The packaged pharmaceutical composition of claim 12, wherein the composition is formulated for oral administration.

16. The packaged pharmaceutical composition of claim 12, wherein the VR1 antagonist is not a vanilloid compound.

17. The packaged pharmaceutical composition of claim 12, wherein the VR1 antagonist exhibits a Kᵢ of 1 micromolar or less in a capsaicin receptor binding assay.

18. The packaged pharmaceutical composition of claim 12, wherein the VR1 antagonist exhibits a Kᵢ of 100 nanomolar or less in a capsaicin receptor binding assay.

19. The packaged pharmaceutical composition of claim 12, wherein the instructions indicate that the VR1 antagonist is to be used for inhibiting the development of tolerance to an addictive substance in a patient.

20. The packaged pharmaceutical composition of claim 12, wherein the instructions indicate that the VR1 antagonist is to be used for inhibiting the development of dependence on an addictive substance in a patient.

21. The packaged pharmaceutical composition of claim 12, wherein the instructions indicate that the VR1 antagonist is to be used for enhancing pain relief upon administration of addictive substance to a patient.

22. The packaged pharmaceutical composition of claim 12, wherein the VR1 antagonist is present in a tolerance-reducing amount.

23. The packaged pharmaceutical composition of claim 12, wherein the VR1 antagonist is present in a dependence-reducing amount.

24. The packaged pharmaceutical composition of claim 12, wherein the VR1 antagonist is present in a pain relief-enhancing amount.

25. A packaged pharmaceutical composition, comprising:
(i) a nontoxic VR1 antagonist;
(ii) a narcotic analgesic; and
(iii) instructions indicating that the VR1 antagonist and narcotic analgesic are to be administered to a patient for the treatment of pain.

26. The packaged pharmaceutical composition of claim 25, wherein the VR1 antagonist and narcotic analgesic are present in the same composition.

27. The packaged pharmaceutical composition of claim 25, wherein the VR1 antagonist and narcotic analgesic are present in different containers.

28. The packaged pharmaceutical composition of claim 25, wherein the VR1 antagonist and narcotic analgesic are formulated for oral administration.

29. The packaged pharmaceutical composition of claim 25, wherein the VR1 antagonist is not a vanilloid compound.

30. The packaged pharmaceutical composition of claim 25, wherein the VR1 antagonist exhibits a Kᵢ of 1 micromolar or less in a capsaicin receptor binding assay.

31. The packaged pharmaceutical composition of claim 25, wherein the VR1 antagonist exhibits a Kᵢ of 100 nanomolar or less in a capsaicin receptor binding assay.

32. The packaged pharmaceutical composition of claim 25, wherein the VR1 antagonist is present in a tolerance-reducing amount.

33. The packaged pharmaceutical composition of claim 25, wherein the VR1 antagonist is present in a dependence-reducing amount.

34. The packaged pharmaceutical composition of claim 25, wherein the VR1 antagonist is present in a pain relief-enhancing amount.

35. The composition of claim 26 wherein the narcotic analgesic is selected from alfentanyl, alphaprodine, anileridine, bezitramide, codeine, dihydrocodeine, diphenoxylate, ethylmor6hine, fentanyl, heroin, hydrocodone, hydromorphone, isomethadone, levomethorphan, levorphanol, meperidine, metazocine, methadone, metopon, morphine, opium, oxycodone, oxymorphone, pethidine, phenazocine, piminodine, racemethorphan, racemorphan, thebaine, their mixtures and their pharmaceutically acceptable salts and hydrates.

36. The packaged pharmaceutical composition of claim 35, wherein the narcotic analgesic is selected from codeine, fentanyl, hydrocodone, meperidine, morphine, oxycodone, their mixtures and their pharmaceutically acceptable salts and hydrates.

37. The packaged pharmaceutical composition of claim 25 in sustained release dosage form.

38. A method of treating pain in a patient, comprising administering to a patient, simultaneously or sequentially in either order;
(i) a narcotic analgesic; and
(ii) a nontoxic VR1 antagonist; and thereby providing pain relief to the patient.

39. The method of claim 38, wherein the narcotic analgesic is selected from alfentanyl, alphaprodine, anileridine, bezitramide, codeine, dihydrocodeine, diphenoxylate, ethylmorphine, fentanyl, heroin, hydrocodone, hydromorphone, isomethadone, levomethorphan, levorphanol, metazocine, methadone, metopon, meperidine, morphine, opium, oxycodone, oxymorphone, pethidine, phenazocine, piminodine, racemethorphan, racemorphan, thebaine, their mixtures and their pharmaceutically acceptable salts and hydrates.

40. The method of claim 38, wherein the VR1 antagonist is not a vanilloid compound.

41. The method of claim 38, wherein the VR1 antagonist exhibits a Kᵢ value of 1 micromolar or less in a capsaicin receptor binding assay

42. The method of claim 38, wherein the VR1 antagonist exhibits a Kᵢ value of 100 nanomolar or less in a capsaicin receptor binding assay.

43. A method for inhibiting the development of tolerance to a narcotic analgesic in a patient, comprising administering to a patient, simultaneously or sequentially in either order;
(i) a narcotic analgesic; and
(ii) a tolerance-reducing amount of a nontoxic VR1 antagonist; and thereby inhibiting the development of tolerance to the narcotic analgesic.

44. The method of claim 43, wherein the narcotic analgesic is selected from alfentanyl, alphaprodine, anileridine, bezitramide, codeine, dihydrocodeine, diphenoxylate, ethylmorphine, fentanyl, heroin, hydrocodone, hydromorphone, isomethadone, levomethorphan, levorphanol, meperidine, metazocine, methadone, metopon, morphine, opium, oxycodone, oxymorphone, pethidine, phenazocine, piminodine, racemethorphan, racemorphan, thebaine, their mixtures and their pharmaceutically acceptable salts and hydrates.

45. The method of claim 43, wherein the VR1 antagonist is not a vanilloid compound.

46. The method of claim 43, wherein the VR1 antagonist exhibits a Kᵢ value of 1 micromolar or less in a capsaicin receptor binding assay

47. The method of claim 43, wherein the VR1 antagonist exhibits a Kᵢ value of 100 nanomolar or less in a capsaicin receptor binding assay.

48. A method for inhibiting the development of dependence on a narcotic analgesic in a patient, comprising administering to a patient, simultaneously or sequentially in either order;
(i) a narcotic analgesic; and
(ii) a dependence-reducing amount of a nontoxic VR1 antagonist; and thereby inhibiting the development of dependence on the narcotic analgesic.

49. The method of claim 48, wherein the narcotic analgesic is selected from alfentanyl, alphaprodine, anileridine, bezitramide, codeine, dihydrocodeine, diphenoxylate, ethylmorphine, fentanyl, heroin, hydrocodone, hydromorphone, isomethadone, levomethorphan, levorphanol, meperidine, metazocine, methadone, metopon, morphine, opium, oxycodone, oxymorphone, pethidine, phenazocine, piminodine, racemethorphan, racemorphan, thebaine, their mixtures and their pharmaceutically acceptable salts and hydrates.

50. The method of claim 48, wherein the VR1 antagonist is not a vanilloid compound.

51. The method of claim 48, wherein the VR1 antagonist exhibits a Kᵢ value of 1 micromolar or less in a capsaicin receptor binding assay.

52. The method of claim 48, wherein the VR1 antagonist exhibits a Kᵢ value of 100 nanomolar or less in a capsaicin receptor binding assay.

53. A method for enhancing narcotic analgesic-induced pain relief in a patient, comprising administering to a patient, simultaneously or sequentially in either order;
(i) a narcotic analgesic; and
(ii) a pain-relief enhancing amount of a nontoxic VR1 antagonist; and thereby enhancing narcotic analgesic-induced pain relief in the patient.

54. The method of claim 53, wherein the narcotic analgesic is selected from alfentanyl, alphaprodine, anileridine, bezitramide, codeine, dihydrocodeine, diphenoxylate, ethylmorphine, fentanyl, heroin, hydrocodone, hydromorphone, isomethadone, levomethorphan, levorphanol, meperidine, metazocine, methadone, metopon, morphine, opium, oxycodone, oxymorphone, pethidine, phenazocine, piminodine, racemethorphan, racemorphan, thebaine, their mixtures and their pharmaceutically acceptable salts and hydrates.

55. The method of claim 53, wherein the VR1 antagonist is not a vanilloid compound.

56. The method of claim 53, wherein the VR1 antagonist exhibits a Kᵢ value of 1 micromolar or less in a capsaicin receptor binding assay.

57. The method of claim 53, wherein the VR1 antagonist exhibits a Kᵢ value of 100 nanomolar or less in a capsaicin receptor binding assay.

58. A method for treating withdrawal symptoms resulting from prior administration of an addictive substance in a patient, comprising administering a nontoxic VR1 antagonist to a patient experiencing or susceptible to withdrawal symptoms, and thereby decreasing severity of the withdrawal symptoms in the patient.

59. A single dose pharmaceutical composition for the treatment of a patient experiencing pain comprising a combination of a VR1 antagonist and at least one analgesic selected from the group consisting of less than about 25 mg of anileridine, less than about 25 mg of codeine, less than about 40 mg of dextroproposyphene, less than about 25 mg of dihydrocodeine, less than about 4 mg of diphenoxylate, less than about 20 μg of fenantyl, less than about 2 mg of hydrocodone, less than about 1.5 mg of hydromorphone, less than about 0.8 mg of levorphanol, less than about 20 mg of meperidine, less than abut 4 mg of methadone, less than about 7.5 mg of morphine, less than about 2 mg of oxycodon, less than about 0.8 mg of oxymorphone, less than about 0.8 mg of oxymorphone, less than about 40 mg of pethidine.
